SEQUENCE LISTING (49 sequences)

SEQ ID NO: 1 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 2 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 3 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 4 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFKGEQMFDVKEAIDTKNHQLDSKLFEYFKEKAFPYLSTKHLGVFPDHVIDMFINGYRLSLTNHGPTPVKRGSKDPRGGIFDAVFTRGNQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 5 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFINGYRLSLTNHGPTPVKKGSKDPRGGIFDAVFTRGNQSKLLTSRHDFKEKNLKEISDLIKKELTEGKALGLSHTYANVRINHVINLWGADFDSNGNLKAIYVTDSDSNASIGMKKYFVGVNKAGKVAISAKEIKEDNIGAQVLGLFTLSTGQDSWNQTN

SEQ ID NO: 6 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLREHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKKELDEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 7 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLKEHPEKQKINFNGEQMFDVKEAIRTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKKELEEGKALGLSHTYANVRINHVINLWGADFDSNGNLKAIYVTDSDSNASIGMKKYFVGVNKAGKVAISAKEIKEDNIGAQVLGLFTLSTGQDSWNQTN

SEQ ID NO: 8 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIERYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKEELTKGKALGLSHTYANVRINHVINLWGADFDSNGNLKAIYVTDSDSNASIGMKKYFVGVNSAGKVAISAKEIKEKNIGAQVLGLFTLSTGQKSWNQTN

SEQ ID NO: 9 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLKEHPEKQKINFRGEQMFDVKEAIRTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKSELENGKALGLSHTYANVRINHVINLWGADFDSNGNLKAIYVTDSDSNASIGMKKYFVGVNKAGKVAISAKEIKEDNIGAQVLGLFTLSTGQDSWNQTN

SEQ ID NO: 10 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLKEHPEKQKINFRGEQMFDVKEAIRTKNHQLDSKLFEYFKEKAFPYLSTKHLGVFPDHVIDMFINGYRLSLTNHGPTPVKKGSKDPRGGIFDAVFTRGNQSKLLTSRHDFKEKNLKEISDLIKKELEEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 11 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIERYLEEHPEKQKINFRGEQMFDVKEAIDTKNHQLDSKLFEYFKEKAFPYLSTKHLGVFPDHVIDMFINGYRLSLTNHGPTPVKKGSKDPRGGIFDAVFTRGNQSKLLTSRHDFKEKNLKEISDLIKEELTKGKALGLSHTYANVRINHVINLWGADFDSNGNLKAIYVTDSDSNASIGMKKYFVGVNSAGKVAISAKEIKEDNIGAQVLGLFTLSTGQKSWNQTN

SEQ ID NO: 12 - protein, 304 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQD

SEQ ID NO: 13 - protein, 290 aa, Streptococcus pyogenes
SVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 14 - protein, 290 aa, Streptococcus pyogenes
SVWTKGVTPPANFTQGEDVFHAPYVANQGWYDITKTFNGKDDLLCGAATAGNMLHWWFDQNKDQIKRYLEEHPEKQKINFKGEQMFDVKEAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 15 - protein, 290 aa, Streptococcus pyogenes
SVWTKGVTPPANFTQGEDVFHAPYVANQGWYDITKTFNGKDDLLCGAATAGNMLHWWFDQNKDQIERYLEEHPEKQKINFKGEQMFDVKKAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKEELTKGKALGLSHTYANVRINHVINLWGADFDSNGNLKAIYVTDSDSNASIGMKKYFVGVNSAGKVAISAKEIKEDNIGAQVLGLFTLSTGQKSWNQTN

SEQ ID NO: 16 - protein, 319 aa, Streptococcus pyogenes
DDYQRNATEAYAKEVPHQITSVWTKGVTPPANFTQGEDVFHAPYVANQGWYDITKTFNGKDDLLCGAATAGNMLHWWFDQNKDQIERYLEEHPEKQKINFKGEQMFDVKKAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKEELTKGKALGLSHTYANVRINHVINLWGADFDSNGNLKAIYVTDSDSNASIGMKKYFVGVNSAGKVAISAKEIKEDNIGAQVLGLFTLSTGQKSWNQTNGGGHHHHHH

SEQ ID NO: 17 - protein, 313 aa, Streptococcus pyogenes
DDYQRNATEAYAKEVPHQITSVWTKGVTPLTPEQFRYNNEDVFHAPYVANQGWYDITKAFDGKDNLLCGAATAGNMLHWWFDQNKDQIKRYLEEHPEKQKINFNGDNMFDVKKAIDTKNHQLDSKLFNYFKEKAFPGLSARRIGVFPDHVIDMFINGYRLSLTNHGPTPVKEGSKDPRGGIFDAVFTRGNQSKLLTSRHDFKNKNLNDISTIIKQELTKGKALGLSHTYANVSINHVINLWGADFNAEGNLEAIYVTDSDSNASIGMKKYFVGVNAHGHVAISAKKIEGENIGAQVLGLFTLSTGQDSWQKLS

SEQ ID NO: 18 - protein, 311 aa, Streptococcus pyogenes
MDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 19 - DNA, 3579 bp, unidentified
  source 1..3579: mol_type = other DNA
atgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtga

SEQ ID NO: 20 - DNA, 3480 bp, unidentified
  source 1..3480: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaa

SEQ ID NO: 21 - DNA, 957 bp, unidentified
  source 1..957: mol_type = other DNA
atggcagcagcagccgccgcagccgccgagcagcagtcaagcaatggaccagtgaaaaaatcaatgagagaaaaagccgtcgagaggagatcagtgaataaggagcacaacagcaatttcaaagccggctacatccctattgacgaagatcgcctgcataagacaggcctgagggggcgcaaaggaaacctggcaatctgcgtcatcattctgctgtttatcctggccgtgattaatctgatcattactctggtgatttgggctgtcatccgcattggcccaaacgggtgtgactctatggagttccacgaaagtggcctgctgcgatttaagcaggtgtccgatatgggggtcatccatccactgtacaaatctactgtcggcgggcggagaaacgagaatctggtgatcaccgggaacaatcagcccattgtgttccagcagggaaccacaaagctgtctgtggaaaacaataaaacatcaatcactagcgacattggcatgcagttctttgatccccggacccagaatatcctgttcagtaccgactatgagacacacgaatttcatctgccttccggggtgaagtctctgaacgtccagaaagccagcactgagagaatcaccagtaacgctacatcagacctgaatatcaaggtggatggacgagctattgtccggggaaatgagggcgtgttcatcatgggcaagacaattgaatttcacatgggaggcaacatggagctgaaagcagaaaacagcatcattctgaatgggagcgtgatggtctccactaccagactgcccagctcctctagtggagaccagctggggtccggagattgggtcaggtataagctgtgcatgtgtgccgatggcaccctgtttaaagtgcaggtcaccagccagaatatgggatgtcagattagcgataacccttgtgggaatactcattaa

SEQ ID NO: 22 - DNA, 2303 bp, unidentified
  source 1..2303: mol_type = other DNA
ctgcgcgctcgctcgctcactgaggccgcccgggcaaagcccgggcgtcgggcgacctttggtcgcccggcctcagtgagcgagcgagcgcgcagagagggagtggggttaaccaattggcggccgcaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccggtaccaccatggcagcagcagccgccgcagccgccgagcagcagtcaagcaatggaccagtgaaaaaatcaatgagagaaaaagccgtcgagaggagatcagtgaataaggagcacaacagcaatttcaaagccggctacatccctattgacgaagatcgcctgcataagacaggcctgagggggcgcaaaggaaacctggcaatctgcgtcatcattctgctgtttatcctggccgtgattaatctgatcattactctggtgatttgggctgtcatccgcattggcccaaacgggtgtgactctatggagttccacgaaagtggcctgctgcgatttaagcaggtgtccgatatgggggtcatccatccactgtacaaatctactgtcggcgggcggagaaacgagaatctggtgatcaccgggaacaatcagcccattgtgttccagcagggaaccacaaagctgtctgtggaaaacaataaaacatcaatcactagcgacattggcatgcagttctttgatccccggacccagaatatcctgttcagtaccgactatgagacacacgaatttcatctgccttccggggtgaagtctctgaacgtccagaaagccagcactgagagaatcaccagtaacgctacatcagacctgaatatcaaggtggatggacgagctattgtccggggaaatgagggcgtgttcatcatgggcaagacaattgaatttcacatgggaggcaacatggagctgaaagcagaaaacagcatcattctgaatgggagcgtgatggtctccactaccagactgcccagctcctctagtggagaccagctggggtccggagattgggtcaggtataagctgtgcatgtgtgccgatggcaccctgtttaaagtgcaggtcaccagccagaatatgggatgtcagattagcgataacccttgtgggaatactcattaaaagcttggccgcaataaaagatctttattttcattagatctgtgtgttggttttttgtgtgtcctgcaggggcgcgcctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 23 - DNA, 1164 bp, unidentified
  source 1..1164: mol_type = other DNA
atggccgagacactgttctggactcctctgctggtggtgctgctggctggactgggagataccgaggctcagcagaccacactgcacccactggtgggccgggtgttcgtgcacaccctggaccatgagacatttctgagtctgccagaacacgtggctgtgccacctgctgtgcatatcacttaccacgcccatctgcagggccatcctgatctgccacggtggctgagatacacccagagatcaccccaccatcctggattcctgtatggaagcgctaccccagaggacaggggactgcaggtgatcgaagtgacagcttacaaccgcgacagttttgatactaccaggcagcgcctggtgctggagattggggatccagaaggacccctgctgccttatcaggccgagttcctggtgcggtcacacgacgctgaggaagtgctgccatcaacacccgccagcagatttctgtccgctctgggaggactgtgggagccaggagaactgcagctgctgaatgtgactagcgctctggataggggaggaagggtgccactgccaatcgagggaaggaaggaaggggtgtacattaaagtgggaagcgcttccccattctccacctgcctgaagatggtggcttctcctgatagtcacgctaggtgcgctcagggacagccaccactgctgtcctgttatgacacactggccccccattttcgcgtggactggtgcaacgtgactctggtggataaatctgtgcctgagccagctgacgaagtgccaacccctggagacggaatcctggagcacgatcctttcttttgtcctccaacagaagccccagacagggatttcctggtggacgctctggtgactctgctggtgcctctgctggtggctctgctgctgaccctgctgctggcttatgtgatgtgctgtcggagagagggacggctgaagagagacctggccacatctgatatccagatggtgcaccattgtactattcacggcaacaccgaggaactgcgccagatggctgcttctagggaggtgccaaggccactgagtacactgcctatgtttaatgtgcacactggcgaacggctgccccctagagtggatagcgcccaggtgccactgattctggaccagcattga

SEQ ID NO: 24 - DNA, 2476 bp, unidentified
  source 1..2476: mol_type = other DNA
ctgcgcgctcgctcgctcactgaggccgcccgggcaaagcccgggcgtcgggcgacctttggtcgcccggcctcagtgagcgagcgagcgcgcagagagggagtggggttaaccaattggcggccgcaaacttgcatgccccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctatgctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggaccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtcctccctggggacagcccctcctggctagtcacaccctgtaggctcctctatataacccaggggcacaggggctgcccccgggtcacctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagcgcgtggtaccaccatggccgagacactgttctggactcctctgctggtggtgctgctggctggactgggagataccgaggctcagcagaccacactgcacccactggtgggccgggtgttcgtgcacaccctggaccatgagacatttctgagtctgccagaacacgtggctgtgccacctgctgtgcatatcacttaccacgcccatctgcagggccatcctgatctgccacggtggctgagatacacccagagatcaccccaccatcctggattcctgtatggaagcgctaccccagaggacaggggactgcaggtgatcgaagtgacagcttacaaccgcgacagttttgatactaccaggcagcgcctggtgctggagattggggatccagaaggacccctgctgccttatcaggccgagttcctggtgcggtcacacgacgctgaggaagtgctgccatcaacacccgccagcagatttctgtccgctctgggaggactgtgggagccaggagaactgcagctgctgaatgtgactagcgctctggataggggaggaagggtgccactgccaatcgagggaaggaaggaaggggtgtacattaaagtgggaagcgcttccccattctccacctgcctgaagatggtggcttctcctgatagtcacgctaggtgcgctcagggacagccaccactgctgtcctgttatgacacactggccccccattttcgcgtggactggtgcaacgtgactctggtggataaatctgtgcctgagccagctgacgaagtgccaacccctggagacggaatcctggagcacgatcctttcttttgtcctccaacagaagccccagacagggatttcctggtggacgctctggtgactctgctggtgcctctgctggtggctctgctgctgaccctgctgctggcttatgtgatgtgctgtcggagagagggacggctgaagagagacctggccacatctgatatccagatggtgcaccattgtactattcacggcaacaccgaggaactgcgccagatggctgcttctagggaggtgccaaggccactgagtacactgcctatgtttaatgtgcacactggcgaacggctgccccctagagtggatagcgcccaggtgccactgattctggaccagcattgaggccgcaataaaagatctttattttcattagatctgtgtgttggttttttgtgtgtcctgcaggggcgcgcctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 25 - DNA, 792 bp, unidentified
  source 1..792: mol_type = other DNA
aagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagc

SEQ ID NO: 26 - DNA, 714 bp, unidentified
  source 1..714: mol_type = other DNA
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctatgctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggaccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtcctccctggggacagcccctcctggctagtcacaccctgtaggctcctctatataacccaggggcacaggggctgcccccgggtcac

SEQ ID NO: 27 - DNA, 150 bp, unidentified
  source 1..150: mol_type = other DNA
aggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgc

SEQ ID NO: 28 - DNA, 148 bp, unidentified
  source 1..148: mol_type = other DNA
aggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtaccc

SEQ ID NO: 29 - DNA, 133 bp, unidentified
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 30 - DNA, 53 bp, unidentified
  source 1..53: mol_type = other DNA
ggccgcaataaaagatctttattttcattagatctgtgtgttggttttttgtg

SEQ ID NO: 31 - DNA, 606 bp, unidentified
  source 1..606: mol_type = other DNA
gaattcggtaccccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagcggccgatccgccacc

SEQ ID NO: 32 - DNA, 759 bp, unidentified
  source 1..759: mol_type = other DNA
gaattcggtaccccactacgggtctaggctgcccatgtaaggaggcaaggcctggggacacccgagatgcctggttataattaaccccaacacctgctgcccccccccccccaacacctgctgcctctaaaaataaccctgtccctggtggatcccctgcatgccccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagcggccgatccgccacc

SEQ ID NO: 33 - DNA, 333 bp, unidentified
  source 1..333: mol_type = other DNA
tctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtat

SEQ ID NO: 34 - DNA, 327 bp, unidentified
  source 1..327: mol_type = other DNA
cagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcaggtcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgac

SEQ ID NO: 35 - DNA, 327 bp, unidentified
  source 1..327: mol_type = other DNA
actttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaa

SEQ ID NO: 36 - DNA, 327 bp, unidentified
  source 1..327: mol_type = other DNA
atgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggt

SEQ ID NO: 37 - DNA, 312 bp, unidentified
  source 1..312: mol_type = other DNA
tgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagc

SEQ ID NO: 38 - DNA, 5417 bp, unidentified
  source 1..5417: mol_type = other DNA
gggattccctcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggatcttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaacgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctaga

SEQ ID NO: 39 - DNA, 5462 bp, unidentified
  source 1..5462: mol_type = other DNA
gggattccctcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatgctcctggactgaccactattggagcctctcctactcagactgttactctggtgacacaacctgtggttactaaggaaactgccatctccaaactagaaatgccatcttccttgatgttggagcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaacgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctaga

SEQ ID NO: 40 - protein, 1270 aa, unidentified
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHSYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 41 - protein, 1325 aa, unidentified
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDQPDLAPGLTTIGASPTQTVTLVTQPVVTKETAISKLEMPSSLMLEVPTHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 42 - protein, 1278 aa, unidentified
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDTHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 43 - protein, 1325 aa, unidentified; identical to SEQ ID NO: 41

SEQ ID NO: 44 - protein, 1192 aa, unidentified
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 45 - protein, 318 aa, unidentified
MAAAAAAAAEQQSSNGPVKKSMREKAVERRSVNKEHNSNFKAGYIPIDEDRLHKTGLRGRKGNLAICVIILLFILAVINLIITLVIWAVIRIGPNGCDSMEFHESGLLRFKQVSDMGVIHPLYKSTVGGRRNENLVITGNNQPIVFQQGTTKLSVENNKTSITSDIGMQFFDPRTQNILFSTDYETHEFHLPSGVKSLNVQKASTERITSNATSDLNIKVDGRAIVRGNEGVFIMGKTIEFHMGGNMELKAENSIILNGSVMVSTTRLPSSSSGDQLGSGDWVRYKLCMCADGTLFKVQVTSQNMGCQISDNPCGNTH

SEQ ID NO: 46 - protein, 387 aa, unidentified
MAETLFWTPLLVVLLAGLGDTEAQQTTLHPLVGRVFVHTLDHETFLSLPEHVAVPPAVHITYHAHLQGHPDLPRWLRYTQRSPHHPGFLYGSATPEDRGLQVIEVTAYNRDSFDTTRQRLVLEIGDPEGPLLPYQAEFLVRSHDAEEVLPSTPASRFLSALGGLWEPGELQLLNVTSALDRGGRVPLPIEGRKEGVYIKVGSASPFSTCLKMVASPDSHARCAQGQPPLLSCYDTLAPHFRVDWCNVTLVDKSVPEPADEVPTPGDGILEHDPFFCPPTEAPDRDFLVDALVTLLVPLLVALLLTLLLAYVMCCRREGRLKRDLATSDIQMVHHCTIHGNTEELRQMAASREVPRPLSTLPMFNVHTGERLPPRVDSAQVPLILDQH

SEQ ID NO: 47 - DNA, 2217 bp, unidentified
  source 1..2217: mol_type = other DNA; note = viral
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 48 - protein, 738 aa, unidentified
  source 1..738: note = viral
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 49 - DNA, 4472 bp, unidentified
  source 1..4472: mol_type = other DNA; note = viral
ctccatcactaggggtaaccgcgaagcgcctcccacgctgccgcgtcagcgctgacgtaaattacgtcataggggagtggtcctgtattagctgtcacgtgagtgcttttgcgacattttgcgacaccacgtggccattcatggtatatatggccgagtgagcgagcaggatctccattttgaccgcgaaatttgaacgagcagcagccatgccgggcttctacgagatcgtgcttaaggtgccgagcgacctggacgagcacctgccgggcatttctgactcgtttgtgaactgggtggcagagaaggaatgggagctgcccccggattctgacatggatcggaatctgattgagcaggcacccctgaccgtggccgagaagctacagcgcgacttcctggtccaatggcgccgcgtgagtaaggccccggaggccctcttctttgttcagttcgagaagggcgagtcctacttccacctccatattctggtagagaccacgggggtcaaatccatggtgctgggccgcttcctgagtcagattcgggacaagctggtgcagaccatctaccgcgggatcgagccgaccctgcccaactggttcgcggtgacaaagacgcgtaatggcgccggaggggggaacaaggtggtggacgagtgctacatccccaactacctgctgcccaagactcagcccgagctgcagtgggcgtggactaacatggaggagtatataagcgcgtgcttgaacctggccgagcgcaaacggctcgtggcgcagcacctgacccacgtcagccagacccaggagcagaacaaggagaatctgaacccgaattctgacgcgcctgtcatccggtcaaaaacctccgcgcgctacatggagctggtcgggtggctggtggaccggggcatcacctccgagaagcagtggatccaggaggaccaggcctcgtacatctccttcaacgccgcctccaactcgcggtctcagatcaaggccgcgctggacaatgccggcaagatcatggcgctgaccaaatccgcgcccgactacctggtaggccccgctctgcccgcggacattaaatccaaccgcatctaccgcatcctggagctgaatggctacgaccctgcctacgccggttccgtctttctcggctgggcccagaaaaagtttggcaaaaggaacaccatctggctgtttgggccggccaccacgggcaagaccaacatcgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagccgttgcaggaccggatgttcaaatttgaacttacccgccgtctggagcacgactttggcaaggtgacaaagcaggaagtcaaagagttcttccgctgggcgcaggatcacgtgaccgaggtggcgcatgagttctacgtcagaaagggtggagctaacaaaagacccgcccccgatgacgcggatataagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacatgcgagagaatgaatcagaatttcaacatttgcttcacgcacgggaccagagactgttcagaatgtttccctggcgtgtcagaatctcaaccggtcgtcagaaaaaagacgtatcggaaactctgtgcgattcatcatctgctggggcgggcacccgagattgcttgctcggcctgcgacctggtcaacgtggacctggatgactgtgtttctgagcaataaatgacttaaaccaggtatggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaattacatgttaatcaataaaccggttaattcgtttcagttgaactttggtctcctgtccttcttatcttatcggttaccatagaaactggttacttattaactgcttggtgcgcttcgcgataaaagacttacgtcatcgggttacccctagtgatgga

(19) United States
(12) Patent Application Publication
(10) Pub. No.: US 2026/0002175 A1
(43) Pub. Date: Jan. 1, 2026

(54) METHODS OF TREATING MUSCULAR DYSTROPHY

(71) Applicant: Sarepta Therapeutics, Inc., Cambridge, MA (US)

(72) Inventor: Louise RODINO-KLAPAC, Cambridge, MA (US)

(73) Assignee: Sarepta Therapeutics, Inc., Cambridge, MA (US)

(21) Appl. No.: 18/876,051

(22) PCT Filed: Jun. 28, 2023
(86) PCT No.: PCT/US2023/069291

§ 371 Date: Dec. 17, 2024

Related U.S. Application Data
(60) Provisional application No. 63/357,029, filed on Jun. 30, 2022.
(60) Provisional application No. 63/382,047, filed on Nov. 2, 2022.

(51) Int. Cl. C12N 15/86; A61K 38/48; A61K 48/00; A61P 21/00; G01N 33/68

(52) U.S. Cl. CPC C12N 15/86; A61K 38/4873; A61K 48/0058; A61P 21/00; G01N 33/6854; C12N 2750/14143; C12N 2830/008; G01N 2800/2878

(57) ABSTRACT

The present disclosure is directed to methods of treating muscular dystrophy in a subject in need thereof. In certain aspects, the method comprises administering an AAV vector, e.g., an AAVrh74 vector, and an enzyme that cleaves IgG to the subject.

### RELATED APPLICATIONS

This application is the U.S. national stage of International Application No. PCT/US2023/069291, filed Jun. 28, 2023, which claims the benefit of U.S. Provisional Application No. 63/357,029, filed Jun. 30, 2022 and U.S. Provisional Application No. 63/382,047, filed Nov. 2, 2022. The entire teachings of the above-referenced applications are incorporated by reference in their entirety.

### INCORPORATION BY REFERENCE OF SEQUENCE LISTING

The content of the electronically submitted sequence listing (Name: 4140_0450002_Sequencelisting_ST26.xml; Size: 93,577 bytes; Date of Creation: Jul. 29, 2025) filed with the application is incorporated herein by reference in its entirety.

### FIELD OF THE DISCLOSURE

The present disclosure provides methods for treating muscular dystrophy in a human subject in need thereof.

### BACKGROUND OF THE DISCLOSURE

Muscular dystrophy (MD) is a rare but highly debilitating class of genetic disorders. Duchenne muscular dystrophy (DMD), for example, is caused by a defect in the expression of the protein dystrophin. The gene encoding the protein contains seventy-nine exons spread out over more than two-million nucleotides of DNA. Any exonic mutation that changes the reading frame of the exon, or introduces a stop codon, or is characterized by removal of an entire out of frame exon or exons, or duplications of one or more exons, has the potential to disrupt production of functional dystrophin, resulting in MD.

Gene therapy offers a lasting means of treating a variety of diseases, and adeno-associated virus (AAV) is one of the most commonly investigated gene therapy vectors. AAV is a protein shell surrounding and protecting a small, single-stranded DNA genome of approximately 4.8 kilobases (kb). Naso et al., BioDrugs, 31(4): 317-334 (2017). AAV belongs to the parvovirus family and is dependent on co-infection with other viruses, mainly adenoviruses, in order to replicate. Id. Its single-stranded genome contains three genes, Rep (Replication), Cap (Capsid), and aap (Assembly). Id. These coding sequences are flanked by inverted terminal repeats (ITRs) that are required for genome replication and packaging. Id. The two cis-acting AAV ITRs are approximately 145 nucleotides in length with interrupted palindromic sequences that can fold into T shaped hairpin structures that function as primers during initiation of DNA replication.

Though early results in animal models indicate that AAV gene therapy may be a viable approach for treating DMD and BMD, AAV copy number was found to drop over time, correlating with a decrease in efficacy. One suspected reason for this decline in AAV copy number is the body's immune response to the AAV viral particle. Following a first AAV therapy, a human subject will generally develop neutralizing antibodies that facilitate the destruction of the foreign AAV vector. As a result, AAV copy number declines over time, and follow-on therapies using the same vector can result in a complete lack of response. Thus, there remains a need for improved therapeutic methods for treating MD.

### SUMMARY OF THE DISCLOSURE

Certain aspects of the present disclosure are directed to methods of treating a subject having a muscular dystrophy, comprising administering to the subject a recombinant AAVrh74 viral vector and an enzyme that cleaves immunoglobulin G (IgG).

In some aspects, the recombinant AAVrh74 viral vector and the enzyme that cleaves IgG are administered to the subject concurrently.

In some aspects, the recombinant AAVrh74 viral vector and the enzyme that cleaves IgG are administered to the subject sequentially.

In some aspects, the subject is determined to have anti-AAVrh74 antibodies.

In some aspects, the anti-AAVrh74 antibodies are neutralizing anti-AAVrh74 antibodies.

In some aspects, the anti-AAVrh74 antibodies are non-neutralizing anti-AAVrh74 antibodies.

In some aspects, the anti-AAVrh74 antibodies are total anti-AAVrh74 antibodies comprising both neutralizing and non-neutralizing antibodies.

In some aspects, the subject has a titer of anti-AAVrh74 antibodies greater than 1:400 in a total anti-AAVrh74 antibody ELISA assay.

In some aspects, the subject has anti-AAVrh74 antibodies in a neutralizing cell assay.

In some aspects, the recombinant AAVrh74 viral vector is administered after the enzyme that cleaves IgG.

In some aspects, the subject is administered the recombinant AAVrh74 viral vector no more than 54 hours after the administration of the enzyme that cleaves IgG.

In some aspects, the method further comprises, prior to administering the recombinant AAVrh74 viral vector, administering a second dose of the enzyme that cleaves IgG to the subject, if the subject is determined to have anti-AAVrh74 antibodies after a first dose of the enzyme that cleaves IgG is administered.

In some aspects, the anti-AAVrh74 antibodies are neutralizing anti-AAVrh74 antibodies.

In some aspects, the anti-AAVrh74 antibodies are non-neutralizing anti-AAVrh74 antibodies.

In some aspects, the anti-AAVrh74 antibodies are total anti-AAVrh74 antibodies comprising both neutralizing and non-neutralizing antibodies.

In some aspects, the subject has a titer of anti-AAVrh74 antibodies greater than 1:400 in a total anti-AAVrh74 antibody ELISA assay.

In some aspects, the subject has anti-AAVrh74 antibodies in a neutralizing cell assay.

In some aspects, the recombinant AAVrh74 viral vector is administered after the second dose of the enzyme that cleaves IgG.

In some aspects, the subject is administered the recombinant AAVrh74 viral vector no more than 54 hours after the administration of the second dose of the enzyme that cleaves IgG.

In some aspects, the subject is administered the second dose of the enzyme that cleaves IgG no more than 60 hours after the first dose of the enzyme.

In some aspects, the subject has a titer of anti-AAVrh74 antibodies of between about 1:1600 and about 1:3200 in a total anti-AAVrh74 antibody ELISA assay.

In some aspects, the method further comprises, prior to administering the recombinant AAVrh74 viral vector, administering a third dose of the enzyme that cleaves IgG to the subject.

In some aspects, the recombinant AAVrh74 viral vector is administered after the third dose of the enzyme that cleaves IgG.

In some aspects, the subject is administered the recombinant AAVrh74 viral vector no more than 54 hours after the administration of the third dose of the enzyme that cleaves IgG.

In some aspects, the subject is administered the third dose of the enzyme that cleaves IgG no more than 60 hours after the second dose of the enzyme.

Further provided is a method of treating a subject having a muscular dystrophy, comprising administering to the subject a recombinant AAVrh74 viral vector, wherein the subject has been previously administered an enzyme that cleaves IgG.

In some aspects, the subject has been administered an enzyme that cleaves IgG no more than 54 hours before administering the recombinant AAVrh74 viral vector.

Provided is a method of preparing a subject for a gene therapy for a muscular dystrophy comprising administering to the subject an enzyme that cleaves IgG prior to administration of a recombinant AAVrh74 viral vector.

Provided is a method of removing anti-AAVrh74 antibodies in a subject comprising administering an enzyme that cleaves IgG and measuring in the subject a titer of anti-rAAVrh74 antibodies after administering the enzyme that cleaves IgG.

In some aspects, the method further comprises measuring a titer of anti-rAAVrh74 antibodies in the subject prior to administering the enzyme that cleaves IgG.

In some aspects, the method further comprises administering a recombinant AAVrh74 viral vector if the measured titer of anti-rAAVrh74 antibodies in the subject is at or below 1:400 in a total anti-AAVrh74 antibody ELISA assay.

In some aspects, the subject is not administered a recombinant AAVrh74 viral vector if the anti-rAAVrh74 antibodies in the subject is above 1:400 in a total anti-AAVrh74 antibody ELISA assay.

In some aspects, the method further comprises administering to the subject a second dose of the enzyme that cleaves IgG.

In some aspects, the method further comprises measuring in the subject a titer of anti-rAAVrh74 antibodies after the second dose of the enzyme that cleaves IgG.

In some aspects, the anti-AAVrh74 antibodies are neutralizing anti-AAVrh74 antibodies.

In some aspects, the anti-AAVrh74 antibodies are non-neutralizing anti-AAVrh74 antibodies.

In some aspects, the anti-AAVrh74 antibodies are total anti-AAVrh74 antibodies comprising both neutralizing and non-neutralizing antibodies.

In some aspects, the subject has a titer of anti-AAVrh74 antibodies greater than 1:400 in a total anti-AAVrh74 antibody ELISA assay.

In some aspects, the subject has anti-AAVrh74 antibodies in a neutralizing cell assay.

In some aspects, the method further comprises administering a recombinant AAVrh74 viral vector if the measured titer of anti-rAAVrh74 antibody in the subject is below 1:400 in a total anti-AAVrh74 antibody ELISA assay.

In some aspects, the recombinant AAVrh74 viral vector is administered no more than 54 hours after the second dose of the enzyme that cleaves IgG.

In some aspects, the subject is not administered a recombinant AAVrh74 viral vector if the measured titer of anti-rAAVrh74 antibody in the subject is above 1:400 in a total anti-AAVrh74 antibody ELISA assay.

In some aspects, the muscular dystrophy is selected from Duchene muscular dystrophy (DMD) and limb-girdle muscular dystrophy (LGMD).

In some aspects, the muscular dystrophy is DMD.

In some aspects, the IgG is a human IgG.

In some aspects, the enzyme that cleaves IgG specifically targets and cleaves a human IgG selected from IgG1, IgG2, IgG3, IgG4, and any combination thereof.

In some aspects, the enzyme that cleaves IgG specifically targets and cleaves human IgG1, human IgG2, human IgG3, and human IgG4.

In some aspects, the IgG binds the recombinant AAVrh74 viral vector.

In some aspects, the enzyme that cleaves immunoglobulin IgG comprises a protease.

In some aspects, the protease comprises a cysteine protease or a thiol protease.

In some aspects, the enzyme that cleaves IgG inactivates the IgG.

In some aspects, the enzyme that cleaves IgG cleaves human IgG at a hinge region.

In some aspects, the protease is isolated or derived from a protease expressed by Streptococcus pyogenes, Streptococcus equi, or Mycoplasma canis.

In some aspects, the enzyme that cleaves IgG comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 1 or 2.

In some aspects, the enzyme that cleaves IgG comprises an amino acid sequence set forth in any of SEQ ID NOs: 3-18.

In some aspects, the enzyme that cleaves IgG comprises the amino acid sequence set forth in SEQ ID NO: 1 or 2.

In some aspects, the enzyme is Imlifidase.

In some aspects, the subject has one or more IgG antibodies that specifically bind a protein present in a recombinant AAVrh74 viral vector.

In some aspects, the subject has one or more IgG antibodies that specifically bind a capsid protein of a recombinant AAVrh74 viral vector.

In some aspects, the one or more IgG antibodies are AAVrh74 neutralizing antibodies.

In some aspects, the one or more IgG antibodies are AAVrh74 non-neutralizing antibodies.

In some aspects, the one or more IgG antibodies are total AAVrh74 antibodies comprising both neutralizing and non-neutralizing antibodies.

In some aspects, the subject has a titer of anti-AAVrh74 antibodies greater than 1:400 in a total anti-AAVrh74 antibody ELISA assay.

In some aspects, the subject has anti-AAVrh74 antibodies in a neutralizing cell assay.

In some aspects, the subject has been previously administered a recombinant AAVrh74 viral vector.

In some aspects, the recombinant AAVrh74 viral vector comprises a genetic cassette encoding a therapeutic molecule.

In some aspects, the therapeutic molecule comprises a polypeptide, an RNA molecule, or a DNA molecule.

In some aspects, the genetic cassette encodes a therapeutic polypeptide selected from a microdystrophin, beta sarcoglycan, alpha sarcoglycan, and any combination thereof.

In some aspects, the genetic cassette comprises a nucleic acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 19.

In some aspects, the genetic cassette comprises the nucleic acid sequence set forth in SEQ ID NO: 19.

In some aspects, the genetic cassette comprises a nucleic acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 21.

In some aspects, the genetic cassette comprises the nucleic acid sequence set forth in SEQ ID NO: 21.

In some aspects, the genetic cassette comprises a nucleic acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 23.

In some aspects, the genetic cassette comprises the nucleic acid sequence set forth in SEQ ID NO: 23.

In some aspects, the recombinant AAVrh74 viral vector further comprises a promoter.

In some aspects, the promoter is a tissue specific promoter.

In some aspects, the promoter is selected from an MHCK7 promoter and a tMCK promoter.

In some aspects, the promoter comprises a nucleic acid sequence having a least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 25 or 26.

In some aspects, the promoter comprises the nucleic acid sequence set forth in SEQ ID NO: 25 or 26.

In some aspects, the recombinant AAVrh74 viral vector further comprises an intron.

In some aspects, the intron comprises a nucleic acid sequence having a least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to a nucleic acid sequence set forth in SEQ ID NOs: 27-29.

In some aspects, the intron comprises a nucleic acid sequence set forth in SEQ ID NOs: 27-29.

In some aspects, the recombinant AAVrh74 viral vector further comprises a 3′UTR poly(A) tail sequence.

In some aspects, the 3′UTR poly(A) tail sequence comprises a nucleic acid sequence having a least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% sequence identity to the nucleic acid sequence set forth in SEQ ID NO: 30.

In some aspects, the AAVrh74 viral vector comprises a nucleic acid sequence having a least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% sequence identity to a nucleic acid sequence selected from the nucleic acid sequences set forth in SEQ ID NOs: 20, 22, and 24.

In some aspects, Imlifidase is administered at a dose of 0.25 mg/kg.

In some aspects, the recombinant AAVrh74 viral vector is delandistrogene moxeparvovec (or delandistrogene moxeparvovec-rokl), bidridistrogene xeboparvovec, or patidistrogene bexoparvovec.

In some aspects, delandistrogene moxeparvovec, bidridistrogene xeboparvovec, or patidistrogene bexoparvovec is administered at a dose of about 1.33×10¹⁴ vg/kg.

In some aspects, the administration of Imlifidase is intravenous.

In some aspects, the administration of delandistrogene moxeparvovec, bidridistrogene xeboparvovec, or patidistrogene bexoparvovec is intravenous.

In some aspects, the recombinant AAVrh74 is delandistrogene moxeparvovec.

Further provided is a kit for treating a muscular dystrophy in a subject in need thereof, comprising (i) an enzyme that cleaves IgG, (ii) a recombinant AAVrh74 viral vector, and (iii) instructions to administer the enzyme and/or the recombinant AAVrh74 viral vector to the subject a described herein.

Provided is a use of a recombinant AAVrh74 viral vector for the manufacture of a medicament for treating a muscular dystrophy in a subject in need thereof, wherein the subject has been previously administered an enzyme that cleaves IgG.

Provided is a use of a recombinant AAVrh74 viral vector for the manufacture of a medicament for treating a muscular dystrophy in a subject in need thereof in combination with an enzyme that cleaves IgG.

In some aspects, the recombinant AAVrh74 viral vector and the enzyme are to be administered concurrently for treating the muscular dystrophy in the subject in need thereof.

In some aspects, the recombinant AAVrh74 viral vector and the enzyme are to be administered sequentially.

In some aspects, the recombinant AAVrh74 viral vector is administered no later than 54 hours after the administration of the enzyme.

In some aspects, the muscular dystrophy is selected from DMD and LGMD.

In some aspects, the muscular dystrophy is DMD.

Also provided is a composition comprising a recombinant AAVrh74 viral vector for use in treating a muscular dystrophy in a subject in need thereof, wherein the subject has been previously administered an enzyme that cleaves IgG.

Provided is a composition comprising a recombinant AAVrh74 viral vector for use in treating a muscular dystrophy in a subject in need thereof in combination with an enzyme that cleaves IgG.

In some aspects, the recombinant AAVrh74 viral vector and the enzyme are to be administered concurrently for use in treating a muscular dystrophy in a subject in need thereof.

In some aspects, the recombinant AAVrh74 viral vector and the enzyme are to be administered sequentially.

In some aspects, the recombinant AAVrh74 viral vector is administered no later than 54 hours after the administration of the enzyme.

In some aspects, the muscular dystrophy is selected from DMD and LGMD.

In some aspects, the muscular dystrophy is DMD.

Further provided is a method of removing anti-AAVrh74 antibodies in treating a subject in need thereof comprising administering Imlifidase and measuring a titer of anti-rAAVrh74 antibodies in the subject after administering Imlifidase.

In some aspects, the anti-AAVrh74 antibodies are neutralizing anti-AAVrh74 antibodies.

In some aspects, the anti-AAVrh74 antibodies are non-neutralizing anti-AAVrh74 antibodies.

In some aspects, the anti-AAVrh74 antibodies are total anti-AAVrh74 antibodies comprising both neutralizing and non-neutralizing antibodies.

In some aspects, the subject has a titer of anti-AAVrh74 antibodies greater than 1:400 in a total anti-AAVrh74 antibody ELISA assay.

In some aspects, the subject has anti-AAVrh74 antibodies in a neutralizing cell assay.

In some aspects, the method further comprises measuring a titer of anti-rAAVrh74 antibodies in the subject prior to administering Imlifidase.

In some aspects, the anti-AAVrh74 antibodies are neutralizing anti-AAVrh74 antibodies.

In some aspects, the anti-AAVrh74 antibodies are non-neutralizing anti-AAVrh74 antibodies.

In some aspects, the anti-AAVrh74 antibodies are total anti-AAVrh74 antibodies comprising both neutralizing and non-neutralizing antibodies.

In some aspects, the subject has a titer of anti-AAVrh74 antibodies greater than 1:400 in a total anti-AAVrh74 antibody ELISA assay.

In some aspects, the subject has anti-AAVrh74 antibodies in a neutralizing cell assay.

In some aspects, the method further comprises administering a recombinant AAVrh74 viral vector if the measured titer of anti-rAAVrh74 antibodies in the subject is at or below 1:400 in a total anti-AAVrh74 antibody ELISA assay.

In some aspects, Imlifidase is administered at a dose of 0.25 mg/kg.

In some aspects, the recombinant AAVrh74 viral vector is delandistrogene moxeparvovec, bidridistrogene xeboparvovec, or patidistrogene bexoparvovec.

In some aspects, the recombinant AAVrh74 is delandistrogene moxeparvovec.

In some aspects, delandistrogene moxeparvovec, bidridistrogene xeboparvovec, or patidistrogene bexoparvovec is administered at a dose of about 1.33×10¹⁴ vg/kg.

In some aspects, delandistrogene moxeparvovec is administered at a dose of about 1.33×10¹⁴ vg/kg.

In some aspects, the administration of Imlifidase is intravenous.

In some aspects, the administration of delandistrogene moxeparvovec, bidridistrogene xeboparvovec, or patidistrogene bexoparvovec is intravenous.

In some aspects, the subject is not administered a recombinant AAVrh74 viral vector if the measured titer of anti-rAAVrh74 antibodies in the subject is above 1:400 in a total anti-AAVrh74 antibody ELISA assay.

In some aspects, the method further comprises administering to the subject a second dose of Imlifidase.

In some aspects, the second dose of Imlifidase is 0.25 mg/kg.

In some aspects, the second dose of Imlifidase is administered no more than 60 hours after the first dose.

In some aspects, the method further comprises measuring in the subject a titer of anti-rAAVrh74 antibodies after the second dose of Imlifidase.

In some aspects, the anti-AAVrh74 antibodies are neutralizing anti-AAVrh74 antibodies.

In some aspects, the anti-AAVrh74 antibodies are non-neutralizing anti-AAVrh74 antibodies.

In some aspects, the anti-AAVrh74 antibodies are total anti-AAVrh74 antibodies comprising both neutralizing and non-neutralizing antibodies.

In some aspects, the method further comprises administering a recombinant AAVrh74 viral vector if the measured titer of anti-rAAVrh74 antibodies in the subject is at or below 1:400 in a total anti-AAVrh74 antibody ELISA assay.

In some aspects, the recombinant AAVrh74 viral vector is administered no more than 54 hours after the second dose of Imlifidase.

### BRIEF DESCRIPTION OF THE DRAWINGS

FIG. 1 shows a schematic of the study design for Imlifidase only recipients, as described in Example 2.

FIG. 2 shows a schematic of the overall study design, as described in Example 2.

FIG. 3 shows a schematic of the study design for the pre-infusion and Imlifidase infusion period, as described in Example 2.

FIG. 4 shows total antibody (TAb) responses to AAVrh74 in non-human primates as described in Example 1.

FIG. 5 shows total antibody (TAb) responses to AAVrh74 after Imlifidase treatment in non-human primates of Groups 4, 5, and 6 as described in Example 1.

FIG. 6A shows total antibody (TAb) responses to AAVrh74 after Imlifidase treatment in non-human primates of Group 7 as described in Example 1. FIG. 6B shows total antibody (TAb) responses to AAVrh74 in non-human primates of Group 7 post AAVrh74 redose as described in Example 1.

FIG. 7A-C show western blots of serum of seronegative non-human primates of Group 3 (P0201, P0202, P0203) as described in Example 1. The animals were not treated with Imlifidase.

FIG. 8A shows a western blot of serum of a seropositive non-human primate of Group 4 (P0301) with an anti-AAVrh74 baseline titer of 1:1,600 as described in Example 1. FIG. 8B shows a western blot of serum of a seropositive non-human primate of Group 4 (P0302) with an anti-AAVrh74 baseline titer of 1:400 as described in Example 1. FIG. 8C shows a western blot of serum of a seropositive non-human primate of Group 4 (P0303) with an anti-AAVrh74 baseline titer of 1:800 as described in Example 1. The animals of Group 4 were not treated with Imlifidase.

FIG. 9A shows a western blot of serum of a seropositive non-human primate of Group 5 (P0401) with an anti-AAVrh74 baseline titer of 1:1,600 and an anti-AAVrh74 titer after Imlifidase of 1:200 as described in Example 1. FIG. 9B shows a western blot of serum of a seropositive non-human primate of Group 5 (P0402) with an anti-AAVrh74 baseline titer of 1:800 and an anti-AAVrh74 titer after Imlifidase of 1:50 as described in Example 1. FIG. 9C shows a western blot of serum of a seropositive non-human primate of Group 5 (P0403) with an anti-AAVrh74 baseline titer of 1:800 and an anti-AAVrh74 titer of 1:50 after Imlifidase as described in Example 1.

FIG. 10A shows a western blot of serum of a high seropositive non-human primate of Group 6 (P0501) with an anti-AAVrh74 baseline titer of 1:6,400 and an anti-AAVrh74 titer after Imlifidase of 1:800 as described in Example 1. FIG. 10B shows a western blot of serum of a high seropositive non-human primate of Group 6 (P0502) with an anti-AAVrh74 baseline titer of 1:3,200 and an anti-AAVrh74 titer after Imlifidase of 1:200 as described in Example 1. FIG. 10C shows a western blot of serum of a high seropositive non-human primate of Group 6 (P0503) with an anti-AAVrh74 baseline titer of 1:3,200 and an anti-AAVrh74 titer of 1:400 after Imlifidase as described in Example 1.

FIG. 11A shows a western blot of serum of a seronegative non-human primate of Group 7 (P0601) with an anti-AAVrh74 baseline titer of 1:204,800 after a first dose of AAVrh74-eGFP and an anti-AAVrh74 titer after Imlifidase of 1:6,400 as described in Example 1. FIG. 11B shows a western blot of serum of a seronegative non-human primate of Group 7 (P0602) with an anti-AAVrh74 baseline titer of 1:400 after a first dose of AAVrh74-eGFP and an anti-AAVrh74 titer after Imlifidase of 1:200 as described in Example 1. FIG. 11C shows a western blot of serum of a seronegative non-human primate of Group 7 (P0603) with an anti-AAVrh74 baseline titer of 1:25,600 after a first dose of AAVrh74-eGFP and an anti-AAVrh74 titer of 1:3,200 after Imlifidase as described in Example 1.

FIG. 12 shows GFP expression from the heart in Group 3, 4, 5, 6, and 7 animals and control Group 2 animals as described in Example 1.

FIG. 13 shows GFP expression from the skeletal muscle (gastrocnemius) in Group 3, 4, 5, 6, and 7 animals and control Group 2 animals as described in Example 1.

FIG. 14 shows GFP expression from the diaphragm in Group 3, 4, 5, 6, and 7 animals and control Group 2 animals as described in Example 1.

FIG. 15 shows average GFP expression of all tissues in Group 3 animals (AAVr74-eGFP vector control, AAVrh74 seronegative); Group 4 animals (AAVrh74-eGFP vector control, AAVrh74 seropositive with titers of 1:800-1:1,600); Group 5 animals (Imlifidase+AAVrh74-eGFP vector, AAVrh74 seropositive with titers of 1:800-1:1,600); Group 6 animals (Imlifidase+AAVrh74-eGFP vector, AAVrh74 seropositive with titers of 1:3,200-1:25,600); and Group 7 (redose group) as described in Example 1.

FIG. 16 shows GFP expression (% positive area) from different muscle types in Group 3, 4, 5, 6, and 7 animals and saline control Group 2 animals as described in Example 1.

### DETAILED DESCRIPTION OF THE DISCLOSURE

The present disclosure is directed to methods of treating a subject having a muscular dystrophy, comprising administering to the subject a recombinant AAVrh74 viral vector and an enzyme that cleaves immunoglobulin G (IgG). In some aspects, the muscular dystrophy is DMD. In some aspects, the muscular dystrophy is LGMD.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower), unless otherwise indicated.

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures.

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary of Biochemistry and Molecular Biology, 2nd ed., 2006, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

As used herein, the term “an enzyme that cleaves IgG” refers to a polypeptide (e.g., a protease) that facilitates the cleavage of an immunoglobulin G (IgG). In some aspects, the cleavage of the IgG inactivates the IgG. In some aspects, the cleavage of the IgG blocks the ability of the IgG to bind an antigen. Any enzyme capable of cleaving IgG can be used in the methods disclosed herein. Examples of suitable enzymes can be found, for example, in International Publication No. WO2020102740 and European Patent Application EP 3768304B1, which are incorporated by reference herein in their entirety. In some aspects, the enzyme that cleaves IgG comprises IdeS (Immunoglobulin-degrading Enzyme of S. pyrogenes; also known as Imlifidase).

As used herein, the term “adeno-associated virus” (AAV) is a standard abbreviation for adeno-associated virus. Adeno-associated virus is a single-stranded DNA parvovirus that grows only in cells in which certain functions are provided by a co-infecting helper virus. AAV serotypes include, but are not limited to AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh74, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers).

As used herein, the term “adeno-associated vector nucleic acid” or “AAV vector nucleic acid” refers to a vector comprising one or more polynucleotides of interest (or “transgenes,” such as, e.g., micro-dystrophin) that are flanked by AAV terminal repeat sequences (ITRs). Such AAV vector nucleic acids can be replicated and packaged into infectious viral particles when present in a host cell that has been transfected with a vector encoding and expressing rep and cap gene products.

As used herein, the terms “AAV vector,” “AAV virion,” “AAV viral particle,” “AAV vector particle,” “AAVrh74 vector,” “AAVrh74 virion,” “AAVrh74 viral particle,” or “AAVrh74 vector particle” refer to a viral particle composed of at least one AAV capsid protein or AAVrh74 capsid protein and an encapsidated polynucleotide AAV vector. If the particle comprises a heterologous polynucleotide (i.e., a polynucleotide other than a wild-type AAV genome, such as, e.g., a transgene to be delivered to a mammalian cell), it is typically referred to as an “AAV vector particle” “AAVrh74 vector particle” or, in some instances, simply an “AAV vector” or “AAVrh74 vector.” Thus, production of an AAV vector particle necessarily includes packaging of an AAV vector nucleic acid into an AAV capsid.

“Serotype,” with respect to a vector or virus capsid, is defined by a distinct immunological profile based on the capsid protein sequences and capsid structure.

“AAV Cap” means AAV Cap proteins, VP1, VP2, and VP3 and analogs thereof.

“AAV Rep” means AAV Rep proteins and analogs thereof.

As used herein, “flanked,” with respect to a sequence that is flanked by other elements, indicates the presence of one or more the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the transgene and a flanking element. A sequence (e.g., a transgene) that is “flanked” by two other elements (e.g., ITRs), indicates that one element is located 5′ to the sequence and the other is located 3′ to the sequence; however, there may be intervening sequences between.

In some aspects, an “AAV” includes a derivative of a known AAV. In some aspects, an “AAV” includes a modified or an artificial AAV. In certain aspects, the AAV is an AAVrh74. As used herein, “AAVrh74” refers to adeno-associated virus rhesus serotype 74, which is endogenous to the rhesus macaque and was originally isolated from mesenteric lymph nodes and subsequently from the spleen.

The term “derived from,” as used herein, refers to a component that is isolated from or made using a specified molecule or organism, or information (e.g., amino acid or nucleic acid sequence) from the specified molecule or organism. For example, a nucleic acid sequence that is derived from a second nucleic acid sequence can include a nucleotide sequence that is identical or substantially similar to the nucleotide sequence of the second nucleic acid sequence. In the case of nucleotides or polypeptides, the derived species can be obtained by, for example, naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. Mutagenesis of a polypeptide typically entails manipulation of the polynucleotide that encodes the polypeptide. In some aspects, a nucleotide or amino acid sequence that is derived from a second nucleotide or amino acid sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% to the second nucleotide or amino acid sequence, respectively, wherein the first nucleotide or amino acid sequence retains the biological activity of the second nucleotide or amino acid sequence.

The terms “nucleic acids,” “nucleic acid molecules,” “nucleotides,” “nucleotide(s) sequence,” and “polynucleotide” are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

As used herein, the term “expression cassette” refers to any type of genetic construct containing a nucleic acid in which part or all of the nucleic acid encoding sequence is capable of being transcribed. Typically, an expression cassette comprises a promoter operably linked to a nucleic acid (e.g., transgene of interest). In some aspects, the “expression cassette” includes a polynucleotide sequence encoding human micro-dystrophin. In some aspects, the “expression cassette” includes a polynucleotide sequence which is an intron. In some aspects, the “expression cassette” includes a polynucleotide sequence directing the addition of a poly-adenosine (poly(A)) tail.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. Two or more coding regions can be present in a single polynucleotide construct, e.g., on a single vector, or in separate polynucleotide constructs, e.g., on separate (different) vectors. It follows, then, that a single vector can contain just a single coding region, or comprise two or more coding regions. A “transcript,” as used herein, refers to an mRNA sequence formed during DNA transcription, e.g., by RNA polymerase.

The term “downstream” refers to a nucleotide sequence that is located 3′ to a reference nucleotide sequence. In certain aspects, downstream nucleotide sequences relate to sequences that follow the starting point of transcription. For example, the translation initiation codon of a gene is located downstream of the start site of transcription. The term “upstream” refers to a nucleotide sequence that is located 5′ to a reference nucleotide sequence. In certain aspects, upstream nucleotide sequences relate to sequences that are located on the 5′ side of a coding region or starting point of transcription. For example, most promoters are located upstream of the start site of transcription.

A polynucleotide, e.g., a genetic cassette disclosed herein, which encodes a product, e.g., a micro-dystrophin, can include a promoter and/or other expression (e.g., transcription or translation) control elements operably associated with one or more coding regions. In an operable association a coding region for a gene product, e.g., a polypeptide, is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other expression control elements, besides a promoter, for example enhancers, operators, repressors, and transcription termination signals, can also be operably associated with a coding region to direct gene product expression.

“Transcriptional control sequences” refer to DNA regulatory sequences, such as promoters, enhancers, terminators, and the like, that provide for the expression of a coding sequence in a host cell. A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit B-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

“Promoter” and “promoter sequence” are used interchangeably and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

In some aspects, the nucleic acid molecule comprises a tissue specific promoter. In certain aspects, the tissue specific promoter drives expression of the therapeutic protein, e.g., a dystrophin or a micro-dystrophin, in the brain, muscle, kidney, lung, testis, or any combination thereof. In particular, aspects, the promoter is selected from the group consisting of enhancer 358 bp muscle creatine kinase proximal promoter (Enh358MCK), muscle creatine kinase (CK) promoter (e.g., hCK (SEQ ID NO: 31) or hCK Plus (SEQ ID NO: 32), truncated muscle creatine kinase promoter (tMCK; SEQ ID NO:26), myosin heavy chain (MHC), MHCK7 (a hybrid version of MHC and MCK; SEQ ID NO:25), C5-12 (synthetic promoter), a muscle- and heart-specific enhancer (MHCK), CK8, SPc5-12, human desmin (Des) promoter, human alpha-myosin heavy chain (a-MHC) promoter, rat myosin light chain 2 (MLC-2) promoter, and human cardiac troponin C (cTnC) promoter. In certain aspects, the promoter comprises a tMCK promoter. In certain aspects, the promoter comprises an MHCK7 promoter.

TABLE 1
Promoter Sequences

| Promoter | Nucleotide Sequence |
|---|---|
| MHCK7 | AAGCTTGCATGTCTAAGCTAGACCCTTCAGATTAAAAATAACTGAGGTAAGGGCCTGG |
| | GTAGGGGAGGTGGTGTGAGACGCTCCTGTCTCTCCTCTATCTGCCCATCGGCCCTTTG |
| | GGGAGGAGGAATGTGCCCAAGGACTAAAAAAAGGCCATGGAGCCAGAGGGGCGAGGGC |
| | AACAGACCTTTCATGGGCAAACCTTGGGGCCCTGCTGTCTAGCATGCCCCACTACGGG |
| | TCTAGGCTGCCCATGTAAGGAGGCAAGGCCTGGGGACACCCGAGATGCCTGGTTATAA |
| | TTAACCCAGACATGTGGCTGCCCCCCCCCCCCCAACACCTGCTGCCTCTAAAAATAAC |
| | CCTGTCCCTGGTGGATCCCCTGCATGCGAAGATCTTCGAACAAGGCTGTGGGGGACTG |
| | AGGGCAGGCTGTAACAGGCTTGGGGGCCAGGGCTTATACGTGCCTGGGACTCCCAAAG |
| | TATTACTGTTCCATGTTCCCGGCGAAGGGCCAGCTGTCCCCCGCCAGCTAGACTCAGC |
| | ACTTAGTTTAGGAACCAGTGAGCAAGTCAGCCCTTGGGGCAGCCCATACAAGGCCATG |
| | GGGCTGGGCAAGCTGCACGCCTGGGTCCGGGGTGGGCACGGTGCCCGGGCAACGAGCT |
| | GAAAGCTCATCTGCTCTCAGGGGCCCCTCCCTGGGGACAGCCCCTCCTGGCTAGTCAC |
| | ACCCTGTAGGCTCCTCTATATAACCCAGGGGCACAGGGGCTGCCCTCATTCTACCACC |
| | ACCTCCACAGCACAGACAGACACTCAGGAGCAGCCAGC (SEQ ID NO: 25) |
| tMCK | CCACTACGGGTCTAGGCTGCCCATGTAAGGAGGCAAGGCCTGGGGACACCCGAGATGC |
| | CTGGTTATAATTAACCCCAACACCTGCTGCCCCCCCCCCCCCAACACCTGCTGCCTGA |
| | GCCTGAGCGGTTACCCCACCCCGGTGCCTGGGTCTTAGGCTCTGTACACCATGGAGGA |
| | GAAGCTCGCTCTAAAAATAACCCTGTCCCTGGTGGATCCACTACGGGTCTATGCTGCC |
| | CATGTAAGGAGGCAAGGCCTGGGGACACCCGAGATGCCTGGTTATAATTAACCCCAAC |
| | ACCTGCTGCCCCCCCCCCCCCAACACCTGCTGCCTGAGCCTGAGCGGTTACCCCACCC |
| | CGGTGCCTGGGTCTTAGGCTCTGTACACCATGGAGGAGAAGCTCGCTCTAAAAATAAC |
| | CCTGTCCCTGGTGGACCACTACGGGTCTAGGCTGCCCATGTAAGGAGGCAAGGCCTGG |
| | GGACACCCGAGATGCCTGGTTATAATTAACCCCAACACCTGCTGCCCCCCCCCCCCAA |
| | CACCTGCTGCCTGAGCCTGAGCGGTTACCCCACCCCGGTGCCTGGGTCTTAGGCTCTG |
| | TACACCATGGAGGAGAAGCTCGCTCTAAAAATAACCCTGTCCCTGGTCCTCCCTGGGG |
| | ACAGCCCCTCCTGGCTAGTCACACCCTGTAGGCTCCTCTATATAACCCAGGGGCACAG |
| | GGGCTGCCCCCGGGTCAC (SEQ ID NO: 26) |
| hCK | GAATTCGGTACCCCACTACGGGTTTAGGCTGCCCATGTAAGGAGGCAAGGCCTGGGGA |
| | CACCCGAGATGCCTGGTTATAATTAACCCAGACATGTGGCTGCCCCCCCCCCCCCCAA |
| | CACCTGCTGCCTCTAAAAATAACCCTGTCCCTGGTGGATCCCCTGCATGCGAAGATCT |
| | TCGAACAAGGCTGTGGGGGACTGAGGGCAGGCTGTAACAGGCTTGGGGGCCAGGGCTT |
| | ATACGTGCCTGGGACTCCCAAAGTATTACTGTTCCATGTTCCCGGCGAAGGGCCAGCT |
| | GTCCCCCGCCAGCTAGACTCAGCACTTAGTTTAGGAACCAGTGAGCAAGTCAGCCCTT |
| | GGGGCAGCCCATACAAGGCCATGGGGCTGGGCAAGCTGCACGCCTGGGTCCGGGGTGG |
| | GCACGGTGCCCGGGCAACGAGCTGAAAGCTCATCTGCTCTCAGGGGCCCCTCCCTGGG |
| | GACAGCCCCTCCTGGCTAGTCACACCCTGTAGGCTCCTCTATATAACCCAGGGGCACA |
| | GGGGCTGCCCTCATTCTACCACCACCTCCACAGCACAGACAGACACTCAGGAGCCAGC |
| | CAGCGTCGAGCGGCCGATCCGCCACC (SEQ ID NO: 31) |
| hCK | GAATTCGGTACCCCACTACGGGTCTAGGCTGCCCATGTAAGGAGGCAAGGCCTGGGGA |
| Plus | CACCCGAGATGCCTGGTTATAATTAACCCCAACACCTGCTGCCCCCCCCCCCCCAACA |
| | CCTGCTGCCTCTAAAAATAACCCTGTCCCTGGTGGATCCCCTGCATGCCCCACTACGG |
| | GTTTAGGCTGCCCATGTAAGGAGGCAAGGCCTGGGGACACCCGAGATGCCTGGTTATA |
| | ATTAACCCAGACATGTGGCTGCCCCCCCCCCCCCCAACACCTGCTGCCTCTAAAAATA |
| | ACCCTGTCCCTGGTGGATCCCCTGCATGCGAAGATCTTCGAACAAGGCTGTGGGGGAC |
| | TGAGGGCAGGCTGTAACAGGCTTGGGGGCCAGGGCTTATACGTGCCTGGGACTCCCAA |
| | AGTATTACTGTTCCATGTTCCCGGCGAAGGGCCAGCTGTCCCCCGCCAGCTAGACTCA |
| | GCACTTAGTTTAGGAACCAGTGAGCAAGTCAGCCCTTGGGGCAGCCCATACAAGGCCA |
| | TGGGGCTGGGCAAGCTGCACGCCTGGGTCCGGGGTGGGCACGGTGCCCGGGCAACGAG |
| | CTGAAAGCTCATCTGCTCTCAGGGGCCCCTCCCTGGGGACAGCCCCTCCTGGCTAGTC |
| | ACACCCTGTAGGCTCCTCTATATAACCCAGGGGCACAGGGGCTGCCCTCATTCTACCA |
| | CCACCTCCACAGCACAGACAGACACTCAGGAGCCAGCCAGCGTCGAGCGGCCGATCCG |
| | CCACC (SEQ ID NO: 32) |

In some aspects, the expression of a protein product, e.g., a dystrophin or a micro-dystrophin, can be increased using one or more enhancers. Enhancers can be located, e.g., in an AAV, 5′ or 3′ of a coding region or within an intron. In some aspects, the one or more enhancers are tissue specific enhancers. In certain aspects, the one or more enhancers are selected from a human skeletal actin gene element, a cardiac actin gene element, a myocyte-specific enhancer binding factor MEF (e.g, MEF2), a MyoD enhancer element, a cardiac enhancer factor (CEF) site, murine creatine kinase enhancer element, skeletal fast-twitch troponin C gene element, slow-twitch cardiac troponin C gene element, the slow-twitch troponin I gene element, hypozia-inducible nuclear factors, steroid-inducible element, glucocorticoid response element (GRE) and any combination thereof.

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage. The term “yield,” as used herein, refers to the amount of a polypeptide produced by the expression of a gene.

The term “dystrophin,” “dys,” or “dystrophin polypeptide,” as used herein, is intended to refer to the full-length dystrophin protein and fragments and/or variants thereof. Human dystrophin is encoded by the DMD gene, which is 2.4 megabases (mb) in size, comprising 79 exons. Full-length human dystrophin comprises an N-terminus (NT) domain, four hinge regions (H1-H4), twenty-four spectrin-like repeat regions (R1-R24), a cysteine rich (CR) domain, and a C-terminus (CT) domain. Dystrophin can further comprise various binding domains, including one or more dystroglycan binding domains (Dg BD) located, e.g., within the H4 and CR domains; one or more neuronal nitric oxide synthase binding domains located, e.g., within one or more spectrin-like repeat regions, e.g., within R16 and R17; one or more syntrophin binding domains (Syn BD) located, e.g., within the CT domain; and one or more dystrobrevin binding domains (DB BD) located, e.g., within the CT domain. Structurally, the spectrin-like repeats form central rod-domains, giving dystrophin a modular structure. In some aspects, the region of dystrophin comprising R1-R24 is collectively referred to as the “rod domain.”

As used herein, a “dystrophin” includes fragments of the full-length dystrophin sequence, including a “micro-dystrophin” or a “mini-dystrophin,” which can be used interchangeably. A “micro-dystrophin” is used to refer to any dystrophin polypeptide that includes a deletion relative to the full-length dystrophin sequence. A dystrophin, e.g., a micro-dystrophin, can include a deletion at the N-terminus (e.g., a deletion of all or part of the NT domain), at the C-terminus (e.g., a deletion of all or part of the CT domain), in between the N-terminus and the C-terminus (e.g., a deletion of one or more hinge regions or a portion thereof and/or one or more spectrin-like repeat regions or a portion thereof), or any combination thereof. A dystrophin, e.g., a micro-dystrophin, can include a deletion within one or more of the NT domain, a hinge region, a spectrin-like repeat region, a CR domain, and the CT domain. In some aspects, the deletion comprises a partial deletion of one or more of the NT domain, a hinge region, a spectrin-like repeat region, a CR domain, and the CT domain. In some aspects, the deletion comprises a full deletion of one or more of the NT domain, a hinge region, a spectrin-like repeat region, a CR domain, and the CT domain. Examples of dystrophin and/or micro-dystrophin useful in the present disclosure can be found, e.g., in international publication numbers WO/2002/029056, WO/2008/088895, WO/2010/093784, WO/2011/088081, WO/2014/193716, WO/2016/004319, WO/2016/115543, WO/2017/077451, WO/2017/221145; WO/2019/012336; WO/2019/245973; U.S. Publication No. US 2017/0368198; and U.S. Pat. No. 7,655,467, each of which is incorporated by reference herein in its entirety. In certain aspects, the micro-dystrophin retains a function of full-length dystrophin. In certain aspects, the dystrophin comprises an amino acid sequence selected from the amino acid sequences set forth in SEQ ID NOs: 40-44. In certain aspects, the dystrophin comprises an amino acid sequence encoded by a nucleic acid sequence selected from the nucleic acid sequences set forth in SEQ ID NOs: 19 and 33-39. In some aspects, the dystrophin comprises an amino acid sequence encoded by a nucleic acid sequence as set forth in SEQ ID NO: 19. In certain aspects, the AAVrh74 comprises a genetic cassette comprising the nucleic acid sequence set forth in SEQ ID NO: 19.

TABLE 2
Micro-Dystrophin Sequences.

ATGCTGTGGTGGGAGGAGGTGGAGGATTGTTATGAAAGGGAGGACGTGCAGAAGAAGACTTTTAC
CAAGTGGGTGAACGCTCAGTTCAGCAAATTTGGGAAGCAGCACATCGAGAATCTGTTTTCCGACC
TGCAGGATGGGAGACGGCTGCTGGATCTGCTGGAAGGACTGACTGGCCAGAAGCTGCCCAAAGAG
AAGGGGAGCACTAGGGTGCACGCCCTGAACAACGTGAACAAAGCTCTGAGAGTGCTGCAGAACAA
CAACGTGGATCTGGTGAATATTGGCAGTACTGATATCGTGGACGGGAACCACAAACTGACACTGG
GCCTGATCTGGAACATTATTCTGCACTGGCAGGTGAAAAATGTGATGAAGAACATCATGGCCGGG
CTGCAGCAGACCAATTCCGAGAAGATCCTGCTGTCTTGGGTGCGGCAGAGCACCCGCAACTATCC
CCAGGTGAACGTGATTAACTTCACTACATCCTGGAGCGACGGGCTGGCCCTGAATGCTCTGATTC
ACAGCCACAGGCCTGATCTGTTCGACTGGAATAGCGTGGTGTGCCAGCAGTCTGCCACACAGCGC
CTGGAACATGCCTTCAATATCGCTCGGTACCAGCTGGGGATCGAAAAACTGCTGGACCCAGAGGA
TGTGGACACTACATACCCAGATAAAAAGTCTATTCTGATGTACATTACTAGCCTGTTCCAGGTGC
TGCCACAGCAGGTGTCTATTGAAGCCATTCAGGAGGTGGAAATGCTGCCCCGCCCCCCCAAAGTG
ACTAAAGAGGAGCATTTTCAGCTGCATCATCAGATGCATTACAGCCAGCAGATTACCGTGAGCCT
GGCTCAGGGATATGAGCGCACCAGTAGTCCAAAACCACGGTTCAAGTCCTACGCTTATACCCAGG
CTGCCTACGTGACAACTAGCGACCCTACTAGATCCCCCTTTCCATCCCAGCACCTGGAGGCCCCA
GAGGACAAGAGCTTTGGGTCCAGCCTGATGGAAAGCGAGGTGAATCTGGATCGGTACCAGACAGC
CCTGGAGGAGGTGCTGAGCTGGCTGCTGAGTGCTGAAGACACACTGCAGGCCCAGGGCGAAATTT
CCAATGACGTGGAAGTGGTGAAGGATCAGTTCCACACACACGAGGGCTATATGATGGACCTGACA
GCTCACCAGGGGCGCGTGGGCAATATCCTGCAGCTGGGCTCTAAACTGATCGGCACCGGGAAACT
GAGTGAGGACGAGGAAACAGAAGTGCAGGAGCAGATGAACCTGCTGAACAGCCGCTGGGAGTGTC
TGAGAGTGGCTAGTATGGAGAAGCAGTCCAACCTGCACCGGGTGCTGATGGACCTGCAGAACCAG
AAACTGAAAGAGCTGAACGACTGGCTGACAAAGACTGAGGAACGCACAAGGAAGATGGAGGAGGA
GCCACTGGGACCCGACCTGGAGGATCTGAAGAGACAGGTGCAGCAGCATAAGGTGCTGCAGGAGG
ATCTGGAACAGGAGCAGGTGCGGGTGAACTCCCTGACACATATGGTGGTGGTGGTGGACGAATCT
AGTGGAGATCACGCCACCGCCGCCCTGGAGGAACAGCTGAAGGTGCTGGGGGACCGGTGGGCCAA
CATTTGCCGGTGGACCGAGGACAGGTGGGTGCTGCTGCAGGACATCCTGCTGAAATGGCAGAGGC
TGACCGAGGAGCAGTGTCTGTTTAGTGCTTGGCTGAGCGAGAAAGAGGACGCCGTGAACAAGATC
CACACAACCGGCTTTAAGGATCAGAACGAAATGCTGTCTAGCCTGCAGAAACTGGCTGTGCTGAA
GGCCGATCTGGAGAAAAAGAAGCAGAGCATGGGCAAACTGTATAGCCTGAAACAGGACCTGCTGA
GCACCCTGAAGAACAAGAGCGTGACCCAGAAGACAGAAGCCTGGCTGGATAACTTTGCCCGCTGC
TGGGACAACCTGGTGCAGAAACTGGAGAAAAGTACAGCTCAGATCTCTCAGGCTGTGACCACAAC
CCAGCCTAGCCTGACCCAGACAACCGTGATGGAAACCGTGACCACCGTGACAACCCGCGAACAGA
TCCTGGTGAAACATGCCCAGGAAGAGCTGCCACCTCCACCTCCCCAGAAGAAGAGAACCCTGGAG
CGGCTGCAGGAGCTGCAGGAAGCCACTGACGAACTGGACCTGAAGCTGAGGCAGGCCGAAGTGAT
TAAGGGGTCTTGGCAGCCTGTGGGCGATCTGCTGATTGATTCCCTGCAGGACCACCTGGAAAAGG
TGAAGGCTCTGAGAGGCGAAATTGCTCCACTGAAGGAGAACGTGAGTCATGTGAACGATCTGGCT
AGACAGCTGACAACACTGGGCATCCAGCTGAGCCCATACAATCTGAGCACACTGGAGGACCTGAA
TACCAGGTGGAAGCTGCTGCAGGTGGCTGTGGAAGACCGGGTGCGGCAGCTGCATGAGGCCCATC
GCGACTTCGGACCAGCCAGCCAGCACTTTCTGAGCACATCCGTGCAGGGGCCCTGGGAGAGGGCC
ATTTCTCCCAACAAGGTGCCCTACTATATTAATCACGAGACCCAGACCACTTGTTGGGACCATCC
CAAGATGACAGAACTGTACCAGTCCCTGGCCGATCTGAACAACGTGAGGTTTAGCGCTTACAGAA
CCGCTATGAAGCTGAGACGGCTGCAGAAGGCCCTGTGCCTGGATCTGCTGTCCCTGTCCGCCGCC
TGCGATGCCCTGGATCAGCATAATCTGAAGCAGAACGATCAGCCAATGGATATCCTGCAGATCAT
CAACTGCCTGACCACTATCTACGACAGGCTGGAGCAGGAGCACAACAACCTGGTGAACGTGCCTC
TGTGCGTGGATATGTGCCTGAACTGGCTGCTGAACGTGTATGACACTGGGCGCACCGGCCGGATC
AGAGTGCTGAGTTTTAAAACTGGGATTATCTCCCTGTGTAAGGCCCACCTGGAGGACAAGTACAG
GTACCTGTTCAAGCAGGTGGCTAGTAGCACTGGATTTTGTGACCAGCGCCGCCTGGGACTGCTGC
TGCATGATAGTATCCAGATTCCTAGACAGCTGGGAGAGGTGGCTAGTTTCGGAGGATCTAACATC
GAACCCAGCGTGCGCAGCTGTTTCCAGTTTGCCAATAACAAACCTGAAATCGAGGCTGCTCTGTT
CCTGGATTGGATGCGCCTGGAACCACAGAGCATGGTGTGGCTGCCTGTGCTGCACAGAGTGGCTG
CCGCCGAAACTGCCAAGCACCAGGCTAAATGCAACATCTGCAAGGAATGTCCCATTATCGGCTTT
CGCTACAGGAGTCTGAAACATTTTAACTACGATATTTGCCAGAGCTGCTTCTTTTCCGGAAGAGT
GGCCAAAGGACACAAGATGCACTACCCTATGGTGGAATATTGCACCCCAACTACATCTGGCGAAG
ATGTGCGCGATTTTGCCAAGGTGCTGAAGAATAAGTTTCGGACTAAGAGGTACTTCGCCAAGCAC
CCCCGCATGGGGTATCTGCCAGTGCAGACAGTGCTGGAAGGAGACAATATGGAGACCGATACAAT
GTGA (SEQ ID NO: 19)
TCTGAAATTAGGAAAAGGTTGGATGTTGATATAACTGAACTTCACAGCTGGATTACTCGCTCAGA
AGCTGTGTTGCAGAGTCCTGAATTTGCAATCTTTCGGAAGGAAGGCAACTTCTCAGACTTAAAAG
AAAAAGTCAATGCCATAGAGCGAGAAAAAGCTGAGAAGTTCAGAAAACTGCAAGATGCCAGCAGA
TCAGCTCAGGCCCTGGTGGAACAGATGGTGAATGAGGGTGTTAATGCAGATAGCATCAAACAAGC
CTCAGAACAACTGAACAGCCGGTGGATCGAATTCTGCCAGTTGCTAAGTGAGAGACTTAACTGGC
TGGAGTAT (SEQ ID NO: 33)
CAGAACAACATCATCGCTTTCTATAATCAGCTACAACAATTGGAGCAGATGACAACTACTGCTGA
AAACTGGTTGAAAATCCAACCCACCACCCCATCAGAGCCAACAGCAATTAAAAGTCAGTTAAAAA
TTTGTAAGGATGAAGTCAACCGGCTATCAGGTCTTCAACCTCAAATTGAACGATTAAAAATTCAA
AGCATAGCCCTGAAAGAGAAAGGACAAGGACCCATGTTCCTGGATGCAGACTTTGTGGCCTTTAC
AAATCATTTTAAGCAAGTCTTTTCTGATGTGCAGGCCAGAGAGAAAGAGCTACAGACAATTTTTG
AC (SEQ ID NO: 34)
ACTTTGCCACCAATGCGCTATCAGGAGACCATGAGTGCCATCAGGACATGGGTCCAGCAGTCAGA
AACCAAACTCTCCATACCTCAACTTAGTGTCACCGACTATGAAATCATGGAGCAGAGACTCGGGG
AATTGCAGGCTTTACAAAGTTCTCTGCAAGAGCAACAAAGTGGCCTATACTATCTCAGCACCACT
GTGAAAGAGATGTCGAAGAAAGCGCCCTCTGAAATTAGCCGGAAATATCAATCAGAATTTGAAGA
AATTGAGGGACGCTGGAAGAAGCTCTCCTCCCAGCTGGTTGAGCATTGTCAAAAGCTAGAGGAGC
AA (SEQ ID NO: 35)
ATGAATAAACTCCGAAAAATTCAGAATCACATACAAACCCTGAAGAAATGGATGGCTGAAGTTGA
TGTTTTTCTGAAGGAGGAATGGCCTGCCCTTGGGGATTCAGAAATTCTAAAAAAGCAGCTGAAAC
AGTGCAGACTTTTAGTCAGTGATATTCAGACAATTCAGCCCAGTCTAAACAGTGTCAATGAAGGT
GGGCAGAAGATAAAGAATGAAGCAGAGCCAGAGTTTGCTTCGAGACTTGAGACAGAACTCAAAGA
ACTTAACACTCAGTGGGATCACATGTGCCAACAGGTCTATGCCAGAAAGGAGGCCTTGAAGGGAG
GT (SEQ ID NO: 36)
TGGGCATGTTGGCATGAGTTATTGTCATACTTGGAGAAAGCAAACAAGTGGCTAAATGAAGTAGA
ATTTAAACTTAAAACCACTGAAAACATTCCTGGCGGAGCTGAGGAAATCTCTGAGGTGCTAGATT
CACTTGAAAATTTGATGCGACATTCAGAGGATAACCCAAATCAGATTCGCATATTGGCACAGACC
CTAACAGATGGCGGAGTCATGGATGAGCTAATCAATGAGGAACTTGAGACATTTAATTCTCGTTG
GAGGGAACTACATGAAGAGGCTGTAAGGAGGCAAAAGTTGCTTGAACAGAGC (SEQ ID NO:
37)
GGGATTCCCTCACTTTCCCCCTACAGGACTCAGATCTGGGAGGCAATTACCTTCGGAGAAAAACG
AATAGGAAAAACTGAAGTGTTACTTTTTTTAAAGCTGCTGAAGTTTGTTGGTTTCTCATTGTTTT
TAAGCCTACTGGAGCAATAAAGTTTGAAGAACTTTTACCAGGTTTTTTTTATCGCTGCCTTGATA
TACACTTTTCAAAATGCTTTGGTGGGAAGAAGTAGAGGACTGTTATGAAAGAGAAGATGTTCAAA
AGAAAACATTCACAAAATGGGTAAATGCACAATTTTCTAAGTTTGGGAAGCAGCATATTGAGAAC
CTCTTCAGTGACCTACAGGATGGGAGGCGCCTCCTAGACCTCCTCGAAGGCCTGACAGGGCAAAA
ACTGCCAAAAGAAAAAGGATCCACAAGAGTTCATGCCCTGAACAATGTCAACAAGGCACTGCGGG
TTTTGCAGAACAATAATGTTGATTTAGTGAATATTGGAAGTACTGACATCGTAGATGGAAATCAT
AAACTGACTCTTGGTTTGATTTGGAATATAATCCTCCACTGGCAGGTCAAAAATGTAATGAAAAA
TATCATGGCTGGATTGCAACAAACCAACAGTGAAAAGATTCTCCTGAGCTGGGTCCGACAATCAA
CTCGTAATTATCCACAGGTTAATGTAATCAACTTCACCACCAGCTGGTCTGATGGCCTGGCTTTG
AATGCTCTCATCCATAGTCATAGGCCAGACCTATTTGACTGGAATAGTGTGGTTTGCCAGCAGTC
AGCCACACAACGACTGGAACATGCATTCAACATCGCCAGATATCAATTAGGCATAGAGAAACTAC
TCGATCCTGAAGATGTTGATACCACCTATCCAGATAAGAAGTCCATCTTAATGTACATCACATCA
CTCTTCCAAGTTTTGCCTCAACAAGTGAGCATTGAAGCCATCCAGGAAGTGGAAATGTTGCCAAG
GCCACCTAAAGTGACTAAAGAAGAACATTTTCAGTTACATCATCAAATGCACTATTCTCAACAGA
TCACGGTCAGTCTAGCACAGGGATATGAGAGAACTTCTTCCCCTAAGCCTCGATTCAAGAGCTAT
GCCTACACACAGGCTGCTTATGTCACCACCTCTGACCCTACACGGAGCCCATTTCCTTCACAGCA
TTTGGAAGCTCCTGAAGACAAGTCATTTGGCAGTTCATTGATGGAGAGTGAAGTAAACCTGGACC
GTTATCAAACAGCTTTAGAAGAAGTATTATCGTGGCTTCTTTCTGCTGAGGACACATTGCAAGCA
CAAGGAGAGATTTCTAATGATGTGGAAGTGGTGAAAGACCAGTTTCATACTCATGAGGGGTACAT
GATGGATTTGACAGCCCATCAGGGCCGGGTTGGTAATATTCTACAATTGGGAAGTAAGCTGATTG
GAACAGGAAAATTATCAGAAGATGAAGAAACTGAAGTACAAGAGCAGATGAATCTCCTAAATTCA
AGATGGGAATGCCTCAGGGTAGCTAGCATGGAAAAACAAAGCAATTTACATAGAGTTTTAATGGA
TCTCCAGAATCAGAAACTGAAAGAGTTGAATGACTGGCTAACAAAAACAGAAGAAAGAACAAGGA
AAATGGAGGAAGAGCCTCTTGGACCTGATCTTGAAGACCTAAAACGCCAAGTACAACAACATAAG
GTGCTTCAAGAAGATCTAGAACAAGAACAAGTCAGGGTCAATTCTCTCACTCACATGGTGGTGGT
AGTTGATGAATCTAGTGGAGATCACGCAACTGCTGCTTTGGAAGAACAACTTAAGGTATTGGGAG
ATCGATGGGCAAACATCTGTAGATGGACAGAAGACCGCTGGGTTCTTTTACAAGACATCCTTCTC
AAATGGCAACGTCTTACTGAAGAACAGTGCCTTTTTAGTGCATGGCTTTCAGAAAAAGAAGATGC
AGTGAACAAGATTCACACAACTGGCTTTAAAGATCAAAATGAAATGTTATCAAGTCTTCAAAAAC
TGGCCGTTTTAAAAGCGGATCTAGAAAAGAAAAAGCAATCCATGGGCAAACTGTATTCACTCAAA
CAAGATCTTCTTTCAACACTGAAGAATAAGTCAGTGACCCAGAAGACGGAAGCATGGCTGGATAA
CTTTGCCCGGTGTTGGGATAATTTAGTCCAAAAACTTGAAAAGAGTACAGCACAGATTTCACAGG
CTGTCACCACCACTCAGCCATCACTAACACAGACAACTGTAATGGAAACAGTAACTACGGTGACC
ACAAGGGAACAGATCCTGGTAAAGCATGCTCAAGAGGAACTTCCACCACCACCTCCCCAAAAGAA
GAGGCAGATTACTGTGGATCTTGAAAGACTCCAGGAACTTCAAGAGGCCACGGATGAGCTGGACC
TCAAGCTGCGCCAAGCTGAGGTGATCAAGGGATCCTGGCAGCCCGTGGGCGATCTCCTCATTGAC
TCTCTCCAAGATCACCTCGAGAAAGTCAAGGCACTTCGAGGAGAAATTGCGCCTCTGAAAGAGAA
CGTGAGCCACGTCAATGACCTTGCTCGCCAGCTTACCACTTTGGGCATTCAGCTCTCACCGTATA
ACCTCAGCACTCTGGAAGACCTGAACACCAGATGGAAGCTTCTGCAGGTGGCCGTCGAGGACCGA
GTCAGGCAGCTGCATGAAGCCCACAGGGACTTTGGTCCAGCATCTCAGCACTTTCTTTCCACGTC
TGTCCAGGGTCCCTGGGAGAGAGCCATCTCGCCAAACAAAGTGCCCTACTATATCAACCACGAGA
CTCAAACAACTTGCTGGGACCATCCCAAAATGACAGAGCTCTACCAGTCTTTAGCTGACCTGAAT
AATGTCAGATTCTCAGCTTATAGGACTGCCATGAAACTCCGAAGACTGCAGAAGGCCCTTTGCTT
GGATCTCTTGAGCCTGTCAGCTGCATGTGATGCCTTGGACCAGCACAACCTCAAGCAAAATGACC
AGCCCATGGATATCCTGCAGATTATTAATTGTTTGACCACTATTTATGACCGCCTGGAGCAAGAG
CACAACAATTTGGTCAACGTCCCTCTCTGCGTGGATATGTGTCTGAACTGGCTGCTGAATGTTTA
TGATACGGGACGAACAGGGAGGATCCGTGTCCTGTCTTTTAAAACTGGCATCATTTCCCTGTGTA
AAGCACATTTGGAAGACAAGTACAGATACCTTTTCAAGCAAGTGGCAAGTTCAACAGGATTTTGT
GACCAGCGCAGGCTGGGCCTCCTTCTGCATGATTCTATCCAAATTCCAAGACAGTTGGGTGAAGT
TGCATCCTTTGGGGGCAGTAACATTGAGCCAAGTGTCCGGAGCTGCTTCCAATTTGCTAATAATA
AGCCAGAGATCGAAGCGGCCCTCTTCCTAGACTGGATGAGACTGGAACCCCAGTCCATGGTGTGG
CTGCCCGTCCTGCACAGAGTGGCTGCTGCAGAAACTGCCAAGCATCAGGCCAAATGTAACATCTG
CAAAGAGTGTCCAATCATTGGATTCAGGTACAGGAGTCTAAAGCACTTTAATTATGACATCTGCC
AAAGCTGCTTTTTTTCTGGTCGAGTTGCAAAAGGCCATAAAATGCACTATCCCATGGTGGAATAT
TGCACTCCGACTACATCAGGAGAAGATGTTCGAGACTTTGCCAAGGTACTAAAAAACAAATTTCG
AACCAAAAGGTATTTTGCGAAGCATCCCCGAATGGGCTACCTGCCAGTGCAGACTGTCTTAGAGG
GGGACAACATGGAAACGCCTGCCTCGTCCCCTCAGCTTTCACACGATGATACTCATTCACGCATT
GAACATTATGCTAGCAGGCTAGCAGAAATGGAAAACAGCAATGGATCTTATCTAAATGATAGCAT
CTCTCCTAATGAGAGCATAGATGATGAACATTTGTTAATCCAGCATTACTGCCAAAGTTTGAACC
AGGACTCCCCCCTGAGCCAGCCTCGTAGTCCTGCCCAGATCTTGATTTCCTTAGAGAGTGAGGAA
AGAGGGGAGCTAGAGAGAATCCTAGCAGATCTTGAGGAAGAAAACAGGAATCTGCAAGCAGAATA
TGACCGTCTAAAGCAGCAGCACGAACATAAAGGCCTGTCCCCACTGCCGTCCCCTCCTGAAATGA
TGCCCACCTCTCCCCAGAGTCCCCGGGATGCTGAGCTCATTGCTGAGGCCAAGCTACTGCGTCAA
CACAAAGGCCGCCTGGAAGCCAGGATGCAAATCCTGGAAGACCACAATAAACAGCTGGAGTCACA
GTTACACAGGCTAAGGCAGCTGCTGGAGCAACCCCAGGCAGAGGCCAAAGTGAATGGCACAACGG
TGTCCTCTCCTTCTACCTCTCTACAGAGGTCCGACAGCAGTCAGCCTATGCTGCTCCGAGTGGTT
GGCAGTCAAACTTCGGACTCCATGGGTGAGGAAGATCTTCTCAGTCCTCCCCAGGACACAAGCAC
AGGGTTAGAGGAGGTGATGGAGCAACTCAACAACTCCTTCCCTAGTTCAAGAGGAAGAAATACCC
CTGGAAAGCCAATGAGAGAGGACACAATGTAGGAAGTCTTTTCCACATGGCAGATGATTTGGGCA
GAGCGATGGAGTCCTTAGTATCAGTCATGACAGATGAAGAAGGAGCAGAATAAATGTTTTACAAC
TCCTGATTCCCGCATGGTTTTTATAATATTCATACAACAAAGAGGATTAGACAGTAAGAGTTTAC
AAGAAATAAATCTATATTTTTGTGAAGGGTAGTGGTATTATACTGTAGATTTCAGTAGTTTCTAA
GTCTGTTATTGTTTTGTTAACAATGGCAGGTTTTACACGTCTATGCAATTGTACAAAAAAGTTAT
AAGAAAACTACATGTAAAATCTTGATAGCTAAATAACTTGCCATTTCTTTATATGGAACGCATTT
TGGGTTGTTTAAAAATTTATAACAGTTATAAAGAAAGATTGTAAACTAAAGTGTGCTTTATAAAA
AAAAGTTGTTTATAAAAACCCCTAAAAACAAAACAAACACACACACACACACATACACACACACA
CACAAAACTTTGAGGCAGCGCATTGTTTTGCATCCTTTTGGCGTGATATCCATATGAAATTCATG
GCTTTTTCTTTTTTTGCATATTAAAGATAAGACTTCCTCTACCACCACACCAAATGACTACTACA
CACTGCTCATTTGAGAACTGTCAGCTGAGTGGGGCAGGCTTGAGTTTTCATTTCATATATCTATA
TGTCTATAAGTATATAAATACTATAGTTATATAGATAAAGAGATACGAATTTCTATAGACTGACT
TTTTCCATTTTTTAAATGTTCATGTCACATCCTAATAGAAAGAAATTACTTCTAGTCAGTCATCC
AGGCTTACCTGCTTGGTCTAGA (SEQ ID NO: 38)
GGGATTCCCTCACTTTCCCCCTACAGGACTCAGATCTGGGAGGCAATTACCTTCGGAGAAAAACG
AATAGGAAAAACTGAAGTGTTACTTTTTTTAAAGCTGCTGAAGTTTGTTGGTTTCTCATTGTTTT
TAAGCCTACTGGAGCAATAAAGTTTGAAGAACTTTTACCAGGTTTTTTTTATCGCTGCCTTGATA
TACACTTTTCAAAATGCTTTGGTGGGAAGAAGTAGAGGACTGTTATGAAAGAGAAGATGTTCAAA
AGAAAACATTCACAAAATGGGTAAATGCACAATTTTCTAAGTTTGGGAAGCAGCATATTGAGAAC
CTCTTCAGTGACCTACAGGATGGGAGGCGCCTCCTAGACCTCCTCGAAGGCCTGACAGGGCAAAA
ACTGCCAAAAGAAAAAGGATCCACAAGAGTTCATGCCCTGAACAATGTCAACAAGGCACTGCGGG
TTTTGCAGAACAATAATGTTGATTTAGTGAATATTGGAAGTACTGACATCGTAGATGGAAATCAT
AAACTGACTCTTGGTTTGATTTGGAATATAATCCTCCACTGGCAGGTCAAAAATGTAATGAAAAA
TATCATGGCTGGATTGCAACAAACCAACAGTGAAAAGATTCTCCTGAGCTGGGTCCGACAATCAA
CTCGTAATTATCCACAGGTTAATGTAATCAACTTCACCACCAGCTGGTCTGATGGCCTGGCTTTG
AATGCTCTCATCCATAGTCATAGGCCAGACCTATTTGACTGGAATAGTGTGGTTTGCCAGCAGTC
AGCCACACAACGACTGGAACATGCATTCAACATCGCCAGATATCAATTAGGCATAGAGAAACTAC
TCGATCCTGAAGATGTTGATACCACCTATCCAGATAAGAAGTCCATCTTAATGTACATCACATCA
CTCTTCCAAGTTTTGCCTCAACAAGTGAGCATTGAAGCCATCCAGGAAGTGGAAATGTTGCCAAG
GCCACCTAAAGTGACTAAAGAAGAACATTTTCAGTTACATCATCAAATGCACTATTCTCAACAGA
TCACGGTCAGTCTAGCACAGGGATATGAGAGAACTTCTTCCCCTAAGCCTCGATTCAAGAGCTAT
GCCTACACACAGGCTGCTTATGTCACCACCTCTGACCCTACACGGAGCCCATTTCCTTCACAGCA
TTTGGAAGCTCCTGAAGACAAGTCATTTGGCAGTTCATTGATGGAGAGTGAAGTAAACCTGGACC
GTTATCAAACAGCTTTAGAAGAAGTATTATCGTGGCTTCTTTCTGCTGAGGACACATTGCAAGCA
CAAGGAGAGATTTCTAATGATGTGGAAGTGGTGAAAGACCAGTTTCATACTCATGAGGGGTACAT
GATGGATTTGACAGCCCATCAGGGCCGGGTTGGTAATATTCTACAATTGGGAAGTAAGCTGATTG
GAACAGGAAAATTATCAGAAGATGAAGAAACTGAAGTACAAGAGCAGATGAATCTCCTAAATTCA
AGATGGGAATGCCTCAGGGTAGCTAGCATGGAAAAACAAAGCAATTTACATGCTCCTGGACTGAC
CACTATTGGAGCCTCTCCTACTCAGACTGTTACTCTGGTGACACAACCTGTGGTTACTAAGGAAA
CTGCCATCTCCAAACTAGAAATGCCATCTTCCTTGATGTTGGAGCATAGATTACTGCAACAGTTC
CCCCTGGACCTGGAAAAGTTTCTTGCCTGGCTTACAGAAGCTGAAACAACTGCCAATGTCCTACA
GGATGCTACCCGTAAGGAAAGGCTCCTAGAAGACTCCAAGGGAGTAAAAGAGCTGATGAAACAAT
GGCAAGACCTCCAAGGTGAAATTGAAGCTCACACAGATGTTTATCACAACCTGGATGAAAACAGC
CAAAAAATCCTGAGATCCCTGGAAGGTTCCGATGATGCAGTCCTGTTACAAAGACGTTTGGATAA
CATGAACTTCAAGTGGAGTGAACTTCGGAAAAAGTCTCTCAACATTAGGTCCCATTTGGAAGCCA
GTTCTGACCAGTGGAAGCGTCTGCACCTTTCTCTGCAGGAACTTCTGGTGTGGCTACAGCTGAAA
GATGATGAATTAAGCCGGCAGGCACCTATTGGAGGCGACTTTCCAGCAGTTCAGAAGCAGAACGA
TGTACATAGGGCCTTCAAGAGGGAATTGAAAACTAAAGAACCTGTAATCATGAGTACTCTTGAGA
CTGTACGAATATTTCTGACAGAGCAGCCTTTGGAAGGACTAGAGAAACTCTACCAGGAGCCCAGA
GAGCTGCCTCCTGAGGAGAGAGCCCAGAATGTCACTCGGCTTCTACGAAAGCAGGCTGAGGAGGT
CAATACTGAGTGGGAAAAATTGAACCTGCACTCCGCTGACTGGCAGAGAAAAATAGATGAGACCC
TTGAAAGACTCCAGGAACTTCAAGAGGCCACGGATGAGCTGGACCTCAAGCTGCGCCAAGCTGAG
GTGATCAAGGGATCCTGGCAGCCCGTGGGCGATCTCCTCATTGACTCTCTCCAAGATCACCTCGA
GAAAGTCAAGGCACTTCGAGGAGAAATTGCGCCTCTGAAAGAGAACGTGAGCCACGTCAATGACC
TTGCTCGCCAGCTTACCACTTTGGGCATTCAGCTCTCACCGTATAACCTCAGCACTCTGGAAGAC
CTGAACACCAGATGGAAGCTTCTGCAGGTGGCCGTCGAGGACCGAGTCAGGCAGCTGCATGAAGC
CCACAGGGACTTTGGTCCAGCATCTCAGCACTTTCTTTCCACGTCTGTCCAGGGTCCCTGGGAGA
GAGCCATCTCGCCAAACAAAGTGCCCTACTATATCAACCACGAGACTCAAACAACTTGCTGGGAC
CATCCCAAAATGACAGAGCTCTACCAGTCTTTAGCTGACCTGAATAATGTCAGATTCTCAGCTTA
TAGGACTGCCATGAAACTCCGAAGACTGCAGAAGGCCCTTTGCTTGGATCTCTTGAGCCTGTCAG
CTGCATGTGATGCCTTGGACCAGCACAACCTCAAGCAAAATGACCAGCCCATGGATATCCTGCAG
ATTATTAATTGTTTGACCACTATTTATGACCGCCTGGAGCAAGAGCACAACAATTTGGTCAACGT
CCCTCTCTGCGTGGATATGTGTCTGAACTGGCTGCTGAATGTTTATGATACGGGACGAACAGGGA
GGATCCGTGTCCTGTCTTTTAAAACTGGCATCATTTCCCTGTGTAAAGCACATTTGGAAGACAAG
TACAGATACCTTTTCAAGCAAGTGGCAAGTTCAACAGGATTTTGTGACCAGCGCAGGCTGGGCCT
CCTTCTGCATGATTCTATCCAAATTCCAAGACAGTTGGGTGAAGTTGCATCCTTTGGGGGCAGTA
ACATTGAGCCAAGTGTCCGGAGCTGCTTCCAATTTGCTAATAATAAGCCAGAGATCGAAGCGGCC
CTCTTCCTAGACTGGATGAGACTGGAACCCCAGTCCATGGTGTGGCTGCCCGTCCTGCACAGAGT
GGCTGCTGCAGAAACTGCCAAGCATCAGGCCAAATGTAACATCTGCAAAGAGTGTCCAATCATTG
GATTCAGGTACAGGAGTCTAAAGCACTTTAATTATGACATCTGCCAAAGCTGCTTTTTTTCTGGT
CGAGTTGCAAAAGGCCATAAAATGCACTATCCCATGGTGGAATATTGCACTCCGACTACATCAGG
AGAAGATGTTCGAGACTTTGCCAAGGTACTAAAAAACAAATTTCGAACCAAAAGGTATTTTGCGA
AGCATCCCCGAATGGGCTACCTGCCAGTGCAGACTGTCTTAGAGGGGGACAACATGGAAACGCCT
GCCTCGTCCCCTCAGCTTTCACACGATGATACTCATTCACGCATTGAACATTATGCTAGCAGGCT
AGCAGAAATGGAAAACAGCAATGGATCTTATCTAAATGATAGCATCTCTCCTAATGAGAGCATAG
ATGATGAACATTTGTTAATCCAGCATTACTGCCAAAGTTTGAACCAGGACTCCCCCCTGAGCCAG
CCTCGTAGTCCTGCCCAGATCTTGATTTCCTTAGAGAGTGAGGAAAGAGGGGAGCTAGAGAGAAT
CCTAGCAGATCTTGAGGAAGAAAACAGGAATCTGCAAGCAGAATATGACCGTCTAAAGCAGCAGC
ACGAACATAAAGGCCTGTCCCCACTGCCGTCCCCTCCTGAAATGATGCCCACCTCTCCCCAGAGT
CCCCGGGATGCTGAGCTCATTGCTGAGGCCAAGCTACTGCGTCAACACAAAGGCCGCCTGGAAGC
CAGGATGCAAATCCTGGAAGACCACAATAAACAGCTGGAGTCACAGTTACACAGGCTAAGGCAGC
TGCTGGAGCAACCCCAGGCAGAGGCCAAAGTGAATGGCACAACGGTGTCCTCTCCTTCTACCTCT
CTACAGAGGTCCGACAGCAGTCAGCCTATGCTGCTCCGAGTGGTTGGCAGTCAAACTTCGGACTC
CATGGGTGAGGAAGATCTTCTCAGTCCTCCCCAGGACACAAGCACAGGGTTAGAGGAGGTGATGG
AGCAACTCAACAACTCCTTCCCTAGTTCAAGAGGAAGAAATACCCCTGGAAAGCCAATGAGAGAG
GACACAATGTAGGAAGTCTTTTCCACATGGCAGATGATTTGGGCAGAGCGATGGAGTCCTTAGTA
TCAGTCATGACAGATGAAGAAGGAGCAGAATAAATGTTTTACAACTCCTGATTCCCGCATGGTTT
TTATAATATTCATACAACAAAGAGGATTAGACAGTAAGAGTTTACAAGAAATAAATCTATATTTT
TGTGAAGGGTAGTGGTATTATACTGTAGATTTCAGTAGTTTCTAAGTCTGTTATTGTTTTGTTAA
CAATGGCAGGTTTTACACGTCTATGCAATTGTACAAAAAAGTTATAAGAAAACTACATGTAAAAT
CTTGATAGCTAAATAACTTGCCATTTCTTTATATGGAACGCATTTTGGGTTGTTTAAAAATTTAT
AACAGTTATAAAGAAAGATTGTAAACTAAAGTGTGCTTTATAAAAAAAAGTTGTTTATAAAAACC
CCTAAAAACAAAACAAACACACACACACACACATACACACACACACACAAAACTTTGAGGCAGCG
CATTGTTTTGCATCCTTTTGGCGTGATATCCATATGAAATTCATGGCTTTTTCTTTTTTTGCATA
TTAAAGATAAGACTTCCTCTACCACCACACCAAATGACTACTACACACTGCTCATTTGAGAACTG
TCAGCTGAGTGGGGCAGGCTTGAGTTTTCATTTCATATATCTATATGTCTATAAGTATATAAATA
CTATAGTTATATAGATAAAGAGATACGAATTTCTATAGACTGACTTTTTCCATTTTTTAAATGTT
CATGTCACATCCTAATAGAAAGAAATTACTTCTAGTCAGTCATCCAGGCTTACCTGCTTGGTCTA
GA (SEQ ID NO: 39)
MLWWEEVEDCYEREDVQKKTFTKWVNAQFSKFGKQHIENLFSDLQDGRRLLDLLEGLTGQKLPKE
KGSTRVHALNNVNKALRVLQNNNVDLVNIGSTDIVDGNHKLTLGLIWNIILHWQVKNVMKNIMAG
LQQTNSEKILLSWVRQSTRNYPQVNVINFTTSWSDGLALNALIHSHRPDLFDWNSVVCQQSATQR
LEHAFNIARYQLGIEKLLDPEDVDTTYPDKKSILMYITSLFQVLPQQVSIEAIQEVEMLPRPPKV
TKEEHFQLHHQMHYSQQITVSLAQGYERTSSPKPRFKSYAYTQAAYVTTSDPTRSPFPSQHLEAP
EDKSFGSSLMESEVNLDRYQTALEEVLSWLLSAEDTLQAQGEISNDVEVVKDQFHTHEGYMMDLT
AHQGRVGNILQLGSKLIGTGKLSEDEETEVQEQMNLLNSRWECLRVASMEKQSNLHSYVPSTYLT
EITHVSQALLEVEQLLNAPDLCAKDFEDLFKQEESLKNIKDSLQQSSGRIDIIHSKKTAALQSAT
PVERVKLQEALSQLDFQWEKVNKMYKDRQGRFDRSVEKWRRFHYDIKIFNQWLTEAEQFLRKTQI
PENWEHAKYKWYLKELQDGIGQRQTVVRTLNATGEEIIQQSSKTDASILQEKLGSLNLRWQEVCK
QLSDRKKRLEEQSDQWKRLHLSLQELLVWLQLKDDELSRQAPIGGDFPAVQKQNDVHRAFKRELK
TKEPVIMSTLETVRIFLTEQPLEGLEKLYQEPRELPPEERAQNVTRLLRKQAEEVNTEWEKLNLH
SADWQRKIDETLERLQELQEATDELDLKLRQAEVIKGSWQPVGDLLIDSLQDHLEKVKALRGEIA
PLKENVSHVNDLARQLTTLGIQLSPYNLSTLEDLNTRWKLLQVAVEDRVRQLHEAHRDFGPASQH
FLSTSVQGPWERAISPNKVPYYINHETQTTCWDHPKMTELYQSLADLNNVRFSAYRTAMKLRRLQ
KALCLDLLSLSAACDALDQHNLKQNDQPMDILQIINCLTTIYDRLEQEHNNLVNVPLCVDMCLNW
LLNVYDTGRTGRIRVLSFKTGIISLCKAHLEDKYRYLFKQVASSTGFCDQRRLGLLLHDSIQIPR
QLGEVASFGGSNIEPSVRSCFQFANNKPEIEAALFLDWMRLEPQSMVWLPVLHRVAAAETAKHQA
KCNICKECPIIGFRYRSLKHFNYDICQSCFFSGRVAKGHKMHYPMVEYCTPTTSGEDVRDFAKVL
KNKFRTKRYFAKHPRMGYLPVQTVLEGDNMETDTM (SEQ ID NO: 40)
MLWWEEVEDCYEREDVQKKTFTKWVNAQFSKFGKQHIENLFSDLQDGRRLLDLLEGLTGQKLPKE
KGSTRVHALNNVNKALRVLQNNNVDLVNIGSTDIVDGNHKLTLGLIWNIILHWQVKNVMKNIMAG
LQQTNSEKILLSWVRQSTRNYPQVNVINFTTSWSDGLALNALIHSHRPDLFDWNSVVCQQSATQR
LEHAFNIARYQLGIEKLLDPEDVDTTYPDKKSILMYITSLFQVLPQQVSIEAIQEVEMLPRPPKV
TKEEHFQLHHQMHYSQQITVSLAQGYERTSSPKPRFKSYAYTQAAYVTTSDPTRSPFPSQHLEAP
EDKSFGSSLMESEVNLDRYQTALEEVLSWLLSAEDTLQAQGEISNDVEVVKDQFHTHEGYMMDLT
AHQGRVGNILQLGSKLIGTGKLSEDEETEVQEQMNLLNSRWECLRVASMEKQSNLHRVLMDLQNQ
KLKELNDWLTKTEERTRKMEEEPLGPDLEDLKRQVQQHKVLQEDLEQEQVRVNSLTHMVVVVDES
SGDHATAALEEQLKVLGDRWANICRWTEDRWVLLQDQPDLAPGLTTIGASPTQTVTLVTQPVVTK
ETAISKLEMPSSLMLEVPTHRLLQQFPLDLEKFLAWLTEAETTANVLQDATRKERLLEDSKGVKE
LMKQWQDLQGEIEAHTDVYHNLDENSQKILRSLEGSDDAVLLQRRLDNMNFKWSELRKKSLNIRS
HLEASSDQWKRLHLSLQELLVWLQLKDDELSRQAPIGGDFPAVQKQNDVHRAFKRELKTKEPVIM
STLETVRIFLTEQPLEGLEKLYQEPRELPPEERAQNVTRLLRKQAEEVNTEWEKLNLHSADWQRK
IDETLERLQELQEATDELDLKLRQAEVIKGSWQPVGDLLIDSLQDHLEKVKALRGEIAPLKENVS
HVNDLARQLTTLGIQLSPYNLSTLEDLNTRWKLLQVAVEDRVRQLHEAHRDFGPASQHFLSTSVQ
GPWERAISPNKVPYYINHETQTTCWDHPKMTELYQSLADLNNVRFSAYRTAMKLRRLQKALCLDL
LSLSAACDALDQHNLKQNDQPMDILQIINCLTTIYDRLEQEHNNLVNVPLCVDMCLNWLLNVYDT
GRTGRIRVLSFKTGIISLCKAHLEDKYRYLFKQVASSTGFCDQRRLGLLLHDSIQIPRQLGEVAS
FGGSNIEPSVRSCFQFANNKPEIEAALFLDWMRLEPQSMVWLPVLHRVAAAETAKHQAKCNICKE
CPIIGFRYRSLKHFNYDICQSCFFSGRVAKGHKMHYPMVEYCTPTTSGEDVRDFAKVLKNKFRTK
RYFAKHPRMGYLPVQTVLEGDNMET (SEQ ID NO: 41)
MLWWEEVEDCYEREDVQKKTFTKWVNAQFSKFGKQHIENLFSDLQDGRRLLDLLEGLTGQKLPKE
KGSTRVHALNNVNKALRVLQNNNVDLVNIGSTDIVDGNHKLTLGLIWNIILHWQVKNVMKNIMAG
LQQTNSEKILLSWVRQSTRNYPQVNVINFTTSWSDGLALNALIHSHRPDLFDWNSVVCQQSATQR
LEHAFNIARYQLGIEKLLDPEDVDTTYPDKKSILMYITSLFQVLPQQVSIEAIQEVEMLPRPPKV
TKEEHFQLHHQMHYSQQITVSLAQGYERTSSPKPRFKSYAYTQAAYVTTSDPTRSPFPSQHLEAP
EDKSFGSSLMESEVNLDRYQTALEEVLSWLLSAEDTLQAQGEISNDVEVVKDQFHTHEGYMMDLT
AHQGRVGNILQLGSKLIGTGKLSEDEETEVQEQMNLLNSRWECLRVASMEKQSNLHRVLMDLQNQ
KLKELNDWLTKTEERTRKMEEEPLGPDLEDLKRQVQQHKVLQEDLEQEQVRVNSLTHMVVVVDES
SGDHATAALEEQLKVLGDRWANICRWTEDRWVLLQDTHRLLQQFPLDLEKFLAWLTEAETTANVL
QDATRKERLLEDSKGVKELMKQWQDLQGEIEAHTDVYHNLDENSQKILRSLEGSDDAVLLQRRLD
NMNFKWSELRKKSLNIRSHLEASSDQWKRLHLSLQELLVWLQLKDDELSRQAPIGGDFPAVQKQN
DVHRAFKRELKTKEPVIMSTLETVRIFLTEQPLEGLEKLYQEPRELPPEERAQNVTRLLRKQAEE
VNTEWEKLNLHSADWQRKIDETLERLQELQEATDELDLKLRQAEVIKGSWQPVGDLLIDSLQDHL
EKVKALRGEIAPLKENVSHVNDLARQLTTLGIQLSPYNLSTLEDLNTRWKLLQVAVEDRVRQLHE
AHRDFGPASQHFLSTSVQGPWERAISPNKVPYYINHETQTTCWDHPKMTELYQSLADLNNVRFSA
YRTAMKLRRLQKALCLDLLSLSAACDALDQHNLKQNDQPMDILQIINCLTTIYDRLEQEHNNLVN
VPLCVDMCLNWLLNVYDTGRTGRIRVLSFKTGIISLCKAHLEDKYRYLFKQVASSTGFCDQRRLG
LLLHDSIQIPRQLGEVASFGGSNIEPSVRSCFQFANNKPEIEAALFLDWMRLEPQSMVWLPVLHR
VAAAETAKHQAKCNICKECPIIGFRYRSLKHFNYDICQSCFFSGRVAKGHKMHYPMVEYCTPTTS
GEDVRDFAKVLKNKFRTKRYFAKHPRMGYLPVQTVLEGDNMET (SEQ ID NO: 42)
MLWWEEVEDCYEREDVQKKTFTKWVNAQFSKFGKQHIENLFSDLQDGRRLLDLLEGLTGQKLPKE
KGSTRVHALNNVNKALRVLQNNNVDLVNIGSTDIVDGNHKLTLGLIWNIILHWQVKNVMKNIMAG
LQQTNSEKILLSWVRQSTRNYPQVNVINFTTSWSDGLALNALIHSHRPDLFDWNSVVCQQSATQR
LEHAFNIARYQLGIEKLLDPEDVDTTYPDKKSILMYITSLFQVLPQQVSIEAIQEVEMLPRPPKV
TKEEHFQLHHQMHYSQQITVSLAQGYERTSSPKPRFKSYAYTQAAYVTTSDPTRSPFPSQHLEAP
EDKSFGSSLMESEVNLDRYQTALEEVLSWLLSAEDTLQAQGEISNDVEVVKDQFHTHEGYMMDLT
AHQGRVGNILQLGSKLIGTGKLSEDEETEVQEQMNLLNSRWECLRVASMEKQSNLHRVLMDLQNQ
KLKELNDWLTKTEERTRKMEEEPLGPDLEDLKRQVQQHKVLQEDLEQEQVRVNSLTHMVVVVDES
SGDHATAALEEQLKVLGDRWANICRWTEDRWVLLQDQPDLAPGLTTIGASPTQTVTLVTQPVVTK
ETAISKLEMPSSLMLEVPTHRLLQQFPLDLEKFLAWLTEAETTANVLQDATRKERLLEDSKGVKE
LMKQWQDLQGEIEAHTDVYHNLDENSQKILRSLEGSDDAVLLQRRLDNMNFKWSELRKKSLNIRS
HLEASSDQWKRLHLSLQELLVWLQLKDDELSRQAPIGGDFPAVQKQNDVHRAFKRELKTKEPVIM
STLETVRIFLTEQPLEGLEKLYQEPRELPPEERAQNVTRLLRKQAEEVNTEWEKLNLHSADWQRK
IDETLERLQELQEATDELDLKLRQAEVIKGSWQPVGDLLIDSLQDHLEKVKALRGEIAPLKENVS
HVNDLARQLTTLGIQLSPYNLSTLEDLNTRWKLLQVAVEDRVRQLHEAHRDFGPASQHELSTSVQ
GPWERAISPNKVPYYINHETQTTCWDHPKMTELYQSLADLNNVRFSAYRTAMKLRRLQKALCLDL
LSLSAACDALDQHNLKQNDQPMDILQIINCLTTIYDRLEQEHNNLVNVPLCVDMCLNWLLNVYDT
GRTGRIRVLSFKTGIISLCKAHLEDKYRYLFKQVASSTGFCDQRRLGLLLHDSIQIPRQLGEVAS
FGGSNIEPSVRSCFQFANNKPEIEAALFLDWMRLEPQSMVWLPVLHRVAAAETAKHQAKCNICKE
CPIIGFRYRSLKHFNYDICQSCFFSGRVAKGHKMHYPMVEYCTPTTSGEDVRDFAKVLKNKFRTK
RYFAKHPRMGYLPVQTVLEGDNMET (SEQ ID NO: 43)
MLWWEEVEDCYEREDVQKKTFTKWVNAQFSKFGKQHIENLFSDLQDGRRLLDLLEGLTGQKLPKE
KGSTRVHALNNVNKALRVLQNNNVDLVNIGSTDIVDGNHKLTLGLIWNIILHWQVKNVMKNIMAG
LQQTNSEKILLSWVRQSTRNYPQVNVINFTTSWSDGLALNALIHSHRPDLFDWNSVVCQQSATQR
LEHAFNIARYQLGIEKLLDPEDVDTTYPDKKSILMYITSLFQVLPQQVSIEAIQEVEMLPRPPKV
TKEEHFQLHHQMHYSQQITVSLAQGYERTSSPKPRFKSYAYTQAAYVTTSDPTRSPFPSQHLEAP
EDKSFGSSLMESEVNLDRYQTALEEVLSWLLSAEDTLQAQGEISNDVEVVKDQFHTHEGYMMDLT
AHQGRVGNILQLGSKLIGTGKLSEDEETEVQEQMNLLNSRWECLRVASMEKQSNLHRVLMDLQNQ
KLKELNDWLTKTEERTRKMEEEPLGPDLEDLKRQVQQHKVLQEDLEQEQVRVNSLTHMVVVVDES
SGDHATAALEEQLKVLGDRWANICRWTEDRWVLLQDILLKWQRLTEEQCLFSAWLSEKEDAVNKI
HTTGFKDQNEMLSSLQKLAVLKADLEKKKQSMGKLYSLKQDLLSTLKNKSVTQKTEAWLDNFARC
WDNLVQKLEKSTAQISQAVTTTQPSLTQTTVMETVTTVTTREQILVKHAQEELPPPPPQKKRTLE
RLQELQEATDELDLKLRQAEVIKGSWQPVGDLLIDSLQDHLEKVKALRGEIAPLKENVSHVNDLA
RQLTTLGIQLSPYNLSTLEDLNTRWKLLQVAVEDRVRQLHEAHRDFGPASQHELSTSVQGPWERA
ISPNKVPYYINHETQTTCWDHPKMTELYQSLADLNNVRFSAYRTAMKLRRLQKALCLDLLSLSAA
CDALDQHNLKQNDQPMDILQIINCLTTIYDRLEQEHNNLVNVPLCVDMCLNWLLNVYDTGRTGRI
RVLSFKTGIISLCKAHLEDKYRYLFKQVASSTGFCDQRRLGLLLHDSIQIPRQLGEVASFGGSNI
EPSVRSCFQFANNKPEIEAALFLDWMRLEPQSMVWLPVLHRVAAAETAKHQAKCNICKECPIIGF
RYRSLKHFNYDICQSCFFSGRVAKGHKMHYPMVEYCTPTTSGEDVRDFAKVLKNKFRTKRYFAKH
PRMGYLPVQTVLEGDNMETDTM (SEQ ID NO: 44)

As used here, the term “beta sarcoglycan,” “b-sarcoglycan,” “β-sarcoglcan,” or “SGCB” refers to a subcomplex of the dystrophin-glycoprotein complex which forms a link between the F-actin cytoskeleton and the extracellular matrix. Beta sarcoglycan is highly expressed in the heart and skeletal muscles, and expressed at a lesser extent in the brain, kidneys, placenta, the lungs. The canonical amino acid sequence of beta sarcoglycan is shown in Table 3 as SEQ ID NO: 45 (Uniprot Q16585). In some aspects, a recombinant AAVrh74 viral vector of the present disclosure comprises a genetic cassette comprising a nucleic acid molecule encoding a beta sarcoglycan. Nonlimiting examples of beta sarcoglycan sequences and constructs useful in the present disclosure are disclosed in International Publication No. WO2017180976, which is incorporated by reference herein in its entirety. In some aspects, the nucleic acid molecule encoding a beta sarcoglycan comprises the nucleic acid sequence set forth in SEQ ID NO: 21 (Table 3).

TABLE 3
Beta Sarcoglycan Sequences

MAAAAAAAAEQQSSNGPVKKSMREKAVERRSVNKEHNSNFKAGYIPIDEDRLHKTGLRGRKGN
LAICVIILLFILAVINLIITLVIWAVIRIGPNGCDSMEFHESGLLRFKQVSDMGVIHPLYKST
VGGRRNENLVITGNNQPIVFQQGTTKLSVENNKTSITSDIGMQFFDPRTQNILFSTDYETHEF
HLPSGVKSLNVQKASTERITSNATSDLNIKVDGRAIVRGNEGVFIMGKTIEFHMGGNMELKAE
NSIILNGSVMVSTTRLPSSSSGDQLGSGDWVRYKLCMCADGTLFKVQVTSONMGCQISDNPCG
NTH (SEQ ID NO: 45)
ATGGCAGCAGCAGCCGCCGCAGCCGCCGAGCAGCAGTCAAGCAATGGACCAGTGAAAAAATCA
ATGAGAGAAAAAGCCGTCGAGAGGAGATCAGTGAATAAGGAGCACAACAGCAATTTCAAAGCC
GGCTACATCCCTATTGACGAAGATCGCCTGCATAAGACAGGCCTGAGGGGGCGCAAAGGAAAC
CTGGCAATCTGCGTCATCATTCTGCTGTTTATCCTGGCCGTGATTAATCTGATCATTACTCTG
GTGATTTGGGCTGTCATCCGCATTGGCCCAAACGGGTGTGACTCTATGGAGTTCCACGAAAGT
GGCCTGCTGCGATTTAAGCAGGTGTCCGATATGGGGGTCATCCATCCACTGTACAAATCTACT
GTCGGCGGGCGGAGAAACGAGAATCTGGTGATCACCGGGAACAATCAGCCCATTGTGTTCCAG
CAGGGAACCACAAAGCTGTCTGTGGAAAACAATAAAACATCAATCACTAGCGACATTGGCATG
CAGTTCTTTGATCCCCGGACCCAGAATATCCTGTTCAGTACCGACTATGAGACACACGAATTT
CATCTGCCTTCCGGGGTGAAGTCTCTGAACGTCCAGAAAGCCAGCACTGAGAGAATCACCAGT
AACGCTACATCAGACCTGAATATCAAGGTGGATGGACGAGCTATTGTCCGGGGAAATGAGGGC
GTGTTCATCATGGGCAAGACAATTGAATTTCACATGGGAGGCAACATGGAGCTGAAAGCAGAA
AACAGCATCATTCTGAATGGGAGCGTGATGGTCTCCACTACCAGACTGCCCAGCTCCTCTAGT
GGAGACCAGCTGGGGTCCGGAGATTGGGTCAGGTATAAGCTGTGCATGTGTGCCGATGGCACC
CTGTTTAAAGTGCAGGTCACCAGCCAGAATATGGGATGTCAGATTAGCGATAACCCTTGTGGG
AATACTCATTAA (SEQ ID NO: 21)

As used here, the term “alpha sarcoglycan,” “a-sarcoglycan,” “a-sarcoglcan,” or “SGCA” refers to a subcomplex of the dystrophin-glycoprotein complex which forms a link between the F-actin cytoskeleton and the extracellular matrix. Alpha sarcoglycan is highly expressed in skeletal muscle, and expressed at a lesser extent in cardiac muscle, and, to a much lesser extent, in the lungs. The canonical amino acid sequence of alpha sarcoglycan is shown in Table 4 as SEQ ID NO: 46 (Uniprot Q16586). In some aspects, a recombinant AAVrh74 viral vector of the present disclosure comprises a genetic cassette comprising a nucleic acid molecule encoding a alpha sarcoglycan. Nonlimiting examples of alpha sarcoglycan sequences and constructs useful in the present disclosure are disclosed in International Publication No. WO2013078316, which is incorporated by reference herein in its entirety. In some aspects, the nucleic acid molecule encoding a alpha sarcoglycan comprises the nucleic acid sequence set forth in SEQ ID NO: 23 (Table 4).

TABLE 4
Alpha Sarcoglycan Sequences

MAETLFWTPLLVVLLAGLGDTEAQQTTLHPLVGRVFVHTLDHETFLSLPEHVAVPPAVHITYH
AHLQGHPDLPRWLRYTQRSPHHPGFLYGSATPEDRGLQVIEVTAYNRDSFDTTRORLVLEIGD
PEGPLLPYQAEFLVRSHDAEEVLPSTPASRFLSALGGLWEPGELQLLNVTSALDRGGRVPLPI
EGRKEGVYIKVGSASPFSTCLKMVASPDSHARCAQGQPPLLSCYDTLAPHFRVDWCNVTLVDK
SVPEPADEVPTPGDGILEHDPFFCPPTEAPDRDFLVDALVTLLVPLLVALLLTLLLAYVMCCR
REGRLKRDLATSDIQMVHHCTIHGNTEELRQMAASREVPRPLSTLPMFNVHTGERLPPRVDSA
QVPLILDQH (SEQ ID NO: 46)
ATGGCCGAGACACTGTTCTGGACTCCTCTGCTGGTGGTGCTGCTGGCTGGACTGGGAGATACC
GAGGCTCAGCAGACCACACTGCACCCACTGGTGGGCCGGGTGTTCGTGCACACCCTGGACCAT
GAGACATTTCTGAGTCTGCCAGAACACGTGGCTGTGCCACCTGCTGTGCATATCACTTACCAC
GCCCATCTGCAGGGCCATCCTGATCTGCCACGGTGGCTGAGATACACCCAGAGATCACCCCAC
CATCCTGGATTCCTGTATGGAAGCGCTACCCCAGAGGACAGGGGACTGCAGGTGATCGAAGTG
ACAGCTTACAACCGCGACAGTTTTGATACTACCAGGCAGCGCCTGGTGCTGGAGATTGGGGAT
CCAGAAGGACCCCTGCTGCCTTATCAGGCCGAGTTCCTGGTGCGGTCACACGACGCTGAGGAA
GTGCTGCCATCAACACCCGCCAGCAGATTTCTGTCCGCTCTGGGAGGACTGTGGGAGCCAGGA
GAACTGCAGCTGCTGAATGTGACTAGCGCTCTGGATAGGGGAGGAAGGGTGCCACTGCCAATC
GAGGGAAGGAAGGAAGGGGTGTACATTAAAGTGGGAAGCGCTTCCCCATTCTCCACCTGCCTG
AAGATGGTGGCTTCTCCTGATAGTCACGCTAGGTGCGCTCAGGGACAGCCACCACTGCTGTCC
TGTTATGACACACTGGCCCCCCATTTTCGCGTGGACTGGTGCAACGTGACTCTGGTGGATAAA
TCTGTGCCTGAGCCAGCTGACGAAGTGCCAACCCCTGGAGACGGAATCCTGGAGCACGATCCT
TTCTTTTGTCCTCCAACAGAAGCCCCAGACAGGGATTTCCTGGTGGACGCTCTGGTGACTCTG
CTGGTGCCTCTGCTGGTGGCTCTGCTGCTGACCCTGCTGCTGGCTTATGTGATGTGCTGTCGG
AGAGAGGGACGGCTGAAGAGAGACCTGGCCACATCTGATATCCAGATGGTGCACCATTGTACT
ATTCACGGCAACACCGAGGAACTGCGCCAGATGGCTGCTTCTAGGGAGGTGCCAAGGCCACTG
AGTACACTGCCTATGTTTAATGTGCACACTGGCGAACGGCTGCCCCCTAGAGTGGATAGCGCC
CAGGTGCCACTGATTCTGGACCAGCATTGA (SEQ ID NO: 23)

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced recombinant technology, but is not necessarily translated from a designated nucleic acid sequence. It can be generated in any manner, including by chemical synthesis.

The term “percent identity” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case can be, as determined by the match between strings of such sequences. “Identity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity are codified in publicly available computer programs. Sequence alignments and percent identity calculations can be performed using sequence analysis software such as the Megalign program of the LASERGENE bioinformatics computing suite (DNASTAR Inc., Madison, WI), the GCG suite of programs (Wisconsin Package Version 9.0, Genetics Computer Group (GCG), Madison, WI), BLASTP, BLASTN, BLASTX (Altschul et al., J. Mol. Biol. 215:403 (1990)), and DNASTAR (DNASTAR, Inc. 1228 S. Park St. Madison, WI 53715 USA). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized. For the purposes of determining percent identity between therapeutic protein, e.g., a dystrophin, sequence of the disclosure and a reference sequence, only nucleotides in the reference sequence corresponding to nucleotides in the therapeutic protein, e.g., the dystrophin, sequence of the disclosure are used to calculate percent identity.

As used herein, nucleotides corresponding to nucleotides in a particular sequence of the disclosure are identified by alignment of the sequence of the disclosure to maximize the identity to a reference sequence. The number used to identify an equivalent amino acid in a reference sequence is based on the number used to identify the corresponding amino acid in the sequence of the disclosure.

The term “linked” as used herein refers to a first amino acid sequence or nucleotide sequence covalently or non-covalently joined to a second amino acid sequence or nucleotide sequence, respectively. The first amino acid or nucleotide sequence can be directly joined or juxtaposed to the second amino acid or nucleotide sequence or alternatively an intervening sequence can covalently join the first sequence to the second sequence. The term “linked” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In one aspect, the first amino acid sequence can be linked to a second amino acid sequence by a peptide bond or a linker. The first nucleotide sequence can be linked to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The term “linked” is also indicated by a hyphen (-).

The term “Duchene muscular dystrophy” or “DMD,” as used herein, refers to a class of muscular dystrophy that is caused by mutations in the DMD gene, leading to reductions in mRNA and the absence of dystrophin, a 427 kD sarcolemmal protein associated with the dystrophin-associated protein complex (DAPC) (Hoffman et al., Cell 51(6):919-28, 1987). The DAPC is composed of multiple proteins at the muscle sarcolemma that form a structural link between the extra-cellular matrix (ECM) and the cytoskeleton via dystrophin, an actin binding protein, and alpha-dystroglycan, a laminin-binding protein. These structural links act to stabilize the muscle cell membrane during contraction and protect against contraction-induced damage. With dystrophin loss, membrane fragility results in sarcolemmal tears and an influx of calcium, triggering calcium-activated proteases and segmental fiber necrosis (Straub et al., Curr Opin. Neurol. 10(2): 168-75, 1997). This uncontrolled cycle of muscle degeneration and regeneration ultimately exhausts the muscle stem cell population (Sacco et al., Cell, 2010. 143(7): p. 1059-71; Wallace et al., Annu Rev Physiol, 2009. 71: p. 37-57), resulting in progressive muscle weakness, endomysial inflammation, and fibrotic scarring. En estimated 1 in 5000 newborn males is afflicted with DMD.

The term “limb girdle muscular dsytrophy” or “LGMD” as used herein, refers to a class of muscular dystrophy that usually manifests in the proximal muscles around the hips and shoulders. LGMD is the fourth most common genetic cause of muscle weakness with an estimated prevalence in about 2 in every 100,000 individuals. The unifying features of the LGMDs are the weakness and atrophy of the limb-girdle muscles. However, the age at which symptoms appear, and the speed and severity of disease progression, can vary. There are at least nineteen forms of LGMD, and the forms are classified by their associated genetic defects: LGMD1A (Autosomal dominant; Myotilin gene); LGMD1B (Autosomal dominant; Lamin A/C gene); LGMD1C (Autosomal dominant; Caveolin gene); LGMD1D (Autosomal dominant; Chromosome 7); LGMD1E (Autosomal dominant; Chromosome 6); LGMD1F (Autosomal dominant; Chromosome 7); LGMD1G (Autosomal dominant; Chromosome 4); LGMD2A (Autosomal recessive; Calpain-3 gene); LGMD2B (Autosomal recessive; Dysferlin gene); LGMD2C (Autosomal recessive; Gamma-sarcoglycan gene); LGMD2D (Autosomal recessive; Alpha-sarcoglycan gene); LGMD2E (Autosomal recessive; Beta-sarcoglycan gene); LGMD2F (Autosomal recessive; Delta-sarcoglycan gene); LGMD2G (Autosomal recessive; Telethonin gene); LGMD2H (Autosomal recessive; TRIM32); LGMD2I (Autosomal recessive; FKRP gene); LGMD2J (Autosomal recessive; Titin gene); LGMD2K (Autosomal recessive; POMT1 gene); and LGMD2L (Autosomal recessive; Fukutin gene).

The term “North Star Ambulatory Assessment (NSAA) score,” as used herein, refers to a clinician-administered scale that rates performance on 17 different functional activities including a 10 meter walk/run (10MWR), rising from a sit to a stand, standing on 1 leg, climbing a box step, descending a box step, rising from lying to sitting, rising from the floor, lifting head off floor, standing on heels, and jumping (Mazzone E, at al., North Star Ambulatory Assessment, 6-minute walk test and timed items in ambulant boys with Duchenne muscular dystrophy, Neuromuscul Disord. 20(11):712-6, 2010). Subjects will be graded as follows: 2=normal, no obvious modification of activity; 1=modified method but achieves goal independent of physical assistance from another; and 0=unable to achieve goal independently.

The terms “effective amount” and “therapeutically effective amount” are used interchangeably herein and refer to an amount of a therapeutic compound, such as an AAV vector or enzyme disclosed herein, administered to a mammalian subject, either as a single dose or as part of a series of doses, which is effective to produce a desired therapeutic effect. For an enzyme, this effect is typically brought about cleavage of the IgG target. For an AAV vector, the desired therapeutic effect is typically brought about by expression of a heterologous transgene present in the AAV vector and/or persistence of the AAV vector in the subject.

“Enhance” or “enhancing,” or “increase” or “increasing,” or “stimulate” or “stimulating” refers generally to the ability of one or more AAV, enzyme, or pharmaceutical compositions of any of the foregoing to produce or cause a greater physiological response (i.e., downstream effects) in a cell or a subject, as compared to the response caused by either no AAV, no enzyme, or administration of a control compound.

As used herein, the terms “function” and “functional” and the like refer to a biological, enzymatic, or therapeutic function.

The phrase “pharmaceutically acceptable” means the substance or composition must be compatible, chemically and/or toxicologically, with the other ingredients comprising a formulation, and/or the subject being treated therewith.

The phrase “pharmaceutically-acceptable carrier” as used herein means a non-toxic, inert solid, semi-solid or liquid filler, diluent, encapsulating material, or formulation auxiliary of any type. Some examples of materials which can serve as pharmaceutically acceptable carriers are: sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening agents; flavoring agents; perfuming agents; preservatives; and antioxidants; according to the judgment of the formulator.

The term “restoration” with respect to a particular protein synthesis or production refers generally to the production of the protein including truncated forms of the protein in a patient with muscular dystrophy following treatment with an AAV vector, e.g., an AAVrh74 vector disclosed herein.

In some aspects, treatment with an AAV vector, e.g., an AAVrh74 vector disclosed herein, slows or reduces the progressive respiratory muscle dysfunction and/or failure in patients with DMD or LGMD that would be expected without treatment. In some aspects, treatment with an AAV vector, e.g., an AAVrh74 vector disclosed herein, may reduce or eliminate the need for ventilation assistance that would be expected without treatment. In some aspects, measurements of respiratory function for tracking the course of the disease, as well as the evaluation of potential therapeutic interventions include maximum inspiratory pressure (MIP), maximum expiratory pressure (MEP), and forced vital capacity (FVC). MIP and MEP measure the level of pressure a person can generate during inhalation and exhalation, respectively, and are sensitive measures of respiratory muscle strength. MIP is a measure of diaphragm muscle weakness.

In some aspects, MEP may decline before changes in other pulmonary function tests, including MIP and FVC. In certain aspects, MEP may be an early indicator of respiratory dysfunction. In certain aspects, FVC may be used to measure the total volume of air expelled during forced exhalation after maximum inspiration. In patients with DMD, FVC increases concomitantly with physical growth until the early teens. However, as growth slows or is stunted by disease progression, and muscle weakness progresses, the vital capacity enters a descending phase and declines at an average rate of about 8 to 8.5 percent per year after 10 to 12 years of age. In certain aspects, MIP percent predicted (MIP adjusted for weight), MEP percent predicted (MEP adjusted for age), and FVC percent predicted (FVC adjusted for age and height) are supportive analyses.

The terms “subject” and “patient” as used herein include any animal that exhibits a symptom, or is at risk for exhibiting a symptom, which can be treated with an AAV of the disclosure, e.g., an AAVrh74 vector disclosed herein, such as a subject (or patient) that has or is at risk for having DMD or LGMD, or any of the symptoms associated with these conditions (e.g., muscle fiber loss). Suitable subjects (or patients) include laboratory animals (such as mouse, rat, rabbit, or guinea pig), farm animals, and domestic animals or pets (such as a cat or dog). Non-human primates and, preferably, human patients (or subjects), are included. In certain aspects, the subject is a male.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

“Treatment” of a subject (e.g., a mammal, such as a human) or a cell is any type of intervention used in an attempt to alter the natural course of the subject or cell. Treatment includes, but is not limited to, administration of an AAV vector, e.g., an AAVrh74 vector disclosed herein, and an enzyme that cleaves IgG, and may be performed either prophylactically or subsequent to the initiation of a pathologic event or contact with an etiologic agent. Treatment includes any desirable effect on the symptoms or pathology of a disease or condition associated with the dystrophin protein, as in certain forms of muscular dystrophy, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

In some aspects, treatment with an AAV vector comprising a transgene and an enzyme that cleaves IgG according to the disclosure increases production of the transgene (e.g., micro-dystrophin, beta sarcoglycan, or alpha sarcoglycan), delays disease progression, slows or reduces the loss of ambulation, reduces muscle inflammation, reduces muscle damage, improves muscle function, reduces loss of pulmonary function, and/or enhances muscle regeneration that would be expected without treatment. In some aspects, treatment maintains, delays, or slows disease progression. In some aspects, treatment maintains ambulation or reduces the loss of ambulation. In some aspects, treatment maintains pulmonary function or reduces loss of pulmonary function. In some aspects, treatment maintains or increases a stable walking distance in a patient, as measured by, for example, the 6 Minute Walk Test (6MWT), described by McDonald, et al. (Muscle Nerve, 2010; 42:966-74, herein incorporated by reference). A change in the 6 Minute Walk Distance (6MWD) can be expressed as an absolute value, a percentage change or a change in the %-predicted value. The performance of a DMD patient in the 6MWT relative to the typical performance of a healthy peer can be determined by calculating a %-predicted value. For example, the %-predicted 6MWD may be calculated using the following equation for males: 196.72+(39.81×age)−(1.36×age²)+(132.28×height in meters). For females, the %-oredicted 6MWD may be calculated using the following equation: 188.61+(51.50×age)−(1.86×age²)+(86.10×height in meters) (Henricson et al. PLoS Curr., 2012, version 2, herein incorporated by reference in its entirety).

In some aspects, treatment maintains or reduces the time to walk/run 10 meters (i.e., the 10 meter walk/run test). In some aspects, treatment maintains or reduces the time to stand from supine (i.e., time to stand test). In some aspects, treatment maintains or reduces the time to climb four standard stairs (i.e., the four-stair climb test). In some aspects, treatment maintains or reduces muscle inflammation in the patient, as measured by, for example, MRI (e.g., MRI of the leg muscles). In some aspects, MRI measures T2 and/or fat fraction to identify muscle degeneration. MRI can identify changes in muscle structure and composition caused by inflammation, edema, muscle damage, and fat infiltration.

In some aspects, treatment with an enzyme that cleaves IgG of the disclosure increases the vector copy number (VCN) of an AAV following subsequent administration of an AAV described herein. In some aspects, the VCN of the AAV is increased relative to the VCN of the AAV in a subject that was not administered the enzyme that cleaves IgG or relative to the vector copy number in the subject when the enzyme that cleaves IgG is not administered. In some aspects, the increase in AAV VCN is observed in a muscle.

The term “neutralizing antibody,” as used herein, refers to any antibody that binds to an AAV capsid protein of an AAV viral vector particle and inhibits transduction of a target cell by the AAV viral vector particle.

The term “non-neutralizing antibody,” as used herein, refers to any antibody that binds to an AAV capsid protein of an AAV viral vector particle and/or an AAV capsid protein that is not part of an AAV viral vector particle and does not inhibit transduction of a target cell by the AAV viral vector particle but can elicit a proinflammatory reaction in a subject.

The term “total antibodies,” as used herein, refers to AAV antibodies that include neutralizing AAV antibodies and non-neutralizing AAV antibodies.

The term “neutralizing cell assay,” as used herein refers to an assay that quantifies neutralizing AAV antibodies in a sample of a subject in vitro using cells that bind to and are transduced by an AAV viral particle to which neutralizing antibodies are quantified. In some aspects, a neutralizing cell assay comprises incubating an AAV viral particle to which neutralizing antibodies are quantified with a sample of a subject to prepare an AAV viral particle-sample mixture and incubate cells that bind to and are transduced by the AAV viral particle to which neutralizing antibodies are quantified with the AAV viral particle-sample mixture. In some aspects, the assay further comprises washing the cells and detecting the presence of the AAV viral particle inside the cells, e.g., using an enzyme-conjugated or labeled antibody against an AAV viral particle protein or, if the AAV viral particle used is a recombinant AAV viral vector particle that carries a transgene, detect the expression of the transgene in the cells.

The term “total AAVrh74 antibody ELISA assay,” as used herein refers to an enzyme-linked immunoabsorbent assay that quantifies total AAVrh74 antibodies in a sample of a subject in vitro using AAVrh74 viral particles immobilized on a solid support, and detection of AAVrh74 antibodies bound to the immobilized AAVrh74 viral particles by enzyme conjugated anti-IgG antibodies and a substrate that can be quantified. In some aspects, a total AAVrh74 antibody ELISA assay is used as described in Goedeker et al., Ther. Adv. Neurol. Disord., 6: 1-7, 2023, which is incorporated by reference herein in its entirety. In some aspects, a total AAVrh74 antibody ELISA assay can be an ELECSYS® assay.

### II. Methods of the Disclosure

Certain aspects of the present disclosure are directed to methods of treating a disease or disorder in a subject in need thereof, comprising administering to the subject (i) a recombinant AAV viral vector and (ii) an enzyme that cleaves immunoglobulin G (IgG). Without being bound by any particular theory or mechanism, administration of an enzyme that cleaves IgG before, concurrently, and/or after administration of a therapeutic AAV vector, e.g., a recombinant AAVrh74 viral vector comprising a transgene encoding a therapeutic protein disclosed herein, reduces or eliminates the immune response to the AAV vector. Gene therapy using viral vectors, such as recombinant AAV viral vectors, often elicit an inhibitory immune response in human subjects. Because many human subject have been exposed to an AAV during their lifetime, a subject may already possess inhibitory antibodies prior to a first administration of an AAV gene therapy. One means of mitigating the effects of previously existing inhibitory antibodies is to use an AAV serotype or other viral vector that most humans are unlikely to have been exposed to, such as AAVrh74. However, after a first administration of even the rarest serotypes, the human subject in many cases develops inhibitory antibodies to the administered AAV, which limits the duration of response and all but precludes a follow-on administration using the same serotype. The methods disclosed herein reduce the effects of pre-existing inhibitory antibodies and/or the formation of new inhibitory antibodies by destroying IgG polypeptides in the human subject

Some aspects of the present disclosure are directed to methods of improving efficacy of an AAV vector in a human subject having a muscular dystrophy, comprising administering to the subject (i) an enzyme that cleaves IgG and (ii) the AAV vector. Some aspects of the present disclosure are directed to methods of improving efficacy of an AAV vector in a human subject having a muscular dystrophy, comprising administering to the subject (i) an enzyme that cleaves IgG and (ii) the AAV vector.

In some aspects, the AAV vector and the enzyme that cleaves IgG are administered concurrently. In some aspects, the enzyme that cleaves IgG is administered before the AAV vector. In some aspects, the AAV vector is administered before the enzyme that cleaves IgG.

In some aspects, the methods disclosed herein are directed to treating subjects having a muscular dystrophy. In some aspects, the subjects have Duchenne Muscular Dystrophy (DMD). In some aspects, the subjects with DMD have a definite diagnosis of DMD based on documentation of clinical finding and confirmatory genetic testing. In some aspects, the subjects with DMD are children who are between 4 and 8 years of age (inclusive of age 4 and age 8). In some aspects, the subjects do not have a mutation between or including exons 1-17 of the dystrophin gene. In some aspects, the subjects do not have an in-frame deletion, in-frame insertion, or a variant of uncertain significance in the dystrophin gene.

In some aspects, the subjects have limb-girdle muscular dystrophy (LGMD). In some aspects, the subjects have LGMD caused by a mutation, in-frame deletion, in-frame insertion, or a variant of uncertain significance in an alpha-sarcoglycan gene. In some aspects, the subjects have LGMD caused by a mutation, in-frame deletion, in-frame insertion, or a variant of uncertain significance in a beta-sarcoglycan gene. In some aspects, the subjects have LGMD caused by a mutation, in-frame deletion, in-frame insertion, or a variant of uncertain significance in a non-sarcoglycan gene.

Some aspects of the present disclosure are directed to methods of increasing or restoring a muscle cell condition in a human subject having a muscular dystrophy and eligible for a therapy of an AAV vector, comprising administering to the subject an enzyme that cleaves IgG, wherein following the administration of the enzyme that cleaves IgG, the subject is to be administered the AAV vector. Some aspects of the present disclosure are directed to methods of increasing or restoring a muscle cell condition in a human subject having a muscular dystrophy and eligible for a therapy of an AAV vector, comprising administering to the subject an AAV vector, wherein the subject was previously administered an enzyme that cleaves IgG. Some aspects of the present disclosure are directed to methods of increasing or restoring a muscle cell condition in a human subject having a muscular dystrophy and eligible for a therapy of an AAV vector, comprising administering to the subject an AAV vector, wherein the subject was previously administered an enzyme that cleaves IgG. Some aspects of the present disclosure are directed to methods of increasing or restoring a muscle cell condition in a human subject having a muscular dystrophy and eligible for a therapy of an AAV vector, comprising administering to the subject an enzyme that cleaves IgG, wherein the subject is to be administered the AAV vector after the administration of the enzyme that cleaves IgG.

In some aspects, the enzyme that cleaves IgG reduces the titer of IgG antibodies that specifically bind an antigen expressed on the capsid of the AAV vector, e.g., neutralizing antibodies. By reducing the titer of one or more neutralizing antibodies, the methods disclosed herein allow for repeated administration of the therapeutic AAV vectors with limited or no loss of efficacy in the follow-on administrations.

In some aspects, the disease or disorder is characterized by a deficiency in the expression of a particular protein, and the AAV viral vector comprises a transgene that encodes the particular protein or an analog thereof. In some aspects, the disease or condition is characterized by a lack of expression, mis-expression, or a combination thereof of dystrophin, and the AAV viral vector comprises a transgene encoding dystrophin or a microdystrophin disclosed herein. In some aspects, the disease or condition is characterized by a lack of expression, mis-expression, or a combination thereof of alpha sarcoglycan, and the AAV viral vector comprises a transgene encoding alpha sarcoglycan or an analog thereof disclosed herein. In some aspects, the disease or condition is characterized by a lack of expression, mis-expression, or a combination thereof of beta sarcoglycan, and the AAV viral vector comprises a transgene encoding beta sarcoglycan or an analog thereof disclosed herein.

In some aspects, the disease or disorder comprises a muscular dystrophy. In some aspects, the muscular dystrophy is DMD. In some aspects, the muscular dystrophy is a LGMD.

### IIA. Detection of Anti-AAVrh74 Antibodies

Provided are methods of detecting neutralizing, non-neutralizing and/or total AAVrh74 antibodies in a subject. The presence of neutralizing and/or non-neutralizing AAVrh74 antibodies in a subject to be treated with an AAVrh74 viral vector particle as described herein, can reduce the efficacy of AAVrh74 vector particle transgene transfer to target cells in the subject and/or promote a pro-inflammatory response in the subject. In some aspects, an AAVrh74 antibody present in a subject binds an AAVrh74 capsid protein present in an AAVrh74 viral vector particle. In some aspects, an AAVrh74 antibody in a subject binds an AAVrh74 capsid protein of an AAVrh74 viral vector particle at a location where the AAVrh74 capsid protein interacts with a cellular receptor on a cell to be targeted with the AAVrh74 viral vector particle. In some aspects, an AAVrh74 antibody in a subject that binds an AAVrh74 capsid protein at the location that interacts with a cellular receptor prevents an interaction between the AAVrh74 capsid protein and the target cell and blocks transduction of the target cell by the AAVrh74 viral vector particle; such transduction blocking AAVrh74 antibodies are also called neutralizing antibodies.

In some aspects, an AAVrh74 antibody in a subject binds an AAVrh74 capsid protein at a location on the AAVrh74 capsid protein that does not interact with a cellular receptor on a cell to be targeted and the AAVrh74 antibody does not block AAVrh74 viral vector particle transduction of the target cell; such non-transduction blocking antibodies are also called non-neutralizing antibodies. In some aspects, non-neutralizing AAVrh74 antibody can promote proinflammatory responses in a subject.

In some aspects, a subject has both neutralizing and non-neutralizing AAVrh74 antibodies. In some aspects, total AAVrh74 antibodies are quantified in a subject using assays described herein, which total AAVrh74 antibodies include neutralizing and non-neutralizing AAVrh74 antibodies.

In some aspects, a subject to be treated with the AAVrh74 viral vector particles described herein is tested for the presence of total AAVrh74 antibodies using an assay as described herein and the subject is further tested for the presence of neutralizing AAVrh74 antibodies using an assay as described herein. In some aspects, a quantity of non-neutralizing AAVrh74 antibodies present in a subject is determined by subtracting an amount of neutralizing AAVrh74 antibodies present in the subject from an amount of total AAVrh74 antibodies present in the subject.

In some aspects, total, neutralizing and/or non-neutralizing AAVrh74 antibodies are determined in a subject prior to treatment with an AAVrh74 viral vector particle described herein.

In some aspects, total, neutralizing and/or non-neutralizing AAVrh74 antibodies are determined in a subject after treatment with an AAVrh74 viral vector particle described herein.

In some aspects, total, neutralizing and/or non-neutralizing AAVrh74 antibodies are determined in a subject prior to treatment with an enzyme that cleaves IgG described herein.

In some aspects, total, neutralizing and/or non-neutralizing AAVrh74 antibodies are determined in a subject after treatment with an enzyme that cleaves IgG described herein.

In some aspects, total, neutralizing and/or non-neutralizing AAVrh74 antibodies are determined in a subject prior to treatment with an AAVrh74 viral vector particle and after a first treatment with an enzyme that cleaves IgG described herein.

In some aspects, total, neutralizing and/or non-neutralizing AAVrh74 antibodies are determined in a subject prior to treatment with an AAVrh74 viral vector particle and after a second treatment with an enzyme that cleaves IgG described herein.

In some aspects, total, neutralizing and/or non-neutralizing AAVrh74 antibodies are determined in a subject prior to treatment with an AAVrh74 viral vector particle and after a third treatment with an enzyme that cleaves IgG described herein.

In some aspects, total, neutralizing and/or non-neutralizing AAVrh74 antibodies are determined in a subject prior to a second treatment with an AAVrh74 viral vector particle following a first treatment with an AAVrh74 viral vector particle.

In some aspects, total, neutralizing and/or non-neutralizing AAVrh74 antibodies are determined in a subject prior to a second treatment with an AAVrh74 viral vector particle and after a first treatment with an enzyme that cleaves IgG described herein following a first treatment with an AAVrh74 viral vector particle.

In some aspects, total, neutralizing and/or non-neutralizing AAVrh74 antibodies are determined in a subject prior to a second treatment with an AAVrh74 viral vector particle and after a second treatment with an enzyme that cleaves IgG described herein following a first treatment with an AAVrh74 viral vector particle.

In some aspects, total, neutralizing and/or non-neutralizing AAVrh74 antibodies are determined in a subject prior to a second treatment with an AAVrh74 viral vector particle and after a third treatment with an enzyme that cleaves IgG described herein following a first treatment with an AAVrh74 viral vector particle. In some aspects, total, neutralizing and/or non-neutralizing AAVrh74 antibodies are determined in a subject prior to a third treatment with an AAVrh74 viral vector particle following a second treatment with an AAVrh74 viral vector particle.

In some aspects, total, neutralizing and/or non-neutralizing AAVrh74 antibodies are determined in a subject prior to a third treatment with an AAVrh74 viral vector particle described herein and after a first treatment with an enzyme that cleaves IgG following a second treatment with the AAVrh74 viral vector particle.

In some aspects, total, neutralizing and/or non-neutralizing AAVrh74 antibodies are determined in a subject prior to a third treatment with an AAVrh74 viral vector particle described herein and after a second treatment with an enzyme that cleaves IgG following a second treatment with the AAVrh74 viral vector particle.

In some aspects, total, neutralizing and/or non-neutralizing AAVrh74 antibodies are determined in a subject prior to a third treatment with an AAVrh74 viral vector particle described herein and after a third treatment with an enzyme that cleaves IgG following a second treatment with the AAVrh74 viral vector particle.

### IIB. Detection of Neutralizing Anti-AAVrh74 Antibodies

In some aspects, neutralizing AAVrh74 antibodies are measured using a neutralizing cell based assay as described herein. In some aspects, the neutralizing cell assay is an assay described in Enders et al., Clinical and Vaccine Immunology, 20: 420-426, 2013, incorporated by reference herein in its entirety.

In some aspects, neutralizing AAVrh74 antibodies are quantified in a sample of a subject in vitro using cells that bind to and are transduced by an AAVrh74 viral particle. In some aspects, neutralizing AAVrh74 antibodies are quantified in a sample by incubating an AAVrh74 viral particle with a sample of a subject to prepare an AAVrh74 viral particle-sample mixture. In some aspects, cells that bind to and are transduced by an AAVrh74 viral particle are incubated with the AAVrh74 viral particle-sample mixture. In some aspects, the cells are washed after incubation with the AAVrh74 viral particle-sample mixture and the AAVrh74 viral particles are quantified inside the cells. In some aspects, the AAVrh74 viral particles are quantified inside the cells using an enzyme-conjugated antibody against an AAVrh74 viral particle protein. In some aspects, an AAVrh74 viral vector particle carrying a transgene is used in the neutralizing antibody assay and the AAVrh74 viral vector particles are quantified by quantifying the expression of the transgene in the cells.

In some aspects, neutralizing anti-AAVrh74 antibodies are measured before administration of an AAVrh74 vector particle and before administration of Imlifidase. In some aspects, neutralizing anti-AAVrh74 antibodies are measured before administration of an AAVrh74 vector particle and after administration of a first dose of Imlifidase. In some aspects, neutralizing anti-AAVrh74 antibodies are measured before administration of an AAVrh74 vector particle and after administration of a second dose of Imlifidase. In some aspects, neutralizing anti-AAVrh74 antibodies are measured before administration of an AAVrh74 vector particle and after administration of a third dose of Imlifidase.

In some aspects, neutralizing anti-AAVrh74 antibodies are measured after administration of an AAVrh74 vector particle. In some aspects, neutralizing anti-AAVrh74 antibodies are measured after administration of a first dose of an AAVrh74 vector particle and after administration of a first dose of Imlifidase.

In some aspects, neutralizing anti-AAVrh74 antibodies are measured after administration of a first dose of an AAVrh74 vector particle and after administration of a second dose of Imlifidase.

In some aspects, neutralizing anti-AAVrh74 antibodies are measured after administration of a first dose of an AAVrh74 vector particle and after administration of a third dose of Imlifidase.

In some aspects, neutralizing anti-AAVrh74 antibodies are measured after administration of a second dose of an AAVrh74 vector particle. In some aspects, neutralizing anti-AAVrh74 antibodies are measured after administration of a second dose of an AAVrh74 vector particle and after administration of a first dose of Imlifidase.

In some aspects, neutralizing anti-AAVrh74 antibodies are measured after administration of a second dose of an AAVrh74 vector particle and after administration of a second dose of Imlifidase.

In some aspects, neutralizing anti-AAVrh74 antibodies are measured after administration of a second dose of an AAVrh74 vector particle and after administration of a third dose of Imlifidase.

In some aspects, neutralizing anti-AAVrh74 antibodies are measured after administration of a third dose of an AAVrh74 vector particle. In some aspects, neutralizing anti-AAVrh74 antibodies are measured after administration of a third dose of an AAVrh74 vector particle and after administration of a first dose of Imlifidase.

In some aspects, neutralizing anti-AAVrh74 antibodies are measured after administration of a third dose of an AAVrh74 vector particle and after administration of a second dose of Imlifidase.

In some aspects, neutralizing anti-AAVrh74 antibodies are measured after administration of a third dose of an AAVrh74 vector particle and after administration of a third dose of Imlifidase.

In some aspects, neutralizing anti-AAVrh74 antibodies are measured after administration of a further dose of an AAVrh74 vector particle. In some aspects, neutralizing anti-AAVrh74 antibodies are measured after administration of a further dose of an AAVrh74 vector particle and after administration of a first dose of Imlifidase.

In some aspects, neutralizing anti-AAVrh74 antibodies are measured after administration of a further dose of an AAVrh74 vector particle and after administration of a second dose of Imlifidase.

In some aspects, neutralizing anti-AAVrh74 antibodies are measured after administration of a further dose of an AAVrh74 vector particle and after administration of a third dose of Imlifidase.

A person of ordinary skill in the art will appreciate that the measurements of neutralizing AAVrh74 antibodies are not limited by the number of administrations of AAVrh74 vector particles or the number of dose of Imlifidase. In some aspects, neutralizing anti-AAVrh74 antibodies are measured any time a subject is considered for administration of an AAVrh74 vector particle. In some aspects, neutralizing anti-AAVrh74 antibodies are measured any time a subject is considered for administration of Imlifidase.

In some aspects, the assay used to measure neutralizing AAVrh74 antibodies in a subject is a neutralizing cell assay. In some aspects, a subject is determined to have neutralizing anti-AAVrh74 antibodies if the anti-AAVrh74 antibody titer measured in a neutralizing cell assay is greater than 1:400. In some aspects, a subject is determined to not have neutralizing anti-AAVrh74 antibodies if the anti-AAVrh74 antibody titer measured in a neutralizing cell assay is equal to or less than 1:400.

### II.C. Detection of Total Anti-AAVrh74 Antibodies

In some aspects, total anti-AAVrh74 antibodies comprising neutralizing and non-neutralizing anti-AAVrh74 antibodies are measured. In some aspects, total anti-AAVrh74 antibodies are measured using a total anti-AAVrh74 Antibody ELISA assay. In some aspects, a subject who has a titer of total anti-AAVrh74 antibodies greater than 1:400 in a total anti-AAVrh74 antibody ELISA assay is seropositive. In some aspects, a subject who has a titer of total anti-AAVrh74 antibodies of or less than 1:400 in a total anti-AAVrh74 antibody ELISA assay is seronegative.

In some aspects, the total anti-AAVrh74 Antibody ELISA assay is an assay described in Goedeker et al., Ther. Adv. Neurol. Dis., 16: 1-7, 2023, incorporated by reference herein in its entirety.

In some aspects, total anti-AAVrh74 antibodies are measured before administration of an AAVrh74 vector particle and before administration of Imlifidase. In some aspects, total anti-AAVrh74 antibodies are measured before administration of an AAVrh74 vector particle and after administration of a first dose of Imlifidase. In some aspects, total anti-AAVrh74 antibodies are measured before administration of an AAVrh74 vector particle and after administration of a second dose of Imlifidase. In some aspects, total anti-AAVrh74 antibodies are measured before administration of an AAVrh74 vector particle and after administration of a third dose of Imlifidase.

In some aspects, total anti-AAVrh74 antibodies are measured after administration of an AAVrh74 vector particle. In some aspects, total anti-AAVrh74 antibodies are measured after administration of a first dose of an AAVrh74 vector particle and after administration of a first dose of Imlifidase.

In some aspects, total anti-AAVrh74 antibodies are measured after administration of a first dose of an AAVrh74 vector particle and after administration of a second dose of Imlifidase.

In some aspects, total anti-AAVrh74 antibodies are measured after administration of a first dose of an AAVrh74 vector particle and after administration of a third dose of Imlifidase.

In some aspects, total anti-AAVrh74 antibodies are measured after administration of a second dose of an AAVrh74 vector particle. In some aspects, total anti-AAVrh74 antibodies are measured after administration of a second dose of an AAVrh74 vector particle and after administration of a first dose of Imlifidase.

In some aspects, total anti-AAVrh74 antibodies are measured after administration of a second dose of an AAVrh74 vector particle and after administration of a second dose of Imlifidase.

In some aspects, total anti-AAVrh74 antibodies are measured after administration of a second dose of an AAVrh74 vector particle and after administration of a third dose of Imlifidase.

In some aspects, total anti-AAVrh74 antibodies are measured after administration of a third dose of an AAVrh74 vector particle. In some aspects, total anti-AAVrh74 antibodies are measured after administration of a third dose of an AAVrh74 vector particle and after administration of a first dose of Imlifidase.

In some aspects, total anti-AAVrh74 antibodies are measured after administration of a third dose of an AAVrh74 vector particle and after administration of a second dose of Imlifidase.

In some aspects, total anti-AAVrh74 antibodies are measured after administration of a third dose of an AAVrh74 vector particle and after administration of a third dose of Imlifidase.

In some aspects, total anti-AAVrh74 antibodies are measured after administration of a further dose of an AAVrh74 vector particle. In some aspects, total anti-AAVrh74 antibodies are measured after administration of a further dose of an AAVrh74 vector particle and after administration of a first dose of Imlifidase.

In some aspects, total anti-AAVrh74 antibodies are measured after administration of a further dose of an AAVrh74 vector particle and after administration of a second dose of Imlifidase.

In some aspects, total anti-AAVrh74 antibodies are measured after administration of a further dose of an AAVrh74 vector particle and after administration of a third dose of Imlifidase.

A person of ordinary skill in the art will appreciate that the measurements of total AAVrh74 antibodies are not limited by the number of administrations of AAVrh74 vector particles or the number of dose of Imlifidase. In some aspects, total anti-AAVrh74 antibodies are measured any time a subject is considered for administration of an AAVrh74 vector particle. In some aspects, total anti-AAVrh74 antibodies are measured any time a subject is considered for administration of Imlifidase.

In some aspects, the assay used to measure total AAVrh74 antibodies in a subject is a total AAVrh74 antibody ELISA assay. In some aspects, a subject is determined to have total anti-AAVrh74 antibodies if the anti-AAVrh74 antibody titer measured in a total AAVrh74 antibody ELISA assay is greater than 1:400. In some aspects, a subject is determined to not have total anti-AAVrh74 antibodies if the anti-AAVrh74 antibody titer measured in a total AAVrh74 antibody ELISA assay is equal to or less than 1:400.

### IID. Detection of Non-Neutralizing Anti-AAVrh74 Antibodies

In some aspects, a subject to be treated with AAVrh74 viral vector particles described herein is tested for the presence of non-neutralizing AAVrh74 antibodies. In some aspects, total AAVrh74 antibodies are quantified in a sample of the subject as described herein and neutralizing AAVrh74 antibodies are quantified in the sample as described herein. In some aspects, a quantity of non-neutralizing AAVrh74 antibodies present in the sample of the subject is determined by subtracting an amount of neutralizing AAVrh74 antibodies present in the sample from an amount of total AAVrh74 antibodies present in the sample.

In some aspects, non-neutralizing anti-AAVrh74 antibodies are measured before administration of an AAVrh74 vector particle and before administration of Imlifidase. In some aspects, non-neutralizing anti-AAVrh74 antibodies are measured before administration of an AAVrh74 vector particle and after administration of a first dose of Imlifidase. In some aspects, non-neutralizing anti-AAVrh74 antibodies are measured before administration of an AAVrh74 vector particle and after administration of a second dose of Imlifidase. In some aspects, non-neutralizing anti-AAVrh74 antibodies are measured before administration of an AAVrh74 vector particle and after administration of a third dose of Imlifidase.

In some aspects, non-neutralizing anti-AAVrh74 antibodies are measured after administration of an AAVrh74 vector particle. In some aspects, neutralizing anti-AAVrh74 antibodies are measured after administration of a first dose of an AAVrh74 vector particle and after administration of a first dose of Imlifidase.

In some aspects, non-neutralizing anti-AAVrh74 antibodies are measured after administration of a first dose of an AAVrh74 vector particle and after administration of a second dose of Imlifidase.

In some aspects, non-neutralizing anti-AAVrh74 antibodies are measured after administration of a first dose of an AAVrh74 vector particle and after administration of a third dose of Imlifidase.

In some aspects, non-neutralizing anti-AAVrh74 antibodies are measured after administration of a second dose of an AAVrh74 vector particle. In some aspects, non-neutralizing anti-AAVrh74 antibodies are measured after administration of a second dose of an AAVrh74 vector particle and after administration of a first dose of Imlifidase.

In some aspects, non-neutralizing anti-AAVrh74 antibodies are measured after administration of a second dose of an AAVrh74 vector particle and after administration of a second dose of Imlifidase.

In some aspects, non-neutralizing anti-AAVrh74 antibodies are measured after administration of a second dose of an AAVrh74 vector particle and after administration of a third dose of Imlifidase.

In some aspects, non-neutralizing anti-AAVrh74 antibodies are measured after administration of a third dose of an AAVrh74 vector particle. In some aspects, non-neutralizing anti-AAVrh74 antibodies are measured after administration of a third dose of an AAVrh74 vector particle and after administration of a first dose of Imlifidase.

In some aspects, non-neutralizing anti-AAVrh74 antibodies are measured after administration of a third dose of an AAVrh74 vector particle and after administration of a second dose of Imlifidase.

In some aspects, non-neutralizing anti-AAVrh74 antibodies are measured after administration of a third dose of an AAVrh74 vector particle and after administration of a third dose of Imlifidase.

In some aspects, non-neutralizing anti-AAVrh74 antibodies are measured after administration of a further dose of an AAVrh74 vector particle. In some aspects, neutralizing anti-AAVrh74 antibodies are measured after administration of a further dose of an AAVrh74 vector particle and after administration of a first dose of Imlifidase.

In some aspects, non-neutralizing anti-AAVrh74 antibodies are measured after administration of a further dose of an AAVrh74 vector particle and after administration of a second dose of Imlifidase.

In some aspects, non-neutralizing anti-AAVrh74 antibodies are measured after administration of a further dose of an AAVrh74 vector particle and after administration of a third dose of Imlifidase.

A person of ordinary skill in the art will appreciate that the measurements of non-neutralizing AAVrh74 antibodies are not limited by the number of administrations of AAVrh74 vector particles or the number of dose of Imlifidase. In some aspects, non-neutralizing anti-AAVrh74 antibodies are measured any time a subject is considered for administration of an AAVrh74 vector particle. In some aspects, non-neutralizing anti-AAVrh74 antibodies are measured any time a subject is considered for administration of Imlifidase.

### III. Enzymes that Cleave IgG

Certain aspects of the present disclosure are directed to methods of treating a disease or condition, e.g., a muscular dystrophy, in a subject in need thereof, comprising administering to the subject (i) an AAV vector and (ii) an enzyme that cleaves IgG. In certain aspects, the enzyme targets and cleaves human IgG. In some aspects, the enzyme targets and cleaves a human IgG selected from IgG1, IgG2, IgG3, IgG4 and any combination thereof. In some aspects, the enzyme targets and cleaves human IgG1. In some aspects, the enzyme targets and cleaves human IgG2. In some aspects, the enzyme targets and cleaves human IgG3. In some aspects, the enzyme targets and cleaves human IgG4. In some aspects, the enzyme targets and cleaves human IgG1, human IgG2, human IgG3, and human IgG4. In some aspects, the IgG binds an AAV vector. In certain aspect, the IgG binds an AAVrh74 vector. In some aspects, the subject has one or more IgG antibodies that specifically bind a protein expressed by AAVrh74. In some aspects, the subject has one or more IgG antibodies that specifically bind an AAVrh74 capsid protein. In some aspects, the one or more IgG antibodies are AAVrh74 neutralizing antibodies.

In some aspects, the enzyme that cleaves IgG comprises a protease. In some aspects, the enzyme that cleaves IgG comprises a cysteine protease. In some aspects, the enzyme that cleaves IgG comprises a thiol protease. In some aspects, the enzyme that cleaves IgG inactivates the IgG upon cleavage. In some aspects, the enzyme cleaves IgG at the hinge region of the IgG. In certain aspects, following cleavage the IgG is no longer capable of binding an antigen. In certain aspects, following cleavage the IgG is no longer capable of being recognized by a human immune cell.

Any enzymes known in the art that can facilitate the cleavage of IgG can be used in the methods disclosed herein. In some aspects, the enzyme is a naturally occurring enzyme. In some aspects, the enzyme is derived from a naturally occurring enzyme. In some aspects, the enzyme is a synthetic enzyme. In some aspects, the enzyme is recombinant. In some aspects, the enzyme is isolated or derived from a bacteria. In some aspects, the enzyme is isolated or derived from a bacteria of the genus Streptococcus. In some aspects, the enzyme is isolated or derived from Streptococcus pyogenes. In some aspects, the enzyme is isolated or derived from Streptococcus equi. In some aspects, the enzyme is isolated or derived from a bacteria of the genus Mycoplasma. In some aspects, the enzyme is isolated or derived from Mycoplasma canis.

In certain aspects, the enzyme that cleaves IgG comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to an amino acid sequence selected from the amino acid sequences set forth in SEQ ID NOs: 1-18 (Table 5). In certain aspects, the enzyme that cleaves IgG comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 2. In some aspects, the enzyme that cleaves IgG comprises the amino acid sequence set forth in SEQ ID NO: 2. In some aspects, the enzyme that cleaves IgG comprises the amino acid sequence set forth in SEQ ID NO: 1. In some aspects, the enzyme that cleaves IgG comprises an amino acid sequence selected from an amino acid sequence set forth in SEQ ID NOs 3-18.

TABLE 5
Sample Enzymes the Cleave IgG (Amino Acid Sequences)

| Enzyme | Amino Acid Sequence |
|---|---|
| Imlifidase | MRKRCYSTSAAVLAAVTLFVLSVDRGVIADSFSANQEIRYSEVTPYHVTSVWTKGVT |
| (IdeS) | PPANFTQGEDVFHAPYVANQGWYDITKTFNGKDDLLCGAATAGNMLHWWFDQNKDQI |
| (signal | KRYLEEHPEKQKINFNGEQMFDVKEAIDTKNHQLDSKLFEYFKEKAFPYLSTKHLGV |
| peptide) | FPDHVIDMFINGYRLSLTNHGPTPVKEGSKDPRGGIFDAVFTRGDQSKLLTSRHDFK |
| | EKNLKEISDLIKKELTEGKALGLSHTYANVRINHVINLWGADFDSNGNLKAIYVTDS |
| | DSNASIGMKKYFVGVNSAGKVAISAKEIKEDNIGAQVLGLFTLSTGQDSWNQTN |
| | (SEQ ID NO: 1) |
| Mature | DSFSANQEIRYSEVTPYHVTSVWTKGVTPPANFTQGEDVFHAPYVANQGWYDITKTF |
| Imlifidase | NGKDDLLCGAATAGNMLHWWFDQNKDQIKRYLEEHPEKQKINFNGEQMFDVKEAIDT |
| | KNHQLDSKLFEYFKEKAFPYLSTKHLGVFPDHVIDMFINGYRLSLTNHGPTPVKEGS |
| | KDPRGGIFDAVFTRGDQSKLLTSRHDFKEKNLKEISDLIKKELTEGKALGLSHTYAN |
| | VRINHVINLWGADFDSNGNLKAIYVTDSDSNASIGMKKYFVGVNSAGKVAISAKEIK |
| | EDNIGAQVLGLFTLSTGQDSWNQTN (SEQ ID NO: 2) |
| Variant 1 | DSFSANQEIRYSEVTPYHVTSVWTKGVTPPANFTQGEDVFHAPYVANQGWYDITKTF |
| | NGKDDLLCGAATAGNMLHWWFDQNKDQIKRYLEEHPEKQKINFRGEQMFDVKEAIDT |
| | KNHQLDSKLFEYFKEKAFPYLSTKHLGVFPDHVIDMFINGYRLSLTNHGPTPVKEGS |
| | KDPRGGIFDAVFTRGDQSKLLTSRHDFKEKNLKEISDLIKKELTEGKALGLSHTYAN |
| | VRINHVINLWGADFDSNGNLKAIYVTDSDSNASIGMKKYFVGVNSAGKVAISAKEIK |
| | EDNIGAQVLGLFTLSTGQDSWNQTN (SEQ ID NO: 3) |
| Variant 2 | DSFSANQEIRYSEVTPYHVTSVWTKGVTPPANFTQGEDVFHAPYVANQGWYDITKTF |
| | NGKDDLLCGAATAGNMLHWWFDQNKDQIKRYLEEHPEKQKINFKGEQMFDVKEAIDT |
| | KNHQLDSKLFEYFKEKAFPYLSTKHLGVFPDHVIDMFINGYRLSLTNHGPTPVKRGS |
| | KDPRGGIFDAVFTRGNQSKLLTSRHDFKEKNLKEISDLIKKELTEGKALGLSHTYAN |
| | VRINHVINLWGADFDSNGNLKAIYVTDSDSNASIGMKKYFVGVNSAGKVAISAKEIK |
| | EDNIGAQVLGLFTLSTGQDSWNQTN (SEQ ID NO: 4) |
| Variant 3 | DSFSANQEIRYSEVTPYHVTSVWTKGVTPPANFTQGEDVFHAPYVANQGWYDITKTF |
| | NGKDDLLCGAATAGNMLHWWFDQNKDQIKRYLEEHPEKQKINFRGEQMFDVKEAIDT |
| | KNHQLDSKLFEYFKEKAFPYLSTKHLGVFPDHVIDMFINGYRLSLTNHGPTPVKKGS |
| | KDPRGGIFDAVFTRGNQSKLLTSRHDFKEKNLKEISDLIKKELTEGKALGLSHTYAN |
| | VRINHVINLWGADFDSNGNLKAIYVTDSDSNASIGMKKYFVGVNKAGKVAISAKEIK |
| | EDNIGAQVLGLFTLSTGQDSWNQTN (SEQ ID NO: 5) |
| Variant 4 | DSFSANQEIRYSEVTPYHVTSVWTKGVTPPANFTQGEDVFHAPYVANQGWYDITKTF |
| | NGKDDLLCGAATAGNMLHWWFDQNKDQIKRYLREHPEKQKINFNGEQMFDVKEAIDT |
| | KNHQLDSKLFEYFKEKAFPYLSTKHLGVFPDHVIDMFINGYRLSLTNHGPTPVKEGS |
| | KDPRGGIFDAVFTRGNQSKLLTSRHDFKEKNLKEISDLIKKELDEGKALGLSHTYAN |
| | VRINHVINLWGADFDSNGNLKAIYVTDSDSNASIGMKKYFVGVNSAGKVAISAKEIK |
| | EDNIGAQVLGLFTLSTGQDSWNQTN (SEQ ID NO: 6) |
| Variant 5 | DSFSANQEIRYSEVTPYHVTSVWTKGVTPPANFTQGEDVFHAPYVANQGWYDITKTF |
| | NGKDDLLCGAATAGNMLHWWFDQNKDQIKRYLKEHPEKQKINFNGEQMFDVKEAIRT |
| | KNHQLDSKLFEYFKEKAFPYLSTKHLGVFPDHVIDMFINGYRLSLTNHGPTPVKEGS |
| | KDPRGGIFDAVFTRGNQSKLLTSRHDFKEKNLKEISDLIKKELEEGKALGLSHTYAN |
| | VRINHVINLWGADFDSNGNLKAIYVTDSDSNASIGMKKYFVGVNKAGKVAISAKEIK |
| | EDNIGAQVLGLFTLSTGQDSWNQTN (SEQ ID NO: 7) |
| Variant 6 | DSFSANQEIRYSEVTPYHVTSVWTKGVTPPANFTQGEDVFHAPYVANQGWYDITKTF |
| | NGKDDLLCGAATAGNMLHWWFDQNKDQIERYLEEHPEKQKINFNGEQMFDVKEAIDT |
| | KNHQLDSKLFEYFKEKAFPYLSTKHLGVFPDHVIDMFINGYRLSLTNHGPTPVKEGS |
| | KDPRGGIFDAVFTRGNQSKLLTSRHDFKEKNLKEISDLIKEELTKGKALGLSHTYAN |
| | VRINHVINLWGADFDSNGNLKAIYVTDSDSNASIGMKKYFVGVNSAGKVAISAKEIK |
| | EKNIGAQVLGLFTLSTGQKSWNQTN (SEQ ID NO: 8) |
| Variant 7 | DSFSANQEIRYSEVTPYHVTSVWTKGVTPPANFTQGEDVFHAPYVANQGWYDITKTF |
| | NGKDDLLCGAATAGNMLHWWFDQNKDQIKRYLKEHPEKQKINFRGEQMFDVKEAIRT |
| | KNHQLDSKLFEYFKEKAFPYLSTKHLGVFPDHVIDMFINGYRLSLTNHGPTPVKEGS |
| | KDPRGGIFDAVFTRGNQSKLLTSRHDFKEKNLKEISDLIKSELENGKALGLSHTYAN |
| | VRINHVINLWGADFDSNGNLKAIYVTDSDSNASIGMKKYFVGVNKAGKVAISAKEIK |
| | EDNIGAQVLGLFTLSTGQDSWNQTN (SEQ ID NO: 9) |
| Variant 8 | DSFSANQEIRYSEVTPYHVTSVWTKGVTPPANFTQGEDVFHAPYVANQGWYDITKTF |
| | NGKDDLLCGAATAGNMLHWWFDQNKDQIKRYLKEHPEKQKINFRGEQMFDVKEAIRT |
| | KNHQLDSKLFEYFKEKAFPYLSTKHLGVFPDHVIDMFINGYRLSLTNHGPTPVKKGS |
| | KDPRGGIFDAVFTRGNQSKLLTSRHDFKEKNLKEISDLIKKELEEGKALGLSHTYAN |
| | VRINHVINLWGADFDSNGNLKAIYVTDSDSNASIGMKKYFVGVNSAGKVAISAKEIK |
| | EDNIGAQVLGLFTLSTGQDSWNQTN (SEQ ID NO: 10) |
| Variant 9 | DSFSANQEIRYSEVTPYHVTSVWTKGVTPPANFTQGEDVFHAPYVANQGWYDITKTF |
| | NGKDDLLCGAATAGNMLHWWFDQNKDQIERYLEEHPEKQKINFRGEQMFDVKEAIDT |
| | KNHQLDSKLFEYFKEKAFPYLSTKHLGVFPDHVIDMFINGYRLSLTNHGPTPVKKGS |
| | KDPRGGIFDAVFTRGNQSKLLTSRHDFKEKNLKEISDLIKEELTKGKALGLSHTYAN |
| | VRINHVINLWGADFDSNGNLKAIYVTDSDSNASIGMKKYFVGVNSAGKVAISAKEIK |
| | EDNIGAQVLGLFTLSTGQKSWNQTN (SEQ ID NO: 11) |
| Variant 10 | DSFSANQEIRYSEVTPYHVTSVWTKGVTPPANFTQGEDVFHAPYVANQGWYDITKTF |
| | NGKDDLLCGAATAGNMLHWWFDQNKDQIKRYLEEHPEKQKINFNGEQMFDVKEAIDT |
| | KNHQLDSKLFEYFKEKAFPYLSTKHLGVFPDHVIDMFINGYRLSLTNHGPTPVKEGS |
| | KDPRGGIFDAVFTRGDQSKLLTSRHDFKEKNLKEISDLIKKELTEGKALGLSHTYAN |
| | VRINHVINLWGADFDSNGNLKAIYVTDSDSNASIGMKKYFVGVNSAGKVAISAKEIK |
| | EDNIGAQVLGLFTLSTGQD (SEQ ID NO: 12) |
| Variant 11 | SVWTKGVTPPANFTQGEDVFHAPYVANQGWYDITKTFNGKDDLLCGAATAGNMLHWW |
| | FDQNKDQIKRYLEEHPEKQKINFNGEQMFDVKEAIDTKNHQLDSKLFEYFKEKAFPY |
| | LSTKHLGVFPDHVIDMFINGYRLSLTNHGPTPVKEGSKDPRGGIFDAVFTRGDQSKL |
| | LTSRHDFKEKNLKEISDLIKKELTEGKALGLSHTYANVRINHVINLWGADFDSNGNL |
| | KAIYVTDSDSNASIGMKKYFVGVNSAGKVAISAKEIKEDNIGAQVLGLFTLSTGQDS |
| | WNQTN (SEQ ID NO: 13) |
| Variant 12 | SVWTKGVTPPANFTQGEDVFHAPYVANQGWYDITKTFNGKDDLLCGAATAGNMLHWW |
| | FDQNKDQIKRYLEEHPEKQKINFKGEQMFDVKEAIDTKNHQLDSKLFEYFKEKAFPY |
| | LSTKHLGVFPDHVIDMFINGYRLSLTNHGPTPVKEGSKDPRGGIFDAVFTRGNQSKL |
| | LTSRHDFKEKNLKEISDLIKKELTEGKALGLSHTYANVRINHVINLWGADFDSNGNL |
| | KAIYVTDSDSNASIGMKKYFVGVNSAGKVAISAKEIKEDNIGAQVLGLFTLSTGQDS |
| | WNQTN (SEQ ID NO: 14) |
| Variant 13 | SVWTKGVTPPANFTQGEDVFHAPYVANQGWYDITKTFNGKDDLLCGAATAGNMLHWW |
| | FDQNKDQIERYLEEHPEKQKINFKGEQMFDVKKAIDTKNHQLDSKLFEYFKEKAFPY |
| | LSTKHLGVFPDHVIDMFINGYRLSLTNHGPTPVKEGSKDPRGGIFDAVFTRGNQSKL |
| | LTSRHDFKEKNLKEISDLIKEELTKGKALGLSHTYANVRINHVINLWGADFDSNGNL |
| | KAIYVTDSDSNASIGMKKYFVGVNSAGKVAISAKEIKEDNIGAQVLGLFTLSTGQKS |
| | WNQTN (SEQ ID NO: 15) |
| Variant 14 | DDYQRNATEAYAKEVPHQITSVWTKGVTPPANFTQGEDVFHAPYVANQGWYDITKTF |
| | NGKDDLLCGAATAGNMLHWWFDQNKDQIERYLEEHPEKQKINFKGEQMFDVKKAIDT |
| | KNHQLDSKLFEYFKEKAFPYLSTKHLGVFPDHVIDMFINGYRLSLTNHGPTPVKEGS |
| | KDPRGGIFDAVFTRGNQSKLLTSRHDFKEKNLKEISDLIKEELTKGKALGLSHTYAN |
| | VRINHVINLWGADFDSNGNLKAIYVTDSDSNASIGMKKYFVGVNSAGKVAISAKEIK |
| | EDNIGAQVLGLFTLSTGQKSWNQTNGGGHHHHHH (SEQ ID NO: 16) |
| Variant 15 | DDYQRNATEAYAKEVPHQITSVWTKGVTPLTPEQFRYNNEDVFHAPYVANQGWYDIT |
| | KAFDGKDNLLCGAATAGNMLHWWFDQNKDQIKRYLEEHPEKQKINFNGDNMFDVKKA |
| | IDTKNHQLDSKLFNYFKEKAFPGLSARRIGVFPDHVIDMFINGYRLSLTNHGPTPVK |
| | EGSKDPRGGIFDAVFTRGNQSKLLTSRHDFKNKNLNDISTIIKQELTKGKALGLSHT |
| | YANVSINHVINLWGADFNAEGNLEAIYVTDSDSNASIGMKKYFVGVNAHGHVAISAK |
| | KIEGENIGAQVLGLFTLSTGQDSWQKLS (SEQ ID NO: 17) |
| Variant 16 | MDSFSANQEIRYSEVTPYHVTSVWTKGVTPPANFTQGEDVFHAPYVANQGWYDITKT |
| | FNGKDDLLCGAATAGNMLHWWFDQNKDQIKRYLEEHPEKQKINFNGEQMFDVKEAID |
| | TKNHQLDSKLFEYFKEKAFPYLSTKHLGVFPDHVIDMFINGYRLSLTNHGPTPVKEG |
| | SKDPRGGIFDAVFTRGDQSKLLTSRHDFKEKNLKEISDLIKKELTEGKALGLSHTYA |
| | NVRINHVINLWGADFDSNGNLKAIYVTDSDSNASIGMKKYFVGVNSAGKVAISAKEI |
| | KEDNIGAQVLGLFTLSTGQDSWNQTN (SEQ ID NO: 18) |

In certain aspects, the enzyme that cleaves IgG comprises Imlifidase. In certain aspects, the enzyme that cleaves IgG consists of Imlifidase. Imlifidase (also known as “IdeS” and “Mac-1”; UniProt F8V4V0) is an endopeptidase derived from Streptococcus pyogenes that targets and cleaves all human subclases of IgG, whilst not cleaving IgA, (gD, IgE, or IgM. Imlifidase is a cysteine protease, having an active-site residue consisting of a single Cys residue. Imlifidase cleaves the heavy chain of IgG at the hinge region to produce two Fc and one F(ab¹)₂ fragments. Cleavage of IgG by Imlifidase inhibits complement activation and Fc receptor mediated processes, including antibody-dependent cellular phagocytosis (ADCP), antibody-dependent cell-mediated cytotoxicity (ADCC) and complement-dependent cytotoxicity (CDC). Within hours after dosing Imlifidase, the pool of IgG is cleaved into F(ab′)₂ and Fc fragments, creating a window for the administration of gene therapy.

Administration of Imlifidase to highly HLA-sensitized patients before kidney transplantation was previously shown to eliminate or reduce donor-specific antibodies. Imlifidase is also known to interact with vitronectin and platelet receptors through its RGD motif.

### IV. AAV Viral Vector Particles

Certain aspects of the present disclosure are directed to methods of treating a disease or condition, e.g., a muscular dystrophy, in a subject in need thereof, comprising administering to the subject (i) an AAV vector and (ii) an enzyme that cleaves IgG. The AAV vector can comprise a known vector or can comprise a variant, fragment, or fusion thereof. In some aspects, the AAV vector is selected from the group consisting of AAV type 1 (AAV1), AAV2, AAV3, AAV4, AAV5, AAV6, AAV7, AAV8, AAVrh.74, avian AAV, bovine AAV, canine AAV, equine AAV, primate AAV, non-primate AAV, ovine AAV, and any combination thereof. In some aspects, the AAV vector is derived from an AAV vector selected from the group consisting of AAV type 1 (AAV1), AAV2, AAV3, AAV4, AAV5, AAV6, AAV7, AAV8, AAVrh.74, avian AAV, bovine AAV, canine AAV, equine AAV, primate AAV, non-primate AAV, ovine AAV, and any combination thereof. In certain aspects, the AAV vector comprises regions of at least two different AAV vectors known in the art. In some aspects, the AAV vector comprises an inverted terminal repeat from a first AAV (e.g., AAV1, AAV2, AAV3, AAV4, AAV5, AAV6, AAV7, AAV8, AAVrh.74, avian AAV, bovine AAV, canine AAV, equine AAV, primate AAV, non-primate AAV, or ovine AAV) and a second inverted terminal repeat from a second AAV (e.g., AAV1, AAV2, AAV3, AAV4, AAV5, AAV6, AAV7, AAV8, AAVrh.74, avian AAV, bovine AAV, canine AAV, equine AAV, primate AAV, non-primate AAV, or ovine AAV).

In some aspects, the AAV vector is an AAV1 vector. In some aspects, the AAV vector is derived from an AAV1 vector. In some aspects, the AAV vector comprises a portion of an AAV1 vector. In some aspects, the AAV vector is an AAV2 vector. In some aspects, the AAV vector is derived from an AAV2 vector. In some aspects, the AAV vector comprises a portion of an AAV2 vector. In some aspects, the AAV vector is an AAV3 vector. In some aspects, the AAV vector is derived from an AAV3 vector. In some aspects, the AAV vector comprises a portion of an AAV3 vector. In some aspects, the AAV vector is an AAV8 vector. In some aspects, the AAV vector is derived from an AAV8 vector. In some aspects, the AAV vector comprises a portion of an AAV8 vector. In some aspects, the AAV vector is an AAV9 vector. In some aspects, the AAV vector is derived from an AAV9 vector. In some aspects, the AAV vector comprises a portion of an AAV9 vector. In some aspects, the AAV vector is an AAVrh.74 vector. In some aspects, the AAV vector is derived from an AAVrh.74 vector. In some aspects, the AAV vector comprises a portion of an AAVrh.74 vector. For example, the nucleotide sequence of the AAV serotype 2 (AAV2) genome is presented in Srivastava et al., J Virol. 45: 555-564 (1983) as corrected by Ruffing et al., J Gen Virol. 75: 3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol. 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac et al., Journal of Translational Medicine 5: 45 (2007).

In certain aspects, the AAV vector nucleic acid comprises a nucleic acid sequence of the capsid gene of AAVrh.74. In some aspects, the AAV vector nucleic acid comprises a capsid gene, wherein the capsid gene comprises a nucleic acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 47. In some aspects, the AAV vector nucleic acid comprises a capsid gene, wherein the capsid gene comprises the nucleic acid sequence set forth in SEQ ID NO: 47. In some aspects, the AAV vector nucleic acid comprises a capsid gene, wherein the capsid gene comprises a nucleic acid sequence encoding a polypeptide having at least about 80%, at least about 85%, at least about 90%, at least about 95%, at least about 96%, at least about 97%, at least about 98%, or at least about 99% sequence identity to the amino acid sequence set forth in SEQ ID NO: 48. In some aspects, the AAV vector nucleic acid comprises a capsid gene, wherein the capsid gene comprises a nucleic acid sequence encoding the amino acid sequence set forth in SEQ ID NO: 48.

In certain aspects, the AAV vector nucleic acid comprises a nucleic acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 49. In some aspects, the AAV vector nucleic acid comprises a nucleic acid sequence as set forth in SEQ ID NO: 49. In some aspects, the AAV vector nucleic acid comprises a portion of the nucleic acid sequence set forth in SEQ ID NO: 49. SEQ ID NO: 49 represents the nucleic acid sequence of the AAVrh.74 genome. Nucleotides 210-2147 of SEQ ID NO: 49 are the Rep 78 gene open reading frame. In some aspects, the AAV vector nucleic acid comprises the Rep 78 gene open reading frame of AAVrh.74. Nucleotides 882-2081 of SEQ ID NO: 49 are the Rep52 open reading frame. In some aspects, the AAV vector nucleic acid comprises the Rep52 open reading frame of AAVrh.74. Nucleotides 2079-2081 of SEQ ID NO: 49 are the Rep52 stop. In some aspects, the AAV vector nucleic acid comprises the Rep52 stop of AAVrh.74. Nucleotides 2145-2147 of SEQ ID NO: 49 are the Rep78 stop. In some aspects, the AAV vector nucleic acid comprises the Rep78 stop of AAVrh.74. Nucleotides 1797-1800 of SEQ ID NO: 49 are a splice donor site. In some aspects, the AAV vector nucleic acid comprises a splice donor site of AAVrh.74. Nucleotides 2094-2097 of SEQ ID NO: 49 are a splice acceptor site. In some aspects, the AAV vector nucleic acid comprises a splice acceptor site corresponding to nucleotides 2094-2097 of AAVrh.74 (SEQ ID NO: 49). Nucleotides 2121-2124 of SEQ ID NO: 49 are a splice acceptor site. In some aspects, the AAV vector nucleic acid comprises a splice acceptor site corresponding to nucleotides 2121-2124 of AAVrh.74 (SEQ ID NO: 49). Nucleotides 174-181 of SEQ ID NO: 49 are the p5 promoter +1 predicted. In some aspects, the AAV vector nucleic acid comprises the p5 promoter +1 predicted of AAVrh.74. Nucleotides 145-151 of SEQ ID NO: 49 are the p5 TATA box. In some aspects, the AAV vector nucleic acid comprises the Rep52 open reading frame of AAVrh.74. Nucleotides 758-761 of SEQ ID NO: 49 are the p19 promoter +1 predicted. In some aspects, the AAV vector nucleic acid comprises the Rep52 open reading frame of AAVrh.74. Nucleotides 732-738 of SEQ ID NO: 49 are the p19 TATA box. In some aspects, the AAV vector nucleic acid comprises the Rep52 open reading frame of AAVrh.74. Nucleotides 1711-1716 of SEQ ID NO: 49 are the p40 TATA box. In some aspects, the AAV vector nucleic acid comprises the p40 TATA box of AAVrh.74. Nucleotides 2098-4314 of SEQ ID NO: 49 are the VP1 Cap gene open reading frame. In some aspects, the AAV vector nucleic acid comprises the VP1 Cap gene open reading frame of AAVrh.74. Nucleotides 2509-2511 of SEQ ID NO: 49 are the VP2 start. In some aspects, the AAV vector nucleic acid comprises the VP2 start of AAVrh.74. Nucleotides 2707-2709 of SEQ ID NO: 49 are the VP3 start. In some aspects, the AAV vector nucleic acid comprises the VP3 start of AAVrh.74. Nucleotides 4328-4333 of SEQ ID NO: 49 are a polyA signal. In some aspects, the AAV vector nucleic acid comprises the polyA signal of AAVrh.74.

TABLE 6
AAVrh.74 Sequences

| | |
|---|---|
| Capsid gene | ATGGCTGCCGATGGTTATCTTCCAGATTGGCTCGAGGACAACCTCTCTGAGGGC |
| of AAVrh.74 | ATTCGCGAGTGGTGGGACCTGAAACCTGGAGCCCCGAAACCCAAAGCCAACCAG |
| (SEQ ID NO: | CAAAAGCAGGACAACGGCCGGGGTCTGGTGCTTCCTGGCTACAAGTACCTCGGA |
| 47) | CCCTTCAACGGACTCGACAAGGGGGAGCCCGTCAACGCGGCGGACGCAGCGGCC |
| | CTCGAGCACGACAAGGCCTACGACCAGCAGCTCCAAGCGGGTGACAATCCGTAC |
| | CTGCGGTATAATCACGCCGACGCCGAGTTTCAGGAGCGTCTGCAAGAAGATACG |
| | TCTTTTGGGGGCAACCTCGGGCGCGCAGTCTTCCAGGCCAAAAAGCGGGTTCTC |
| | GAACCTCTGGGCCTGGTTGAATCGCCGGTTAAGACGGCTCCTGGAAAGAAGAGA |
| | CCGGTAGAGCCATCACCCCAGCGCTCTCCAGACTCCTCTACGGGCATCGGCAAG |
| | AAAGGCCAGCAGCCCGCAAAAAAGAGACTCAATTTTGGGCAGACTGGCGACTCA |
| | GAGTCAGTCCCCGACCCTCAACCAATCGGAGAACCACCAGCAGGCCCCTCTGGT |
| | CTGGGATCTGGTACAATGGCTGCAGGCGGTGGCGCTCCAATGGCAGACAATAAC |
| | GAAGGCGCCGACGGAGTGGGTAGTTCCTCAGGAAATTGGCATTGCGATTCCACA |
| | TGGCTGGGCGACAGAGTCATCACCACCAGCACCCGCACCTGGGCCCTGCCCACC |
| | TACAACAACCACCTCTACAAGCAAATCTCCAACGGGACCTCGGGAGGAAGCACC |
| | AACGACAACACCTACTTCGGCTACAGCACCCCCTGGGGGTATTTTGACTTCAAC |
| | AGATTCCACTGCCACTTTTCACCACGTGACTGGCAGCGACTCATCAACAACAAC |
| | TGGGGATTCCGGCCCAAGAGGCTCAACTTCAAGCTCTTCAACATCCAAGTCAAG |
| | GAGGTCACGCAGAATGAAGGCACCAAGACCATCGCCAATAACCTTACCAGCACG |
| | ATTCAGGTCTTTACGGACTCGGAATACCAGCTCCCGTACGTGCTCGGCTCGGCG |
| | CACCAGGGCTGCCTGCCTCCGTTCCCGGCGGACGTCTTCATGATTCCTCAGTAC |
| | GGGTACCTGACTCTGAACAATGGCAGTCAGGCTGTGGGCCGGTCGTCCTTCTAC |
| | TGCCTGGAGTACTTTCCTTCTCAAATGCTGAGAACGGGCAACAACTTTGAATTC |
| | AGCTACAACTTCGAGGACGTGCCCTTCCACAGCAGCTACGCGCACAGCCAGAGC |
| | CTGGACCGGCTGATGAACCCTCTCATCGACCAGTACTTGTACTACCTGTCCCGG |
| | ACTCAAAGCACGGGCGGTACTGCAGGAACTCAGCAGTTGCTATTTTCTCAGGCC |
| | GGGCCTAACAACATGTCGGCTCAGGCCAAGAACTGGCTACCCGGTCCCTGCTAC |
| | CGGCAGCAACGCGTCTCCACGACACTGTCGCAGAACAACAACAGCAACTTTGCC |
| | TGGACGGGTGCCACCAAGTATCATCTGAATGGCAGAGACTCTCTGGTGAATCCT |
| | GGCGTTGCCATGGCTACCCACAAGGACGACGAAGAGCGATTTTTTCCATCCAGC |
| | GGAGTCTTAATGTTTGGGAAACAGGGAGCTGGAAAAGACAACGTGGACTATAGC |
| | AGCGTGATGCTAACCAGCGAGGAAGAAATAAAGACCACCAACCCAGTGGCCACA |
| | GAACAGTACGGCGTGGTGGCCGATAACCTGCAACAGCAAAACGCCGCTCCTATT |
| | GTAGGGGCCGTCAATAGTCAAGGAGCCTTACCTGGCATGGTGTGGCAGAACCGG |
| | GACGTGTACCTGCAGGGTCCCATCTGGGCCAAGATTCCTCATACGGACGGCAAC |
| | TTTCATCCCTCGCCGCTGATGGGAGGCTTTGGACTGAAGCATCCGCCTCCTCAG |
| | ATCCTGATTAAAAACACACCTGTTCCCGCGGATCCTCCGACCACCTTCAATCAG |
| | GCCAAGCTGGCTTCTTTCATCACGCAGTACAGTACCGGCCAGGTCAGCGTGGAG |
| | ATCGAGTGGGAGCTGCAGAAGGAGAACAGCAAACGCTGGAACCCAGAGATTCAG |
| | TACACTTCCAACTACTACAAATCTACAAATGTGGACTTTGCTGTCAATACTGAG |
| | GGTACTTATTCCGAGCCTCGCCCCATTGGCACCCGTTACCTCACCCGTAATCTG |
| | TAA |
| Capsid of | MAADGYLPDWLEDNLSEGIREWWDLKPGAPKPKANQQKODNGRGLVLPGYKYLG |
| AAVrh.74 | PFNGLDKGEPVNAADAAALEHDKAYDQQLQAGDNPYLRYNHADAEFQERLQEDT |
| (SEQ ID NO: | SFGGNLGRAVFQAKKRVLEPLGLVESPVKTAPGKKRPVEPSPORSPDSSTGIGK |
| 48) | KGQQPAKKRLNFGQTGDSESVPDPQPIGEPPAGPSGLGSGTMAAGGGAPMADNN |
| | EGADGVGSSSGNWHCDSTWLGDRVITTSTRTWALPTYNNHLYKQISNGTSGGST |
| | NDNTYFGYSTPWGYFDFNRFHCHFSPRDWORLINNNWGFRPKRLNFKLFNIQVK |
| | EVTQNEGTKTIANNLTSTIQVFTDSEYQLPYVLGSAHQGCLPPFPADVFMIPQY |
| | GYLTLNNGSQAVGRSSFYCLEYFPSQMLRTGNNFEFSYNFEDVPFHSSYAHSQS |
| | LDRLMNPLIDQYLYYLSRTQSTGGTAGTQQLLFSQAGPNNMSAQAKNWLPGPCY |
| | RQQRVSTTLSQNNNSNFAWTGATKYHLNGRDSLVNPGVAMATHKDDEERFFPSS |
| | GVLMFGKOGAGKDNVDYSSVMLTSEEEIKTTNPVATEQYGVVADNLQQQNAAPI |
| | VGAVNSQGALPGMVWQNRDVYLQGPIWAKIPHTDGNFHPSPLMGGFGLKHPPPQ |
| | ILIKNTPVPADPPTTFNQAKLASFITQYSTGQVSVEIEWELQKENSKRWNPEIQ |
| | YTSNYYKSTNVDFAVNTEGTYSEPRPIGTRYLTRNL |
| AAVrh.74 | CTCCATCACTAGGGGTAACCGCGAAGCGCCTCCCACGCTGCCGCGTCAGCGCTG |
| Genomic | ACGTAAATTACGTCATAGGGGAGTGGTCCTGTATTAGCTGTCACGTGAGTGCTT |
| Sequence | TTGCGACATTTTGCGACACCACGTGGCCATTCATGGTATATATGGCCGAGTGAG |
| (SEQ ID NO: | CGAGCAGGATCTCCATTTTGACCGCGAAATTTGAACGAGCAGCAGCCATGCCGG |
| 49) | GCTTCTACGAGATCGTGCTTAAGGTGCCGAGCGACCTGGACGAGCACCTGCCGG |
| | GCATTTCTGACTCGTTTGTGAACTGGGTGGCAGAGAAGGAATGGGAGCTGCCCC |
| | CGGATTCTGACATGGATCGGAATCTGATTGAGCAGGCACCCCTGACCGTGGCCG |
| | AGAAGCTACAGCGCGACTTCCTGGTCCAATGGCGCCGCGTGAGTAAGGCCCCGG |
| | AGGCCCTCTTCTTTGTTCAGTTCGAGAAGGGCGAGTCCTACTTCCACCTCCATA |
| | TTCTGGTAGAGACCACGGGGGTCAAATCCATGGTGCTGGGCCGCTTCCTGAGTC |
| | AGATTCGGGACAAGCTGGTGCAGACCATCTACCGCGGGATCGAGCCGACCCTGC |
| | CCAACTGGTTCGCGGTGACAAAGACGCGTAATGGCGCCGGAGGGGGGAACAAGG |
| | TGGTGGACGAGTGCTACATCCCCAACTACCTGCTGCCCAAGACTCAGCCCGAGC |
| | TGCAGTGGGCGTGGACTAACATGGAGGAGTATATAAGCGCGTGCTTGAACCTGG |
| | CCGAGCGCAAACGGCTCGTGGCGCAGCACCTGACCCACGTCAGCCAGACCCAGG |
| | AGCAGAACAAGGAGAATCTGAACCCGAATTCTGACGCGCCTGTCATCCGGTCAA |
| | AAACCTCCGCGCGCTACATGGAGCTGGTCGGGTGGCTGGTGGACCGGGGCATCA |
| | CCTCCGAGAAGCAGTGGATCCAGGAGGACCAGGCCTCGTACATCTCCTTCAACG |
| | CCGCCTCCAACTCGCGGTCTCAGATCAAGGCCGCGCTGGACAATGCCGGCAAGA |
| | TCATGGCGCTGACCAAATCCGCGCCCGACTACCTGGTAGGCCCCGCTCTGCCCG |
| | CGGACATTAAATCCAACCGCATCTACCGCATCCTGGAGCTGAATGGCTACGACC |
| | CTGCCTACGCCGGTTCCGTCTTTCTCGGCTGGGCCCAGAAAAAGTTTGGCAAAA |
| | GGAACACCATCTGGCTGTTTGGGCCGGCCACCACGGGCAAGACCAACATCGCGG |
| | AAGCCATCGCCCACGCCGTGCCCTTCTACGGCTGCGTCAACTGGACCAATGAGA |
| | ACTTTCCCTTCAACGATTGCGTCGACAAGATGGTGATCTGGTGGGAGGAGGGCA |
| | AGATGACGGCCAAGGTCGTGGAGTCCGCCAAGGCCATTCTCGGCGGCAGCAAGG |
| | TGCGCGTGGACCAAAAGTGCAAGTCGTCCGCCCAGATCGATCCCACCCCCGTGA |
| | TCGTCACCTCCAACACCAACATGTGCGCCGTGATTGACGGGAACAGCACCACCT |
| | TCGAGCACCAGCAGCCGTTGCAGGACCGGATGTTCAAATTTGAACTTACCCGCC |
| | GTCTGGAGCACGACTTTGGCAAGGTGACAAAGCAGGAAGTCAAAGAGTTCTTCC |
| | GCTGGGCGCAGGATCACGTGACCGAGGTGGCGCATGAGTTCTACGTCAGAAAGG |
| | GTGGAGCTAACAAAAGACCCGCCCCCGATGACGCGGATATAAGCGAGCCCAAGC |
| | GGGCCTGCCCCTCAGTCGCGGATCCATCGACGTCAGACGCGGAAGGAGCTCCGG |
| | TGGACTTTGCCGACAGGTACCAAAACAAATGTTCTCGTCACGCGGGCATGCTTC |
| | AGATGCTGTTTCCCTGCAAAACATGCGAGAGAATGAATCAGAATTTCAACATTT |
| | GCTTCACGCACGGGACCAGAGACTGTTCAGAATGTTTCCCTGGCGTGTCAGAAT |
| | CTCAACCGGTCGTCAGAAAAAAGACGTATCGGAAACTCTGTGCGATTCATCATC |
| | TGCTGGGGGGGGCACCCGAGATTGCTTGCTCGGCCTGCGACCTGGTCAACGTGG |
| | ACCTGGATGACTGTGTTTCTGAGCAATAAATGACTTAAACCAGGTATGGCTGCC |
| | GATGGTTATCTTCCAGATTGGCTCGAGGACAACCTCTCTGAGGGCATTCGCGAG |
| | TGGTGGGACCTGAAACCTGGAGCCCCGAAACCCAAAGCCAACCAGCAAAAGCAG |
| | GACAACGGCCGGGGTCTGGTGCTTCCTGGCTACAAGTACCTCGGACCCTTCAAC |
| | GGACTCGACAAGGGGGAGCCCGTCAACGCGGCGGACGCAGCGGCCCTCGAGCAC |
| | GACAAGGCCTACGACCAGCAGCTCCAAGCGGGTGACAATCCGTACCTGCGGTAT |
| | AATCACGCCGACGCCGAGTTTCAGGAGCGTCTGCAAGAAGATACGTCTTTTGGG |
| | GGCAACCTCGGGCGCGCAGTCTTCCAGGCCAAAAAGCGGGTTCTCGAACCTCTG |
| | GGCCTGGTTGAATCGCCGGTTAAGACGGCTCCTGGAAAGAAGAGACCGGTAGAG |
| | CCATCACCCCAGCGCTCTCCAGACTCCTCTACGGGCATCGGCAAGAAAGGCCAG |
| | CAGCCCGCAAAAAAGAGACTCAATTTTGGGCAGACTGGCGACTCAGAGTCAGTC |
| | CCCGACCCTCAACCAATCGGAGAACCACCAGCAGGCCCCTCTGGTCTGGGATCT |
| | GGTACAATGGCTGCAGGCGGTGGCGCTCCAATGGCAGACAATAACGAAGGCGCC |
| | GACGGAGTGGGTAGTTCCTCAGGAAATTGGCATTGCGATTCCACATGGCTGGGC |
| | GACAGAGTCATCACCACCAGCACCCGCACCTGGGCCCTGCCCACCTACAACAAC |
| | CACCTCTACAAGCAAATCTCCAACGGGACCTCGGGAGGAAGCACCAACGACAAC |
| | ACCTACTTCGGCTACAGCACCCCCTGGGGGTATTTTGACTTCAACAGATTCCAC |
| | TGCCACTTTTCACCACGTGACTGGCAGCGACTCATCAACAACAACTGGGGATTC |
| | CGGCCCAAGAGGCTCAACTTCAAGCTCTTCAACATCCAAGTCAAGGAGGTCACG |
| | CAGAATGAAGGCACCAAGACCATCGCCAATAACCTTACCAGCACGATTCAGGTC |
| | TTTACGGACTCGGAATACCAGCTCCCGTACGTGCTCGGCTCGGCGCACCAGGGC |
| | TGCCTGCCTCCGTTCCCGGCGGACGTCTTCATGATTCCTCAGTACGGGTACCTG |
| | ACTCTGAACAATGGCAGTCAGGCTGTGGGCCGGTCGTCCTTCTACTGCCTGGAG |
| | TACTTTCCTTCTCAAATGCTGAGAACGGGCAACAACTTTGAATTCAGCTACAAC |
| | TTCGAGGACGTGCCCTTCCACAGCAGCTACGCGCACAGCCAGAGCCTGGACCGG |
| | CTGATGAACCCTCTCATCGACCAGTACTTGTACTACCTGTCCCGGACTCAAAGC |
| | ACGGGCGGTACTGCAGGAACTCAGCAGTTGCTATTTTCTCAGGCCGGGCCTAAC |
| | AACATGTCGGCTCAGGCCAAGAACTGGCTACCCGGTCCCTGCTACCGGCAGCAA |
| | CGCGTCTCCACGACACTGTCGCAGAACAACAACAGCAACTTTGCCTGGACGGGT |
| | GCCACCAAGTATCATCTGAATGGCAGAGACTCTCTGGTGAATCCTGGCGTTGCC |
| | ATGGCTACCCACAAGGACGACGAAGAGCGATTTTTTCCATCCAGCGGAGTCTTA |
| | ATGTTTGGGAAACAGGGAGCTGGAAAAGACAACGTGGACTATAGCAGCGTGATG |
| | CTAACCAGCGAGGAAGAAATAAAGACCACCAACCCAGTGGCCACAGAACAGTAC |
| | GGCGTGGTGGCCGATAACCTGCAACAGCAAAACGCCGCTCCTATTGTAGGGGCC |
| | GTCAATAGTCAAGGAGCCTTACCTGGCATGGTGTGGCAGAACCGGGACGTGTAC |
| | CTGCAGGGTCCCATCTGGGCCAAGATTCCTCATACGGACGGCAACTTTCATCCC |
| | TCGCCGCTGATGGGAGGCTTTGGACTGAAGCATCCGCCTCCTCAGATCCTGATT |
| | AAAAACACACCTGTTCCCGCGGATCCTCCGACCACCTTCAATCAGGCCAAGCTG |
| | GCTTCTTTCATCACGCAGTACAGTACCGGCCAGGTCAGCGTGGAGATCGAGTGG |
| | GAGCTGCAGAAGGAGAACAGCAAACGCTGGAACCCAGAGATTCAGTACACTTCC |
| | AACTACTACAAATCTACAAATGTGGACTTTGCTGTCAATACTGAGGGTACTTAT |
| | TCCGAGCCTCGCCCCATTGGCACCCGTTACCTCACCCGTAATCTGTAATTACAT |
| | GTTAATCAATAAACCGGTTAATTCGTTTCAGTTGAACTTTGGTCTCCTGTCCTT |
| | CTTATCTTATCGGTTACCATAGAAACTGGTTACTTATTAACTGCTTGGTGCGCT |
| | TCGCGATAAAAGACTTACGTCATCGGGTTACCCCTAGTGATGGA |

In some aspects, the AAV vector nucleic acid comprises a promoter. Any promoter known in the art can be used in the AAV vector nucleic acid of the present disclosure. In some aspects, the AAV vector nucleic acid comprises a constitutively active promoter. Any constitutively active promoter can be used in the AAV vector nucleic acid, including, but not limited to a cytomegalovirus immediate-early gene (CMV) promoter, an EF1a promoter, an SV40 promoter, a PGK1 promoter, a Ubc promoter, a human beta actin promoter, a CAG promoter, a TRE promoter, a UAS promoter, a Ac5 promoter, a polyhedrin promoter, a CaMKIIa promoter, a GAL1 promoter, a GAL10 promoter, a TEF promoter, a GDS promoter, a ADH1 promoter, a CaMV35S promoter, a Ubi promoter, an H1 promoter, a U6, or any combination thereof. In certain aspects, the AAV vector comprises a CMV promoter. In certain aspects, the AAV vector nucleic acid comprises a SV40 promoter.

In certain aspects, the AAV vector nucleic acid comprises a tissue specific promoter. In certain aspects, the tissue specific promoter drives expression of the therapeutic molecule encoded by the AAV, e.g., micro-dystrophin, alpha sarcoglycan, and/or beta sarcoglycan, in the brain, muscle, kidney, lung, testis, or any combination thereof. In some aspects, the tissue specific promoter drives expression in a cell of a quadriceps muscle of the subject, a heart muscle of the subject, a diaphragm muscle of the subject, or any combination thereof. In particular aspects, the tissue specific promoter drives expression of the therapeutic molecule encoded by the AAV, e.g., micro-dystrophin, alpha sarcoglycan, and/or beta sarcoglycan, in a cell of a quadriceps muscle of the subject. In particular aspects, the tissue specific promoter drives expression of the therapeutic molecule encoded by the AAV, e.g., micro-dystrophin, alpha sarcoglycan, and/or beta sarcoglycan, in a cell of a heart muscle of the subject. In particular aspects, the tissue specific promoter drives expression of the therapeutic molecule encoded by the AAV, e.g., micro-dystrophin, alpha sarcoglycan, and/or beta sarcoglycan, in a cell of a diaphragm muscle of the subject.

In particular aspects, the promoter is selected from the group consisting of enhancer 358 bp muscle creatine kinase proximal promoter (Enh358MCK), muscle creatine kinase (CK) promoter (e.g., hCK (SEQ ID NO: 31) or hCK Plus (SEQ ID NO: 32)), truncated muscle creatine kinase promoter (tMCK; SEQ ID NO: 26), myosin heavy chain (MHC), MHCK7 (a hybrid version of MHC and MCK; SEQ ID NO: 25), C5-12 (synthetic promoter), a muscle- and heart-specific enhancer (MHCK), CK8, SPc5-12, human desmin (Des) promoter, human alpha-myosin heavy chain (a-MHC) promoter, rat myosin light chain 2 (MLC-2) promoter, and human cardiac troponin C (cTnC) promoter. In certain aspects, the promoter comprises a MCK promoter. In certain aspects, the promoter comprises a MHCK promoter. In some aspects, the tissue specific promoter is an Enh358MCK. In some aspects, the tissue specific promoter is a CK promoter. In some aspects, the tissue specific promoter is a muscle- and heart-specific enhancer 7 (MHCK7) promoter. In some aspects, the tissue specific promoter is a CK8 promoter. In some aspects, the tissue specific promoter is a SPc5-12 promoter.

In some aspects, the promoter comprises a nucleic acid sequence having a least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 25. In some aspects, the promoter comprises the nucleic acid sequence set forth in SEQ ID NO: 25.

In some aspects, the promoter comprises a nucleic acid sequence having a least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 26. In some aspects, the promoter comprises the nucleic acid sequence set forth in SEQ ID NO: 26.

In some aspects, the AAV vector nucleic acid comprises one or more enhancers. In some aspects, the one or more enhancers are present in the AAV alone or together with a promoter disclosed herein. In some aspects, at least one of the one or more enhancers is a tissue specific enhancer. In certain aspects, the one or more enhancers are selected from a human skeletal actin gene element, a cardiac actin gene element, a myocyte-specific enhancer binding factor MEF (e.g, MEF2), a MyoD enhancer element, a cardiac enhancer factor (CEF) site, murine creatine kinase enhancer element, skeletal fast-twitch troponin C gene element, slow-twitch cardiac troponin C gene element, the slow-twitch troponin I gene element, hypozia-inducible nuclear factors, steroid-inducible element, glucocorticoid response element (GRE) and any combination thereof. In some aspects, the AAV vector nucleic acid comprises an MEF2. In some aspects, the AAV vector nucleic acid comprises a MyoD enhancer element. In some aspects, the AAV vector comprises a CEF site.

In some aspects, the AAV vector nucleic acid further comprises an intronic sequence, i.e., an intron. In some aspects, the intronic sequence is positioned 5′ to the nucleic acid sequence encoding the dystrophin. In some aspects, the intronic sequence is positioned 3′ to the promoter. In some aspects, the intronic sequence comprises a synthetic intronic sequence. In some aspects, the intron comprises a nucleic acid sequence having a least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to a nucleic acid sequence selected from SEQ ID NOs: 27-29. In some aspects, the intron comprises the nucleic acid sequence set forth in SEQ ID NO: 27. In some aspects, the intron comprises the nucleic acid sequence set forth in SEQ ID NO: 28. In some aspects, the intron comprises the nucleic acid sequence set forth in SEQ ID NO: 29.

TABLE 7
Sample Intronic Sequences

AGGTAAGTTTAGTCTTTTTGTCTTTTATTTCAGGTCCCGGATCCGGTGGTGGTGCAAATCAAA
GAACTGCTCCTCAGTGGATGTTGCCTTTACTTCTAGGCCTGTACGGAAGTGTTACTTCTGCTC
TAAAAGCTGCGGAATTGTACCCGC (SEQ ID NO: 27)
AGGTAAGTTTAGTCTTTTTGTCTTTTATTTCAGGTCCCGGATCCGGTGGTGGTGCAAATCAAA
GAACTGCTCCTCAGTGGATGTTGCCTTTACTTCTAGGCCTGTACGGAAGTGTTACTTCTGCTC
TAAAAGCTGCGGAATTGTACCC (SEQ ID NO: 28)
GTAAGTATCAAGGTTACAAGACAGGTTTAAGGAGACCAATAGAAACTGGGCTTGTCGAGACAG
AGAAGACTCTTGCGTTTCTGATAGGCACCTATTGGTCTTACTGACATCCACTTTGCCTTTCTC
TCCACAG (SEQ ID NO: 29)

In some aspects, the AAV vector nucleic acid comprises a post-transcriptional regulatory element. In some aspects, the post-transcriptional regulatory element is positioned 3′ to the nucleic acid sequence encoding the dystrophin. In some aspects, the post-transcriptional regulatory element comprises a mutated woodchuck hepatitis virus post-transcriptional regulatory element (WPRE), a microRNA binding site, a DNA nuclear targeting sequence (DTS), or any combination thereof. In some aspects, the microRNA binding site comprises a binding site to miR142-3p. In other aspects, the miRNA binding site is selected from a miRNA binding site disclosed by Rennie et al., RNA Biol. 13(6):554-560 (2016), and STarMirDB, available at http://sfold.wadsworth.org/starmirDB.php, which are incorporated by reference herein in their entirety. In certain aspects, the DTS comprises an SV40 enhancer sequence. In certain aspects, the DTS comprises a c-Myc enhancer sequence.

In some aspects, the AAV vector nucleic acid comprises a 3′UTR poly(A) tail sequence. In some aspects, the 3′UTR poly(A) tail sequence is selected from the group consisting of bGH poly(A), actin poly(A), hemoglobin poly(A), and any combination thereof. In some aspects, the 3′UTR poly(A) tail sequence comprises bGH poly(A). in some aspects, the 3′UTR poly(A) tail sequence comprises a nucleic acid sequence having a least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 30 (GGCCGCAATAAAAGATCTTTATTTTCATTAGATCTGTGTGTTGGTTTTTTGTG). In some aspects, the 3′UTR poly(A) tail sequence comprises the nucleic acid sequence set forth in SEQ ID NO: 30.

In certain aspect, the AAV vector nucleic acid of the present disclosure further comprises a genetic cassette encoding a therapeutic molecule. In some aspects, the therapeutic molecule is selected from a polypeptide, an RNA molecule, or a DNA molecule. In certain aspects, the therapeutic molecule encoded by the genetic cassette is a therapeutic RNA molecule. In some aspects, the therapeutic RNA molecule is selected from a miRNA, an siRNA, an mRNA, or any combination thereof. In some aspects, the therapeutic molecule is an antisense oligomer.

In some aspects, the therapeutic molecule comprises a polypeptide. In some aspects, the polypeptide is a polypeptide that is not expressed or mis-expressed in the human subject. In certain aspects, the genetic cassette encodes a therapeutic polypeptide selected from a micro-dystrophin, beta sarcoglycan, alpha sarcoglycan, and any combination thereof.

### IV.A. Micro-Dystrophin Transgenes

In some aspects, the AAV vector, e.g., the AAVrh74 vector, comprises a genetic cassette encoding a micro-dystrophin polypeptide. In some aspects, the genetic cassette comprises a nucleic acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 19. In some aspects, the genetic cassette comprises the nucleic acid sequence set forth in SEQ ID NO: 19.

In some aspects, the genetic cassette comprises a nucleic acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to a nucleic acid sequence selected from the nucleic acid sequences set forth in SEQ ID NOs: 33-39. In some aspects, the genetic cassette comprises a nucleic acid sequence selected from the nucleic acid sequences set forth in SEQ ID NOs: 33-39.

In some aspects, the genetic cassette comprises a nucleic acid sequence encoding a micro-dystrophin polypeptide, wherein the micro-dystrophin polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to an amino acid sequence selected from the amino acid sequences set forth in SEQ ID NOs: 40-44. In some aspects, the genetic cassette comprises a nucleic acid sequence encoding a micro-dystrophin polypeptide, wherein the micro-dystrophin polypeptide comprises the amino acid sequence set forth in SEQ ID NO: 40. In some aspects, the genetic cassette comprises a nucleic acid sequence encoding a micro-dystrophin polypeptide, wherein the micro-dystrophin polypeptide comprises the amino acid sequence set forth in SEQ ID NO: 41. In some aspects, the genetic cassette comprises a nucleic acid sequence encoding a micro-dystrophin polypeptide, wherein the micro-dystrophin polypeptide comprises the amino acid sequence set forth in SEQ ID NO: 42. In some aspects, the genetic cassette comprises a nucleic acid sequence encoding a micro-dystrophin polypeptide, wherein the micro-dystrophin polypeptide comprises the amino acid sequence set forth in SEQ ID NO: 43. In some aspects, the genetic cassette comprises a nucleic acid sequence encoding a micro-dystrophin polypeptide, wherein the micro-dystrophin polypeptide comprises the amino acid sequence set forth in SEQ ID NO: 44.

In certain aspects, the AAV vector, e.g., the AAVrh74 vector, comprises a genetic cassette encoding a micro-dystrophin polypeptide and a promoter, e.g., any promoter disclosed herein. In certain aspects, the AAV vector, e.g., the AAVrh74 vector, comprises (i) a genetic cassette comprising a nucleic acid sequence encoding a micro-dystrophin disclosed herein and (ii) an MHCK7 promoter. In certain aspects, the AAV vector, e.g., the AAVrh74 vector, comprises (i) a genetic cassette comprising the nucleic acid sequence set forth in SEQ ID NO: 19 and (ii) an MHCK7 promoter. In certain aspects, the AAV vector, e.g., the AAVrh74 vector, comprises (i) a genetic cassette comprising the nucleic acid sequence set forth in SEQ ID NO: 19 and (ii) a promoter comprising the nucleic acid sequence set forth in SEQ ID NO: 25.

In certain aspects, the AAV vector, e.g., the AAVrh74 vector, comprises a genetic cassette encoding a micro-dystrophin polypeptide and an intronic sequence, e.g., any intronic sequence disclosed herein. In certain aspects, the AAV vector, e.g., the AAVrh74 vector, comprises (i) a genetic cassette comprising a nucleic acid sequence encoding a micro-dystrophin disclosed herein and (ii) an intronic sequence comprising the nucleic acid sequence set forth in SEQ ID NO: 27. In certain aspects, the AAV vector, e.g., the AAVrh74 vector, comprises (i) a genetic cassette comprising the nucleic acid sequence set forth in SEQ ID NO: 19 and (ii) an intronic sequence comprising the nucleic acid sequence set forth in SEQ ID NO: 27. In certain aspects, the AAV vector, e.g., the AAVrh74 vector, comprises (i) a genetic cassette comprising the nucleic acid sequence set forth in SEQ ID NO: 19; (ii) a promoter comprising the nucleic acid sequence set forth in SEQ ID NO: 25; and (iii) an intronic sequence comprising the nucleic acid sequence set forth in SEQ ID NO: 27.

In certain aspects, the AAV vector, e.g., the AAVrh74 vector, comprises a genetic cassette encoding a micro-dystrophin polypeptide and a 3′UTR poly(A) tail sequence, e.g., any 3′UTR poly(A) tail sequence disclosed herein. In certain aspects, the AAV vector, e.g., the AAVrh74 vector, comprises (i) a genetic cassette comprising a nucleic acid sequence encoding a micro-dystrophin disclosed herein and (ii) a 3′UTR poly(A) tail sequence comprising the nucleic acid sequence set forth in SEQ ID NO: 30. In certain aspects, the AAV vector, e.g., the AAVrh74 vector, comprises (i) a genetic cassette comprising the nucleic acid sequence set forth in SEQ ID NO: 19 and (ii) a 3′UTR poly(A) tail sequence comprising the nucleic acid sequence set forth in SEQ ID NO: 30. In certain aspects, the AAV vector, e.g., the AAVrh74 vector, comprises (i) a genetic cassette comprising the nucleic acid sequence set forth in SEQ ID NO: 19; (ii) a promoter comprising the nucleic acid sequence set forth in SEQ ID NO: 25; (iii) an intronic sequence comprising the nucleic acid sequence set forth in SEQ ID NO: 27; and (iv) a 3′UTR poly(A) tail sequence comprising the nucleic acid sequence set forth in SEQ ID NO: 30.

In certain aspects, the AAV vector, e.g., the AAVrh74 vector, comprises a genetic cassette encoding a micro-dystrophin polypeptide, wherein the AAV vector, e.g., the AAVrh74 vector, comprises a nucleic acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 20. In certain aspects, the AAV vector, e.g., the AAVrh74 vector, comprises a genetic cassette encoding a micro-dystrophin polypeptide, wherein the AAV vector, e.g., the AAVrh74 vector, comprises the nucleic acid sequence set forth in SEQ ID NO: 20.

TABLE 8
Example AAVrh74-Micro-Dystrophin Nucleic Acid Sequence

GCGCGCTCGCTCGCTCACTGAGGCCGCCCGGGCAAAGCCCGGGCGTCGGGCGACCTTTGGTCG
CCCGGCCTCAGTGAGCGAGCGAGCGCGCAGAGAGGGAGTGGCCAACTCCATCACTAGGGGTTC
CTTGTAGTTAATGATTAACCCGCCATGCTACTTATCTACGTAGCCATGCTCTAGAGTTTAAAC
AAGCTTGCATGTCTAAGCTAGACCCTTCAGATTAAAAATAACTGAGGTAAGGGCCTGGGTAGG
GGAGGTGGTGTGAGACGCTCCTGTCTCTCCTCTATCTGCCCATCGGCCCTTTGGGGAGGAGGA
ATGTGCCCAAGGACTAAAAAAAGGCCATGGAGCCAGAGGGGCGAGGGCAACAGACCTTTCATG
GGCAAACCTTGGGGCCCTGCTGTCTAGCATGCCCCACTACGGGTCTAGGCTGCCCATGTAAGG
AGGCAAGGCCTGGGGACACCCGAGATGCCTGGTTATAATTAACCCAGACATGTGGCTGCCCCC
CCCCCCCCAACACCTGCTGCCTCTAAAAATAACCCTGTCCCTGGTGGATCCCCTGCATGCGAA
GATCTTCGAACAAGGCTGTGGGGGACTGAGGGCAGGCTGTAACAGGCTTGGGGGCCAGGGCTT
ATACGTGCCTGGGACTCCCAAAGTATTACTGTTCCATGTTCCCGGCGAAGGGCCAGCTGTCCC
CCGCCAGCTAGACTCAGCACTTAGTTTAGGAACCAGTGAGCAAGTCAGCCCTTGGGGCAGCCC
ATACAAGGCCATGGGGCTGGGCAAGCTGCACGCCTGGGTCCGGGGTGGGCACGGTGCCCGGGC
AACGAGCTGAAAGCTCATCTGCTCTCAGGGGCCCCTCCCTGGGGACAGCCCCTCCTGGCTAGT
CACACCCTGTAGGCTCCTCTATATAACCCAGGGGCACAGGGGCTGCCCTCATTCTACCACCAC
CTCCACAGCACAGACAGACACTCAGGAGCAGCCAGCGGCGCGCCCAGGTAAGTTTAGTCTTTT
TGTCTTTTATTTCAGGTCCCGGATCCGGTGGTGGTGCAAATCAAAGAACTGCTCCTCAGTGGA
TGTTGCCTTTACTTCTAGGCCTGTACGGAAGTGTTACTTCTGCTCTAAAAGCTGCGGAATTGT
ACCCGCGGCCGCCACCATGCTGTGGTGGGAGGAGGTGGAGGATTGTTATGAAAGGGAGGACGT
GCAGAAGAAGACTTTTACCAAGTGGGTGAACGCTCAGTTCAGCAAATTTGGGAAGCAGCACAT
CGAGAATCTGTTTTCCGACCTGCAGGATGGGAGACGGCTGCTGGATCTGCTGGAAGGACTGAC
TGGCCAGAAGCTGCCCAAAGAGAAGGGGAGCACTAGGGTGCACGCCCTGAACAACGTGAACAA
AGCTCTGAGAGTGCTGCAGAACAACAACGTGGATCTGGTGAATATTGGCAGTACTGATATCGT
GGACGGGAACCACAAACTGACACTGGGCCTGATCTGGAACATTATTCTGCACTGGCAGGTGAA
AAATGTGATGAAGAACATCATGGCCGGGCTGCAGCAGACCAATTCCGAGAAGATCCTGCTGTC
TTGGGTGCGGCAGAGCACCCGCAACTATCCCCAGGTGAACGTGATTAACTTCACTACATCCTG
GAGCGACGGGCTGGCCCTGAATGCTCTGATTCACAGCCACAGGCCTGATCTGTTCGACTGGAA
TAGCGTGGTGTGCCAGCAGTCTGCCACACAGCGCCTGGAACATGCCTTCAATATCGCTCGGTA
CCAGCTGGGGATCGAAAAACTGCTGGACCCAGAGGATGTGGACACTACATACCCAGATAAAAA
GTCTATTCTGATGTACATTACTAGCCTGTTCCAGGTGCTGCCACAGCAGGTGTCTATTGAAGC
CATTCAGGAGGTGGAAATGCTGCCCCGCCCCCCCAAAGTGACTAAAGAGGAGCATTTTCAGCT
GCATCATCAGATGCATTACAGCCAGCAGATTACCGTGAGCCTGGCTCAGGGATATGAGCGCAC
CAGTAGTCCAAAACCACGGTTCAAGTCCTACGCTTATACCCAGGCTGCCTACGTGACAACTAG
CGACCCTACTAGATCCCCCTTTCCATCCCAGCACCTGGAGGCCCCAGAGGACAAGAGCTTTGG
GTCCAGCCTGATGGAAAGCGAGGTGAATCTGGATCGGTACCAGACAGCCCTGGAGGAGGTGCT
GAGCTGGCTGCTGAGTGCTGAAGACACACTGCAGGCCCAGGGCGAAATTTCCAATGACGTGGA
AGTGGTGAAGGATCAGTTCCACACACACGAGGGCTATATGATGGACCTGACAGCTCACCAGGG
GCGCGTGGGCAATATCCTGCAGCTGGGCTCTAAACTGATCGGCACCGGGAAACTGAGTGAGGA
CGAGGAAACAGAAGTGCAGGAGCAGATGAACCTGCTGAACAGCCGCTGGGAGTGTCTGAGAGT
GGCTAGTATGGAGAAGCAGTCCAACCTGCACCGGGTGCTGATGGACCTGCAGAACCAGAAACT
GAAAGAGCTGAACGACTGGCTGACAAAGACTGAGGAACGCACAAGGAAGATGGAGGAGGAGCC
ACTGGGACCCGACCTGGAGGATCTGAAGAGACAGGTGCAGCAGCATAAGGTGCTGCAGGAGGA
TCTGGAACAGGAGCAGGTGCGGGTGAACTCCCTGACACATATGGTGGTGGTGGTGGACGAATC
TAGTGGAGATCACGCCACCGCCGCCCTGGAGGAACAGCTGAAGGTGCTGGGGGACCGGTGGGC
CAACATTTGCCGGTGGACCGAGGACAGGTGGGTGCTGCTGCAGGACATCCTGCTGAAATGGCA
GAGGCTGACCGAGGAGCAGTGTCTGTTTAGTGCTTGGCTGAGCGAGAAAGAGGACGCCGTGAA
CAAGATCCACACAACCGGCTTTAAGGATCAGAACGAAATGCTGTCTAGCCTGCAGAAACTGGC
TGTGCTGAAGGCCGATCTGGAGAAAAAGAAGCAGAGCATGGGCAAACTGTATAGCCTGAAACA
GGACCTGCTGAGCACCCTGAAGAACAAGAGCGTGACCCAGAAGACAGAAGCCTGGCTGGATAA
CTTTGCCCGCTGCTGGGACAACCTGGTGCAGAAACTGGAGAAAAGTACAGCTCAGATCTCTCA
GGCTGTGACCACAACCCAGCCTAGCCTGACCCAGACAACCGTGATGGAAACCGTGACCACCGT
GACAACCCGCGAACAGATCCTGGTGAAACATGCCCAGGAAGAGCTGCCACCTCCACCTCCCCA
GAAGAAGAGAACCCTGGAGCGGCTGCAGGAGCTGCAGGAAGCCACTGACGAACTGGACCTGAA
GCTGAGGCAGGCCGAAGTGATTAAGGGGTCTTGGCAGCCTGTGGGCGATCTGCTGATTGATTC
CCTGCAGGACCACCTGGAAAAGGTGAAGGCTCTGAGAGGCGAAATTGCTCCACTGAAGGAGAA
CGTGAGTCATGTGAA (SEQ ID NO: 20)

### IV.A.1. Beta Sarcoglycan Transgenes

In some aspects, the AAV vector, e.g., the AAVrh74 vector comprises a genetic cassette encoding a beta sarcoglycan polypeptide. In some aspects, the genetic cassette comprises a nucleic acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 21. In some aspects, the genetic cassette comprises the nucleic acid sequence set forth in SEQ ID NO: 21.

In some aspects, the genetic cassette comprises a nucleic acid sequence encoding a beta sarcoglycan polypeptide, wherein the beta sarcoglycan polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 45. In some aspects, the genetic cassette comprises a nucleic acid sequence encoding a beta sarcoglycan polypeptide, wherein the beta sarcoglycan polypeptide comprises the amino acid sequence set forth in SEQ ID NO: 45.

In certain aspects, the AAV vector, e.g., the AAVrh74 vector, comprises a genetic cassette encoding a beta sarcoglycan polypeptide and a promoter, e.g., any promoter disclosed herein. In certain aspects, the AAV vector, e.g., the AAVrh74 vector, comprises (i) a genetic cassette comprising a nucleic acid sequence encoding a beta sarcoglycan disclosed herein and (ii) an MHCK7 promoter. In certain aspects, the AAV vector, e.g., the AAVrh74 vector, comprises (i) a genetic cassette comprising the nucleic acid sequence set forth in SEQ ID NO: 21 and (ii) an MHCK7 promoter. In certain aspects, the AAV vector, e.g., the AAVrh74 vector, comprises (i) a genetic cassette comprising the nucleic acid sequence set forth in SEQ ID NO: 21 and (ii) a promoter comprising the nucleic acid sequence set forth in SEQ ID NO: 25.

In certain aspects, the AAV vector, e.g., the AAVrh74 vector, comprises a genetic cassette encoding a beta sarcoglycan polypeptide and an intronic sequence, e.g., any intronic sequence disclosed herein. In certain aspects, the AAV vector, e.g., the AAVrh74 vector, comprises (i) a genetic cassette comprising a nucleic acid sequence encoding a beta sarcoglycan disclosed herein and (ii) an intronic sequence comprising the nucleic acid sequence set forth in SEQ ID NO: 28. In certain aspects, the AAV vector, e.g., the AAVrh74 vector, comprises (i) a genetic cassette comprising the nucleic acid sequence set forth in SEQ ID NO: 21 and (ii) an intronic sequence comprising the nucleic acid sequence set forth in SEQ ID NO: 28. In certain aspects, the AAV vector, e.g., the AAVrh74 vector, comprises (i) a genetic cassette comprising the nucleic acid sequence set forth in SEQ ID NO: 21; (ii) a promoter comprising the nucleic acid sequence set forth in SEQ ID NO: 25; and (iii) an intronic sequence comprising the nucleic acid sequence set forth in SEQ ID NO: 28.

In certain aspects, the AAV vector, e.g., the AAVrh74 vector, comprises a genetic cassette encoding a beta sarcoglycan polypeptide and a 3′UTR poly(A) tail sequence, e.g., any 3′UTR poly(A) tail sequence disclosed herein. In certain aspects, the AAV vector, e.g., the AAVrh74 vector, comprises (i) a genetic cassette comprising a nucleic acid sequence encoding a beta sarcoglycan disclosed herein and (ii) a 3′UTR poly(A) tail sequence comprising the nucleic acid sequence set forth in SEQ ID NO: 30. In certain aspects, the AAV vector, e.g., the AAVrh74 vector, comprises (i) a genetic cassette comprising the nucleic acid sequence set forth in SEQ ID NO: 21 and (ii) a 3′UTR poly(A) tail sequence comprising the nucleic acid sequence set forth in SEQ ID NO: 30. In certain aspects, the AAV vector, e.g., the AAVrh74 vector, comprises (i) a genetic cassette comprising the nucleic acid sequence set forth in SEQ ID NO: 21; (ii) a promoter comprising the nucleic acid sequence set forth in SEQ ID NO: 25; (iii) an intronic sequence comprising the nucleic acid sequence set forth in SEQ ID NO: 28; and (iv) a 3′UTR poly(A) tail sequence comprising the nucleic acid sequence set forth in SEQ ID NO: 30.

In certain aspects, the AAV vector, e.g., the AAVrh74 vector, comprises a genetic cassette encoding a beta sarcoglycan polypeptide, wherein the AAV vector, e.g., the AAVrh74 vector, comprises a nucleic acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 22. In certain aspects, the AAV vector, e.g., the AAVrh74 vector, comprises a genetic cassette encoding a beta sarcoglycan polypeptide, wherein the AAV vector, e.g., the AAVrh74 vector, comprises the nucleic acid sequence set forth in SEQ ID NO: 22.

TABLE 9
Example AA Vrh74-Beta Sarcoglycan Nucleic Acid Sequence

CTGCGCGCTCGCTCGCTCACTGAGGCCGCCCGGGCAAAGCCCGGGCGTCGGGCGACCTTTGGT
CGCCCGGCCTCAGTGAGCGAGCGAGCGCGCAGAGAGGGAGTGGGGTTAACCAATTGGCGGCCG
CAAGCTTGCATGTCTAAGCTAGACCCTTCAGATTAAAAATAACTGAGGTAAGGGCCTGGGTAG
GGGAGGTGGTGTGAGACGCTCCTGTCTCTCCTCTATCTGCCCATCGGCCCTTTGGGGAGGAGG
AATGTGCCCAAGGACTAAAAAAAGGCCATGGAGCCAGAGGGGCGAGGGCAACAGACCTTTCAT
GGGCAAACCTTGGGGCCCTGCTGTCTAGCATGCCCCACTACGGGTCTAGGCTGCCCATGTAAG
GAGGCAAGGCCTGGGGACACCCGAGATGCCTGGTTATAATTAACCCAGACATGTGGCTGCCCC
CCCCCCCCCAACACCTGCTGCCTCTAAAAATAACCCTGTCCCTGGTGGATCCCCTGCATGCGA
AGATCTTCGAACAAGGCTGTGGGGGACTGAGGGCAGGCTGTAACAGGCTTGGGGGCCAGGGCT
TATACGTGCCTGGGACTCCCAAAGTATTACTGTTCCATGTTCCCGGCGAAGGGCCAGCTGTCC
CCCGCCAGCTAGACTCAGCACTTAGTTTAGGAACCAGTGAGCAAGTCAGCCCTTGGGGCAGCC
CATACAAGGCCATGGGGCTGGGCAAGCTGCACGCCTGGGTCCGGGGTGGGCACGGTGCCCGGG
CAACGAGCTGAAAGCTCATCTGCTCTCAGGGGCCCCTCCCTGGGGACAGCCCCTCCTGGCTAG
TCACACCCTGTAGGCTCCTCTATATAACCCAGGGGCACAGGGGCTGCCCTCATTCTACCACCA
CCTCCACAGCACAGACAGACACTCAGGAGCAGCCAGCGGCGCGCCCAGGTAAGTTTAGTCTTT
TTGTCTTTTATTTCAGGTCCCGGATCCGGTGGTGGTGCAAATCAAAGAACTGCTCCTCAGTGG
ATGTTGCCTTTACTTCTAGGCCTGTACGGAAGTGTTACTTCTGCTCTAAAAGCTGCGGAATTG
TACCCGGTACCACCATGGCAGCAGCAGCCGCCGCAGCCGCCGAGCAGCAGTCAAGCAATGGAC
CAGTGAAAAAATCAATGAGAGAAAAAGCCGTCGAGAGGAGATCAGTGAATAAGGAGCACAACA
GCAATTTCAAAGCCGGCTACATCCCTATTGACGAAGATCGCCTGCATAAGACAGGCCTGAGGG
GGCGCAAAGGAAACCTGGCAATCTGCGTCATCATTCTGCTGTTTATCCTGGCCGTGATTAATC
TGATCATTACTCTGGTGATTTGGGCTGTCATCCGCATTGGCCCAAACGGGTGTGACTCTATGG
AGTTCCACGAAAGTGGCCTGCTGCGATTTAAGCAGGTGTCCGATATGGGGGTCATCCATCCAC
TGTACAAATCTACTGTCGGCGGGCGGAGAAACGAGAATCTGGTGATCACCGGGAACAATCAGC
CCATTGTGTTCCAGCAGGGAACCACAAAGCTGTCTGTGGAAAACAATAAAACATCAATCACTA
GCGACATTGGCATGCAGTTCTTTGATCCCCGGACCCAGAATATCCTGTTCAGTACCGACTATG
AGACACACGAATTTCATCTGCCTTCCGGGGTGAAGTCTCTGAACGTCCAGAAAGCCAGCACTG
AGAGAATCACCAGTAACGCTACATCAGACCTGAATATCAAGGTGGATGGACGAGCTATTGTCC
GGGGAAATGAGGGCGTGTTCATCATGGGCAAGACAATTGAATTTCACATGGGAGGCAACATGG
AGCTGAAAGCAGAAAACAGCATCATTCTGAATGGGAGCGTGATGGTCTCCACTACCAGACTGC
CCAGCTCCTCTAGTGGAGACCAGCTGGGGTCCGGAGATTGGGTCAGGTATAAGCTGTGCATGT
GTGCCGATGGCACCCTGTTTAAAGTGCAGGTCACCAGCCAGAATATGGGATGTCAGATTAGCG
ATAACCCTTGTGGGAATACTCATTAAAAGCTTGGCCGCAATAAAAGATCTTTATTTTCATTAG
ATCTGTGTGTTGGTTTTTTGTGTGTCCTGCAGGGGCGCGCCTCTAGAGCATGGCTACGTAGAT
AAGTAGCATGGCGGGTTAATCATTAACTACAAGGAACCCCTAGTGATGGAGTTGGCCACTCCC
TCTCTGCGCGCTCGCTCGCTCACTGAGGCCGGGCGACCAAAGGTCGCCCGACGCCCGGGCTTT
GCCCGGGCGGCCTCAGTGAGCGAGCGAGCGCGCAG (SEQ ID NO: 22)

### IV.A.2. Alpha Sarcoglycan Transgenes

In some aspects, the AAV vector, e.g., the AAVrh74 vector, comprises a genetic cassette encoding an alpha sarcoglycan polypeptide. In some aspects, the genetic cassette comprises a nucleic acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 23. In some aspects, the genetic cassette comprises the nucleic acid sequence set forth in SEQ ID NO: 23.

In some aspects, the genetic cassette comprises a nucleic acid sequence encoding an alpha sarcoglycan polypeptide, wherein the alpha sarcoglycan polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 46. In some aspects, the genetic cassette comprises a nucleic acid sequence encoding an alpha sarcoglycan polypeptide, wherein the alpha sarcoglycan polypeptide comprises the amino acid sequence set forth in SEQ ID NO: 46.

In certain aspects, the AAV vector, e.g., the AAVrh74 vector, comprises a genetic cassette encoding an alpha sarcoglycan polypeptide and a promoter, e.g., any promoter disclosed herein. In certain aspects, the AAV vector, e.g., the AAVrh74 vector, comprises (i) a genetic cassette comprising a nucleic acid sequence encoding an alpha sarcoglycan disclosed herein and (ii) a tMCK promoter. In certain aspects, the AAV vector, e.g., the AAVrh74 vector, comprises (i) a genetic cassette comprising the nucleic acid sequence set forth in SEQ ID NO: 23 and (ii) a tMCK promoter. In certain aspects, the AAV vector, e.g., the AAVrh74 vector, comprises (i) a genetic cassette comprising the nucleic acid sequence set forth in SEQ ID NO: 23 and (ii) a promoter comprising the nucleic acid sequence set forth in SEQ ID NO: 26.

In certain aspects, the AAV vector, e.g., the AAVrh74 vector, comprises a genetic cassette encoding an alpha sarcoglycan polypeptide and an intronic sequence, e.g., any intronic sequence disclosed herein. In certain aspects, the AAV vector, e.g., the AAVrh74 vector, comprises (i) a genetic cassette comprising a nucleic acid sequence encoding an alpha sarcoglycan disclosed herein and (ii) an intronic sequence comprising the nucleic acid sequence set forth in SEQ ID NO: 29. In certain aspects, the AAV vector, e.g., the AAVrh74 vector, comprises (i) a genetic cassette comprising the nucleic acid sequence set forth in SEQ ID NO: 23 and (ii) an intronic sequence comprising the nucleic acid sequence set forth in SEQ ID NO: 29. In certain aspects, the AAV vector, e.g., the AAVrh74 vector, comprises (i) a genetic cassette comprising the nucleic acid sequence set forth in SEQ ID NO: 23; (ii) a promoter comprising the nucleic acid sequence set forth in SEQ ID NO: 26; and (iii) an intronic sequence comprising the nucleic acid sequence set forth in SEQ ID NO: 29.

In certain aspects, the AAV vector, e.g., the AAVrh74 vector, comprises a genetic cassette encoding an alpha sarcoglycan polypeptide and a 3′UTR poly(A) tail sequence, e.g., any 3′UTR poly(A) tail sequence disclosed herein. In certain aspects, the AAV vector, e.g., the AAVrh74 vector, comprises (i) a genetic cassette comprising a nucleic acid sequence encoding an alpha sarcoglycan disclosed herein and (ii) a 3′UTR poly(A) tail sequence comprising the nucleic acid sequence set forth in SEQ ID NO: 30. In certain aspects, the AAV vector, e.g., the AAVrh74 vector, comprises (i) a genetic cassette comprising the nucleic acid sequence set forth in SEQ ID NO: 23 and (ii) a 3′UTR poly(A) tail sequence comprising the nucleic acid sequence set forth in SEQ ID NO: 30. In certain aspects, the AAV vector, e.g., the AAVrh74 vector, comprises (i) a genetic cassette comprising the nucleic acid sequence set forth in SEQ ID NO: 23; (ii) a promoter comprising the nucleic acid sequence set forth in SEQ ID NO: 26; (iii) an intronic sequence comprising the nucleic acid sequence set forth in SEQ ID NO: 29; and (iv) a 3′UTR poly(A) tail sequence comprising the nucleic acid sequence set forth in SEQ TD NO: 30.

In certain aspects, the AAV vector, e.g., the AAVrh74 vector, comprises a genetic cassette encoding an alpha sarcoglycan polypeptide, wherein the AAV vector, e.g., the AAVrh74 vector, comprises a nucleic acid sequence having at least about 500%, at least about 55% o, at least about 6000, at least about 65% o, at least about 7000, at least about 75% o, at least about 800%, at least about 850%, at least about 90% o, at least about 9500 at least about 96% o, at least about 9700 at least about 98% o, or at least about 9900 sequence identity to SEQ ID NO: 24. In certain aspects, the AAV vector, e.g., the AAVrh74 vector, comprises a genetic cassette encoding an alpha sarcoglycan polypeptide, wherein the AAV vector, e.g., the AAVrh74 vector, comprises the nucleic acid sequence set forth in SEQ ID NO: 24.

TABLE 10
Example AAVrh74-Alpha Sarcoglycan Nucleic Acid Sequence

CTGCGCGCTCGCTCGCTCACTGAGGCCGCCCGGGCAAAGCCCGGGCGTCGGGCGACCTTTGGT
CGCCCGGCCTCAGTGAGCGAGCGAGCGCGCAGAGAGGGAGTGGGGTTAACCAATTGGCGGCCG
CAAACTTGCATGCCCCACTACGGGTCTAGGCTGCCCATGTAAGGAGGCAAGGCCTGGGGACAC
CCGAGATGCCTGGTTATAATTAACCCCAACACCTGCTGCCCCCCCCCCCCCAACACCTGCTGC
CTGAGCCTGAGCGGTTACCCCACCCCGGTGCCTGGGTCTTAGGCTCTGTACACCATGGAGGAG
AAGCTCGCTCTAAAAATAACCCTGTCCCTGGTGGATCCACTACGGGTCTATGCTGCCCATGTA
AGGAGGCAAGGCCTGGGGACACCCGAGATGCCTGGTTATAATTAACCCCAACACCTGCTGCCC
CCCCCCCCCCAACACCTGCTGCCTGAGCCTGAGCGGTTACCCCACCCCGGTGCCTGGGTCTTA
GGCTCTGTACACCATGGAGGAGAAGCTCGCTCTAAAAATAACCCTGTCCCTGGTGGACCACTA
CGGGTCTAGGCTGCCCATGTAAGGAGGCAAGGCCTGGGGACACCCGAGATGCCTGGTTATAAT
TAACCCCAACACCTGCTGCCCCCCCCCCCCAACACCTGCTGCCTGAGCCTGAGCGGTTACCCC
ACCCCGGTGCCTGGGTCTTAGGCTCTGTACACCATGGAGGAGAAGCTCGCTCTAAAAATAACC
CTGTCCCTGGTCCTCCCTGGGGACAGCCCCTCCTGGCTAGTCACACCCTGTAGGCTCCTCTAT
ATAACCCAGGGGCACAGGGGCTGCCCCCGGGTCACCTGCAGAAGTTGGTCGTGAGGCACTGGG
CAGGTAAGTATCAAGGTTACAAGACAGGTTTAAGGAGACCAATAGAAACTGGGCTTGTCGAGA
CAGAGAAGACTCTTGCGTTTCTGATAGGCACCTATTGGTCTTACTGACATCCACTTTGCCTTT
CTCTCCACAGGTGTCCACTCCCAGTTCAATTACAGCGCGTGGTACCACCATGGCCGAGACACT
GTTCTGGACTCCTCTGCTGGTGGTGCTGCTGGCTGGACTGGGAGATACCGAGGCTCAGCAGAC
CACACTGCACCCACTGGTGGGCCGGGTGTTCGTGCACACCCTGGACCATGAGACATTTCTGAG
TCTGCCAGAACACGTGGCTGTGCCACCTGCTGTGCATATCACTTACCACGCCCATCTGCAGGG
CCATCCTGATCTGCCACGGTGGCTGAGATACACCCAGAGATCACCCCACCATCCTGGATTCCT
GTATGGAAGCGCTACCCCAGAGGACAGGGGACTGCAGGTGATCGAAGTGACAGCTTACAACCG
CGACAGTTTTGATACTACCAGGCAGCGCCTGGTGCTGGAGATTGGGGATCCAGAAGGACCCCT
GCTGCCTTATCAGGCCGAGTTCCTGGTGCGGTCACACGACGCTGAGGAAGTGCTGCCATCAAC
ACCCGCCAGCAGATTTCTGTCCGCTCTGGGAGGACTGTGGGAGCCAGGAGAACTGCAGCTGCT
GAATGTGACTAGCGCTCTGGATAGGGGAGGAAGGGTGCCACTGCCAATCGAGGGAAGGAAGGA
AGGGGTGTACATTAAAGTGGGAAGCGCTTCCCCATTCTCCACCTGCCTGAAGATGGTGGCTTC
TCCTGATAGTCACGCTAGGTGCGCTCAGGGACAGCCACCACTGCTGTCCTGTTATGACACACT
GGCCCCCCATTTTCGCGTGGACTGGTGCAACGTGACTCTGGTGGATAAATCTGTGCCTGAGCC
AGCTGACGAAGTGCCAACCCCTGGAGACGGAATCCTGGAGCACGATCCTTTCTTTTGTCCTCC
AACAGAAGCCCCAGACAGGGATTTCCTGGTGGACGCTCTGGTGACTCTGCTGGTGCCTCTGCT
GGTGGCTCTGCTGCTGACCCTGCTGCTGGCTTATGTGATGTGCTGTCGGAGAGAGGGACGGCT
GAAGAGAGACCTGGCCACATCTGATATCCAGATGGTGCACCATTGTACTATTCACGGCAACAC
CGAGGAACTGCGCCAGATGGCTGCTTCTAGGGAGGTGCCAAGGCCACTGAGTACACTGCCTAT
GTTTAATGTGCACACTGGCGAACGGCTGCCCCCTAGAGTGGATAGCGCCCAGGTGCCACTGAT
TCTGGACCAGCATTGAGGCCGCAATAAAAGATCTTTATTTTCATTAGATCTGTGTGTTGGTTT
TTTGTGTGTCCTGCAGGGGCGCGCCTCTAGAGCATGGCTACGTAGATAAGTAGCATGGCGGGT
TAATCATTAACTACAAGGAACCCCTAGTGATGGAGTTGGCCACTCCCTCTCTGCGCGCTCGCT
CGCTCACTGAGGCCGGGCGACCAAAGGTCGCCCGACGCCCGGGCTTTGCCCGGGCGGCCTCAG
TGAGCGAGCGAGCGCGCAG (SEQ ID NO: 24)

### V. Methods of Producing an AAV Viral Vector Particles

The present disclosure provides a composition comprising a recombinant adeno-associated virus (rAAV) viral vector particle, wherein the rAAV vector particle is produced in adherent mammalian cells, and wherein the adherent cells are cultured in an N-1 container under suspension conditions. In some aspects, the rAAV vector particle is of the serotype AAVrh.74 (e.g., rAAVrh74.MHCK7.micro-dystrophin).

The present disclosure also provides a method of producing a rAAV viral vector particle, e.g., a rAAVrh74.MHCK7.microdystrophin in adherent mammalian cells by a suspension seed process. In some aspects, the suspension and seed process comprising: (a) culturing cells with a first growth medium comprising serum in a N-2 container; (b) removing the cells from the first medium; (c) inoculating the cells from step (b) into a second medium comprising no serum or serum at a concentration less than the first medium in a N-1 container; (d) culturing the cells in the N-1 container under suspension conditions; and (e) inoculating a third medium in a bioreactor with the cells from step (d).

In some aspects, the suspension seed process further comprises: (f) transfecting the adherent cells with a transgene plasmid comprising a recombinant MHCK7.microdystrophin construct between AAV ITRs, a plasmid comprising an AAV rep gene and an AAV cap gene, and an adenovirus helper plasmid.

In some aspects, the plasmid comprising an AAV rep gene and an AAV cap gene comprises an AAV2 rep gene and a AAVrh74 cap gene. In some aspects, the plasmid comprising an AAV rep gene comprises an AAV rep gene of the same AAV serotype as the AAV ITRs in a plasmid comprising the recombinant MHCK7.microdystrophin construct. For example, a recombinant MHCK7.microdystrophin construct comprises AAV2 ITRs and the plasmid comprising the AAV rep gene comprises an AAV2 rep gene. In some aspects, the plasmid comprising the AAV2 rep gene further comprises an AAVrh74 cap gene. In some aspects, the adenovirus helper plasmid comprises an adenovirus 5 E2A, E4ORF6, and a VA RNA gene.

In some aspects, the suspension seed process further comprises: (g) lysing the adherent cells. In some aspects, the adherent cells are lysed by freeze-thaw, solid shear, hypertonic and/or hypotonic lysis, liquid shear, sonication, high-pressure extrusion, detergent lysis, or combinations thereof.

In some aspects, the suspension seed process further comprises (h) purifying the rAAVrh74 viral vector particles by at least one column chromatography step. In some aspects, the at least one column chromatography step comprises an anion exchange chromatography, a size exclusion chromatography, or a combination thereof.

In some aspects, the suspension seed process further comprises culturing cells with the first growth medium in a N-3 container. In some aspects, the suspension seed process further comprises culturing cells with the first growth medium in a N-4 container.

In some aspects, the bioreactor is an adherent bioreactor. In some aspects, the rAAVrh74 viral vector particles are purified from the culture produced in the adherent bioreactor.

In some aspects, the third medium in the bioreactor comprises at least one factor that promotes cell adherence. In some aspects, the at least one factor that promotes cell adherence is selected from the group consisting of serum, FBS, fibronectin, collagen, laminin, calcium ions, proteoglycans or non-proteoglycan polysaccharides of the extracellular matrix, and combinations thereof. In some aspects, the third medium in the bioreactor comprises DMEM and 10% FBS.

In some aspects, the adherent cells are cultured under suspension conditions for about 48-72 hours.

In some aspects, the N-1 container is a suspension shake flask.

In some aspects, the adherent cells are selected from the group consisting of HeLa cells, CHO cells, HEK-293 cells, VERO cells, BHK cells, MDCK cells, MDBK cells, and COS cells. In some aspects, the adherent cells are HeLa cells or HEK-293 cells. In some aspects, the adherent cells are HEK-293 cells. In some aspects, the adherent cells are not suspension-adapted. In some aspects, culturing the cells under suspension conditions does not alter the adherent-dependency of the cells. In some aspects, the culturing does not alter the cells to create a new cell line.

In some aspects, the suspension seed process used to produce a rAAVrh74.MHCK7.microdystrophin in adherent mammalian cells comprises: (a) culturing cells with a first growth medium comprising serum in a N-2 container; (b) removing the cells from the first medium; (c) inoculating the cells from step (b) into a second medium comprising no serum or serum at a concentration less than the first medium in a N-1 container; (d) culturing the cells in the N-1 container under suspension conditions; (e) inoculating a third medium in a bioreactor with the cells from step (d); (f) transfecting the cells with a transgene plasmid comprising a recombinant MHCK7.microdystrophin construct between AAV ITRs, a plasmid comprising an AAV rep gene and an AAV cap gene, and an adenovirus helper plasmid; (g) lysing the cells, and (h) purifying the rAAV by at least one column chromatography step. In some aspects, the transgene plasmid comprises a recombinant MHCK7.microdystrophin construct between AAV2 ITRs, the plasmid comprising an AAV rep gene and an AAV cap gene comprises an AAV2 rep gene and an AAVrh74 cap gene.

In some aspects, DNA plasmids of the present disclosure comprise a recombinant AAV (rAAV) genome of the present disclosure. In some aspects, the DNA plasmids are transferred to cells permissible for infection with a helper virus of AAV (e.g., adenovirus, E1-deleted adenovirus or herpesvirus) for assembly of the rAAV genome into infectious rAAV viral vector particles. Techniques to produce rAAV viral vector particles, in which a rAAV genome to be packaged, rep and cap genes, and helper virus functions are provided to a cell are standard in the art. Production of rAAV vector particles requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs (resulting in a pseudotyped rAAV viral vector particles), including, but not limited to, AAV serotypes AAV-1, AAV-2, AAV-3, AAV-4, AAV-5, AAV-6, AAV-7, AAVrh.74, AAV-8, AAV-9, AAV-10, AAV-11, AAV-12 and AAV-13. Production of pseudotyped rAAV vector particles is disclosed in, for example, WO 01/83692 which is incorporated by reference herein in its entirety.

In some aspects, a method of generating a packaging cell is to create a cell line that stably expresses all the necessary components for rAAV viral vector particle production. For example, a plasmid (or multiple plasmids) comprising a rAAV genome nucleic acid lacking AAV rep and cap genes, AAV rep and cap genes separate from the rAAV genome nucleic acid, and a selectable marker, such as a neomycin resistance gene, are integrated into the genome of a cell. In some aspects, the AAV rep gene is an AAV2 rep gene and the AAV cap gene is an AAVrh74 cap gene. In some aspects, an AAV rep gene is of the same AAV serotype as the AAV ITRs in the rAAV plasmid comprising the rAAV genomes lacking AAV rep and AAV cap genes. In some aspects, an AAV rep gene is of the same AAV serotype as the AAV ITRs in the recombinant MHCK7.micro-dystrophin plasmid and the AAV cap gene is an AAVrh74 cap gene.

AAV genomes have been introduced into bacterial plasmids by procedures such as GC tailing (Samulski et al., Proc. Natl. Acad. S6. USA 79:2077-2081 (1982), addition of synthetic linkers containing restriction endonuclease cleavage sites (Laughlin et al., Gene 23:65-73 (1983)) or by direct, blunt-end ligation (Senapathy & Carter, J. Biol. Chem. 259:4661-4666 (1984)). In some aspects, a packaging cell line is infected with a helper virus such as adenovirus. The advantages of this method are that the cells are selectable and are suitable for large-scale production of rAAV viral vector particles. Other examples of suitable methods employ adenovirus or baculovirus rather than plasmids to introduce rAAV genomes and/or rep and cap genes into packaging cells.

General principles of rAAV viral vector particle production are reviewed in, for example, Carter, Current Opinions in Biotechnology 1533-539 (1992); and Muzyczka, N., Curr. Topics Microbial. Immunol. 158: 97-129 (1992). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995); Paul et al. Human Gene Therapy 4:609-615 (1993); Clark et al. Gene Therapy 3:1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV vector particle production.

In some aspects, at least one of a plasmid comprising a transgene between AAV ITRs, a plasmid comprising AAV rep genes and AAV cap genes and/or an adenovirus helper plasmid used for generating AAVrh74 viral vector particles as described herein is stably integrated in a genome of a cell, which cell generates the AAVrh74 viral vector particles.

In some aspects, a transgene plasmid comprises an expression cassette comprising an enhanced green fluorescent protein (eGFP) between AAV2 ITRs and is used to prepare an AAVrh74 viral vector particle.

### VI. Combination Therapies

In some aspects, provided are methods of treatment comprising administering to a subject in need thereof, an initial dose of Imlifidase followed by rAAVrh74 vector particles. In some aspects, rAAVrh74 vector particles are administered no later than 54 hours after an initial dose of Imlifidase. In some aspects, the method further comprises measuring a titer of antibodies to rAAVrh74 in the subject prior to administering Imlifidase. In some aspects, the method further comprises measuring a titer of antibodies to rAAVrh74 in the subject after administering Imlifidase. A useful rAAVrh74 vector particle is delandistrogene moxeparvovec.

In some aspects, a method of treatment is provided, the method comprising administering intravenously to a subject in need thereof an initial dose of Imlifidase of about 0.25 mg/kg. In some aspects, the method further comprises measuring a titer of antibodies to rAAVrh74 in the subject prior to administering Imlifidase. In some aspects, the method further comprises measuring a titer of antibodies to rAAVrh74 in the subject after administering Imlifidase. In some aspects, the method further comprises administering rAAVrh74 vector particles if the titer of anti-rAAVrh74 antibodies is at or below 1:400 after administration of Imlifidase. In some aspects, rAAVrh74 vector particles are administered no later than 54 hours after the administration of Imlifidase. In some aspects, the rAAVrh74 vector particle is delandistrogene moxeparvovec.

In some aspects, the method further comprises administering a second dose of Imlifidase if the titer of anti-rAAVrh74 antibodies is above 1:400 after the first administration of Imlifidase. In some aspects, the method further comprises measuring the titer of anti-rAAVrh74 antibodies after the second Imlifidase administration and if the titer of anti-rAAVrh74 antibodies is at or below 1:400, the method further comprises administering rAAVrh74. In some aspects, rAAVrh74 vector particles is administered no later than 54 hours after the second Imlifidase administration. In some aspects, the rAAVrh74 vector particle is delandistrogene moxeparvovec.

In some aspects, if the titer of anti-rAAVrh74 antibodies is above 1:400 after administration of the second dose of Imlifidase, the subject will not be administered any rAAVrh74 vector particles.

In some aspects, provided is a method of treatment comprising intravenously administering an initial dose of Imlifidase of 0.25 mg/kg. In some aspects, the method further comprises measuring a titer of antibodies to rAAVrh74 in the subject after the administration of imflifidase. In some aspects, the method further comprises administering intravenously 1.33×10¹⁴ vg/kg rAAVr74 if the titer of antibodies to rAAVrh74 in the subject measured after the Imlifidase administration is at or a below 1:400. In some aspects, rAAVrhH74 is administered within 48 hours from the administration of the first dose of Imlifidase. In some aspects, rAAVrh74 vector particles are administered no later than 54 hours from the first dose of Imlifidase. In some aspects, RAAVRH74 is administered within 48 hours from the time point when the titer of anti-rAAVrh74 antibodies is measured as being at or below 1:400. In some aspects, rAAVrh74 is administered within 48 hours from the time point when the titer of anti-rAAVrh74 antibodies is measured as being at or below 1:400 after a first dose of Imlifidase. In some aspects, rAAVrh74 is administered within 48 hours from the time point when the titer of anti-rAAVrh74 antibodies is measured as being at or below 1:400 after a second dose of Imlifidase. In some aspects, rAAVrh74 vector particles are administered no later than 54 hours after the first or the second dose of Imlifidase. In some aspects, the rAAVrh74 vector particle is delandistrogene moxeparvovec.

In some aspects, the method comprises intravenously administering an initial dose of Imlifidase of 0.25 mg/kg. In some aspects, the method further comprises measuring a titer of antibodies to rAAVrh74 in the subject after the administration of the initial dose of Imlifidase. In some aspects, the method further comprises administering intravenously a second dose of 0.25 mg/kg of Imlifidase within 60 hours of measuring a titer of antibodies to rAAVrh74 in the subject above 1:400. In some aspects, the method further comprises measuring a titer of antibodies to rAAVrh74 in the subject after the administration of the second dose of Imlifidase. In some aspects, the method further comprises administering intravenously 1.33×10¹⁴ vg/kg rAAVrh74 if the titer of antibodies to rAAVrh74 in the subject measured within 48 hours after the administration of the second dose of Imlifidase is at or below 1:400. In some aspects, rAAVrh74 vector particles are administered no later than 54 hours after the second dose of Imlifidase. In some aspects, if the titer of antibodies to rAAVrh74 in the subject measured within 48 hours after the administration of the second dose of Imlifidase is above 1:400, the subject will not be administered rAAVrh74.

In some aspects, the method further comprises administering a steroid. In some aspects, the method comprises administering an oral steroid. In some aspects, the oral steroid is administered daily for at least 12 weeks before the administration of AAVrh74. In some aspects, additional steroid is administered at least one day before the administration of AAVrh74. In some aspects, additional steroid is administered at least one day before the administration of AAVrh74 and is continued to be administered for approximately 60 days after the administration of AAVrh74. In some aspects, the oral steroid is prednisone or prednisolone.

### VII. Kits

Certain aspects of the present disclosure are directed to kits for treating a muscular dystrophy in a human subject in need thereof. In some aspects, the kit comprises (i) an enzyme that cleaves IgG disclosed herein, (ii) an AAV vector disclosed herein, e.g., an AAVrh74 vector; and (iii) instructions to administer the enzyme and the AAV vector according to any method disclosed herein. In some aspects, the kit comprises (i) an AAV vector disclosed herein, e.g., an AAVrh74 vector; and (ii) instructions to administer the AAV vector to a subject that has previously been administered an enzyme that cleaves an IgG, according to any method disclosed herein.

All of the various aspects, aspects, and options described herein can be combined in any and all variations.

### EXAMPLES

### Example 1: A Safety and Efficacy Evaluation of AAVrh74.CMV.eGFP (rAAVrh74.CMV.eGFP) Following a Single or Repeat Dose Intravenous Injection to Female Cynomolgus Monkeys Preceded by a Single Intravenous Injection of Imlifidase Followed by at Least 59 Days of Observation

The purpose of this study was to evaluate the safety and efficacy of AAVrh74.CMV.eGFP (“Test Article 2,” hereafter AAVrh74-eGFP) when administered as a single (Groups 2 through 6) or repeat (Group 7) intravenous injection(s) to female cynomolgous monkeys, when preceded by a single intravenous injection of Imlifidase (“Test Article 1”). Following administration of AAVrh74.CMV-eGFP, preceded by Imlifidase, animals underwent an observation period of at least 59 days.

### Study Design

Female cynomolgus monkeys were assigned to nine groups, and doses were administered as indicated in Table 19. Animals in Phase I were administered a single intravenous injection of Imlifidase at a volume of 1 mL/kg to examine initial tolerability of Imlifidase in monkeys. As a standard immuno-suppressive treatment with AAV gene therapies, animals in Phase II were administered a single dose of prednisolone via oral gavage on Day 1 (Groups 2 through 7) or once daily for 14 days (Groups 8 and 9 only) at a volume of 0.33 mL/kg/day. To achieve the main objectives of the study, animals in Phase III were administered a daily dose of prednisolone via oral gavage for 62 (Groups 3 through 6) or 96 (Groups 2 and 7) days at a volume of 0.33 mL/kg/day and a single or repeated dose of control article, Imlifidase, and/or AAVrh74.eGFP via intravenous injection at a volume of 1 mg/mL (control article or Imlifidase) or 5.47 mL/kg (control article or AAVrh74.eGFP). The control article for Imlifidase and AAVrh74.eGFP was sterile saline. The control article for the Prednisolone was reverse osmosis water. Due to the complexity of the study design, animals are referred to by group number and AAVrh74.CMV.eGFP is referred to as AAVrh74.eGFP in the Summary, Results, Discussion, and Conclusions sections. Additionally, as Dosing Phase II consisted of a single dose of Prednisolone to all animals, and was one day in duration, Dosing Phase II will have little to no discussion in the Summary, Results, Discussion, and Conclusion sections.

### Dosing of Imlifidase and AAVrh74-eGFP Vectors

In this Example, non-human primates (NHP) were dosed with an AAVrh74-eGFP vector and Imlifidase to determine whether Imlifidase, administered pre-treatment, lowered anti-AAVrh74 total antibody titers to safely dose AAV gene therapy.

One caveat of the study was that the efficacy of Imlifidase in NHPs is about 70% compared to humans, where Imlifidase efficacy is about 99%.

NHPs were grouped according to pre-existing antibody titers and treatments. Group 2 and 3 animals were seronegative with total AAVrh74 antibody titers below 1:400.

Group 4 and Group 5 animals had a total AAVrh74 antibody titer between 1:800 and 1:1,600 and were considered AAVrh74 seropositive animals.

Group 6 animals had a total AAVrh74 antibody titer between 1:3,200 and 1:25,600 and were considered AAVrh74 high seropositive. Group 7 animals (redose group) had total AAVrh74 antibody titers below 1:400 prior to any treatment and were considered AABrh74 seronegative. After a first dose of AAVrh74 vector, one Group 7 animal had a total AAVrh74 antibody titer of below 1:400, while the other two Group 7 animals had total AAVrh74 antibody titers of 1:204,800 and 1:25,600, respectively.

The treatments were as follows: Group 2 animals received saline only and Group 3 and 4 animals received AAVrh74-eGFP vector but no Imlifidase (vector control).

Group 5 and 6 animals received Imlifidase (IDEFIRIX®, Hansa Biopharma AB, Sweden) and AAVrh74-eGFP vectors.

Group 7 animals received AAVrh74-eGFP vector, followed by Imlifidase, followed by a second dose of AAVrh74-eGFP vector.

In Groups 5 and 6, Imlifidase was dosed on day 1 at 10 mg/kg/dose and AAVrh74-EGFP was dosed on day 3 at 1.33×10¹⁴ vector genomes/kg/dose.

In Group 7, AAVrh74-eGFP was dosed on day 3 and day 38 at 1.33×10¹⁴ vector genomes/kg/dose and Imlifidase was dosed on day 36 at 10 mg/kg/dose.

The dosing scheme is shown in Table 19

TABLE 19
Dosing Phase III: Test Article 1 (Imlifidase), Test Article 2 (AAVrh74.CMV.eGFP), and Control Article

| Group Assignment and | | | | | | | | |
|---|---|---|---|---|---|---|---|---|
| Anti-AAVrh74 | | | | | | | | |
| Antibody Status | | | | | | | | |
| | Anti- | Test Article 1 or Control Article | | | Test Article 2 or Control Article | | | Number |
| | AAVrh74 | Dosing | Dose | Dose | Dosing | Dose | Concentration^{e} | of |
| | Antibody | Phase | Levels^{d} | Concentrations | Phase | Level^{e} | Dose | Animals^{f} |
| Group^{a} | Status^{b} | Days^{c} | (mg/kg/dose) | (mg/mL) | Days^{c, e} | (vg/kg/dose) | (vg/mL) | Females |
| 2 (Saline | ≤1:400 | 1/36 | 0/0 | 0/0 | 3/38 | 0 | 0 | 3 |
| Control, AAV−) | | | | | | | | |
| 3 (TA2, AAV−) | ≤1:400 | 1 | 0 | 0 | 3 | 1.33 × 10¹⁴ | 2.43 × 10¹³ | 3 |
| 4 (TA2, AAV+) | 1:800-1:1600 | 1 | 0 | 0 | 3 | 1.33 × 10¹⁴ | 2.43 × 10¹³ | 3 |
| 5 (TA1 + TA2) | 1:800-1:1600 | 1 | 10 | 10 | 3 | 1.33 × 10¹⁴ | 2.43 × 10¹³ | 3 |
| 6 (TA1 + TA2) | 1:3200-1:25600 | 1 | 10 | 10 | 3 | 1.33 × 10¹⁴ | 2.43 × 10¹³ | 3 |
| 7 (TA2 + | ≤1:400/≥1:51200 | 1/36 | 0/10 | 0/10 | 3/38 | 1.33 × 10¹⁴ | 2.43 × 10¹³ | 3 |
| TA1 + TA2) | | | | | | | | |

AAV− = Anti-AAVrh74 negative animals,
AAV+ = Anti-AAVrh74 positive animals,
TA1 = Test Article 1 (Imlifidase),
TA2 = Test Article 2 (AAVrh74.CMV.eGFP);
vg = vector genome
^{a}Animals in Groups 2 through 7 were administered prednisolone at a dose level of 1 mg/kg/day, a dose volume of 0.33 mL/kg, and a dose concentration of 3 mg/mL throughout the duration of the dosing phase through the day prior to the terminal sacrifice. On days of control or test article administration, prednisolone was administered approximately 1 hour prior to dose administration.
^{b}Anti-AAVrh74 antibody status was based on the titer values obtained during the predose phase. Anti-AAVrh74 negative animals in Groups 2 and 3 were defined as those with Anti-AAVrh74 antibody titers ≤1:400. Anti-AAVrh74 positive animals in Groups 4 and 5 were defined as those with Anti-AAVrh74 antibody titers of over 1:400 (i.e. 1:800-1:1600). Anti-AAVrh74 positive animals in Group 6 were defined as those with Anti-AAVrh74 antibody titers of 1:3200-1:25600. Anti-AAVrh74 negative animals in Group 7 were defined as those with Anti-AAVrh74 antibody titers ≤1:400 and Anti-AAVrh74 antibody titers levels of ≥1:51200, within 7 days prior to administration of Test Article 1 on Day 36.
^{c}Animals in Groups 3, 4, and 7 received control article (sterile saline) only on Day 1. Animals in Group 2 received control article (sterile saline) only on Days 1, 3, 36, and 38.
^{d}On Days 1 (Groups 2 through 7) and 36 (Groups 2 and 7), animals were dosed at a volume of 1 mL/kg with either control article or Test Article 1.
^{e}On Days 3 (Groups 2 through 7) and 38 (Groups 2 and 7), animals were dosed at a volume of 5.47 mL/kg, with either control article or Test Article 2.
^{f}Animals in Groups 3, 4, 5, and 6 underwent an observation period of 60 days following control article or Test Article 2 administration on Day 3 and were sacrificed on Day 63. Animals in Groups 2 and 7 underwent an observation period of 59 days following control article or Test Article 2 administration on Day 38 and were sacrificed on Day 97.

In conclusion, animals at three different seropositive levels were administered control article (saline) or 10 mg/kg/dose Imlifidase via bolus intravenous injection once on Day 1 (Groups 3 through 6) or once on Days 1 and 36 (Groups 2 and 7) followed by control article or 1.33×10¹⁴ vg/kg/dose AAVrh74.eGFP via slow bolus intravenous injection once on Day 3 (Groups 3 through 6) or once on Days 3 and 38 (Groups 2 and 7). As a standard immuno-suppressive treatment, all animals were also administered a single dose of prednisolone via oral gavage once daily for the duration of the study. AAVrh74.eGFP-related clinical observations included fast respiration, corresponding to macroscopic observations of fluid in the abdominal and thoracic cavities noted in one animal (anti-AAVrh74 titer≤1:400) dosed with AAVrh74.eGFP (Group 3).

Assessment of safety and efficacy was based on mortality, clinical observations, body weights, body weight change, qualitative food consumption, nerve conduction velocity (NCV) measurements, and clinical and anatomic pathology. Blood samples were collected for clinical pathology, anti-AAVrh74 antibody, Imlifidase exposure (toxicokinetics) and efficacy (pharmacodynamics), cytokine, peripheral blood mononuclear cell, and complement analyses. Muscle biopsies and select tissues were collected for vector genome analysis and direct fluorescence evaluation.

All animals survived to their scheduled sacrifice. No AAVrh74.eGFP-related changes in body weight or food consumption, alterations to sensory NCV, or peripheral blood mononuclear cell analysis were noted in animals administered AAVrh74.eGFP, whether preceded with Imlifidase or not. AAVrh74.eGFP-related clinical observations of fast respiration, corresponding to macroscopic observations of fluid in the thoracic and abdominal cavities, were noted in one Group 3 animal.

### Total Anti-AAVrh74 Antibody Responses in AAVrh74-eGFP and Imlifidase Treated Animals

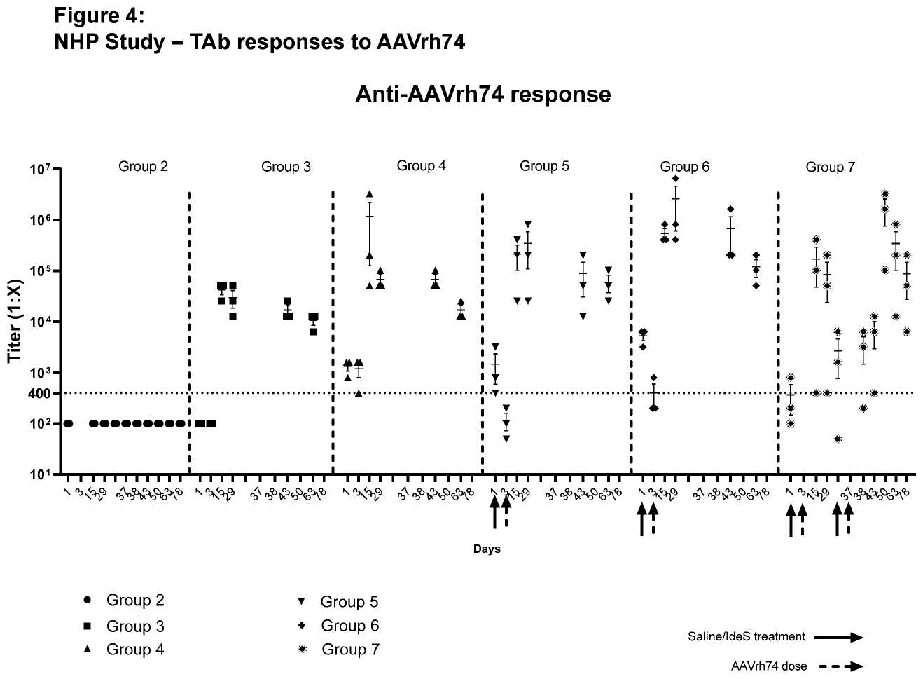

Total antibody responses to AAVrh74-eGFP are shown in FIG. 4. As expected, Group 2 animals having no pre-existing antibodies and receiving only saline did not develop anti-AAVrh74 antibodies. Group 3 animals with total AAVrh74 antibody titers below 1:400 prior to any treatment showed an increase in total anti-AAVrh74 antibody titers after receiving the AAVrh74-eGFP vector (FIG. 4, Group 3).

Group 4 animals with total AAVrh74 antibody titers between 1:800 and 1:1,600 pre-treatment, also showed increased anti-AAVrh74 antibody titers after receiving the AAVrh74-eGFP vector (FIG. 4, Group 4).

Group 5 animals with total AAVrh74 antibody titers between 1:800 and 1:1,600 pre-treatment showed a reduction of total AAVrh74 antibody titers below 1:400 upon administration of Imlifidase on day 1. Following AAVrh74-eGFP administration on day 3, the total AAVrh74 antibody titers increased (FIG. 4, Group 5).

Group 6 animals with total AAVrh74 antibody titers between 1:3,200 and 1:25,600 pre-treatment showed a reduction of AArh74 antibody titers to about 1:400 upon administration of Imlifidase on day 1. Following AAVrh74-eGFP administration on day 3, the total AAVrh74 antibody titers increased (FIG. 4, Group 6).

Group 7 animals with total AAVrh74 antibody titers at/below 1:400 pre-treatment developed anti-AAVrh74 antibody titers after receiving a first dose of AAVrh74-eGFP vectors on day 3. The animals were treated with Imlifidase on day 36, which Imlifidase treatment reduced the total anti-AAVrh74 antibody titers. On day 38, the animals received a second dose of AAVrh74-eGFP, which led to a moderate increase in anti-AAVrh74 antibody titers until about day 50, after which time point the total anti-AAVrh74 antibody titers rose further.

Taken together these results demonstrated that Imlifidase when administered to NHPs on day 1 maintained total AAVrh74 antibody titers at or below 1:400 until at least day 3. Further the data demonstrated that a second dose of Imlifidase administered prior to a second dose of AAVrh74-eGFP resulted in a less pronounced increase in anti-AAVrh74 antibody titers.

Total Anti-AAVrh74 Antibody Titers after Imlifidase

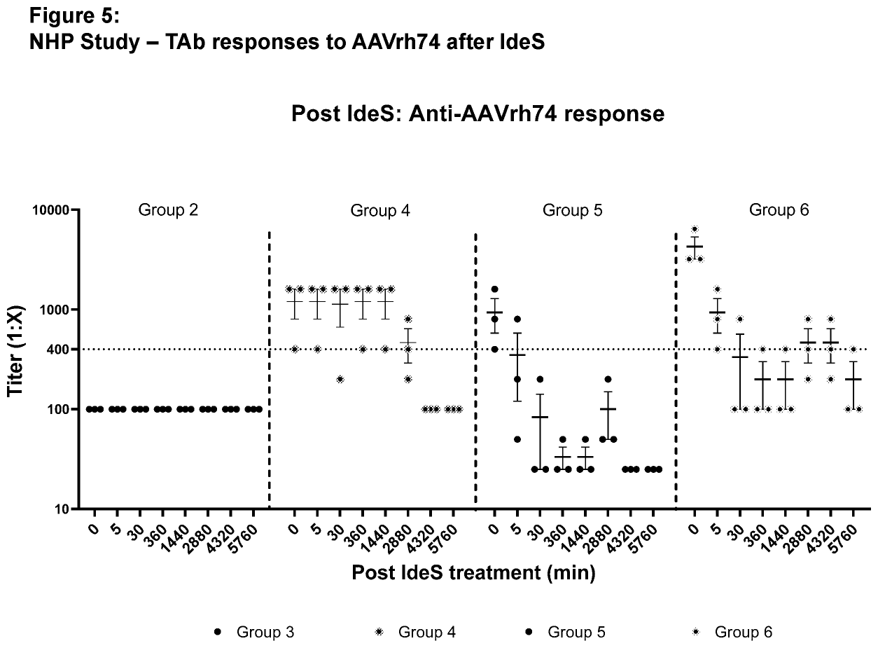

Animals of Groups 2, 4, 5, and 6 were treated with Imlifidase and the total anti-AAVrh74 antibody titers were measured starting at 5 minutes to 5760 minutes (96 h) after Imlifidase treatment (FIG. 5). No AAVrh74 antibodies were detected in Group 2 control animals. In Group 4 animals with pre-treatment AAVrh74 antibody titers between 1:800 and 1:1,600, Imlifidase treatment reduced the titers to below 1:400 after about 2880 minutes (48 h) (FIG. 5). In Group 5 animals with pre-treatment AAVrh74 antibody titers between 1:800 and 1:1,600 and Group 6 animals with pre-treatment AAVrh74 antibody titers between 1:3,200 and 1:25,600, Imlifidase treatment reduced the AAVrh74 antibody titers to below 1:400 after 5 minutes and 30 minutes, respectively (FIG. 5).

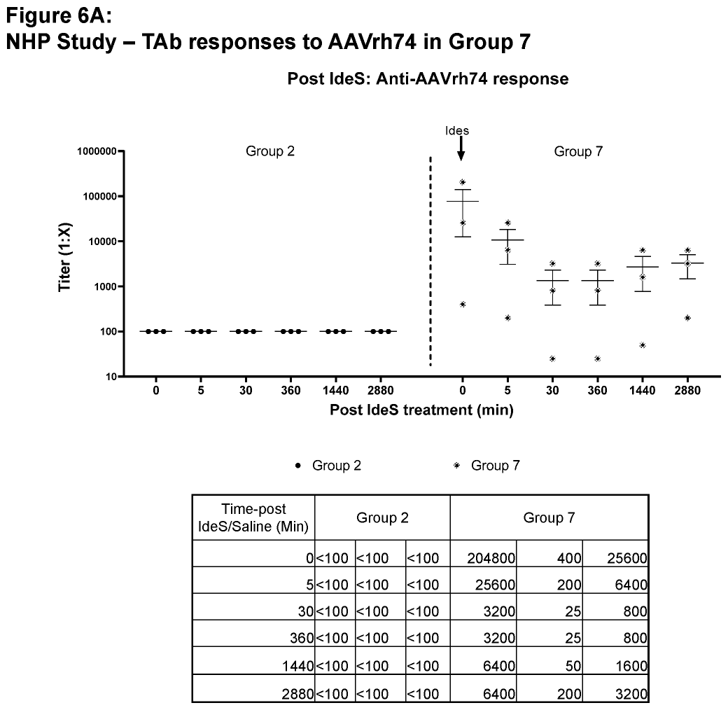

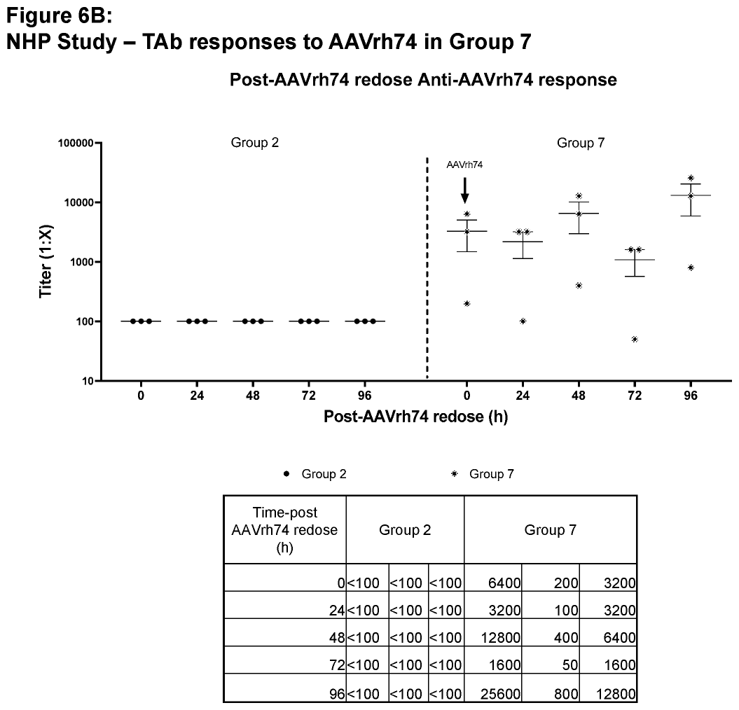

In Group 7 animals with anti-AAVrh74 antibody titers of 1:204,800; 1:25,600; and 1:400, respectively, Imlifidase treatment reduced the anti-AAVrh74 antibody titers in all animals after 5 minutes and lowest titers were measured between 30 minutes and 360 minutes (6 h) (FIG. 6A). Importantly, Group 7 animals did not develop significant increases in anti-AAVrh74 antibody titers for at least 72 hours after anti-AAVrh74-eGFP redosing (FIG. 6B).

Taken together, animals administered a single dose of AAVrh74-eGFP, had increased anti-AAVrh74 capsid antibody titers noted in both anti-AAVrh74 negative and positive animals, with a slightly higher titers observed in seropositive animals (Group 4). The Imlifidase treatment reduced the titer to cut-off (titer cut off is defined as ≤1:400) in the animals with low (titer level≤1:400) and medium (titer level 1:800-1:1600) seropositivity. However, animals with high seropositive levels (titer≥1:51200, Group 7) that were re-administered AAVrh74-eGFP, Imlifidase was unable to reduce high seropositive titers down to the cut-off, with the exception of 1 out of 3 animals. Dosing of low and moderately seropositive animals was able to reduce the titers to cut-off following Imlifidase treatment. The ability of the Imlifidase treatment to reduce the titer down to ≤1:400, was dependent on the total burden of anti-AAVrh74 antibody prior to AAV administration.

### Cleavage Efficiency of Imlifidase

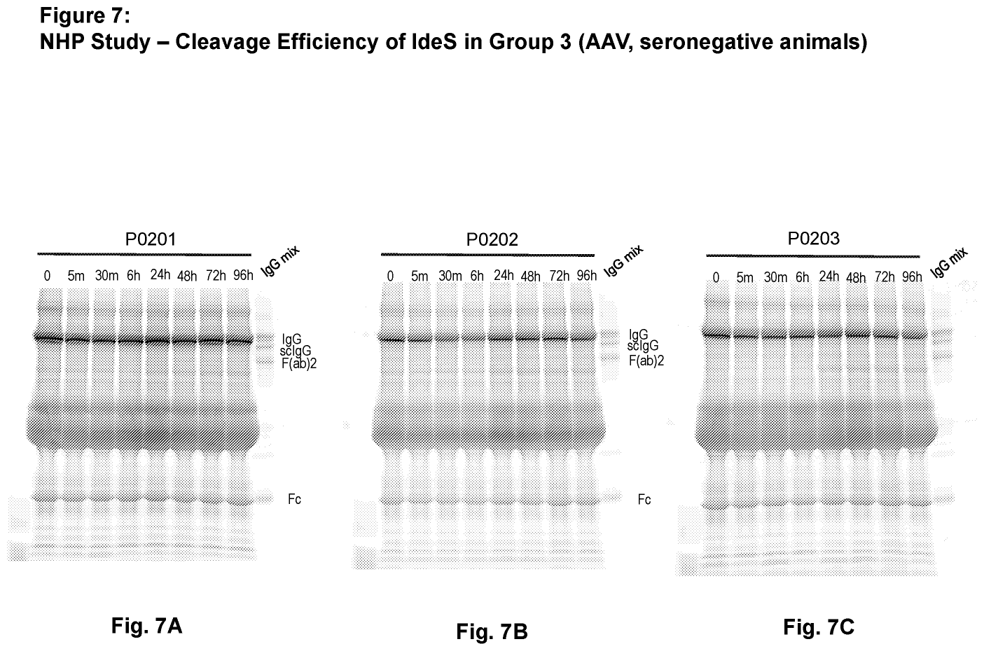

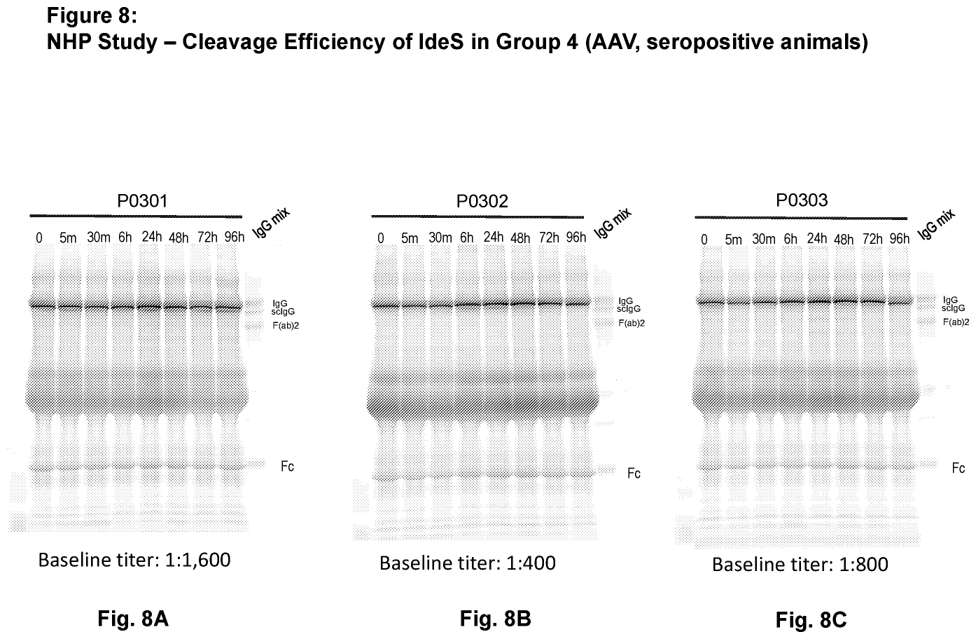

The cleavage efficiency of Imlifidase was measured in serum samples of all animals using Western Blots. Serum samples of seronegative Group 3 animals (P0201, P0202, P0203) and seropositive Group 4 animals (ID numbers: P0301, P0302, P0303) without Imlifidase treatment are shown in FIGS. 7 and 8.

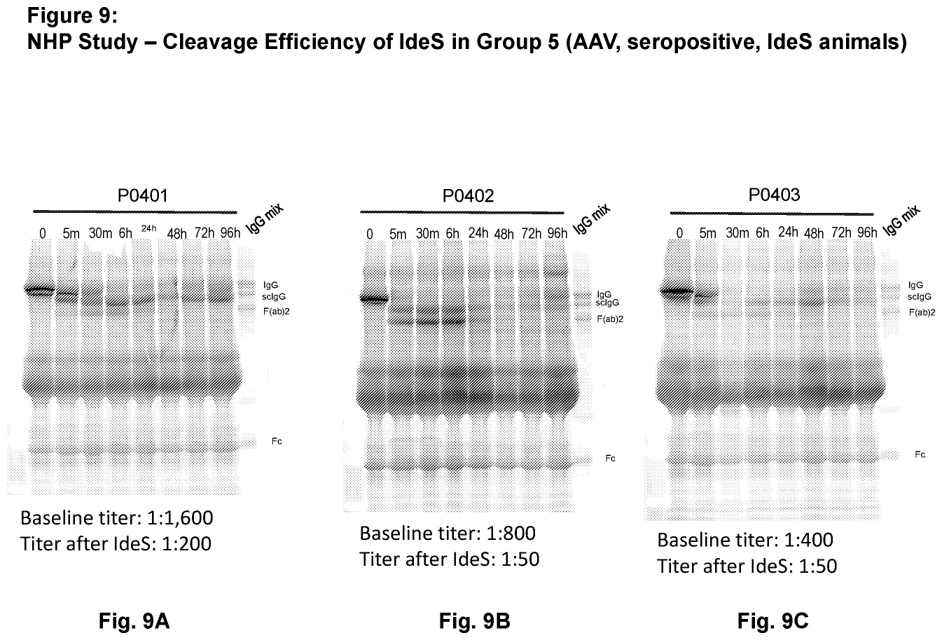

Seropositive Group 5 animals with baseline AAVrh74 antibody titers of 1:400, 1:800, and 1:1,600 (P0401, P0402, P0402) when treated with Imlifidase demonstrated scIgG, F(ab)2 and Fc fragments starting at 5 minutes after Imlifidase treatment and resulting in AAVrh74 antibody titers of 1:200, 1:50, and 1:50, respectively, after Imlifidase treatment (FIG. 9). Furthermore, scIgG, F(ab)2 and Fc fragments were predominant over whole IgG for at least 24 hours in all animals.

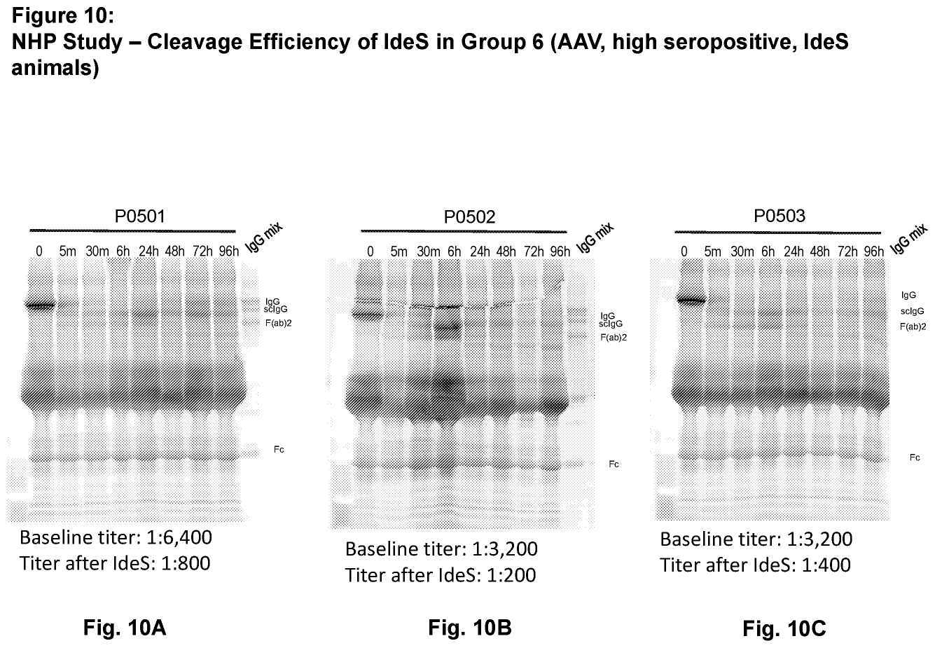

Similarly, seropositive Group 6 animals with baseline AAVrh74 antibody titers of 1:6,400, 1:3,200, and 1:3,200 (P0501, P0502, P0503) when treated with Imlifidase demonstrated scIgG, F(ab)2 and Fc fragments starting at 5 minutes after Imlifidase treatment and resulting in AAVrh74 antibody titers of 1:800, 1:200, and 1:400, respectively, after Imlifidase treatment (FIG. 10). ScIgG, F(ab)2 and Fc fragments were predominant over whole IgG for at least 24 hours in all animals.

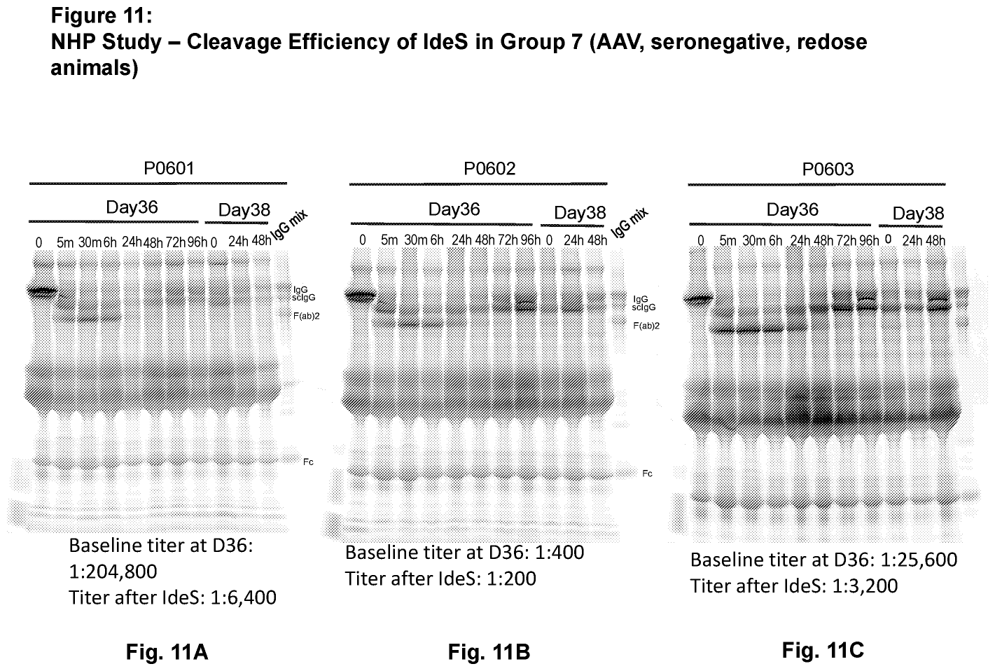

Seronegative Group 7 animals that received a first dose of AAVrh74-eGFP vectors had AAVrh74 antibody titers at day 36 of 1:204,800, 1:400, and 1:25,600, respectively (P0601, P0602, P0603) (FIG. 11). Following Imlifidase treatment at day 36, the Group 7 animals demonstrated scIgG, F(ab)2 and Fc fragments starting at 5 minutes after Imlifidase treatment and resulting in AAVrh74 antibody titers of 1:6,400, 1:200, and 1:3,200, respectively, after Imlifidase treatment (FIG. 11). ScIgG, F(ab)2 and Fc fragments were predominant over whole IgG for at least 24 hours in all animals.

eGFP Transgene Expression

Immunolabeling for green fluorescent protein (GFP) was used for the detection of eGFP transgene expression in cells and was noted in the heart (cardiac myocytes), skeletal muscle (myocytes and cells within the inflammatory infiltrate), liver (hepatocytes, Kupffer cells, and/or in cells within small clusters of infiltrates), spleen (mononuclear cells in germinal centers and/or randomly scattered mononuclear cells), and kidney (tubular epithelium, macula densa, and/or interstitial cells of the renal papilla, occasionally in the collecting duct epithelium, and/or rarely in the parietal epithelium of the glomerulus) of animals from all AAVrh74-eGFP treated groups and in the gall bladder (reactive mesothelium) of one Group 7 animal.

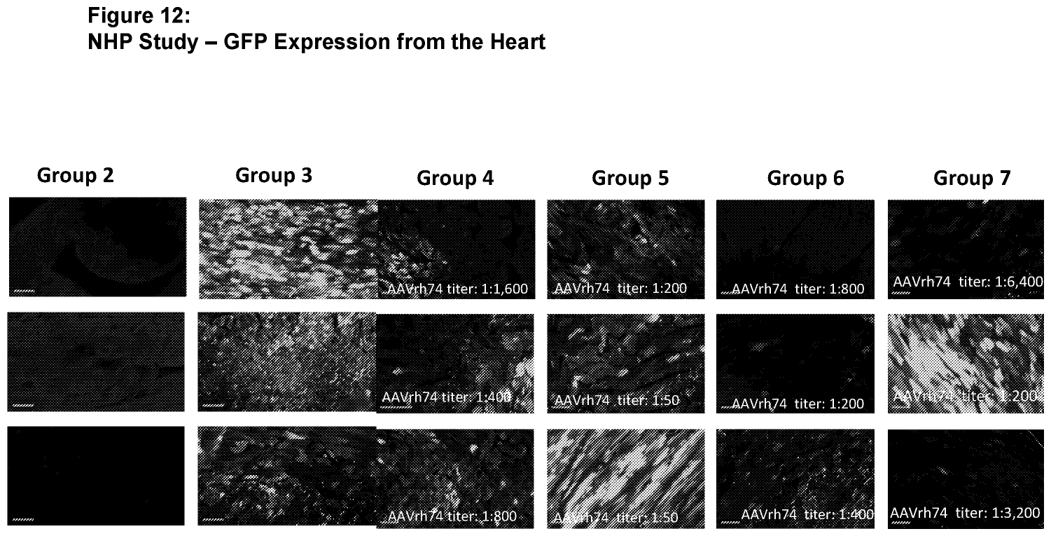

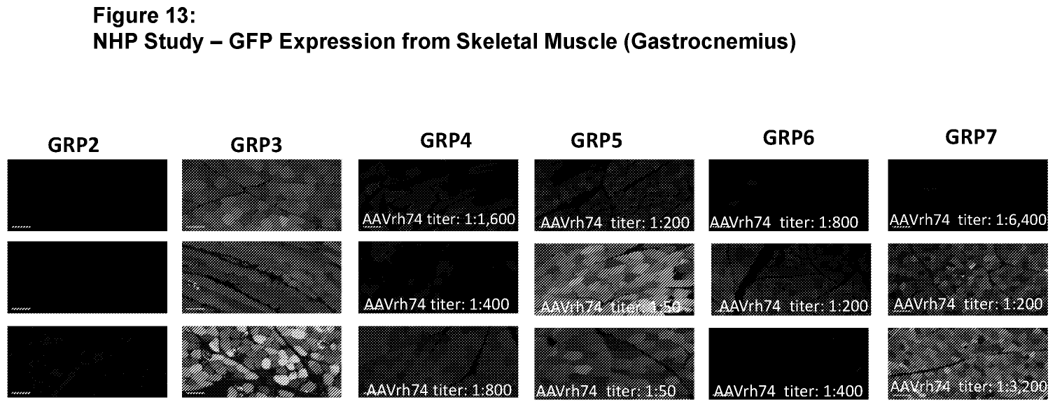

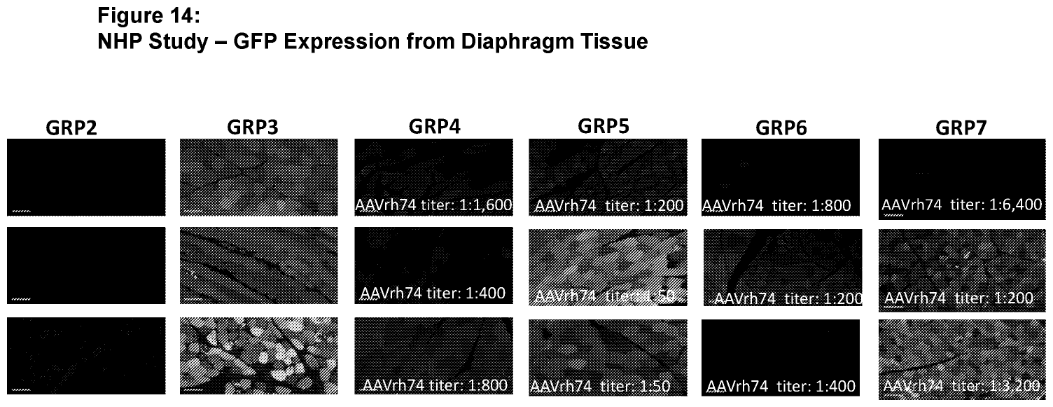

eGFP transgene expression was assessed by fluorescence microscopy. Seronegative Group 3 animals administered AAVrh74-eGFP vector showed high GFP expression in heart (FIG. 12), skeletal muscle (FIG. 13) and diaphragm (FIG. 14).

Seropositive Group 4 animals administered AAVrh74-eGFP vector showed moderate GFP expression in heart (FIG. 12) and diaphragm (FIG. 14) and low GFP expression in skeletal muscle (FIG. 13). A trend towards higher GFP expression in animals with low anti-AAVrh74 antibody titers was observed.

Seropositive Group 5 animals treated with Imlifidase and administered AAVrh74-eGFP vector showed high GFP expression in heart (FIG. 12), skeletal muscle (FIG. 13) and diaphragm (FIG. 14) with the highest GFP expression in animals with anti-AAVrh74 antibody titers of 1:50 after Imlifidase and slightly lower GFP expression in animals with titers of 1:200 after Imlifidase treatment.

In contrast, high seropositive Group 6 animals administered AAVrh74-eGFP vector after Imlifidase treatment showed low GFP expression in heart (FIG. 12), skeletal muscle (FIG. 13) and diaphragm (FIG. 14). As discussed above, Group 6 animals had post-Imlifidase anti-AAVrh74 antibody titers of 1:800, 1:200 and 1:400, respectively.

The one Group 7 animal with an anti-AAVrh74 antibody titer of 1:400 after the first dose of AAVrh74-eGFP and anti-AAVrh74 antibody titer of 1:200 after Imlifidase treatment showed high GFP expression in heart (FIG. 12), skeletal muscle (FIG. 13), and diaphragm (FIG. 14). In contrast, the two Group 7 animals with high anti-AAVrh74 antibody titers after the first dose of AAVrh74-eGFP and anti-AAVrh74 antibody titers of 1:6,400 and 1:3,200, respectively, after Imlifidase treatment showed low GFP expression in the heart (FIG. 12), and moderate GFP expression in skeletal muscle (FIG. 13), and diaphragm (FIG. 14).

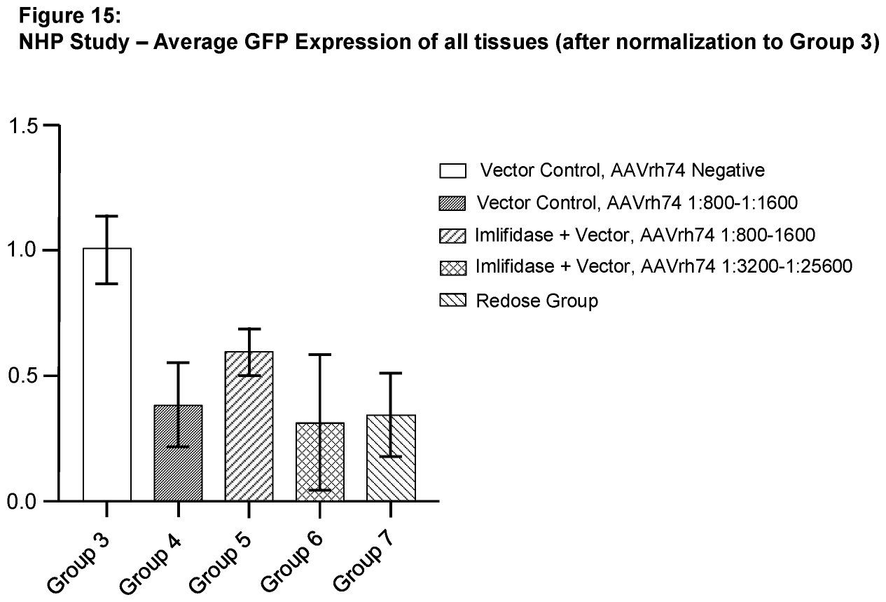

To determine transduction levels across all tissues, average GFP expression in all tissues was normalized to Group 3 (anti-AAVrh74 antibody negative and administered AAVrh74eGFP). Groups 4, 5, 6, and 7 showed reduced GFP expression compared to Group 3 with Group 5 (anti-AAVrh74 antibody titers of 1:800-1:1,600 and imlifidase+AAVrh74-eGFP treatments) showing the highest GFP in all tissues compared to Groups 4, 6, and 7 (FIG. 15).

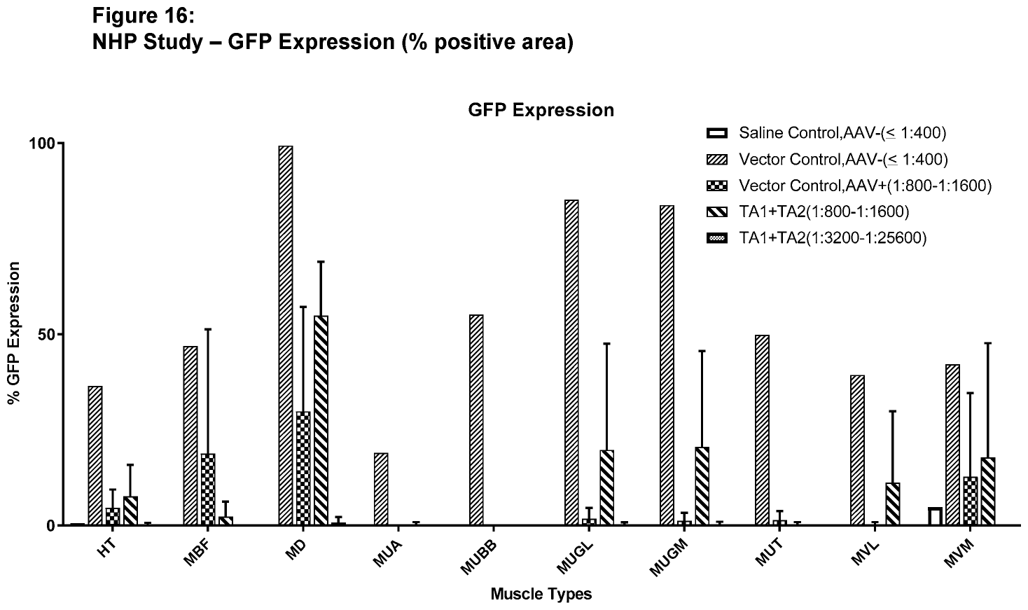

GFP expression was also measured in difference muscle tissues. GFP expression was highest in all tested muscle tissues for seronegative, AAVrh74-eGFP treated Group 3 animals (FIG. 16). Seropositive Group 4 animals treated with AAVrh74-eGFP showed moderate GFP expression in the heart, bicep femoralis, diaphragm, and vastus medialis (FIG. 16). Thus, animals with moderate levels of pre-existing anti-AAVrh74 antibodies (1:800-1:1,600 anti-AAVrh74 antibody titer) prior to treatment with AAVrh74-eGFP, had low levels of eGFP expression consistent with the understanding that pre-existing antibodies prevent the mechanism of AAVrh74-eGFP expression. Low level seropositive Group 5 animals treated with Imlifidase prior to AAVrh74-eGFP showed moderate GFP expression in the heart, diaphragm, lateral and medial gastrocnemius muscle, and vastus lateralis and medialis (FIG. 16). Thus, administration of Imlifidase to animals with moderate levels (1:800-1:1600 anti-AAVrh74 antibody titer) of pre-existing antibodies (Group 5) led to an increase in eGFP expression compared to those with moderate levels of pre-existing antibodies which did not receive Imlifidase (Group 4). In contrast, animals with high levels (1:3,200-1:25,600 anti-AAVrh74 antibody titer) of pre-existing antibodies (Group 6) treated with Imlifidase prior to AAVrh74-eGFP showed only low levels of GFP expression in the heart, diaphragm, triceps, lateral and medial gastrocnemius muscle and no GFP expression in other tissues (FIG. 16).

Additionally, those animal with pre-existing low antibody titer treated twice with Imlifidase and AAVrh74-eGFP yielded moderate levels of GFP expression (Group 7). These data demonstrate the effectiveness of the Imlifidase in enhancing the transduction rate of AAVrh74-eGFP in seropositive animals, dependent upon the antibody titer.

### Vector Genome Analysis

Data from vector genome analysis of tissues showed that animals without pre-existing anti-AAVrh74 antibodies (titer levels≤1:400, Group 3), achieved the highest levels of vector genome across the animals. Administration of Imlifidase prior to the administration of AAVrh74-aeGFP for animals with pre-existing moderate antibodies (1:800-1:1600 anti-nti-AAVrh74 antibody titer range, Group 5) led to an increase in GFP expression compared to animals with the same pre-existing antibody range which did not receive Imlifidase (Group 4), demonstrating the effectiveness of Imlifidase in lowering pre-existing antibodies, enhancing AAV transduction and subsequent GFP expression in seropositive animals.

However, animals with a pre-existing high anti-AAVrh74 antibody titer levels (1:3200-1:25600 titer) resulted in the second lowest vector genome transduction across the groups, despite administration with Imlifidase. One animal (Group 7) showed evidence of seroconversion prior to administration of Imlifidase resulting in the lowest transduction levels of the Group 7 animals. After 2 doses of AAVrh74-eGFP, Group 7 demonstrated the lowest transduction levels across all tissues analyzed and was most similar in transduction to Group 6.

### Chemokine Analysis

No changes in any of the cytokines examined were noted at 24 or 48 hours postdose for animals administered Imlifidase, compared with predose phase values, controls, or animals not administered Imlifidase on Day 1.

### Interleukin-1 Receptor Antagonist (IL-1RA) and Monocyte Chemoattractant Protein-1 (MCP-1)

### AAVrh74-eGFP—Single Dose

Robust increases in IL-1RA (70.75 fold) and MCP-1 (4.38 fold) levels were noted on Day 43 (40 days post the AAVrh74-eGFP) of Dosing Phase III in AAVrh74 negative animals (Group 3) administered AAVrh74-eGFP, compared with predose phase values. The magnitude of the increases was noted to be several fold higher in two animals within Group 3. IL-1RA levels decreased at subsequent time points, compared with Day 43 of Dosing Phase III values, but remained over 18 fold higher compared with predose values. MCP 1 levels continued to remain similar to Day 43 of Dosing Phase III values for the remainder of the study.

Less robust increases in IL-1RA (over 4.5 fold) and MCP-1 levels (over 0.96 fold) were noted at 72 hours postdose on Day 1 of Dosing Phase III (24 hours post the AAVrh74-eGFP dose) in AAVrh74-positive animals administered control article or Imlifidase and AAVrh74-eGFP (Groups 4, 5, and 6). IL 1RA and MCP-1 levels trended back towards predose levels and remained at levels similar to, or slightly higher than, predose values on Day 43 of Dosing Phase III and at subsequent time points in anti-AAVrh74 antibody positive animals administered control article and AAVrh74-eGFP (Group 4) or Imlifidase and AAVrh74-eGFP (Group 6). However, in anti-AAVrh74 antibody-positive animals administered Imlifidase and AAVrh74-eGFP (Group 5), IL 1RA and MCP-1 levels remained increased in 2 of 3 animals and in 1 of 3 animals, respectively, on Day 43 of Dosing Phase III and at subsequent time points, compared with baseline values.

Taken together, AAVrh74-eGFP-related increases in IL-1RA and MCP-1 were noted in AAVrh74 negative and positive animals (Groups 3 through 6), with delayed but robust and prolonged increases noted in AAVrh74-negative animals administered AAVrh74-eGFP (Group 3). Changes in the levels of IL 1RA and MCP-1 in AAVrh74-positive animals administered Imlifidase and AAVrh74-eGFP (Groups 5 and 6), compared with anti-AAVrh74 antibody positive animals administered Imlifidase and AAVrh74-eGFP (Group 4), were small in magnitude and inconsistent in animals within the same group; as such, these were considered not related to Imlifidase.

### AAVrh74-eGFP—Repeat Dose

Increases in IL-1RA levels were noted at 72 hours postdose on Day 1 of Dosing Phase III (24 hours post the first AAVrh74-eGFP dose) in one anti-AAVrh74 antibody negative animal. More robust increases in IL-1RA (17.87 fold) were noted on Day 15 of Dosing Phase III (12 days post the first AAVrh74-eGFP dosing) in all anti-AAVrh74 antibody negative animals administered AAVrh74-eGFP (Group 7), compared to predose values. IL-1RA levels trended back towards predose values but remained higher (2.58 fold) than predose IL-1RA values on Day 29 of Dosing Phase III (26 days post the AAVrh74-eGFP dose). Further increases in IL-1RA levels were noted on Day 57 of Dosing Phase III (21 and 19 days post the Imlifidase dose and the second AAVrh74-eGFP dose, respectively) in only one animal administered a second dose of AAVrh74-GFP, while the IL-1RA values in the other animals administered a second dose of AAVrh74-eGFP (Group 7) remained close to levels noted on Day 29 of Dosing Phase III (prior to Imlifidase administration) for the remainder of the study. Overall, AAVrh74-eGFP-related increases in IL-1RA were noted in anti-AAVrh74 antibody negative animals administered the first dose of AAVrh74.eGFP. Changes in IL-1RA after the Imlifidase dose and the second AAVrh74-eGFP dose were small in magnitude and inconsistent in animals within this same group; hence, they were considered not related to AAVrh74-eGFP or Imlifidase.

Decreases in MCP-1 levels were noted on Day 29 of the dosing phase (26 days post the first AAVrh74-eGFP dose) in one animal administered AAVrh74.eGFP, compared with predose values. MCP-1 levels continued to remain decreased in this animal at all subsequent time points. Increases in MCP-1 levels were noted on Day 43 of Dosing Phase III (7 and 5 days post the Imlifidase dose and the second AAVrh74-eGFP dose, respectively) and at all subsequent time points during Dosing Phase III in one animal administered the second dose of AAVrh74-eGFP, compared with baseline values. Overall, changes in MCP-1 levels were inconsistent in animals after the first and second doses of AAVrh74-eGFP (Group 7) and were considered unrelated to AAVrh74-eGFP.

### Other Cytokines

Levels of IL-8 were detectable or above the upper limit of quantitation for all animals at all time points evaluated, even at predose. Changes in IL-8 were comparable with predose phase values and noted in controls; as such, these changes were attributed to normal biological variation and considered independent of the test articles.

Increases in MIP-1β were noted on Day 15 of Dosing Phase III (12 days post the first AAVrh74-eGFP dose) in animals administered AAVrh74.eGFP (Group 7). These increases were small in magnitude and not observed at subsequent time points and, hence, were considered unrelated to the test articles.

Levels of GM-CSF, IFN-γ, L-1β, IL-2, IL-5, IL-6, IL-10, IL2/IL23p40, IL-13, IL 15, IL 17A, MIP-1α and TGF-α were sporadically elevated above the lower limit of quantitation in animals administered control article or AAVrh74.eGFP administered AAVrh74-eGFP. Changes in these cytokines were small in magnitude, observed in controls and inconsistent in animals within the same group; therefore, they were considered normal biological variations and unrelated to Imlifidase or the AAVrh74-eGFP.

In conclusion, in animals that were administered a single dose of AAVrh74-eGFP, AAVrh74-eGFP-related increases in IL-1RA and MCP-1 were noted in both anti-AAVrh74 antibody negative (Group 3) and positive animals (Groups 4, 5, and 6), with delayed but prolonged and more robust increases noted in the negative animals. No consistent Imlifidase-related changes were noted in IL-1RA or MCP-1 levels in anti-AAVrh74 antibody positive animals administered Imlifidase and AAVrh74-eGFP (Groups 5 and 6), compared with animals administered saline and AAVrh74-eGFP (Group 4). In animals that were administered repeat doses of AAVrh74-eGFP (Group 7), AAVrh74-eGFP-related increases in IL-1RA, but not MCP-1, were noted in anti-AAVrh74 antibody negative animals after the first dose of AAVrh74.eGFP. No consistent AAVrh74-eGFP-related changes were noted in IL-1RA or MCP-1 levels in these animals following administration of Imlifidase and the second dose of AAVrh74-eGFP.

No Imlifidase or AAVrh74.eGFP-related changes in GM-CSF, IFN-γ, IL-1β, IL-2, IL-5, IL-6, IL-8, IL-10, IL12/IL23p40, IL-13, IL-15, IL-17A, MIP-1α, β, or TGF-α levels were noted in animals administered single or a repeat dose of AAVrh74-eGFP.

Thus, AAVrh74-eGFP-related changes were noted in both anti-AAVrh74 antibody negative and positive animals only in levels of IL-IRA and MCP-1. The increases were more robust and prolonged in anti-AAVrh74 antibody negative animals when compared to anti-AAVrh74 antibody positive animals. No Imlifidase related changes were noted in levels of any of the cytokines examined. No AAVrh74-eGFP or Imlifidase related impacts on levels of other cytokines and complement activation were noted.

### Clinical Pathology

AAVrh74-eGFP-related clinical pathology findings in animals from Groups 2 through 7 were minimal to moderate in magnitude and primarily consisted of increased alanine aminotransferase (ALT) and aspartate aminotransferase (AST) activities, often associated with increased creatine kinase (CK) activity. Clinical pathology were consistent with a striated muscle injury, which correlated with the microscopic findings of degeneration/necrosis in the heart and skeletal muscles. Cardiomyocyte degeneration/necrosis in the heart likely also contributed to the aforementioned increased CK and/or AST activities. Enzyme activities (ALT, AST, and CK) were generally of the highest magnitude on Days 22 and/or 36 in all AAVrh74-eGFP-treated groups, regardless of antibody titers or co-administration of Imlifidase, and generally demonstrated a trend of declining values over time (Days 36 through 63) in Groups 4, 5, and 6, compared with animals in Groups 3 and 7.

Additional AAVrh74-eGFP-related effects were noted in one animal in Groups 3 and 7, with decreased albumin and total protein concentrations; decreased albumin concentration, cardiomyocyte degeneration, necrosis and inflammation likely resulting in the pleural effusion was considered a contributory mechanism for fluid accumulation in two or more body cavities in these animals. AAVrh74-eGFP-related effects limited to the Group 7 animal consisted of decreased red cell mass, mean cell volume (decreased red blood cell [RBC]size), mean corpuscular hemoglobin concentration, and mean corpuscular hemoglobin (decreased RBC hemoglobin content); increased RBC distribution width and moderately increased absolute reticulocyte count, which indicated an erythroid regeneration; and increased platelet count, considered secondary to the decreased red cell mass and regenerative response. Decreased food consumption likely contributed to these findings and correlated with decreased cholesterol concentration in this animal. Further, the Group 7 animal was noted with evidence of a hepatobiliary perturbation (increased gamma glutamyltransferase and alkaline phosphatase activities and total bilirubin concentration) in the absence of microscopic evidence of hepatobiliary changes. An additional, AAVrh74-eGFP-related effect limited to the Group 3 animal consisted of increased urea nitrogen and inorganic phosphorus concentration, indicative of a kidney perturbation, which were unaccompanied by microscopic correlates in the kidney.

In anatomic pathology, AAVrh74-eGFP-treated groups, vector-related microscopic findings at the terminal sacrifice were noted in the peripheral nervous system (sciatic nerve, left and right plantar nerves, and left and right radial nerves), heart, skeletal muscle (biceps femoris, left gastrocnemius [medial and lateral], right gastrocnemius [medial and lateral], anterior tibialis, and skeletal muscle of the tongue and esophagus), and liver. Vector-related microscopic findings in the central nervous system (brainstem and spinal cord) and lumbar dorsal root ganglion were noted in animals from Groups 3, 5, and 7. These microscopic findings in the spinal cord and dorsal root ganglion are well characterized changes observed with administration of AAV vectors (Hinderer et al., 2018; Hutt et al., 2022). Additionally, these findings were not associated with clinical observations or observations during nerve conduction velocity testing.

AAVrh74-eGFP-related microscopic findings noted in all AAVrh74-eGFP-treated groups, whether preceded by Imlifidase or not, included nerve fiber degeneration (minimal to moderate) in the peripheral nerves (sciatic, left and right plantar, and left and right radial); oval cell hyperplasia (minimal to moderate) in the liver; degeneration/necrosis (minimal to moderate) and mononuclear cell inflammation (minimal to marked) in the heart; and degeneration and necrosis (minimal to marked), mononuclear cell inflammation (minimal to marked), regeneration (minimal to slight), and/or fibrosis (minimal to moderate) in the skeletal muscle (biceps femoris, left gastrocnemius [medial and lateral], right gastrocnemius [medial and lateral], anterior tibialis, and/or skeletal muscle of the tongue and esophagus).

The microscopic findings in the skeletal muscle are consistent with a recognized effect of GFP report gene expression in skeletal muscle and oval cell hyperplasia in the liver and correlated with the clinical pathology findings of increased aspartate aminotransferase (AST), creatine kinase (CK), and alanine aminotransferase (ALT). Degeneration and necrosis in the heart likely also contributed to the increased AST and/or CK activities. AAVrh74 vector-related macroscopic findings were noted at the intravenous injection site (thickened tissue surrounding the right saphenous vein) and right medial gastroenemius (discolored tan) of one Group 5 animal and correlated with the microscopic findings of skeletal muscle degeneration, necrosis, and mononuclear cell inflammation in the skeletal muscle deep to the right saphenous vein and right medial gastrocnemius.

Taken together, these results indicated that animals with higher AAVrh74 titers were noted with a lesser severity of AAVrh74-eGFP-related effects in clinical pathology test results consistent with a well-recognized effect that pre-existing anti-AAV antibodies reduce AAV transduction. No AAVrh74-eGFP-related effects, with or without Imlifidase, were noted in coagulation test results during Dosing Phase III. AAVrh74-eGFP-related hematology effects during Dosing Phase III were limited to the aforementioned Group 7 animal.

### SUMMARY

Animals that received a single dose of AAVrh74eGFP were noted with increased anti-capsid antibody titers, regardless of seropositivity levels. Treatment with Imlifidase reduced the anti-AAVrh74 antibody titer levels to the defined cut-off (≤1:400) in animals with low and medium pre-existing titer levels. Imlifidase was unable to reduce the high anti-AAVrh74 titer levels to the cut off in 2 of 3, Group 7 animals after the second dose series. The ability of the Imlifidase treatment to reduce the titer to the cut-off, was dependent on the total burden of anti-AAVrh74 antibody prior to AAV administration. Likewise, vector genome analysis of tissues and quantification of percent GFP has also shown that Imlifidase was effective in lowering pre-existing antibodies, enhancing AAV transduction and subsequent eGFP expression and enhancing the transduction rate of AAVrh74-eGFP in seropositive animals, dependent upon the pre-existing antibody titer level.

Overall, Imlifidase was found to be a safe and effective means to reduce the pre-existing anti-AAVrh74 titer levels in seropositive animals administered AAVrh74-eGFP, however, reduction was dependent upon the levels of anti-AAVrh74 titer pre-existing prior to the administration of Imlifidase. Repeat dosing of Imlifidase on Day 36 followed by a second administration of AAVrh74-eGFP on Day 38 reduced the anti-AAVrh74 antibody titer levels in 1 of 3 animals and thus was less efficacious in a repeat dose scenario.

Taken together, this NHP study demonstrated proof of concept that Imlifidase, administered pre-treatment, lowered total AAVrh74 antibody titers to safely dose AAVrh74 gene therapy. No adverse clinical events, no Imlifidase or AAV-related mortality, and no immunotoxicology or histopathology, including in reproductive organs, were observed in this NHP study.

Lowering AAVrh74 antibody titers by Imlifidase pre-treatment led to efficient transduction and expression of the GFP transgene in muscle, heart, and diaphragm.

### Example 2: Open-Label, Systemic Gene Delivery Study to Evaluate the Safety, Tolerability and Expression of Delandistrogene Moxeparvovec in Association with Imlifidase in Subjects with Duchenne Muscular Dystrophy with Pre-Existing Antibodies to rAAVrh74

### Objectives and Endpoints

### Primary Objectives

1) To evaluate micro-dystrophin expression at 12 weeks post dosing of delandistrogene moxeparvovec in subjects pre-treated with Imlifidase. Endpoint: Change in quantity of micro-dystrophin protein expression in biopsied muscle tissue from Baseline to Week 12 (Part 1) as measured by Western blot; immunofluorescence (IF) fiber intensity; and IF percent dystrophin-positive fibers (PDPF).
2) To evaluate micro-dystrophin transduction at 12 weeks post dosing of delandistrogene moxeparvovec in subjects pre-treated with Imlifidase. Endpoint: Vector genome copies using polymerase chain reaction in muscle tissue biopsy.

### Secondary Objectives

1) To establish the pharmacokinetic (PK) profile of Imlifidase. Endpoint: Imlifidase PK in serum up to 7 days after imflifidase administration.
2) To establish the pharmacodynamic (PD) profile of Imlifidase (cleavage and recovery of IgG). Endpoint: Imlifidase PD (total IgG) in serum up to 12 weeks after Imlifidase administration.
3) To evaluate rAAVrh74 antibody titers following Imlifidase administration. Endpoint: rAAVrh74 antibody titers up to 120 hours after Imlifidase administration.
4) To assess rAAVrh74 genome concentration in systemic circulation. Endpoint: Vector genome copies using polymerase chain reaction in serum up to 7 days after delandistrogene moxeparvovec administration.
5) To evaluate the safety of Imlifidase. Endpoints: Incidence of treatment-emergent adverse events; worsening of vitals or physical examination findings; incidence of adverse events of special interest; incidence of serious adverse events; and clinically significant abnormalities in safety laboratory assessments.
6) To evaluate the safety of Imlifidase followed by delandistrogene moxeparvovec in combination. Endpoints: Incidence of treatment-emergent adverse events; worsening of vitals or physical examination findings; incidence of adverse events of special interest; incidence of serious adverse events; clinically significant abnormalities in safety laboratory assessments; electrocardiograms (ECGs); and echocardiograms (ECHOs).

### Exploratory Objectives

1) To evaluate the effect of delandistrogene moxeparvovec on creatine kinase (CK) levels. Endpoint: Change in CK from Baseline over 104 weeks.
2) To evaluate the effect of delandistrogene moxeparvovec on physical functional assessments as assessed by the North Star Ambulatory Assessment (NSAA) score. Endpoint: Change in NSAA total score from Baseline to 104 weeks.
3) To evaluate the effect of delandistrogene moxeparvovec on timed function tests as assessed by measuring: 100-meter walk/run (100MWR); time to ascend 4 steps; time to rise from the floor; and 10-meter walk/run (10MWR). Endpoints: Change in time of 100MWR from Baseline to 104 weeks; change in time to ascend 4 steps from Baseline to 104 weeks; change in time to rise from the floor from Baseline to 104 weeks; change in time of 10MWR from Baseline to 104 weeks.
4) To evaluate the immunogenicity of delandistrogene moxeparvovec as assessed by the detection of cellular immune response to rAAVrh74 and the micro-dystrophin transgene; and antibodies to rAAVrh74 and micro-dystrophin. Endpoints: Cellular immune response to rAAVrh74 and the micro-dystrophin transgene as measured by enzyme-linked immunospot (ELISpot), see Slota et al., Expert Rev Vaccines 10(3): 299-206, 2011) over 104 weeks; and antibody titers to rAAVrh74 and the micro-dystrophin transgene over 104 weeks.
5) To establish the immunogenicity profile of Imlifidase (anti-drug antibodies [ADA]). Endpoint: Imlifidase ADA serum levels over 104 weeks after Imlifidase administration.

### Methodology

This is an open-label study of ambulatory male subjects with DMD and pre-existing antibodies to rAAVrh74 who are ≥4 to ≤8 years of age. Approximately 6 subjects will be treated with Imlifidase followed by delandistrogene moxeparvovec. In order to ensure that 6 subjects receive both therapies, additional subjects (up to a maximum of 6) may be enrolled to account for subjects who receive Imlifidase but do not meet criteria for delandistrogene moxeparvovec dosing.

Subjects will receive an initial IV dose of Imlifidase (0.25 mg/kg) followed by delandistrogene moxeparvovec (1.33×10¹⁴ vg/kg) IV infusion approximately 48 hours later if their post-Imlifidase rAAVrh74 antibody results are not reactive or a second IV dose of Imlifidase (0.25 mg/kg) within 60 hours of their initial Imlifidase dose if their post-Imlifidase rAAVrh74 results remain reactive. For subjects who receive a second IV dose of Imlifidase, if the post-Imlifidase rAAVrh74 result is not reactive, they will receive IV delandistrogene moxeparvovec (1.33×10¹⁴ vg/kg) approximately 48 hours later. Subjects with rAAVrh74 results returning as reactive after the second dose of Imlifidase, will not receive delandistrogene moxeparvovec.

The first three subjects who are dosed with both Imlifidase and delandistrogene moxeparvovec are sentinel subjects and will enter the Imlifidase infusion period at least one week apart. The safety, PK/PD, and 12-week expression data of the three sentinel subjects will be reviewed by the SRC prior to additional subjects entering the infusion period.

The study will consist of 4 periods:
An up to approximately 3-week Screening Period (pre-infusion) during which disease characteristics, baseline therapy, Imlifidase antibody concentration, and rAAVrh74 antibody titer will be assessed, and the pre-infusion evaluation is completed.
An approximately 1-week Baseline Period (pre-infusion) during which baseline assessments will be completed. This period begins the day of enrollment and ends on the day prior to the first Imlifidase infusion.
An up to 5-day Imlifidase Infusion Period during which up to 2 IV doses of Imlifidase will be administered.
   Starting the day of the first Imlifidase infusion, subjects will begin a 4-week course of prophylactic antibiotics.
   Subjects will receive antihistamines before each Imlifidase infusion.
A follow-up period:
   Subjects with rAAVrh74 antibody results that remained reactive despite two doses of Imlifidase infusion will not receive delandistrogene moxeparvovec and will be followed for 52 weeks during which safety parameters will be monitored. For these subjects, the last study procedure will occur at Week 52.
   Subjects with rAAVrh74 antibody results that return as not-reactive after Imlifidase infusion will receive a single dose of delandistrogene moxeparvovec (1.33×10¹⁴ vg/kg) IV infusion on D1 of a 104-week Follow-Up Period during which safety and expression parameters will be evaluated.
      Starting at least 1 day prior to delandistrogene moxeparvovec infusion, subjects will receive at least 1 mg/kg of a glucocorticoid (prednisone equivalent) daily until at least 60 days after the delandistrogene moxeparvovec infusion, unless earlier tapering is required to manage an adverse event.
      Part 1 of the Follow-Up Period will begin with delandistrogene moxeparvovec infusion (Day 1) through Week 12.
      Part 2 of the Follow-Up Period will begin post-Week 12 through Week 104. For subjects who complete the study, the last study visit will occur at Week 104.

Efficacy, safety, and immunogenicity assessments will occur throughout the study and are presented in the Schedule of Events tables, Tables 11-14.

### Number of Subjects:

Up to 12 subjects may be enrolled; however, enrollment will stop once approximately 6 ambulatory subjects ≥4 to ≤8 years of age are dosed with both Imlifidase and delandistrogene moxeparvovec. At least three subjects enrolled in the study will have delandistrogene moxeparvovec antibody titer ≥1:1600.

### Inclusion Criteria:

A subject must meet all of the following criteria to be eligible to participate in this study:
1. Is male at birth, ambulatory, and ≥4 to ≤8 years of age at the time of Screening.
2. Has a definitive diagnosis of DMD prior to Screening based on documentation of clinical findings and confirmatory genetic testing using a clinical diagnostic genetic test. Genetic report must describe a frameshift deletion, frameshift duplication, premature stop (“nonsense”), canonical splice site mutation, or other pathogenic variant in the DMD gene fully contained between exons 18 to 79 (inclusive) that is expected to lead to absence of dystrophin protein.
   a. Mutations between or including exons 1-17 are not eligible.
   b. In-frame deletions, in-frame duplications, and variants of uncertain significance (VUS) are not eligible.
3. Able to cooperate with motor assessment testing.
4. Is up to date with all regionally recommended immunizations for encapsulated organisms.
5. Stable dose of oral corticosteroids for at least 12 weeks before Screening and the dose is expected to remain constant (except for potential modifications to accommodate changes in weight) up to week 52 of the study.
6. Has total rAAVrh74 antibody results at Screening that are reactive by a total anti-AAVrh74 antibody ELISA assay.
7. Subjects who are sexually active must agree to use, for the entire duration of the study, a condom and the female sexual partner must also use a medically acceptable form of birth control (eg, oral contraceptive).
8. If under the age of consent (≤18 years old) has (a) parent(s) or legal guardian(s) who is (are) able to understand and comply with the study visit schedule and all other protocol requirements.
9. Is willing to provide informed consent (if applicable) and has (a) parent(s) or legal guardian(s) who is (are) willing to provide written informed consent/assent for the subject to participate in the study.

### Exclusion Criteria

A subject who meets any of the following criteria will be excluded from this study:
1. Previous treatment with Imlifidase.
2. High dose IVIG treatment (2 g/kg BW) within 28 days prior to Imlifidase treatment.
3. Has left ventricular ejection fraction <40% on the screening ECHO or clinical signs and/or symptoms of cardiomyopathy.
4. Major surgery within 3 months prior to Day 1 or planned surgery for any time during this study.
5. Presence of any other clinically significant illness, including cardiac, pulmonary, hepatic, renal, hematologic, immunologic, or behavioral disease, or infection or malignancy or concomitant illness or requirement for chronic drug treatment that in the opinion of the Investigator creates unnecessary risks for receiving the study drugs or a medical condition or extenuating circumstance that, in the opinion of the Investigator, might compromise the subject's ability to comply with the protocol required testing or procedures or compromise the subject's wellbeing, safety, or clinical interpretability.
6. Has serological evidence of current, chronic, or active human immunodeficiency virus, hepatitis C, or hepatitis B infection.
7. Subjects with a history of major thrombotic events, active peripheral vascular disease, or proven hypercoagulable conditions.
8. Subjects with active tuberculosis.
9. Presents or has a history of thrombotic thrombocytopenic purpura (TTP), or known familial history of TTP.
10. Has a medical condition or extenuating circumstance that, in the opinion of the Investigator, might compromise the subject's ability to comply with the protocol required testing or procedures or compromise the subject's wellbeing, safety, or clinical interpretability.
11. Has a symptomatic infection (eg, upper respiratory tract infection, pneumonia, pyelonephritis, meningitis, tuberculosis) within 4 weeks prior to Day 1.
12. Demonstrates cognitive delay or impairment that could confound motor development in the opinion of the Investigator.
13. Treatment with any of the following therapies according to the time frames specified:
   Any time: Gene therapy, cell based therapy (e.g., stem cell transplantation), CRISPR/Cas9, or any other form of gene editing.
   Within 12 weeks of Day 1 and any time during study: Use a human growth factor.
   Within 6 months of Day 1: Any investigational medication; Any treatment designed to increase dystrophin expression (e.g., Translarna™).
14. Has received a live virus vaccine within 4 weeks or inactive vaccine within 2 weeks of the Day 1 visit or expects to receive a vaccination during the first 3 months after Day 1.
15. Has abnormal laboratory values considered clinically significant by the Investigator including but not limited to:
   Gamma-glutamyl transferase >2x upper limit of normal (ULN).
   Total bilirubin >ULN. Note; elevations in total bilirubin confirmed to be due to Gilbert's syndrome are not exclusionary.
   White blood cell count >18,500 per pl.
   Platelets <lower limit of normal.
16. Known hypersensitivity to DELANDISTROGENE MOXEPARVOVEC or its excipients or to Imlifidase or its excipients.
17. Family does not want to disclose subject's study participation with general practitioner/primary care physician and other medical providers.

### Study Drugs, Dosage, and Mode of Administration:

Imlifidase (0.25 mg/kg) IV infusion; and delandistrogene moxeparvovec (1.33×10¹⁴ vg/kg) by single IV infusion.

### Duration of Study:

The duration of each subject's participation in the study is expected to be approximately 108 weeks; a pre-infusion period of approximately 4 weeks and treatment and follow-up period of 104 Weeks. For subjects who do not receive delandistrogene moxeparvovec, the duration of participation is expected to be approximately 56 weeks; a pre-infusion period of approximately 4 weeks and an infusion and follow-up period of 52 weeks.

### Statistical Methods:

### Sample Size Determination:

The analysis of the primary endpoints in this study will be descriptive. Up to 12 subjects in total may be enrolled in order to have approximately 6 ambulatory subjects ≥4 to ≤8 years of age dosed with both Imlifidase and delandistrogene moxeparvovec.

### Statistical Analysis for the Primary Endpoints:

The statistical analysis for micro-dystrophin protein expression and transduction will be performed after the assays for micro-dystrophin protein expression at Baseline and Week 12 and the assay for micro-dystrophin transduction at Week 12 have been completed. The observed values and change from baseline values for the primary endpoints will be summarized descriptively.

### Statistical Analysis for the Secondary Endpoints:

### PK &/PD Data Analyses

The PK parameter Cmax will be estimated based on actual values. The remaining PK parameters will be estimated by non-compartmental or compartmental analysis based on measurements of the serum concentration-time data of Imlifidase, and the following parameters will be estimated, if possible (but not limited to): AUC, Tmax, t½, CL, and Vz.

The PD analyses will be descriptive based on measurements of the serum concentration-time data of intact IgG, absolute concentration of IgG (intact+single-cleaved IgG [scIgG]) as well as proportion of remaining IgG.

### Safety Analyses

Adverse events, clinical laboratory assessments, vital signs, ECGs, ECHO, physical examination findings, and weight and height will be summarized descriptively.

rAAVrh74 Antibodies and Vector Genome Analyses

Antibody titers to rAAVrh74 and vector genome copies in serum will be summarized descriptively.

### Study Design

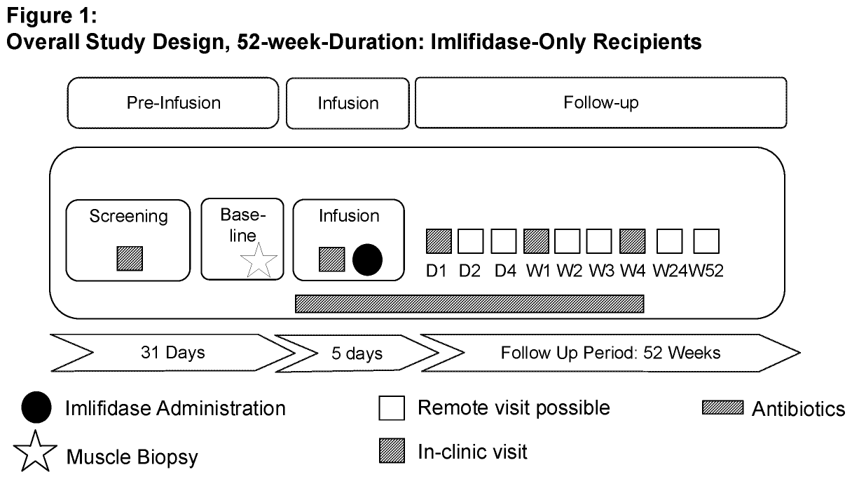

The study design for subjects that received Imlifidase only is shown in FIG. 1.

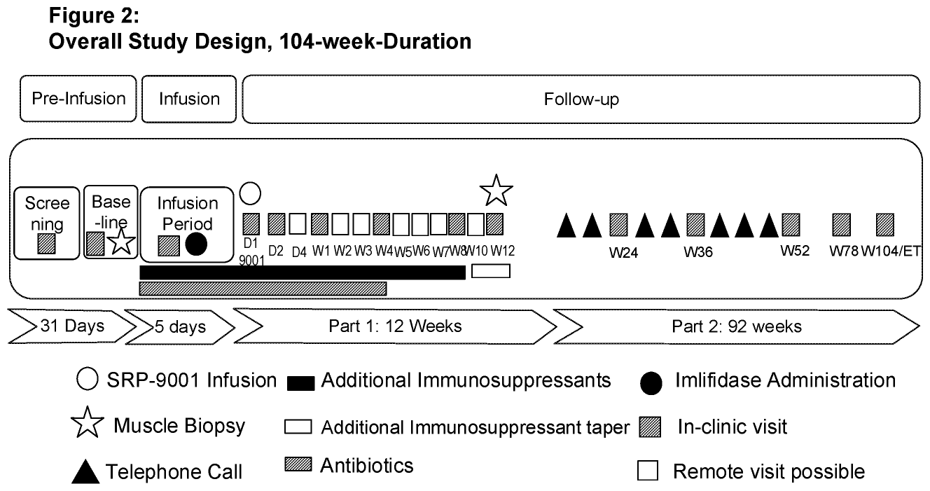

The overall study design is shown in FIG. 2.

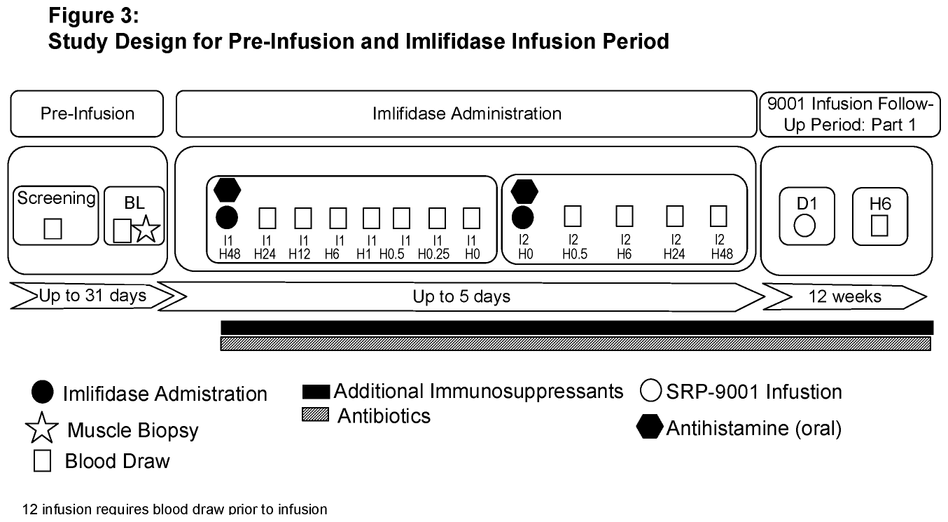

The design for the pre-infusion period and Imlifidase infusion period is shown in FIG. 3. During the pre-infusion and Imlifidase infusion periods, certain events were scheduled as shown in Table 11. The events scheduled during the follow-up period for subjects that did not receive delandistrogene moxeparvovec are shown in Table 12. The events scheduled during follow-up period for subjects that did receive delandistrogene moxeparvovec are shown in Table 13 (part 1) and Table 14 (part 2).

### Screening/Baseline Assessments

### Demographic and Medical History

Demographic information (e.g., age, gender at birth, race, ethnicity, body weight, height, body mass index) and medical history will be obtained for all subjects.

### Genetic Diagnostics

Subjects must have a definitive diagnosis of DMD prior to Screening based on documentation of clinical findings and prior confirmatory genetic testing using a clinical diagnostic genetic test. Genetic report must describe a frameshift deletion, frameshift duplication, premature stop, or other pathogenic variant in the DMD gene fully contained between exons 18 to 79 (inclusive) that is expected to lead to absence of dystrophin protein.

Mutations between or including exons 1-17 are not eligible.

In-frame deletions, in-frame duplications, and variants of uncertain significance (“VUS”) are not eligible.

Mutations that are fully contained within exon 45 (inclusive) are not eligible.

### Efficacy Assessments

### Micro-Dystrophin Expression

A muscle biopsy for evaluation of micro-dystrophin expression will be collected from all subjects at Baseline and Week 12 as specified in Tables 11 and 13. Muscle biopsies should be collected using open biopsy or a VACORA core biopsy with Sponsor approval. The biopsy requires collection of muscle tissue from the medial gastrocnemius muscle. If this muscle is not viable, prior approval from the Sponsor is required for using an alternate muscle.

The biopsy sample will be used to quantify transgene expression by Sarepta's western blot adjusted for muscle content, IF fiber intensity, and PDPF. Refer to the Surgical and Laboratory Biopsy Manual for additional details on the handling and processing of biopsy tissues.

### Functional Assessments

Every effort must be made to have each subject assessed by the same clinical evaluator for physical functional assessments (NSAA and timed-function tests) throughout the entire study. Functional assessments should be scheduled in the morning and should be the first assessments done during a specific visit.

Functional assessments will be administered at the time points indicated in Tables 11, 13, and 14.

### North Star Ambulatory Assessment Scale

The NSAA is a clinician-administered scale that rates performance on various functional activities (Mazzone E, et al., Neuromuscul Disord. 20(11):712-6 (2010), incorporated by reference herein in its entirety). It was designed to be used in boys with DMD who are able to stand, and it has been used in DMD boys of this study's age range (≥3 and <8 years, and ≥8 years of age) (Connolly et al. Neuromuscul Disord. 2013; 23(7):529-39); Mrecuri E, et al., PLoS One. 016; 11(8):e0160195; Muntoni F, et al., PLoS One. 2019 Sep. 3; 14(9):e0221097, incorporated by reference herein in its entirety).

During this assessment, subjects perform 17 different functional activities, including the 10MWR, rising from a sit to a stand, standing on 1 leg, climbing a box step, descending a box step, rising from lying to sitting, rising from the floor, lifting head off floor, standing on heels, and jumping.

Subjects will be graded as follows: 2=normal, no obvious modification of activity; 1=modified method but achieves goal independent of physical assistance from another; and 0=unable to achieve goal independently.

Time to Rise from the Floor

The time to rise from the floor test is part of the NSAA (item 11) and quantifies the time required for the subject to stand in an upright position with arms by sides, starting from the supine position with arms by sides (Henricson 2013, incorporated by reference herein in its entirety). The time required for the subject to complete the task will be recorded during the NSAA administration.

### 10-Meter Walk/Run

The timed 10MWR is part of the NSAA (item 17) and quantifies the time required for the subject to run or walk 10 meters (on a straight walkway) from a standing position (Mc Donald C M, et al., Muscle Nerve. 2013 September; 48(3):357-68, incorporated by reference herein in its entirety). The subject is encouraged to run past the 10-meter mark. The time required for the subject to cover the distance will be recorded during the NSAA administration.

### Time to Ascend 4 Steps

The timed 4-step test quantifies the time required for the subject to ascend 4 standard steps (each step 6 inches in height) (Bushby K, et al., Clin Investig (Lond). 2011; 1(9):1217-35, incorporated by reference herein in its entirety) The time required for the subject to climb up 4 standard-sized steps will be recorded.

### 100-Meter Walk/Run

The 100MWR quantifies the time required for the subject to run or walk 100 meters (on a straight walkway) from a standing position (Alfano L N, et al., Neuromuscul Disord. 2017; 27(5):452-7, incorporated by reference herein in its entirety). The subject is encouraged to run past the 100-meter mark. The time required for the subject to cover the distance will be recorded.

### Vector Genome and Quantification

Vector quantification in serum will be performed using polymerase chain reaction in all subjects at study visits as indicated in Tables 11, 13, and 14. On Day 2, the sample should be collected approximately 22 to 26 hours following the end of the delandistrogene moxeparvovec infusion.

Vector genome copies will be measured in muscle biopsy samples at Baseline and Week 12 using polymerase chain reaction.

### Creatine Kinase

Creatine kinase levels following delandistrogene moxeparvovec infusion will serve as an exploratory efficacy measure.

### Safety Assessments

### Physical Examination

A full or brief (symptom-directed) physical examination will be performed by the Investigator or qualified study staff at the time points specified in Tables 11-14.

A full physical examination will include examination of general appearance, head, ears, eyes, nose, throat (HEENT), heart, chest (respiratory), abdomen (gastrointestinal), skin, lymph nodes, extremities, and the musculoskeletal and neurological systems.

A brief physical examination will include general appearance; HEENT; heart; chest; abdomen; and skin evaluation.

### Electrocardiograms and Echocardiograms

A 12-lead ECG will be obtained in triplicate at the time points specified Tables 11, 13, and 14 using an ECG machine that automatically calculates the heart rate and measures PR, QRS, and QT intervals. All ECGs should be performed before any invasive procedures (eg, blood sampling, study drug infusion, or biopsy). All ECGs should be performed only after the subject has been resting, and quiet for approximately 5 minutes. On Day 1, triplicate ECGs will be taken both before and following the end of the delandistrogene moxeparvovec infusion. The Investigator (or a designee) will review the ECG results and determine if the findings are clinically significant. The Day 1 pre-infusion review should be completed and documented prior to delandistrogene moxeparvovec infusion.

A standard 2-dimensional ECHO will be obtained at the time points specified in Tables 11 and 14. Echocardiograms will be performed before any invasive procedures (eg, blood sampling, study drug infusion, or biopsy). The ECHO will be reviewed and interpreted by local medically qualified personnel. Left ventricular ejection fraction will be noted.

TABLE 11
Schedule of Events Pre-infusion and Imlifidase Infusion Periods

| | Infusion Period | | | | | | | | | | | | | | |
|---|---|---|---|---|---|---|---|---|---|---|---|---|---|---|---|
| | Pre- | | | | | | | | | | | | | | |
| | Infusion | | Infusion | | | | | | | | | | | | |
| | Trial Period | | | | | | | | | | | | | | |
| | SCR | BL | Imlifidase dose #1 | | | | | | | | Imlifidase dose #2 ^{a} | | | | |
| | Visit Name: | | | | | | | | | | | | | | |
| | | | I1 | I1 | I1 | I1 | I1 | I1 | I1 | I1 | I2 | I2 | I2 | I2 | I2 |
| | SCR | BL | H 0 | 15 M | 30 M | H 1 | H 6 | H 12 | H 24 | H 48 | H 0 | 30 M | H 6 | H 24 | H 48 |
| | Visit Window (Days) | | | | | | | | | | | | | | |
| | | | | ±3 | ±5 | ±10 | ±30 | ±60 | ±60 | ±60 | | ±5 | ±30 | ±60 | ±60 |
| | NA | NA | NA | min | min | min | min | min | min | min | NA | min | min | min | min |
| | Visit Type ^{b} | | | | | | | | | | | | | | |
| | C | C | C | C | C | C | C | C | C | C | C | C | C | C | C |
| Physical Exam ^{c} | X | X | | | | | | | | | | | | | |
| Vital signs ^{d} | X | X | X | X | X | X | X | | | | X | X | X | | |
| Height | | X | | | | | | | | | | | | | |
| Weight | X | | X^{e} | | | | | | | | X^{e} | | | | |
| NSAA (including | | X | | | | | | | | | | | | | |
| time to rise from | | | | | | | | | | | | | | | |
| the floor and | | | | | | | | | | | | | | | |
| 10MWR) | | | | | | | | | | | | | | | |
| Timed 4-step | | X | | | | | | | | | | | | | |
| test & 100MWR | | | | | | | | | | | | | | | |
| anti-AAVrh74 | X | | | | | | X | X | X | X | | X | X | X | X |
| antibodies ^{f} | | | | | | | | | | | | | | | |
| Total IgG | X | | | | | | X | X | X | X | | X | X | X | X |
| Immunology ^{g} | X | | | | | | | | | | | | | | |
| Anti-Imlifidase | | X | | | | | | | | X | | | | | X |
| IgG antibodies | | | | | | | | | | | | | | | |
| (ADA) | | | | | | | | | | | | | | | |
| PK/PD sampling | | X | | X | X | X | X | | X | X | X ^{h} | X | X | X | X |
| Hematology ^{j} | X | | | | | | | | | | | | | | |
| Hepatitis B&C | X | | | | | | | | | | | | | | |
| Serology, HIV | | | | | | | | | | | | | | | |
| Electrolytes ^{j} | | X | | | | | | | | | | | | | |
| Troponin I | | X | | | | | | | | X | | | | | X |
| CK | X | | | | | | | | | X | | | | | X |
| Liver function ^{j} | X | | | | | | | | | X | | | | | X |
| Renal function ^{j} | X | | | | | | | | | | | | | | |
| Urinalysis ^{j} | X | | | | | | | | | | | | | | |
| Complement (C3, | | X | | | | | | | | X | | | | | X |
| C4) | | | | | | | | | | | | | | | |
| Vector | X | | | | | | | | | | | | | | |
| quantification | | | | | | | | | | | | | | | |
| RNA sequence | X | | | | | | | | | | | | | | |
| Muscle Biopsy ^{k, l} | | X | | | | | | | | | | | | | |
| ECG ^{m} | X | | | | | | | | | | | | | | |
| ECHO | X | | | | | | | | | | | | | | |
| Imlifidase ^{m, n} | | | X | | | | | | | | X | | | | |
| Antihistamine | | | X | | | | | | | | X | | | | |
| Antibiotic ^{o} | | | 4-week course from first dose of Imlifidase | | | | | | | | | | | | |
| Add-on | | | Starting at least 1 day prior to delandistrogene moxeparvovec | | | | | | | | | | | | |
| corticosteroid | | | | | | | | | | | | | | | |
| (1 mg/kg) | | | | | | | | | | | | | | | |
| Adverse Event | <---Continuous (ongoing collection beginning at informed consent/assent)-→ | | | | | | | | | | | | | | |
| Reporting | | | | | | | | | | | | | | | |
| Concomitant | <---Continuous (ongoing collection beginning at informed consent/assent)-→ | | | | | | | | | | | | | | |
| Medications and | | | | | | | | | | | | | | | |
| Procedures | | | | | | | | | | | | | | | |

10MWR = 10-meter walk run; 100MWR = 100-meter walk run test; AE = adverse event; B = baseline; C = clinic; CK = creatine kinase; D = day; DNA = deoxyribonucleic acid; ET = early termination; H = hour; HEENT = head, ears, eyes, nose, and throat; HIV = human immunodeficiency virus;; NA = not applicable; NSAA = North Star Ambulatory Assessment; PD = Pharmacodynamics; PE = physical examination; PK = Pharmacokinetics; Scr = screening; R = remote; RN = ribonucleic acid; T = telephone; W = week.
^{a} Imlifidase Dose #2 and the associated blood draws may not occur depending on patient Anti-AAVrh74 antibody results after dose #1. If administered, Imlifidase dose #2 should be administered by approximately 60 hours after completion of the first Imlifidase dose.
^{b} Visits indicated as “R” are visits that can be conducted at the clinic or remotely. “T” visits indicate telephone follow-up
^{c} A full physical examination will be performed at screening Week 52, and Week 104/ET and include: general appearance; HEENT; cardiovascular; dermatologic; abdominal; genito-urinary; lymph nodes; hepatic; musculoskeletal; respiratory; and neurological. A brief physical examination will be performed at all other visits indicated and include general appearance; HEENT; heart; chest; abdomen; and skin.
^{d} Vital signs to be collected include pulse rate, respiratory rate, blood pressure, and temperature (oral, tympanic, or axillary). On Imlifidase and delandistrogene moxeparvovec infusion days, vital signs will be measured at the timepoints indicated.
^{e}Weight may be obtained the day of or day prior to infusion.
^{f} Antibodies to rAAVrh74 can be detected by a neutralizing anti-AAVrh74 cell-based assay and/or quantified by a total anti-AAVrh74 antibody enzyme-linked immunosorbent assay (ELISA). Samples obtained prior to delandistrogene moxeparvovec infusion may undergo analysis by one or both methods depending on whether titer quantification is needed. delandistrogene moxeparvovec dosing decisions will be done using a total anti-AAVrh74 antibody ELISA assay. Samples collected after delandistrogene moxeparvovec infusion will be analyzed using ELISA only.
^{g} Immunology studies include ELISpot and antibodies to microdystrophin. Immunology studies are described in Section 5.4.4..
^{h} Blood draw occurs prior to infusion.
i. List of specific analytes as described herein.
^{j} List of specific analytes assessing liver function and renal function as described herein.
^{k} List of analytes specific to urinalysis as described herein.
^{l} Baseline and Week 12 functional assessments must be performed prior to the biopsy procedure. The baseline biopsy will be of the medial gastrocnemius muscle, preferably on the right leg. If the medial gastrocnemius muscle is not viable, prior approval from the Sponsor is required for using an alternate muscle of the lower extremity. If possible, the biopsy for Week 12 will be of the same muscle group as that used at Baseline on the contralateral side, preferably on the left leg. Refer to the Surgical and Laboratory Biopsy Manual.
^{m} All ECGs should be performed in triplicate at a consistent time of day throughout the study and before any invasive procedures (eg, blood sampling, study drug infusion, or biopsy). On Day 1, triplicate ECGs will also be taken following the end of infusion.
ⁿ Imlifidase will be administered by intravenous infusion (approximately 15 minutes). Subjects are to be closely monitored following completion of the infusion. A topical anesthetic cream (eg, lidocaine 2.5%, prilocaine 2.5%, LMX4 cream) may be applied prior to infusions per site and subject preference.
^{o} Antibiotic selection/dosing/schedule as described herein.

TABLE 12
Schedule of events: Follow-up period for subjects
who do not receive delandistrogene moxeparvovec

| Infusion Period | | | | | | | | |
|---|---|---|---|---|---|---|---|---|
| Post-Infusion | | | | | | | | |
| Trial Period | | | | | | | | |
| Follow-up Period | | | | | | | | |
| Visit Name: | | | | | | | | |
| D 1 | D 2 | D 4 | W 1 | W 2 | W 3 | W 4 | W 24 | W 52 |
| Visit Window (Days) | | | | | | | | |
| | | | | | | | +/−21 | +/21 |
| NA | NA | +/−1 | +/−1 | +/−1 | +/−1 | +/−3 | days | days |
| Visit Type ^{a} | | | | | | | | |
| C | R | R | C | R | R | C | R | R |

| | | | | | | | |
|---|---|---|---|---|---|---|---|
| Physical Exam ^{b} | | | | X | X | | |
| Vital signs ^{c} | | | | X | X | | |
| anti-AAVrh74 antibodies ^{d} | X | X | X | X | X | X | X |
| Anti-Imlifidase IgG | | X | X | X | X | X | X |
| antibodies (ADA) | | | | | | | |
| Total IgG | X | X | X | X | X | X | X |
| PK/PD sampling | X | X | X | X | X | | |
| Hematology ^{e} | | | | X | | | |
| Electrolytes | | | | X | | | |
| Troponin I | | | | X | | | |
| CK | | | | X | | | X |
| Liver function ^{f} | | | | X | | | |
| Complement (C3, C4) | | X | | X | | | |
| DMD gene sequence analysis | | | | | X | | |

| | |
|---|---|
| Antibiotic ^{g} | 4-week course from first dose of Imlifidase |

| | |
|---|---|
| Adverse Event Reporting | ←---Continuous (ongoing collection beginning |
| | at informed consent/assent)---→ |
| Concomitant Medications | ←---Continuous (ongoing collection beginning |
| and Procedures | at informed consent/assent)---→ |

10MWR = 10-meter walk run; 100MWR = 100-meter walk run test; AE = adverse event; B = baseline; C = clinic; CK = creatine kinase; D = day; DNA = deoxyribonucleic acid; ET = early termination; H = hour; HEENT = head, ears, eyes, nose, and throat; HIV = human immunodeficiency virus; NA = not applicable; NSAA = North Star Ambulatory Assessment; PD = Pharmacodynamics; PE = physical examination; PK = Pharmacokinetics; Scr = screening; R = remote; RN = ribonucleic acid; T = telephone; W = week.
^{a} Visits indicated as “R” are visits that can be conducted at the clinic or remotely. “T” visits indicate telephone follow-up
^{b} A full physical examination will be performed at screening Week 52, and Week 104/ET and include: general appearance; HEENT; cardiovascular; dermatologic; abdominal; genito-urinary; lymph nodes; hepatic; musculoskeletal; respiratory; and neurological. A brief physical examination will be performed at all other visits indicated and include general appearance; HEENT; heart; chest; abdomen; and skin.
^{c} Vital signs to be collected include pulse rate, respiratory rate, blood pressure, and temperature (oral, tympanic, or axillary). On Imlifidase and delandistrogene moxeparvovec infusion days, vital signs will be measured at the timepoints indicated.
^{d} Antibodies to rAAVrh74, measured by ELISA.
^{e} List of specific analytes as described herein.
^{f} List of specific analytes assessing liver function and renal function as described herein.
^{g} Antibiotic selection/dosing/schedule as described herein.

TABLE 13
Schedule of Events: Follow-Up Period for subjects who receive delandistrogene moxeparvovec,
Part 1

| | Infusion Period | | | | | | | | | | | | | |
|---|---|---|---|---|---|---|---|---|---|---|---|---|---|---|
| | Post-Infusion | | | | | | | | | | | | | |
| | Trial Period | | | | | | | | | | | | | |
| | Follow-Up Period: Part 1 | | | | | | | | | | | | | |
| | Visit Name: | | | | | | | | | | | | | |
| | D 1 | H 6 | D 2 | D 4 | W 1 | W 2 | W 3 | W 4 | W 5 | W 6 | W 7 | W 8 | W 10 | W 12 |
| | Visit Window (Days) | | | | | | | | | | | | | |
| | NA | ±30 min | NA | +/−1 | +/−1 | +/−1 | +/−1 | +/−3 | +/−3 | +/−3 | +/−3 | +/−3 | +/−3 | +/−7 |
| | Visit Type ^{a} | | | | | | | | | | | | | |
| | C | C | C | R | C | R | R | C | R | R | R | C | R | C |
| Physical Exam ^{b} | X | | X | | X | | | X | | | | X | | X |
| Vital signs ^{c} | X | | X | | X | | | X | | | | X | | X ^{d} |
| Height | | | | | | | | | | | | | | X ^{d} |
| Weight | X ^{e} | | | | X | | | X | | | | X | | X |
| NSAA (including time to | | | | | | | | | | | | | | X |
| rise from the floor and | | | | | | | | | | | | | | |
| 10MWR) | | | | | | | | | | | | | | |
| Timed 4-step test & | | | | | | | | | | | | | | X |
| 100MWR | | | | | | | | | | | | | | |
| anti-AAVrh74 antibodies ^{f} | | X | X | X | X | | | X | | | | X | | X |
| Immunology ^{g} | | X | | | | | | X | | | | X | | X |
| Anti-Imlifidase IgG | | | X | X | X | | | X | | | | | | X |
| antibodies (ADA) | | | | | | | | | | | | | | |
| Total IgG | | X | X | X | X | | | X | | | | X | | X |
| PK/PD sampling | | | X | X | X | | | X | | | | | | X |
| Hematology ^{h} | | | | | X | X | X | X | | X | | X | X | X |
| Electrolytes ^{h} | | | X | X | | | | | | | | | | X |
| Troponin I | | | X | X | X | X | | X | | | | X | | X |
| CK | | X | X | X | X | | | X | | X | | X | X | X |
| Liver function ⁱ | | | | | X | | | X | X | X | X | X | X | X |
| Renal function ⁱ | | | | | X | | | | | | | | | X |
| Complement (C3, C4) | | | X | | X | X | | X | | | | X | | X |
| Vector quantification | | X | X ^{j} | X | X | | | | | | | | | |
| DMD gene sequence | | | | | | | | | | | | X | | |
| analysis | | | | | | | | | | | | | | |
| Whole-genome DNA | | | | | | | | | | | | X | | |
| sequence ^{k} | | | | | | | | | | | | | | |
| RNA sequence | | | | | | | | X | | | | X | | X |
| Urinalysis ^{l} | | X | X | | X | | | X | | | | X | | X |
| Muscle Biopsy ^{m} | | | | | | | | | | | | | | X |
| ECG ⁿ | | | X | X | X | X | | | | | | | | X |
| delandistrogene | X | | | | | | | | | | | | | |
| moxeparvovec ^{d, o} | | | | | | | | | | | | | | |
| Add-on corticosteroid | X | | X | X | X | X | X | X | X | X | X | X | | |
| (1 mg/kg) | | | | | | | | | | | | | | |
| Add-on corticosteroid | | | | | | | | | | | | | X | X |
| Tapering | | | | | | | | | | | | | | |

| | |
|---|---|
| Antibiotic ^{p} | 4-week-course starting with first day of Imlifidase |

| | |
|---|---|
| Adverse Event | ← ---Continuous (ongoing collection beginning |
| Reporting | at informed consent/assent)--- → |
| Concomitant Medications | ← ---Continuous (ongoing collection beginning |
| and Procedures | at informed consent/assent)--- → |

10MWR = 10-meter walk run; 100MWR = 100-meter walk run test; AE = adverse event; B = baseline; C = clinic; CK = creatine kinase; D = day; DNA = deoxyribonucleic acid; ET = early termination; H = hour; HEENT = head, ears, eyes, nose, and throat; HIV = human immunodeficiency virus;; NA = not applicable; NSAA = North Star Ambulatory Assessment; PD = Pharmacodynamics; PE = physical examination; PK = Pharmacokinetics; Scr = screening; R = remote; RN = ribonucleic acid; T = telephone; W = week.
^{a} Visits indicated as “R” are visits that can be conducted at the clinic or remotely. “T” visits indicate telephone follow-up
^{b} A full physical examination will be performed at screening Week 52, and Week 104/ET and include: general appearance; HEENT; cardiovascular; dermatologic; abdominal; genito-urinary; lymph nodes; hepatic; musculoskeletal; respiratory; and neurological. A brief physical examination will be performed at all other visits indicated and include general appearance; HEENT; heart; chest; abdomen; and skin.
^{c} Vital signs to be collected include pulse rate, respiratory rate, blood pressure, and temperature (oral, tympanic, or axillary). On Imlifidase and delandistrogene moxeparvovec infusion days, vital signs will be measured at the timepoints indicated.
^{d} delandistrogene moxeparvovec will be administered by intravenous infusion (approximately 1-2 hours). Subjects are to be closely monitored for at least 6 hours following completion of the infusion. A topical anesthetic cream (eg, lidocaine 2.5%, prilocaine 2.5%, LMX4 cream) may be applied prior to infusions per site and subject preference.
^{e} Weight may be obtained the day of or day prior to infusion.
^{f} Antibodies to rAAVrh74 capsid as measured by ELISA.
^{g} Immunology studies include enzyme-linked immunospot (ELISpot) and antibodies to microdystrophin.
^{h} List of specific analytes as described herein.
ⁱ List of specific analytes assessing liver function and renal function as described herein.
^{j} Sample to be taken approximately 22 to 26 hours post delandistrogene moxeparvovec infusion.
^{k} Blood sample for whole-genome sequencing is optional based upon local regulations and Institutional Review Board/Ethics. Committee approval. An additional informed consent/assent form must be signed prior to collection of samples.
^{l} List of analytes specific to urinalysis as described herein.
^{m} Baseline and Week 12 functional assessments must be performed prior to the biopsy procedure. The baseline biopsy will be of the medial gastrocnemius muscle, preferably on the right leg. If the medial gastrocnemius muscle is not viable, prior approval from the Sponsor is required for using an alternate muscle of the lower extremity. If possible, the biopsy for Week 12 will be of the same muscle group as that used at Baseline on the contralateral side, preferably on the left leg. Refer to the Surgical and Laboratory Biopsy Manual.
ⁿ All ECGs should be performed in triplicate at a consistent time of day throughout the study and before any invasive procedures (eg, blood sampling, study drug infusion, or biopsy). On Day 1, triplicate ECGs will also be taken following the end of infusion.
^{o} delandistrogene moxeparvovec infusion must begin no later than 54 hours after the most recent Imlifidase dose completes.
^{p} Antibiotic selection/dosing/schedule as described herein.

TABLE 14
Schedule of Events: Follow-Up Period for subjects
who receive delandistrogene moxeparvovec, Part 2

| Infusion Period | | | | | |
|---|---|---|---|---|---|
| Post-Infusion | | | | | |
| Trial Period | | | | | |
| Follow-Up Period: Part 2 | | | | | |
| Visit Name: | | | | | |
| W 16, 20, 28, | | | | | W 104/ |
| 32, 40, 44, 48 | W 24 | W 36 | W 52 | W 78 | ET ^{a} |
| Visit Window (Days) | | | | | |
| +/−7 | +/−7 | +/−7 | +/−7 | +/−21 | +/−21 |
| Visit Type ^{b} | | | | | |
| T | C | C | C | C | C |

| | | | | | |
|---|---|---|---|---|---|
| Physical Exam ^{c} | X | X | X | X | X |
| Vital signs ^{d} | X | X | X | X | X |
| Height | X | | X | | X |
| Weight | X | X | X | X | X |
| NSAA (including time to rise | X | X | X | X | X |
| from the floor and 10MWR) | | | | | |
| Timed 4-step test | X | X | X | X | X |
| 100MWR | X | X | X | X | X |
| anti-AAVrh74 antibodies ^{e} | X | | X | | X |
| Immunology ^{f} | X | | X | | X |
| Anti-Imlifidase IgG | X | | X | | X |
| antibodies (ADA) | | | | | |
| Total IgG | X | | X | | X |
| Hematology ^{g} | X | X | X | X | X |
| Electrolytes ^{g} | X | | X | | X |
| Troponin I | X | X | X | X | X |
| CK | X | X | X | X | X |
| Liver function ^{h} | X | X | X | X | X |
| Renal function ^{h} | X | | X | | X |
| Complement (C3, C4) | X | | X | | X |
| RNA sequence | X | X | X | | X |
| ECG ^{j} | | | X | | X |
| ECHO | | | X | | X |

| | |
|---|---|
| Adverse Event Reporting | <---Continuous (ongoing collection beginning |
| | at informed consent/assent)---> |
| Concomitant Medications | <---Continuous (ongoing collection beginning |
| and Procedures | at informed consent/assent)---> |

10MWR = 10-meter walk run; 100MWR = 100-meter walk run test; AE = adverse event; B = baseline; C = clinic; CK = creatine kinase; D = day; DNA = deoxyribonucleic acid; ET = early termination; H = hour; HEENT = head, ears, eyes, nose, and throat; HIV = human immunodeficiency virus;; NA = not applicable; NSAA = North Star Ambulatory Assessment; PD = Pharmacodynamics; PE = physical examination; PK = Pharmacokinetics; Scr = screening; R = remote; RN = ribonucleic acid; T = telephone; W = week.
^{a} In case of subject withdrawal, Week 104 assessments should be performed at the ET visit.
^{b} Visits indicated as “R” are visits that can be conducted at the clinic or remotely. “T” visits indicate telephone follow-up
^{c} A full physical examination will be performed at screening Week 52, and Week 104/ET and include: general appearance; HEENT; cardiovascular; dermatologic; abdominal; genito-urinary; lymph nodes; hepatic; musculoskeletal; respiratory; and neurological. A brief physical examination will be performed at all other visits indicated and include general appearance; HEENT; heart; chest; abdomen; and skin.
^{d} Vital signs to be collected include pulse rate, respiratory rate, blood pressure, and temperature (oral, tympanic, or axillary). On Imlifidase and delandistrogene moxeparvovec infusion days, vital signs will be measured at the time points indicated.
^{e} Antibodies to rAAVrh74 capsid as measured by a total anti-AAVrh74 enzyme-linked immunosorbent assay (ELISA)
^{f} Immunology studies include ELISpot and antibodies to microdystrophin.
^{g} List of specific analytes as described herein.
^{h} List of specific analytes assessing liver function and renal function as described herein.
i. All ECGs should be performed in triplicate at a consistent time of day throughout the study and before any invasive procedures (eg, blood sampling, study drug infusion, or biopsy). On Day 1, triplicate ECGs will also be taken following the end of infusion.

TABLE 15
List of Abbreviations and Definitions of Terms

| Abbreviation | Definition |
|---|---|
| 2-D | 2-dimensional |
| ADA | Anti-drug antibodies |
| ADCC | Antibody-dependent cell-mediated cytotoxicity |
| ADCP | Antibody-dependent cellular phagocytosis |
| ADR | Adverse drug reaction |
| AE | Adverse event |
| AESI | Adverse events of special interest |
| ALT | Alanine aminotransferase |
| AST | Aspartate aminotransferase |
| AUC | Area under the concentration-time curve |
| CDC | Complement-dependent cytotoxicity |
| CFR | Code of Federal Regulations |
| CK | Creatine kinase |
| Cₘₐₓ | Maximum concentration observed |
| CMV | Cytomegalovirus |
| CRISPR | Clustered regularly interspaced short palindromic repeats |
| CRO | Contract research organization |
| CTCAE | Common terminology criteria for adverse events |
| DMC | Data monitoring committee |
| DMD | Duchenne muscular dystrophy |
| DNA | Deoxyribonucleic acid |
| EBV | Epstein-Barr virus |
| ECG | Electrocardiogram |
| ECHO | Echocardiogram |
| eCRF | Electronic case report form |
| ELISA | Enzyme-linked immunosorbent assay |
| ELISPOT | Enzyme-linked immunospot |
| ET | Early termination |
| EUDRACT | European Union Drug Regulating Authorities |
| | Clinical Trials Database |
| FDA | Food and Drug Administration |
| GCP | Good clinical practice |
| GGT | Gamma-glutamyl transferase |
| GLDH | Glutamate dehydrogenase |
| HEENT | Head, ears, eyes, nose, throat |
| HH6 | Human herpesvirus 6 |
| hsCRP | High-sensitivity C-reactive protein |
| IB | Investigator's Brochure |
| ICH | International Council for Harmonisation |
| IEC | Independent Ethics Committee |
| IF | Immunofluorescence |
| IgG | Immunoglobulin G |
| IgM | Immunoglobulin M |
| IMP | Investigational medicinal product |
| Injection | US nomenclature equivalent to Concentrate |
| | for Solution for Infusion |
| INR | International normalized ratio |
| IRB | Institutional review board |
| MRI | Magnetic resonance imagining |
| NOAEL | No-observed-adverse-effect level |
| NSAA | North Star Ambulatory Assessment |
| PD | Pharmacodynamics |
| PDPF | Percent dystrophin-positive fibers |
| PK | Pharmacokinetic(s) |
| PT | Preferred term |
| rAAVrh74 | Recombinant adeno-associated virus rhesus serotype 74 |
| RNA | Ribonucleic acid |
| SAE | Serious adverse event |
| SAP | Statistical Analysis Plan |
| SRC | Safety Review Committee |
| SUSAR | Suspected unexpected serious adverse reaction |
| t_{1/2} | Elimination half-life |
| TEAE | Treatment-emergent adverse event |
| TMA | Thrombotic microangiopathy |
| tₘₐₓ | Time of maximum observed concentration (Cₘₐₓ) |
| TPP | Thrombocytopenia purpura |
| ULN | Upper limit of normal |
| US | United States |
| VUS | Variants of uncertain significance |
| V_{z} | Volume of distribution associated with the terminal phase |

### Example 3: Design of Study in Example 2

### Overall Study Design

This is an open-label study of male subjects with DMD and pre-existing antibodies to rAAVrh74 who are ≥4 to ≤8 years of age. Approximately 6 subjects will be treated with Imlifidase followed by delandistrogene moxeparvovec.

To ensure that at least 6 subjects receive both therapies, additional subjects (up to a maximum of 6) may be enrolled to account for subjects who receive Imlifidase but do not meet criteria for delandistrogene moxeparvovec dosing.

The first three subjects who are dosed with both Imlifidase and delandistrogene moxeparvovec are sentinel subjects and will enter the Infusion period at least one week apart. The safety, PK/PD, and expression data of the three sentinel subjects will be reviewed by an SRC prior to additional subjects entering the infusion period.

The total duration of participation for subjects who receive both Imlifidase and delandistrogene moxeparvovec is expected to be approximately 108 weeks, inclusive of an approximately 4 week pre-infusion period, and up to approximately 5-day Imlifidase infusion period, and a 104-week delandistrogene moxeparvovec treatment and follow-up period. The total duration of participation for subjects who do not receive delandistrogene moxeparvovec is expected to be approximately 56 weeks, inclusive of an approximately 4-week pre-infusion period and up to approximately 5-day Imlifidase infusion period, and a 52-week follow-up period.

Schematics of the study design are provided in FIGS. 1-3. The schedules of events are provided in Tables 11-14.

### Pre-Infusion Period

Prior to undergoing any study procedures, subjects will provide informed consent/assent and parent(s), or legal guardian(s) will provide written informed consent (for subjects <18 years of age) for the subject to participate in the study.

### Screening Period

During the up to approximately three-week Screening Period, various assessments to confirm eligibility will be performed, including collection of the subject's medical history, demographics, documentation of DMD genotyping, vital signs, physical examination, and a blood draw for enzyme-linked immunosorbent assay (ELISA) and select clinical laboratory assessments.

During the Screening Period, additional assessments include ECG, ECHO, a ribonucleic acid (RNA) sample, ELISpot sample, and a review of prior and concomitant medications. Refer to the Schedule of Events in Tables 11-14 for a complete outline of assessments performed during the Screening Period.

All laboratory samples obtained will be stored for future research on DMD if permitted by the informed consent/assent form regardless of Screening outcome and maintained for up to 15 years following the end of the study or per local regulations.

### Baseline Period

The Baseline Period will start when eligibility is confirmed and end on the day prior to the first Imlifidase infusion. During this period, vital signs, select clinical laboratory assessments, and a brief physical examination will be performed.

Physical functional assessments to be performed include the NSAA (including the timed function tests of time to rise from the floor and the 10MWR, although the timed components are not part of the NSAA), time to ascend 4 steps, and the 100MWR. Functional assessment data collected at Baseline visits will be used for the efficacy endpoint analysis as described in Section 5.2 and detailed in the statistical analysis plan (SAP).

All subjects will have a muscle biopsy performed after all other pre-infusion assessments have been completed and eligibility has been confirmed. The Baseline biopsy will be of the medial gastrocnemius muscle, preferably on the right leg. If the medial gastrocnemius muscle is not viable, prior approval from the Sponsor is required for using an alternate muscle of the lower extremity. If possible, the biopsy at Week 12 will be of the same muscle group as that used at Baseline on the contralateral side. For subjects that may have had a pre-infusion biopsy prior to study entry, this may be used as the “baseline” biopsy. The outline of assessments performed during the Baseline Period is shown in Table 11.

### Imlifdase Infusion Period

The Imlifidase infusion period consists of up to two doses of Imlifidase administered up to 60 hours apart. All enrolled subjects will receive one dose of Imlifidase. Approximately six hours later a blood sample will be drawn to test for antibodies to rAAVrh74. If the rAAVrh74 antibody titer is ≤1:400 and no more than 48 hours have passed since the completion of the Imlifidase infusion, the subject will enter the 260-week delandistrogene moxeparvovec treatment and follow-up period at approximately 48 hours post-Imlifidase infusion. If the antibody titer result is received after 48 hours or are >1:400, the subject will receive a second infusion of Imlifidase approximately 60 hours after the first infusion. Antibody titers will be drawn approximately 6 hours after the second dose of Imlifidase; if the antibody titer result is received within 48 hours and is ≤1:400, the subject will enter the delandistrogene moxeparvovec treatment and follow-up period at approximately 48 hours post-Imlifidase infusion. If the antibody titer result is received after 48 hours or are >1:400, the subject will not receive delandistrogene moxeparvovec and will enter the 52-week follow-up period at 48 hours post second Imlifidase infusion.

On the day of the first Imlifidase infusion, subjects will start a 4-week course of prophylactic antibiotics to mitigate the potential increased risk of infection from temporary IgG depletion. Since respiratory tract infections are the most common infections in patients with hypogammaglobulinemia, the selected antibiotic should have coverage upper respiratory pathogens, for example amoxicillin 10 mg/kg twice daily (up to a maximum dose of 500 mg twice daily). An alternative agent with appropriate coverage (eg cephalosporin, macrolide) may be used at the Investigator's discretion and may be based on regional or institutional resistance patterns and guidelines.

To reduce the risk of infusion reactions, an antihistamine (for example, diphenhydramine 1.25 mg/kg orally up to a maximum of 50 mg) will be administered up to approximately one hour prior to each Imlifidase infusion. The choice and dose of antihistamine is at the discretion of the Investigator and may be based on local or institutional guidelines.

Each Imlifidase infusion is administered as an intravenous infusion of 0.25 mg/kg over 15 minutes using an infusion pump. Refer to the Pharmacy Manual for additional information. The body weight recorded on the day prior to (or the day of) infusion should be used for the dose calculation.

During the Imlifidase infusion period subjects will undergo select laboratory studies to monitor for safety and evaluate the PK/PD of Imlifidase as well as the effect of Imlifidase on rAAVrh74 titers. The outline of assessments performed during the Imlifidase infusion period is shown in Table 11.

### Delandistrogene Moxeparvovec Treatment and 260-Week Follow-Up Period: Part 1 and Part 2

The Follow-Up period will consist of 2 parts: Part 1 and Part 2. Part 1 of the Follow-Up period will begin with delandistrogene moxeparvovec infusion (Day 1) and last through Week 12. Part 2 of the Follow-Up period will begin post Week 12 and continue through Week 104.

### Follow-Up Period: Part 1

At least one day prior to delandistrogene moxeparvovec infusion, subjects will start the add-on corticosteroid treatment (prednisone or prednisolone) at 1 mg/kg, in addition to their chronic DMD-related steroid dose (Tables 11-14). The maximum total daily dose (including both chronic and add-on) is 60 mg per day. The additional steroids will continue through D60; however, earlier tapering to manage an AE may be permitted with prior Medical Monitor approval. Post-infusion added glucocorticoid for immunosuppression should be increased to 2 mg/kg daily if gamma-glutamyl transferase (GGT) level is confirmed to be ≥150 U/L or there are other clinically significant liver function abnormalities following infusion. For subjects on 2 mg/kg daily of additional corticosteroid, the maximum total daily dose is 120 mg per day. A tapering dose of glucocorticoid will be implemented based on individual subject's response to the infusion as assessed by liver function monitoring with GGT.

Delandistrogene moxeparvovec infusion will occur on Day 1 via a peripheral limb vein over 1-2 hours. The weight obtained the day prior to or on the day of dosing should be used for the dose calculation.

On Day 1, vital signs and blood samples for select laboratory studies will be collected. A brief physical examination and an ECG will be performed. An outline of assessments performed on Day 1 is shown in Tables 11-14.

During Part 1, safety will be assessed by monitoring of vital signs, physical examinations, ECGs, TEAEs, SAEs, and select laboratory assessments. Other assessments during this period include a blood sample for deoxyribonucleic acid (DNA) analysis (obtained from those who consent to this assessment), RNA analyses, PK/PD modeling, functional assessments, and a muscle biopsy. An outline of assessments performed during the Follow Up Period, Part 1, is shown in Tables 11-14.

### Follow-Up Period: Part 2

Part 2 of the Follow-Up period will begin post Week 12 and continue through Week 104 with follow-up visits. From Week 16 through Week 48, monthly telephone contacts will be performed between the in-clinic visits that take place every 12 weeks.

Safety will be assessed by monitoring of vital signs, physical examinations, ECGs, ECHOs, TEAEs, SAEs, and select laboratory assessments.

Immunogenicity will be assessed by measuring antibody titers to rAAVrh74 using the ELISA. Additionally, interferon gamma ELISpot assays will be performed to detect cellular immune responses to the rAAVrh74 capsid and the micro-dystrophin transgene.

Physical functional assessments will be performed throughout the study and will include the NSAA (which will also be used to measure time to rise from the floor and time of 10MWR, although the timed components are not part of the NSAA), time to ascend 4 steps, and the 100MWR.

### Follow-Up Period for Subjects not Receiving Delandistrogene Moxeparvovec

Subjects whose antibody titers are received more than 48 hours after the second dose of Imlifidase is completed or whose antibody titers are >1:400, will not receive delandistrogene moxeparvovec and will enter the 52-week follow-up period at 48 hours post Imlifidase infusion. Administering the delandistrogene moxeparvovec infusion more than approximately 48 hours after the Imlifidase infusion completes increases the potential for administering delandistrogene moxeparvovec outside of the anticipated Imlifidase-induced IgG nadir.

Safety monitoring of vital signs, physical examinations, TEAEs, SAEs, and select laboratory assessments will continue for 52 weeks as outlined in Tables 11-14 to ensure appropriate reconstitution of IgG levels and follow-up anti-Imlifidase antibodies.

### End of Study Early Termination

For subjects who receive both Imlifidase and delandistrogene moxeparvovec and complete the study, the last study visit will occur at Week 104. For subjects who terminate the study early, an early termination visit will be required as shown in the Schedule of Events (Table 14).

After completion of 104 weeks of follow-up, subjects will be enrolled into an extension study to assess the long-term safety and efficacy of delandistrogene moxeparvovec. Subjects will be followed for at least five years following their delandistrogene moxeparvovec infusion.

For subjects who do not receive delandistrogene moxeparvovec and complete the study, the last study procedure will occur at Week 52. Subjects who do not receive delandistrogene moxeparvovec will not be enrolled into the extension study.

### Completion of the Study

The study will be considered complete when all subjects have completed the Week 104 or Week 52 visit, as appropriate, or otherwise discontinued from the study.

### Scientific Rationale for Study Design

Delandistrogene moxeparvovec is designed to treat the underlying biological cause of DMD, however the presence of pre-existing antibodies to rAAVrh74 has thus far limited its potential for benefit in this population as they interfere with AAV-mediated gene delivery and diminish or abrogate its therapeutic effect. Imlifidase represents a well-tolerated therapy with an appropriate mechanism of action to deplete the pre-existing antibodies to rAAVrh74 and provide a safe therapeutic window for treating seropositive patients with gene therapy.

The subject dosing schedule allows for evaluation of acute safety on a by-subject basis, and this small, open-label study enables collection of acute and subacute clinical safety data with commercially representative process material. Subjects will be allowed to continue standard of care steroid therapy as well as non-pharmacological interventions such as physiotherapy during the study. The 12-week duration of Part 1 provides sufficient time to obtain primary endpoint expression data. The total study duration of 104 weeks allows for long-term safety follow-up after delandistrogene moxeparvovec infusion.

### Study Dose Selection Rationale

### Delandistrogene Moxeparvovec

Based on the extensive dose-ranging evaluation (4.43×10¹³ vg/kg to 4.01×10¹⁴ vg/kg) in the dystrophic animal model (DMD^{MDX} mouse model), a dose-dependent increase in tissue vector exposure was observed. A robust and non-linear relationship with a saturable response is demonstrated between tissue vector exposure and treatment response (delandistrogene moxeparvovec protein expression and functional improvement) in the DMD^{MDX} mice. Significant overlap was observed in the ER profile for both protein expression and functional improvement between 1.33×10¹⁴ vg/kg (clinically proposed dose) and the highest dose studied (4.01×10¹⁴ vg/kg), where the treatment response associated with 1.33×10¹⁴ vg/kg approached the plateau achieved at the highest dose studied. The increased vector exposure associated with 4.01×10¹⁴ vg/kg produced marginal increase (less than 17%) in delandistrogene moxeparvovec protein expression relative to the clinically proposed dose of 1.33×10¹⁴ vg/kg, which did not translate into functional improvement in the dystrophic animal model.

The general PK, PD, and population-based exposure-response (efficacy and safety) of delandistrogene moxeparvovec have been characterized in patients with DMD across 3 clinical studies (Delandistrogene Moxeparvovec-101, Delandistrogene Moxeparvovec-102, Delandistrogene Moxeparvovec-103) and 3 clinical doses including 6.29×10¹³ vg/kg and 8.94×10¹³ vg/kg that were studied in Study Delandistrogene Moxeparvovec-102, and the clinically proposed dose of 1.33×10¹⁴ vg/kg that was evaluated in Studies Delandistrogene Moxeparvovec-101, Delandistrogene Moxeparvovec-102 and Delandistrogene Moxeparvovec-103.

The clinically proposed dose of 1.33×10¹⁴ vg/kg achieved robust tissue transduction and delandistrogene moxeparvovec-dystrophin protein expression that translated into clinically meaningful benefit in motor functional outcome as measured by NSAA total score at 1-year post-dose. Importantly, similar to the evidence demonstrated in the foundational nonclinical studies, the dose of 1.33×10¹⁴ vg/kg was also shown to approach the plateau of biological efficacy (delandistrogene moxeparvovec-dystrophin protein expression measured as IF PDPF) in patients with DMD. These findings support that delandistrogene moxeparvovec at 1.33×10¹⁴ vg/kg is approaching the maximization of membrane-localized and functional delandistrogene moxeparvovec-dystrophin protein expression, and a higher dose (above 1.33×10¹⁴ vg/kg) is not expected to produce significant increase in biological efficacy.

Nonclinical GLP toxicology studies using commercially representative material in wildtype and DMD^{MDX} mice can be found in the delandistrogene moxeparvovec Investigator's Brochure. Using clinical data, an evaluation of the relationship between delandistrogene moxeparvovec administered drug (total capsid load and serum vector genome exposure) and clinical safety biomarkers has been conducted. In general, no apparent relationship was identified between delandistrogene moxeparvovec administered drug and changes in the liver injury biomarker levels (GGT, GLDH), cardiac injury biomarker (troponin) and importantly, immune system response inclusive of complement components and platelet counts. This provides further support to the totality of the safety evidence demonstrated for delandistrogene moxeparvovec at 1.33×10¹⁴ vg/kg to date.

### Imlifidase

This study uses the commercially indicated dose of 0.25 mg/kg, up to 2 doses.

During the clinical development of Imlifidase, a dose range inclusive of 0.12, 0.25 and 0.50 mg/kg was studied in healthy adults and patients with end stage renal disease. Clinical results indicated that 0.12 mg/kg was not sufficiently efficacious in cleaving IgG, and 0.50 mg/kg did not provide additional efficacy compared with 0.25 mg/kg. Additionally, a dose of 0.25 mg/kg once or twice is supported by toxicology studies in nonclinical species with a NOAEL (No Observed Adverse Effect Level) of 2 mg/kg in rabbits and dogs. Furthermore, 0.25 mg/kg (up to 2 doses) demonstrated a favourable safety profile in clinical studies.

The dose extrapolation to pediatrics patients was supported by population PK/PD modeling and simulations, which showed that following 0.25 mg/kg, pediatric patients at different age and weight groups between 0-18 years are predicted to have consistent PK/PD profiles as those in adults. Further, IgG cleaving assays comparing Imlifidase efficacy in paediatric serum pools (pools covering age 3-11, 12-17 or 3-17 years) and sera from adult healthy donors showed no difference in efficacy. Clinical evaluation of the proposed dose of 0.25 mg/kg is planned in Study 20-HMedIdeS-21 in the target pediatric population of highly sensitized children aged >1 to <18 who have a positive cross-match test and are intending to undergo kidney transplantation.

The same dose of 0.25 mg/kg can be extended to patients with DMD as mutations in the DMD gene and disease progression are not expected to confer renal or hepatic impairments that would alter the metabolism, excretion, or clearance of the Imlifidase protein and affect its overall PK profile. Therefore it stands to reason that the pediatric dose extrapolation stated above can be extended and support initial evaluation of Imlifidase to patients with DMD. Further, Imlifidase pharmacokinetics are similar to other protein therapeutics, therefore systemic clearance and volume of distribution parameters for Imlifidase are expected to scale with body weight and that a dose of 0.25 mg/kg will also be sufficient to reach an effective concentration in the target pediatric population. If a single dose of Imlifidase fails to reduce the titer of total anti-rAAVrh74 below 1:400, a second dose can be given within 60 hours of the first infusion. In the present study, imilifidase PK and PD will be evaluated in patients to DMD at 0.25 mg/kg to confirm the dose extrapolation.

### Stopping Rules or Completion of the Study Described in Example 2

Study enrollment will be interrupted when any of the following sentinel events occur: Occurrence of a New Serious Safety (ADR) Alert or Signal. Serious patient outcomes: death, life-threatening event a, hospitalization or important medical event.

Subjects will be considered to have completed their participation in the study after they have completed the last study visit of their Follow-Up period (either Week 104 or Week 52 depending on whether they received delandistrogene moxeparvovec) or have withdrawn from the study.

### Example 4: Treatment(s) of Patients in the Study Described in Example 2

### Study Treatment(s) Administered

### Delandistrogene Moxeparvovec

Delandistrogene moxeparvovec is supplied as a sterile, single use, frozen liquid for IV infusion. The frozen drug product must be thawed prior to the clinical administration.

Delandistrogene moxeparvovec administration will be through a peripheral limb vein. Subjects are to be closely monitored for at least 6 hours following completion of the infusion (see Table 13). A topical anesthetic cream (eg, lidocaine 2.5%, prilocaine 2.5%, LMX4 cream) may be applied to the skin prior to insertion of the intravenous catheter for infusions per site and subject preference.

### Imlifidase

The Imlifidase IMP is supplied as a freeze-dried (lyophilized) powder for concentrate for solution for infusion, 11 mg per vial. After reconstitution with sterile water for injection, each mL of the concentrate contains 10 mg Imlifidase. The concentrate should then be diluted in sodium chloride 9 mg/mL (0.90%) solution for infusion and administered as an infusion. Refer to the study-specific Pharmacy Manual for further details.

TABLE 16
Investigational Treatment(s) Administered

| Drug Name | Delandistrogene Moxeparvovec | Imlifidase |
|---|---|---|
| Description | A non-replicating recombinant | A recombinant cysteine protease |
| | AAV (rAAVrh74) containing a | and IgG endopeptidase originating |
| | micro-dystrophin transgene under | from S. pyogenes |
| | the control of the MHCK7 | |
| | promoter | |
| Dose Formulation | Sterile, single use, frozen liquid, | A freeze-dried (lyophilized) |
| | thawed for IV infusion | powder concentrate, reconstituted |
| | | with sterile water for IV infusion |
| Dosage Level(s) | 1.33 × 10¹⁴ vg/kg | 0.25 mg/kg |
| | Single dose | Single dose |
| | | Up to two doses |
| Route of | Peripheral limb IV infusion | Peripheral limb IV infusion |
| Administration | | |
| Sourcing | Provided centrally by the Sponsor | Provided centrally by the Sponsor |
| Packaging and | Study drug will be provided in | Study drug will be provided in |
| Labeling | container. Each container will be | container. Each container will be |
| | labeled as required per country | labeled as required per country |
| | requirement | requirement |

### Non-Investigational Treatments

The precise choice and formulation of the non-investigational medicines is at the discretion of the Investigator and will be sourced from the Investigational site pharmacy supply.

### Corticosteroids

As required by the inclusion criterion, all subjects will be on a stable daily dose of oral corticosteroids for at least 12 weeks before the Screening visit. The day before delandistrogene moxeparvovec infusion and continuing for approximately 60 days after delandistrogene moxeparvovec infusion, each subject will be started on additional oral steroid (prednisone or prednisolone) for immunosuppression.

### Antibiotics

On the day of the first Imlifidase infusion, subjects will start a 4-week course of prophylactic antibiotics to mitigate the potential increased risk of infection from IgG depletion. The selected antibiotic should have coverage for upper respiratory pathogens, for example amoxicillin 10 mg/kg twice daily (up to a maximum dose of 500 mg twice daily). An alternative agent (eg cephalosporin, macrolide) may be used at the Investigator's discretion and may be based on local or institutional resistance patterns or guidelines.

### Antihistamines

To reduce the risk of infusion reaction, an antihistamine (for example, diphenhydramine 1.25 mg/kg orally up to a maximum of 50 mg) will be administered up to approximately one hour prior to each Imlifidase infusion. The choice and dose of antihistamine is at the discretion of the Investigator and may be based on local or institutional guidelines.

### Study Drug Handling/Storage

### Storage

Delandistrogene moxeparvovec must be transported and stored at ≤−60° C.

Prior to reconstitution, Imlifidase should be stored refrigerated at +2° C. to +8° C. protected from light.

### Safety Monitoring

### Safety Monitoring for Liver Chemistry Tests

Liver chemistry tests need to be monitored as specified in Tables 11-14. Abnormal liver chemistry test result(s) need to be confirmed if:
GGT is >3×ULN at any time during the study
AST or ALT measurement is >2×Baseline value if the baseline value is >ULN; or >3×ULN if baseline value is within normal range

Subjects with confirmed liver chemistry test results (as above) should have their liver chemistry tests (GGT, ALT, AST, alkaline phosphatase, and total and direct bilirubin) retested at a minimum of 2 times weekly and additional testing beyond that is at the discretion of the Investigator. Frequency of retesting can decrease to once a week or less if abnormalities stabilize and the subject is asymptomatic.

### Additional Investigations

Subjects with confirmed abnormal liver chemistry test results (as above) are recommended to have the following evaluations performed:
Obtaining a more detailed history of symptoms and prior or concurrent diseases;
Obtaining a history of concomitant drug use (including nonprescription medications and herbal and dietary supplement preparations), alcohol use, recreational drug use, and special diets;
Ruling out acute viral hepatitis types A, B, C, D, and E; autoimmune hepatitis; nonalcoholic steatohepatitis; hypoxic/ischemic hepatopathy; and biliary tract disease;
Consideration of other viral illnesses that have been associated with hepatitis (eg, Epstein-Barr virus [EBV], cytomegalovirus [CMV], parvovirus B19, human herpesvirus 6 [HH6], varicella zoster virus);
Obtaining a history of exposure to environmental chemical agents.

Additional liver evaluations, including gastroenterology/hepatology consultations, hepatic computed tomography or magnetic resonance imaging scans, may be performed at the discretion of the Investigator, in consultation with the Sponsor Medical Monitor.

### Safety Monitoring for Immune-mediated Myositis

Subjects will be monitored for occurrence of immune-mediated myositis by monitoring AEs as specified in Tables 11-14. In the limited number of cases reported to date, the time to onset has been 24-42 days (Bonnemann et al. 2022 Muscular Dystrophy Association Clinical & Scientific Conference; Mar. 13-16, 2022; Nashville, TN. Poster 44.). Subjects will be instructed to promptly report any new muscular weakness, and the Investigator needs to closely evaluate all potential causes, including concomitant illness.

### Additional Investigations

Subjects who experience rapidly progressing muscular weakness need to be discussed with the Sponsor Medical Monitor to determine whether additional monitoring or laboratory tests are required. Additional evaluations including immunology consultation, ELISpot and ELISA tests against transgene micro-dystrophin protein, muscle biopsy, muscle MRI, and/or electromyography-nerve conduction studies may be performed at the discretion of the Investigator in consultation with the Sponsor Medical Monitor.

Adverse Events of Special Interest related to immune-mediated myositis include any myositis, including immune-mediated myositis or autoimmune myositis; any severe (CTCAE Grade≥3) asthenia or muscular weakness; dysphagia or dysphonia secondary to muscular weakness; rhabdomyolysis, myoglobinuria; chromaturia; acute onset or exacerbation of myalgia.

### Safety Monitoring for Hypersensitivity

Subjects will be monitored for occurrence of allergic reactions by monitoring AEs as specified in Tables 11-14. Subjects will be instructed to promptly report any signs or symptoms of fever or constitutional symptoms that may arise during the study, and the Investigator needs to closely evaluate all potential causes, including concomitant illness.

### Additional Investigations

Subjects who experience significant or persistent constitutional symptoms, including any CTCAE Grade≥3 hypersensitivity reactions, need to be discussed with the Sponsor Medical Monitor to determine whether additional monitoring or laboratory tests are required. Additional evaluations including immunology consultation, tests for allergic reactions (eg, absolute eosinophils, eosinophilic cationic protein, serum/plasma tryptase) may be performed at the discretion of the Investigator in consultation with the Sponsor Medical Monitor.

### Safety Monitoring for Thrombotic Microangiopathy (TMA)

Subjects should be monitored for occurrence of complement-mediated reactions primarily via monitoring AEs and complement levels. Subjects will be instructed to promptly report any signs or symptoms of fever or constitutional symptoms that may arise during the study, and the Investigator needs to closely evaluate all potential causes, including concomitant illness. In addition to monitoring AEs, routine laboratory monitoring for complement-mediated reactions should be performed per protocol including complement levels (refer to Tables 11-14).

### Additional Investigations

Subjects diagnosed with TMA need to be discussed with the Sponsor Medical Monitor to determine additional monitoring and laboratory testing. Additional evaluations including nephrology consultation and tests for complement activation and alternative etiology (C3a, C5a, sC5b-9, Bb, prothrombin time, activated partial thromboplastin time, FDPs, D-dimer, ADAMTS13, CFH Ab, STEC, stool culture swab, antiphospholipid antibody, ECG, echocardiogram, ANA, anticentromere antibodies, anti-scl-70, anti-ds-DNA, plasma homocysteine, plasma and urine methylmalonic acid, G6PD activity, T antigen, testing for influenza, CMV, and/or EBV) may be performed at the discretion of the Investigator in consultation with the Sponsor Medical Monitor.

### Safety Monitoring for Platelet Count Results

Platelet counts will be monitored as specified in Tables 11-14.

Subjects with a confirmed occurrence of platelets <75,000/mm³ should have the following evaluations performed:
Complete blood count with reticulocytes
Peripheral blood smear
Coagulation panel (prothrombin time/INR, activated partial thromboplastin time)
hsCRP

### Additional Investigations

Additional platelet evaluations for confirmed, unexplained significant platelet count reductions, including hematology consultation, fibrinogen, fibrinogen split products/D-dimer, von Willebrand factor, total immunoglobulins, complement levels, viral serologies, auto-antibody screen, antiplatelet antibodies, antiplatelet factor 4 assay, and platelet function tests may be performed at the discretion of the Investigator in consultation with the Sponsor Medical Monitor.

### Safety Monitoring for Rhabdomyolysis

Rhabdomyolysis must be monitored by urine dipstick and AEs as specified in Tables 11-14.

Subjects who have confirmed heme+ dipstick urinalysis will need to be evaluated for urine microscopy and the following AEs:
Rhabdomyolysis
Myoglobinuria
Chromaturia
Acute onset or exacerbation of myalgia

### Additional Investigations

In case of rhabdomyolysis, myoglobinuria, or chromaturia, subjects will need to have evaluations of myoglobinuria, CK, renal function (eg, serum cystatin C), and serum chemistry 2 or 3 times weekly until values reach usual/pre-event levels or stabilize. In case of acute onset or exacerbation of myalgia, in the absence of rhabdomyolysis, myoglobinuria, or chromaturia, these evaluations should be undertaken at the discretion of the Investigator.

In addition, Investigators should obtain a more detailed history of symptoms, preceding activity and hydration status, concomitant drug use, and recent or concurrent infections. Additional evaluations, including rheumatology/immunology consultations, anti-muscle antibodies, or ELISpot, may be performed at the discretion of the Investigator in consultation with the Sponsor Medical Monitor.

If acute onset or exacerbation of weakness is a feature of the rhabdomyolysis event, the subject should be assessed for rapidly progressive weakness, as in Section 4.4.2.

### Risk of Rhabdomyolysis due to Anesthesia

Participation in this study involves biopsies (Section 9,2,1,) which may require anesthesia. Investigators should be aware that subjects with DMD have specific risks and requirements when undergoing anesthesia (American Academy of Pediatrics 2005, Birnkrant 2009, Bushby 2010). For example, there is an absolute contraindication against the use of depolarizing muscle relaxants (eg, succinylcholine, also known as suxamethonium) due to the risk of rhabdomyolysis; and a strong recommendation to avoid inhalational anesthetics (eg, sevoflurane, desflurane, enflurane, halothane, isoflurane) due to the risk of malignant hyperthermia-like reactions and rhabdomyolysis. There is no evidence that exposure to delandistrogene moxeparvovec affects this risk.

### Safety Monitoring for Troponin Increase

Cardiac troponin levels need to be monitored as specified in the Schedule of Events. Initial cardiac troponin level result needs to be confirmed if:
Cardiac troponin levels >3×ULN (or 3×baseline for subjects with elevated baseline values)
Subjects with confirmed abnormal cardiac troponin levels need to have their cardiac troponin levels retested until the level returns to baseline or stabilizes for at least 6 weeks. Frequency of retesting is at the discretion of the Investigator.

### Additional Investigations

Additional evaluations including ECG, cardiology consultation, ECHO, cardiac MRI, CKMB, and myoglobin panel may be performed at the discretion of the Investigator in consultation with the Sponsor Medical Monitor (see also Section 5.3.4.).

### Safety Monitoring for Infection

Signs and symptoms of potential infection will be monitored throughout the study using vital signs, physical exams, select laboratories, and patient report. The following signs and symptoms should prompt consideration for infectious work-up:

Fever; chills and sweats; headaches; cough with and without mucus; a stuffy or runny nose; sore throat or new mouth sore; hoarse voice; swollen lymph nodes in neck; shortness of breath, tight chest, or wheezing; burning or pain with urination; increased urination; unusual vaginal discharge or irritation; redness, soreness, or swelling in any area, including surgical wounds and ports; vomiting; diarrhea; pain in the abdomen or rectum; fatigue, muscle aches, feeling generally unwell.

### Additional Investigations

Laboratory studies such as CBC, viral panel, blood, urine, or stool cultures, and imaging studies such as chest x-ray may be performed at the discretion of the Investigator and subjects should be managed according to standard of care and applicable guidelines (see also Section 5.3.4).

### Example 5—Study Assessments Blend into Above

The schedule outlining the study assessments and times of assessments is shown in Tables 11-14.

### Adverse Events of Special Interest

In this study, the AESIs are defined as follows:

### Hepatotoxicity

GGT>8×ULN
GGT>5×ULN and persists for ≥2 weeks
GGT>3×ULN, and either total bilirubin >2×ULN or INR>1.5
GGT>3×ULN and the new appearance (ie, onset coincides with the changes in hepatic enzymes) of fatigue, nausea, vomiting, right upper quadrant pain or tenderness, fever, rash, or eosinophilia (>5%) felt by the Investigator to be potentially related to hepatic inflammation.

### Myositis

Any myositis, including immune-mediated myositis or autoimmune myositis; any severe (CTCAE Grade≥3) asthenia or muscular weakness; dysphagia or dysphonia secondary to muscular weakness.

### Thrombotic Microangiopathy (TMA)

TMA, acute kidney injury, and/or atypical hemolytic uremic syndrome.

### Hypersensitivity

Anaphylaxis, anaphylactoid reaction, or angioedema; any severe (CTCAE Grade≥3) allergic reaction; any severe (CTCAE Grade≥3) inflammation events (eg, myocarditis, pneumonitis, vasculitis etc.); any severe (Grade≥3) infusion related reaction occurring within 24 hours of infusion of delandistrogene moxeparvovec; any infusion related reaction occurring within 48 hours of infusion of Imlifidase.

### Thrombocytopenia

Platelet count <75,000/mm³

### Rhabdomyolysis

Rhabdomyolysis; myoglobinuria; chromaturia; acute onset or exacerbation of myalgia.

### Troponin Elevations

Troponin I>3×ULN (or 3×baseline for subjects with elevated baseline values).

### Infection

Any severe (Grade≥3) infection that occurs within 30 days of Imlifidase infusion; any infection that requires hospitalization that occurs within 30 days of Imlifidase infusion

### Dose Interruption

Any event that results in interruption (pause and or discontinuation) of Imlifidase administration

### Pharmacokinetic, Pharmacodynamic and Laboratory Assessments

### Pharmacokinetic and Pharmacodynamic Assessments

Blood samples for pharmacokinetic and pharmacodynamic analyses will be drawn. Note that the IgG analysis methods generally available at clinical chemistry laboratories (turbidimetry/nephelometry) do not discriminate between the different IgGfragments generated post-Imlifidase treatment. The actual date and time of collection of each sample will be recorded in eCRF. PK samples will be analysed by a validated electroluminescence based immunoassay using the Meso Scale Diagnostics (MSD) Technology. PD samples will be analysed by a validated electroluminescence based immunoassay using the Meso Scale Diagnostics (MSD) Technology and SDS-PAGE.

### Laboratory Assessments

The routine clinical laboratory tests will be performed at the time points specified in Tables 11-14. The protocol-required safety laboratory assessments are listed in Table 18.

Note: At Week 12, samples will be collected before the biopsy.

TABLE 17

| | |
|---|---|
| Electrolytes: | Sodium, chloride, potassium, carbon dioxide |
| Troponin: | Troponin I |
| CK: | CK |
| Liver Function: | Total and direct bilirubin, alkaline phosphatase, |
| | amylase, ALT, AST, GGT, lactate dehydrogenase, |
| | albumin |
| Hematology: | Red blood cells, total white blood cells, |
| | hemoglobin, hematocrit, neutrophils, lymphocytes, |
| | monocytes, eosinophils, basophils, platelets |
| hsCRP and | complement (C3, C4) |
| complement: | |
| Renal Function: | Creatinine, blood urea nitrogen, and serum cystatin C |
| Urinalysis: | pH, specific gravity, protein, glucose, ketones, |
| | hemoglobin |

A laboratory abnormality deemed clinically significant by the Investigator or designee should be recorded as an AE. A clinically significant abnormality is generally an abnormality, confirmed by repeat testing, that is changed sufficiently from Screening/Baseline so that, in the judgment of the Investigator, a change in management is warranted.

### Protocol-Required Safety Laboratory Assessments

The tests detailed in Table 18 will be performed by the central laboratory.

TABLE 18
Protocol-Required Safety Laboratory Assessments

| Test | Analytes |
|---|---|
| Hematology | Red blood cells, total white blood cells, hemoglobin, |
| | hematocrit, neutrophils, lymphocytes, monocytes, |
| | eosinophils, basophils, platelets |
| Blood chemistry | Carbon dioxide, chloride, potassium, sodium |
| Liver function | Total and direct bilirubin, alkaline phosphatase, |
| | amylase, ALT, AST, GGT, lactate dehydrogenase, |
| | albumin |
| Urinalysis | pH, specific gravity, protein, glucose, |
| | ketones, hemoglobin |
| Virus serology | HIV, HBV, HCV |
| Troponin | Troponin I |

Additional tests may be performed at any time during the study as determined necessary by the Investigator or required by local regulations.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All publications, patents, and patent applications mentioned in this specification are herein incorporated by reference to the same extent as if each individual publication, patent, or patent application was specifically and individually indicated to be incorporated by reference.

1. A method of treating a subject having a muscular dystrophy, comprising administering to the subject in need thereof a recombinant AAVrh74 viral vector and an enzyme that cleaves immunoglobulin G (IgG); wherein the recombinant AAVrh74 viral vector comprises a genetic cassette encoding a therapeutic molecule.

2. (canceled)

3. The method of claim 1, wherein the recombinant AAVrh74 viral vector and the enzyme that cleaves IgG are administered to the subject concurrently or sequentially.

4. The method of claim 1, wherein, prior to administering the recombinant AAVrh74 vector, the subject is determined to have anti-AAVrh74 antibodies, wherein the anti-AAVrh74 antibodies are neutralizing antibodies, non-neutralizing antibodies, or total anti-AAVrh74 antibodies comprising both neutralizing and non-neutralizing antibodies.

5.-7. (canceled)

8. The method of claim 4, wherein the subject has a titer of total anti-AAVrh74 antibodies greater than 1:400 in a total anti-AAVrh74 antibody ELISA assay.

9. (canceled)

10. The method of claim 1, wherein the recombinant AAVrh74 viral vector is administered to said subject no more than 54 hours after administration of the enzyme that cleaves IgG.

11. (canceled)

12. The method of claim 10, further comprising, prior to administering the recombinant AAVrh74 viral vector, determining if the subject has anti-AAVrh74 antibodies after administration of the first dose of the enzyme that cleaves IgG, and administering a second dose of the enzyme to the subject the subject is determined to have anti-AAVrh74 antibodies after administration of the first dose of the enzyme, wherein the anti-AAVrh74 antibodies are neutralizing or non-neutralizing anti-AAVrh74 antibodies or total anti-AAVrh74 antibodies comprising both neutralizing and non-neutralizing antibodies.

13.-15. (canceled)

16. The method of claim 12, wherein, after administration of the first dose of the enzyme that cleaves IgG, the subject has a titer of total anti-AAVrh74 antibodies greater than 1:400 in a total anti-AAVrh74 antibody ELISA assay.

17. (canceled)

18. The method of claim 12, wherein the recombinant AAVrh74 viral vector is administered to said subject no more than 54 hours after the administration of the second dose of the enzyme that cleaves IgG.

19. (canceled)

20. The method of claim 12, wherein the subject is administered the second dose of the enzyme that cleaves IgG no more than 60 hours after the first dose of the enzyme.

21. The method of claim 12, wherein after administration of the first dose of the enzyme that cleaves IgG, the subject has a titer of total anti-AAVrh74 antibodies of between about 1:1600 and about 1:3200 in a total anti-AAVrh74 antibody ELISA assay.

22. The method of claim 21, further comprising, prior to administering the recombinant AAVrh74 viral vector, administering a third dose of the enzyme that cleaves IgG to the subject no more than 60 hours after administration of the second dose of the enzyme.

23. The method of claim 22, wherein the recombinant AAVrh74 viral vector is administered to said subject no more than 54 hours after administration of the third dose of the enzyme that cleaves IgG.

24.-25. (canceled)

26. A method of treating a subject having a muscular dystrophy, comprising administering to the subject a recombinant AAVrh74 viral vector, wherein the subject has been previously administered an enzyme that cleaves IgG.

27. (canceled)

28. A method of preparing a subject for a gene therapy for a muscular dystrophy comprising administering to the subject an enzyme that cleaves IgG prior to administration of a recombinant AAVrh74 viral vector.

29.-42. (canceled)

43. The method of claim 1, wherein the muscular dystrophy is Duchenne muscular dystrophy (DMD) or limb-girdle muscular dystrophy (LGMD).

44.-46. (canceled)

47. The method of claim 1, wherein the enzyme that cleaves IgG specifically targets and cleaves human IgG1, human IgG2, human IgG3, and human IgG4.

48. (canceled)

49. The method of claim 1, wherein the enzyme that cleaves immunoglobulin IgG comprises a cysteine or a thiol protease that inactivates the IgG and/or cleaves IgG at a hinge region.

50.-53. (canceled)

54. The method of claim 1, wherein the enzyme that cleaves IgG comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to any of SEQ ID NOs 1-18.

55.-58. (canceled)

59. The method of claim 1, wherein the subject has one or more IgG antibodies that specifically bind a capsid protein of a recombinant AAVrh74 viral vector, wherein the one or more IgG antibodies are AAVrh74 neutralizing or non-neutralizing antibodies or are total AAVrh74 antibodies comprising both neutralizing and non-neutralizing antibodies.

60.-62. (canceled)

63. The method of claim 4, wherein the subject has a titer of total anti-AAVrh74 antibodies greater than 1:400 in a total anti-AAVrh74 antibody ELISA assay.

64.-66. (canceled)

67. The method of claim 1, wherein the therapeutic molecule comprises a polypeptide, an RNA molecule, or a DNA molecule.

68. The method of claim 66, wherein the therapeutic polypeptide is a microdystrophin, a beta sarcoglycan, an alpha sarcoglycan, or any combination thereof.

69. The method of claim 1, wherein the genetic cassette comprises a nucleic acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 19, SEQ ID NO: 21, or SEQ ID NO: 23.

70.-74. (canceled)

75. The method of claim 68, wherein the recombinant AAVrh74 viral vector further comprises a tissue specific promoter.

76. (canceled)

77. The method of claim 75, wherein the tissue specific promoter is an MHCK7 promoter or a tMCK promoter.

78. The method of claim 77, wherein the promoter comprises a nucleic acid sequence having a least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 25 or 26.

79.-86. (canceled)

87. The method of claim 1, wherein the recombinant AAVrh74 viral vector is delandistrogene moxeparvovec, bidridistrogene xeboparvovec, or patidistrogene bexoparvovec.

88.-136. (canceled)
